(19) United States
(12) Patent Application Publication
(10) Pub. No.: US 2026/0317312 A1
(43) Pub. Date: Oct. 15, 2026

(54) ISOXAZOLIDINES AS RIPK1 INHIBITORS AND USE THEREOF

(71) Applicant: GENZYME CORPORATION, Cambridge, MA (US)

(72) Inventors: Elisabeth DEFOSSA, Frankfurt am Main (DE); Niels GRIESANG, Frankfurt am Main (DE); Uwe HEINELT, Frankfurt am Main (DE); Hans MATTER, Frankfurt am Main (DE); Maria MENDEZ-PEREZ, Frankfurt am Main (DE); Nils RACKELMANN, Frankfurt am Main (DE); Lothar SCHWINK, Frankfurt am Main (DE)

(21) Appl. No.: 19/383,236

(22) Filed: Nov. 7, 2025

Related U.S. Application Data
(63) Continuation of application No. PCT/US2024/028164, filed on May 7, 2024.

(30) Foreign Application Priority Data
May 10, 2023 (EP) 23172677.9

(51) Int. Cl. C07D 413/12; A61K 31/422; A61K 31/427; A61K 31/4439; A61K 31/444; A61K 31/497; A61K 31/506; A61K 31/519; C07D 261/02; C07D 413/04; C07D 413/08; C07D 413/14; C07D 417/04; C07D 487/04

(52) U.S. Cl. CPC C07D 413/12; A61K 31/422; A61K 31/427; A61K 31/4439; A61K 31/444; A61K 31/497; A61K 31/506; A61K 31/519; C07D 261/02; C07D 413/04; C07D 413/08; C07D 413/14; C07D 417/04; C07D 487/04

(57) ABSTRACT

The disclosure relates to compound of formula (I)

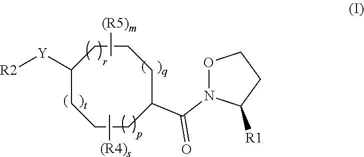

Wherein R1 represents a phenyl or a monocyclic heteroaryl, which is optionally substituted by one, two or three R3; R2 represents an aryl or a heteroaryl, which is optionally substituted; each R3 is independently chosen from a halogen, a cyano, a (C₁-C₄)alkyl group or a (C₁-C₄)alkoxy group; R4 and R5 are independently chosen from a halogen, a (C₁-C₄)alkyl group, a (C₁-C₄)alkoxy group, or R4 and R5 form together a (C₁-C₄)alkylene bridge; m and s are independently 0 or 1; p, q, r and t are independently 0 or 1; Y is a bond or a divalent group chosen from —C(O)—NH—, —C(O)—NH—CH₂—, —NH—, —CH₂—NH—, —NH—CH₂—, —O—, CH₂—O— and —O—CH₂, or a pharmaceutically acceptable salt, solvate, or stereoisomer thereof.

It further relates to the pharmaceutical compositions containing said new compounds.

### RELATED APPLICATIONS

This application is a continuation of International Patent Application No. PCT/US2024/028164, filed May 7, 2024, which claims priority to European Patent Application No. 23172677.9, filed May 10, 2023, the entire disclosures of which are hereby incorporated herein by reference.

### TECHNICAL FIELD

The disclosure relates to new isoxazolidine derivatives useful as a medicament. Said new compounds are in particular useful as kinase inhibitors, and even particularly as RIPK1 inhibitors. They are efficient for treating and/or preventing acute and chronic neurodegenerative diseases like Parkinson's disease, Alzheimer's disease, amyotrophic lateral sclerosis (ALS) or multiple sclerosis (MS).

The disclosure further relates to the pharmaceutical compositions containing said new compounds.

### TECHNICAL BACKGROUND

Although inflammation can be a protective mechanism in response to harmful stimuli such as invasion of pathogens and tissue damages, chronic inflammation is an important underlying factor in many human diseases such as neurodegeneration, rheumatoid arthritis, autoimmune and inflammatory diseases, and cancer. Similarly, the activation of cell death pathways, such as necrosis and apoptosis which are useful in eliminating infected or damaged cells, is also an important underlying mechanism for human diseases, including acute and chronic neurodegenerative diseases. Receptor-interacting protein kinase 1 (UniProtKB Q13546) is a key regulator of inflammation, apoptosis and necroptosis. Receptor-interacting protein kinase 1 has an important role in modulating inflammatory responses mediated by nuclear-factor kappa-light chain enhancer of activated B cells (NF-κB). More recent research has shown that its kinase activity controls necroptosis, a form of necrotic cell death, which was traditionally thought to be passive and unregulated, and is characterized by a unique morphology. Further, receptor-interacting protein kinase 1 is part of a pro-apoptotic complex indicating its activity in regulating apoptosis.

The receptor-interacting protein kinase 1 is subject to complex and intricate regulatory mechanisms, including ubiquitylation, deubiquitylation, and phosphorylation. These regulatory events collectively determine whether a cell will survive and activate an inflammatory response or die through apoptosis or necroptosis. Dysregulation of receptor-interacting protein kinase 1 signalling can lead to excessive inflammation or cell death, and conversely, research has shown that inhibition of receptor-interacting protein kinase 1 can be effective therapies for diseases involving inflammation or cell death.

RIPK1 inhibition has been identified as a promising principle to address different diseases like rheumatoid arthritis (RA), psoriasis, multiple sclerosis, Alzheimer's disease, inflammatory bowel disease such as Crohn's disease, amyotrophic lateral sclerosis (ALS) or ulcerative colitis (UC). To treat some of these diseases like multiple sclerosis (MS) and Alzheimer's disease, access to the central nervous system (CNS) is required, while for other diseases like rheumatoid arthritis, psoriasis, inflammatory bowel disease (IBD) such as Crohn's disease or UC access to the CNS is not essentially required.

Different RIPK1 inhibitors were already described, for example in patent applications WO 2014/125444, WO 2016/185423 or WO 2016/027253 (GSK).

The RIPK1 inhibitor GSK2982772 (oxazepinone derivative disclosed in WO 2014/125444), was evaluated for RA, psoriasis and UC in phase II clinical trials.

Dihydropyrazole compounds with phenyl substituent on dihydropyrazole and a pyrimidine-piperidine element are disclosed as RIPK1 inhibitors by GSK in WO 2018/092089. Other Dihydropyrazole compounds as RIPK1 inhibitors are disclosed in WO2020224656.

Isoxazolidine compounds with phenyl substituent on isoxazolidine and a pyrimidine-piperidine element are disclosed as RIPK1 inhibitors by GSK in WO 2019/130230. Similar isoxazolidine compounds are disclosed in KR 2020-087922 (Voronoi) and in WO 2020/043173.

Isoxazolidine compounds with a reduced ability to cross the blood-brain-barrier as RIPK1 inhibitors are disclosed in WO 2021/245070.

Compounds with a cycloalkyl element as RIPK1 inhibitor are disclosed in WO 2022/194259.

### SUMMARY

According to one of its objects, the present disclosure relates to a compound of formula (I):

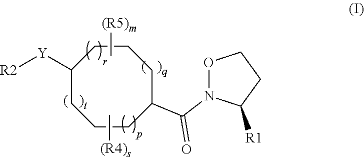

wherein
R1 represents a phenyl or a monocyclic heteroaryl, which is optionally substituted by one, two or three R3;
R2 represents an aryl or a heteroaryl, which is optionally substituted by one, two or three R6;
each R3 is independently chosen from a halogen, a cyano, a (C₁-C₄)alkyl group or a (C₁-C₄)alkoxy group;
R4 and R5 are independently chosen from a halogen, a (C₁-C₄)alkyl group, a (C₁-C₄)alkoxy group, or R4 and R5 form together a (C₁-C₄)alkylene bridge;
m and s are independently 0 or 1;
p, q, r and t are independently 0 or 1;
Y is a bond or a divalent group chosen from —C(O)—NH—, —C(O)—NH—CH₂—, —NH—, —CH₂—NH—, —NH—CH₂—, —O—, CH₂—O— and —O—CH₂—;
each R6 is independently selected from halogen, cyano, —OH, a (C₁-C₄)-alkyl group, —CF₃, —C(O)NH₂, a —C(O)NH—(C₁-C₄)-alkyl group, —C(O)OH, a —C(O)O—(C₁-C₄)-alkyl group, —SO₂NH₂, a (C₁-C₄)-alkoxy group, a —O—(C₁-C₄)alkylene-(C₃-C₆)cycloalkyl group, a —O—(C₃-C₆)cycloalkyl group, a —O—(C₃-C₆)heterocycloalkyl group, a 5- or 6-memembered heterocycloalkyl group, a monocyclic heteroaryl group and oxo, wherein said (C₁-C₄)alkyl group, (C₁-C₄)alkoxy group, —O—(C₃-C₆)cycloalkyl group, (C₁-C₄)alkyl group, 5- or 6-memembered heterocycloalkyl group or monocyclic heteroaryl group is optionally substituted by one, two, three or four R7;
each R7 is independently a halogen, oxo, —OH, a (C₁-C₄)alkyl group or a (C₁-C₄)alkoxy group,
or a pharmaceutically acceptable salt, solvate, or stereoisomer thereof.

In a related aspect, provided herein pharmaceutical composition comprising a compound of formula (I) or a pharmaceutically acceptable salt, solvate, or stereoisomer thereof, and at least one pharmaceutically acceptable excipient.

In another aspect provided herein is a process for manufacturing a compound of formula (I) and intermediates thereof.

In another aspect, is provided herein a compound of formula (I) or a pharmaceutically acceptable salt, solvate, or stereoisomer thereof, for use as medicament.

In another aspect, is provided herein a compound of formula (I) or a pharmaceutically acceptable salt, solvate, or stereoisomer thereof, for use in the treatment and/or prevention of a disease, disorder or condition that is at least partly mediated by receptor-interacting protein kinase 1.

In another aspect, is provided herein a compound of formula (I), or a pharmaceutically acceptable salt, solvate, or stereoisomer thereof, for use in the treatment and/or prevention of a disease selected from Parkinson's disease, Alzheimer's disease, amyotrophic lateral sclerosis (ALS) or multiple sclerosis (MS).

In another aspect, provided herein is a method of inhibiting receptor-interacting protein kinase 1. Further provided are methods for treating a disease, disorder or condition that is at least partly mediated by receptor-interacting protein kinase 1, comprising administering a therapeutically effective amount of a compound of formula (I) or a pharmaceutical composition as described herein to a subject in need thereof. The disclosure also provides uses of the compounds of formula (1) or compositions thereof in the manufacture of a medicament for the treatment of a disease, disorder or condition that is at least partly mediated by receptor-interacting protein kinase 1.

### DETAILED DESCRIPTION

### Definitions

Unless otherwise stated, the following terms used in the specification and claims have the following meanings.

In this specification the term “alkyl” refers to straight or branched, saturated, aliphatic hydrocarbon groups having the number of atoms indicated. More particularly, a (Cₓ-C_{y}) alkyl group, where x and y are integers, x<y, is a linear or branched saturated aliphatic group comprising from x to y carbon atoms. For example (C₁-C₄)alkyl includes, but is not limited to, methyl, ethyl, n-propyl, isopropyl, n-butyl, isobutyl, sec-butyl and tert-butyl groups, and the like.

The term “alkylene” includes both straight and branched chain divalent alkyl groups. For example, “(C₁-C₄)alkylene” comprises, but is not limited to, methylene (—CH₂—), ethylene (—CH₂CH₂—), methyl methylene (—CH(CH₃)—), propylene, and butylene.

The term “alkoxy” designates an alkyl group singularly bonded to oxygen. In particular, “(Cₓ-C_{y})alkoxy”, as used herein, refers to a —O—(Cₓ-C_{y})alkyl, where x and y are integers, x<y. For example, “(C₁-C₄)alkoxy” comprises, but is not limited to, methoxy, ethoxy, isopropoxy, and t-butoxy.

“Cycloalkyl” refers to saturated or partially unsaturated, optionally substituted, cyclic hydrocarbon groups having the number of atoms indicated. More particularly, a (C₃-C_{z})-cycloalkyl group, where z is an integer greater than or equal to 4, comprises from 3 to z carbon atoms. For example, (C₃-C₃)-cycloalkyl groups contain from 3 to 8 carbon atoms and are for example cyclopropyl, cyclobutyl, cyclopentyl, cyclohexyl, cycloheptyl or cyclooctyl.

The term “halogen” refers to a chlorine, fluorine, bromine, or iodine atom, and in particular denotes a chlorine or fluorine atom.

The term “heterocyclyl”, “heterocyclic”, or “heterocycle” means a non-aromatic saturated or partially unsaturated, optionally substituted, monocyclic, fused or bridged bicyclic cyclic ring system(s) wherein one to five (suitably one or two) of the carbon atoms are replaced with a heteroatom, such as oxygen, sulfur or nitrogen atom. For example, “(C₃-C₆)heterocycloalkyl group”, as used herein, refers to a (C₃-C₆)cycloalkyl group wherein one or two of the carbon atoms are replaced with a heteroatom, in particular an oxygen or nitrogen atom. Examples of heterocyclic groups include, but are not limited to, cyclic ethers, such as oxiranyl, oxetanyl, tetrahydrofuranyl, dioxanyl, and substituted cyclic ethers. Heterocycles containing nitrogen include, for example, azetidinyl, pyrrolidinyl, imidazolinyl, dihydroimidazolyl piperidinyl, piperazinyl, tetrahydrotriazinyl, tetrahydropyrazolyl and the like. Typical sulfur containing heterocycles include tetrahydrothienyl, dihydro-1,3-dithiol, tetrahydro-2H-thiopyran, and hexahydrothiepine. Other heterocycles include, oxazolidinyl, tetrahydro oxazolyl, tetrahydro-oxadiazolyl, tetrahydrodioxazolyl, hexahydrotriazinyl, tetrahydro oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Partially unsaturated heterocyclyl rings contain at least one double bond, such as 1 or 2 double bonds. Examples of partially unsaturated heterocyclyl rings include 1,6-dihydropyridinyl, 1,6-dihydropyridazinyl, and 2,3-dihydropyrrolyl. As the skilled person would appreciate, any heterocycle may be linked to another group via any suitable atom, such as via a carbon or nitrogen atom.

By “bridged ring systems” is meant ring systems in which two rings share more than two atoms, see for example Advanced Organic Chemistry, by Jerry March, 4th Edition, Wiley Interscience, pages 131-133, 1992. In particular, a “bridged (C₆-C₁₀)cycloalkyl” group, as used herein, refers to a bi- or tri-cyclic compound where the cycles are cycloalkyls, the rings share three or more atoms and the bridge contains at least one atom, for example 1 also named C₁-alkylene bridge, 2 also named C₂-alkylene bridge or 3 atoms. Such bridged cycloalkyl groups may be substituted by one or more C₁-C₃ alkyl. Examples are, but not limited to norbornyl or bicyclo[2.2.2]octanyl.

The term “heteroaryl” means an aromatic mono, bi, or polycyclic ring incorporating one or more (for example 1 to 4, particularly 1, 2, or 3) heteroatoms selected from nitrogen, oxygen, or sulfur. Examples of heteroaryl groups are monocyclic and bicyclic groups containing from five to twelve ring members, and more usually from five to ten ring members. The heteroaryl group can be, for example, a 5- or 6-membered monocyclic ring (or monocyclic heteroaryl) or a 9- or 10-membered bicyclic ring, for example a bicyclic structure formed from fused five and six membered rings or two fused six membered rings. Each ring may contain up to about four heteroatoms typically selected from nitrogen, sulfur, and oxygen. Typically, the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general, the number of basic nitrogen atoms present in the heteroaryl group, including any amino group substituents of the ring, will be less than five. Heteroaryl groups containing nitrogen atoms may be present as the corresponding N-oxides.

Non-limiting examples of heteroaryl groups include furanyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl (pyridinyl), pyridazinyl, pyrimidinyl, pyrazinyl, 1,3,5 triazenyl, benzofuranyl, indolyl, isoindolyl, isoindolinyl, benzothienyl, benzoxazolyl, benzimidazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, imidazolidinyl, triazolopyridinyl, such as [1,2,4]triazolo[1,5-a]pyridinyl, [1,2,4]triazolo[4,3-a]pyridinyl, pyrrolopyridinyl such as 1H-pyrrolo[2,3-c]pyridinyl, 1H-pyrrolo[3,2-b]pyridinyl or 1H-pyrrolo[3,2-c]pyridinyl, imidazopyridinyl such as imidazo[1,2-a]pyridinyl, imidazopyridazinyl such as imidazo[1,2-b]pyridazinyl, thieno[2,3 b]furanyl, 2H furo[3,2 b]pyranyl, 5H pyrido[2,3 d]o oxazinyl, 1H pyrazolo[4,3 d]oxazolyl, 4H imidazo[4,5 d]thiazolyl, pyrazino[2,3 d]pyridazinyl, imidazo[2,1 b]thiazolyl, imidazo[1,2 b][1,2,4]triazinyl groups, pyrazolopyridinyl such as pyrazolo[4,3-b]pyridinyl and triazolopyridazinyl such as [1,2,4]triazolo[1,5-b]pyridazinyl, benzoimidazolyl such as 1H-benzo[d]imidazolyl.

Non-limiting examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl, and tetrazolyl groups.

Non-limiting examples of six membered heteroaryl groups include but are not limited to pyridinyl (pyridyl), pyrazinyl, pyridazinyl, pyrimidinyl, and triazinyl groups.

Non-limiting examples of bicyclic heteroaryl groups containing a six membered ring fused to a five membered ring include but are not limited to triazolopyridinyl such as [1,2,4]triazolo[1,5-a]pyridinyl or [1,2,4]triazolo[4,3-a]pyridinyl, pyrrolopyridinyl such as 1H-pyrrolo[2,3-c]pyridinyl, 1H-pyrrolo[3,2-b]pyridinyl or 1H-pyrrolo[3,2-c]pyridinyl, pyrrolopyrimidinyl, benzoimidazolyl such as 1H-benzo[d]imidazolyl, benzoxazolyl such as oxobenzo[d]oxazol-3(2H)-yl, imidazopyridinyl such as imidazo[1,2-a]pyridinyl, imidazopyridazinyl such as imidazo[1,2-b]pyridazinyl, imidazolyl, indazolyl such as indazol-1-yl or indazol-2-yl, indolyl such as indol-1-yl, isoindolinyl, pyrazolopyridinyl such as pyrazolo[4,3-b]pyridinyl and triazolopyridazinyl such as [1,2,4]triazolo[1,5-b]pyridazinyl. Particular non-limiting examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl, and pteridinyl groups.

The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl, and the like. Suitably an aryl group is a phenyl.

This specification may also make use of several composite terms to describe groups comprising more than one functionality. Such terms will be understood by a person skilled in the art. For example, heterocyclylC₁-C₄alkyl comprises C₁-C₄alkyl substituted by heterocyclyl.

The term “optionally substituted” refers to either groups, structures, or molecules that are substituted and those that are not substituted.

Where optional substituents are chosen from “one or more” groups it is to be understood that this definition includes all substituents being chosen from one of the specified groups or the substituents being chosen from two or more of the specified groups. It is understood that where there are multiple substituents, the substituents chosen may be the same or different.

Where numerical ranges are given, it is understood that the ranges are inclusive of the endpoints.

The phrase “compound of the disclosure” means those compounds which are disclosed herein, both generically and specifically.

The phrase “pharmaceutically acceptable” is employed herein to refer to those compounds, materials, compositions and/or dosage forms which are, within the scope of sound medical judgment, suitable for use in contact with the tissues of human beings and animals without excessive toxicity, irritation, allergic response or other problem or complication, commensurate with a reasonable benefit/risk ratio.

The term “pharmaceutically acceptable salts” in this respect refers to the relatively non-toxic, inorganic and organic acid addition salts of compounds of the present disclosure. These salts can be prepared in situ in the administration vehicle or the dosage form manufacturing process or by separately reacting a purified compound of the disclosure in its free base form with a suitable organic or inorganic acid and isolating the salt thus formed during subsequent purification.

As used herein, the term “pharmaceutically acceptable excipient” refers to a substance that aids the administration of an active agent to a subject. By “pharmaceutically acceptable”, it is meant that the excipient is compatible with the other ingredients of the formulation and is not deleterious to the recipient thereof. Pharmaceutical excipients useful in the present disclosure include, but are not limited to, binders, fillers, disintegrants, lubricants, glidants, coatings, sweeteners, flavors and colors.

The phrase “pharmaceutically acceptable carrier” as used herein means a pharmaceutically acceptable material, composition or vehicle, such as a liquid or solid filler, diluent, excipient or solvent encapsulating material, involved in carrying or transporting the subject compound from one organ or portion of the body, to another organ or portion of the body.

“It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder, or condition developing in a human that may be afflicted with or predisposed to the state, disorder, or condition but does not yet experience or display clinical or subclinical symptoms of the state, disorder, or condition, (2) inhibiting the state, disorder, or condition, i.e., arresting, reducing, or delaying the development of the disease or a relapse thereof (in case of maintenance treatment) or at least one clinical or subclinical symptom thereof, or (3) relieving or attenuating the disease, i.e., causing regression of the state, disorder, or condition or at least one of its clinical or subclinical symptoms.

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” refers to a human, that has been or will be the object of treatment, observation, or experiment. The methods described herein may be useful in human therapy.

A “therapeutically effective amount” means the amount of a compound that, when administered to a mammal for treating a disease, is sufficient to effect such treatment for the disease. The “therapeutically effective amount” will vary depending on the compound, the disease and its severity, and the age, weight, etc., of the mammal to be treated, which can readily be determined by one of ordinary skill in the art.

As used herein chemical nomenclature as not defined otherwise have the meanings as being used in the technical field.

### Compounds

Disclosed herein is a compound of formula (I):

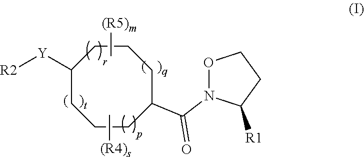

wherein
R1 represents a phenyl or a monocyclic heteroaryl, which is optionally substituted by one, two or three R3;
R2 represents an aryl or a heteroaryl, which is optionally substituted by one, two or three R6;
each R3 is independently chosen from a halogen, a cyano, a (C₁-C₄)alkyl group or a (C₁-C₄)alkoxy group;
R4 and R5 are independently chosen from a halogen, a (C₁-C₄)alkyl group, a (C₁-C₄)alkoxy group, or R4 and R5 form together a (C₁-C₄)alkylene bridge;
m and s are independently 0 or 1;
p, q, r and t are independently 0 or 1;
Y is a bond or a divalent group chosen from —C(O)—NH—, —C(O)—NH—CH₂—, —NH—, —CH₂—NH—, —NH—CH₂—, —O—, CH₂—O— and —O—CH₂—;
each R6 is independently selected from halogen, cyano, —OH, a (C₁-C₄)-alkyl group, —CF₃, —C(O)NH₂, a —C(O)NH—(C₁-C₄)-alkyl group, —C(O)OH, a —C(O)O—(C₁-C₄)-alkyl group, —SO₂NH₂, a (C₁-C₄)-alkoxy group, a —O—(C₁-C₄)alkylene-(C₃-C₆)cycloalkyl group, a —O—(C₃-C₆)cycloalkyl group, a —O—(C₃-C₆)heterocycloalkyl group, a 5- or 6-memembered heterocycloalkyl group, a monocyclic heteroaryl group and oxo, wherein said (C₁-C₄)alkyl group, (C₁-C₄)alkoxy group, —O—(C₃-C₆)cycloalkyl group, (C₁-C₄)alkyl group, 5- or 6-memembered heterocycloalkyl group or monocyclic heteroaryl group is optionally substituted by one, two, three or four R7;
each R7 is independently a halogen, oxo, —OH, a (C₁-C₄)alkyl group or a (C₁-C₄)alkoxy group,
or a pharmaceutically acceptable salt, solvate, or stereoisomer thereof.

According to one embodiment, R1 in formula (1) is phenyl, thiazolyl, pyridinyl or pyrazinyl, optionally substituted by one or two groups independently selected from a halogen, a cyano, a (C₁-C₂)-alkyl group or a (C₁-C₂)-alkoxy group, in particular wherein R1 represents (i) a phenyl substituted by one or two groups independently selected from a halogen, a cyano, a (C₁-C₂)-alkyl group or a (C₁-C₂)-alkoxy group or (ii) a monocyclic heteroaryl selected from a thiazolyl, a pyridinyl and a pyrazinyl, said heteroaryl being optionally substituted by one or two groups independently selected from a halogen, a cyano, a (C₁-C₂)-alkyl group or a (C₁-C₂)-alkoxy group.

According to one embodiment, R1 in formula (1) is phenyl, optionally substituted by one or two groups independently selected from fluorine, chlorine, a cyano, a methyl group or a methoxy group.

According to one embodiment, R1 in formula (1) is thiazolyl, pyridinyl or pyrazinyl, optionally substituted by one or two groups independently selected from fluorine, chlorine, a cyano, a methyl group or a methoxy group.

According to another embodiment, R2 in formula (1) is phenyl, pyrimidinyl, pyridinyl, pyrrolopyrimidinyl, benzimidazolyl, indazolyl or indolyl, optionally substituted by one or two groups independently selected from halogen, cyano, a (C₁-C₂)-alkyl group, —C(O)NH₂, a —C(O)O—(C₁-C₂)-alkyl group, —SO₂NH₂, a 5- or 6-membered heterocycloalkyl group, and a 5-membered heteroaryl, said 5- or 6-membered heterocycloalkyl group and 5-membered heteroaryl being optionally substituted by one, two, three or four groups independently selected from a (C₁-C₂)alkyl group and oxo.

According to another embodiment, R2 in formula (1) is phenyl, pyrimidinyl, pyridinyl, pyrrolopyrimidinyl, benzimidazolyl, indazolyl or indolyl, optionally substituted by one or two groups independently selected from halogen, cyano, a (C₁-C₂)-alkyl group, —C(O)NH₂, a —C(O)O—(C₁-C₂)-alkyl group, —SO₂NH₂, a 5- or 6-membered heterocycloalkyl group, selected from in particular oxazolidinyl, pyrrolidinyl, imidazolinyl or dihydroimidazolyl, and a 5-membered heteroaryl, in particular a pyrazolyl or a triazolyl, said 5- or 6-membered heterocycloalkyl group and 5-membered heteroaryl being optionally substituted by one, two, three or four groups independently selected from a (C₁-C₂)alkyl group and oxo.

According to another embodiment, Y is a bond or a divalent group chosen from —NH—, —NH—CH₂—, —O— and —O—CH₂—, in particular a group chosen from —NH—, —NH—CH₂—, and —O—CH₂—.

According to one embodiment, R4 and R5 in formula (1) are independently chosen from a (C₁-C₂)alkyl group.

According to another embodiment of a compound of formula (1), m is 0 and s is 0.

According to one embodiment, a compound according to the disclosure is of formula (1) wherein alternatively:
p, q, r and t are all equal to 0,
r, q and t are all equal to 0 and p is equal to 1,
r and t are both equal to 0 and q and p are both equal to 1, or
q, t and p are all equal to 1 and r is equal to 0.

According to one embodiment, herein is provided a compound of formula (1) as defined above, wherein R1 represents:
a phenyl, substituted by one, two or three R3 as defined above, or
a monocyclic heteroaryl optionally substituted by one, two or three R3 as defined above.

According to one embodiment, herein is provided a compound of formula (1) as defined above, wherein Y is a divalent group chosen from —C(O)—NH—, —C(O)—NH—CH₂—, —NH—, —CH₂—NH—, —NH—CH₂—, CH₂—O— and —O—CH₂—.

According to one embodiment, herein is provided a compound of formula (1) as defined above, wherein if p, q, r and t are 0, then R4 and R5 do not form together a (C₁-C₄)alkylene bridge.

According to one embodiment, a compound according to the disclosure is of formula (Ia) wherein:

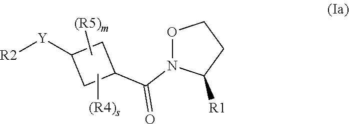

wherein:
Y, R1, R2, R4, R5, m and s are as defined in formula (1),
or a pharmaceutically acceptable salt, solvate, or stereoisomer thereof.

According to another embodiment, a compound according to the disclosure is of formula (Ia) wherein:
Y is —NH—, —O— or a bond;
R1 is phenyl or pyridinyl, which is optionally substituted by one or two R3;
R2 is phenyl, pyrimidinyl, indazolyl, indolyl or benzimidazolyl, which is optionally substituted by one or two R6;
each R3 is independently chosen from a halogen or cyano;
m and s are independently 0 or 1;
each R6 is a halogen, in particular a fluorine, —C(O)NH₂, cyano or a —C(O)O—(C₁-C₂)alkyl;
or a pharmaceutically acceptable salt, solvate, or stereoisomer thereof.

According to this embodiment, a compound of formula (1) is more particularly of following formula (Ib):

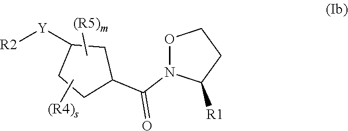

wherein Y, R1, R2, R4, R5, m and s are as defined in formula (1),
or a pharmaceutically acceptable salt, solvate, or stereoisomer thereof.

According to another embodiment, a compound of formula (1) is more particularly of formula (Ib), wherein:
Y is a bond or —O—CH₂—;
R1 is phenyl, which is optionally substituted by one or two R3;
R2 is pyridinyl or pyrimidinyl, which is optionally substituted by —C(O)NH₂;
each R3 is independently chosen from a halogen or cyano;
m and s are 0;
or a pharmaceutically acceptable salt, solvate, or stereoisomer thereof.

According to this embodiment, a compound of formula (1) is more particularly of following formula (Ic):

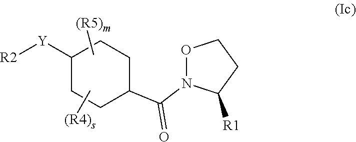

wherein Y, R1, R2, R4, R5, m and s are as defined in formula (1),
or a pharmaceutically acceptable salt, solvate, or stereoisomer thereof.

According to this embodiment, a compound of formula (1) is more particularly of formula (Ic), wherein
Y is —NH—, —NH—CH₂—, a bond, —O— or —O—CH₂—;
R1 is phenyl, pyrazinyl, triazolyl or pyridinyl, which is optionally substituted by one or two R3;
R2 is pyridinyl, phenyl, pyrimidinyl, pyrrolopyrimidinyl, optionally substituted by one or two R6;
each R3 is independently chosen from a halogen, —CH₃, —OCH₃ or cyano;
m and s are 0;
each R6 is independently chosen from a halogen, —C(O)NH₂, cyano, a (C₁-C₂)-alkyl group, —SO₂NH₂, a 5-membered heterocycloalkyl selected from imidazolidinyl, dihydroimidazolyl, pyrrolidinyl and oxazolidinyl or a 5-membered heteroaryl selected from pyrazolyl or triazolyl, said heterocycloalkyl and heteroaryl being optionally substituted by one, two, three or four groups independently selected from a (C₁-C₂)alkyl group and oxo,
or a pharmaceutically acceptable salt, solvate, or stereoisomer thereof.

According to this embodiment, a compound of formula (1) is more particularly of following formula (Id):

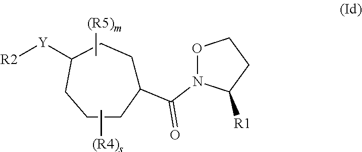

wherein Y, R1, R2, R4, R5, m and s are as defined in formula (I),
or a pharmaceutically acceptable salt, solvate, or stereoisomer thereof.

According to another embodiment, a compound of formula (I) is more particularly of formula (Id), wherein:
Y is —O—CH₂—;
R1 is phenyl, which is optionally substituted by one or two R3;
R2 is pyrimidinyl, optionally substituted by —C(O)NH₂;
each R3 is independently chosen from a halogen or cyano;
m and s are 0;
or a pharmaceutically acceptable salt, solvate, or stereoisomer thereof.

Among the compounds of formula (I), mention may be made in particular of the following compounds:
(1) cis-2-((3-((S)-3-(3,5-difluorophenyl)isoxazolidine-2-carbonyl)cyclobutyl)amino)pyrimidine-4-carboxamide,
(2) cis-2-((3-((S)-3-(3,5-difluorophenyl)isoxazolidine-2-carbonyl)cyclobutyl)amino)pyrimidine-4-carbonitrile,
(3) methyl cis-2-((3-((S)-3-(3,5-difluorophenyl)isoxazolidine-2-carbonyl)cyclobutyl)amino)pyrimidine-4-carboxylate,
(4) trans-2-((3-((S)-3-(3,5-difluorophenyl)isoxazolidine-2-carbonyl)cyclobutyl)amino)pyrimidine-4-carboxamide,
(5) cis-2-((3-((S)-3-(3,5-difluorophenyl)isoxazolidine-2-carbonyl)-3-methylcyclobutyl)amino)pyrimidine-4-carbonitrile,
(6) cis-2-((3-((S)-3-(3,5-difluorophenyl)isoxazolidine-2-carbonyl)-3-methylcyclobutyl)amino)pyrimidine-4-carboxamide,
(7) cis-2-((3-((S)-3-(5-cyanopyridin-3-yl)isoxazolidine-2-carbonyl)-3-methylcyclobutyl)amino)pyrimidine-4-carboxamide,
(8) ethyl cis-6-((3-((S)-3-(3,5-difluorophenyl)isoxazolidine-2-carbonyl)-3-methylcyclobutyl)amino)-5-fluoropyrimidine-4-carboxylate,
(9) ethyl cis-6-chloro-5-((3-((S)-3-(3,5-difluorophenyl)isoxazolidine-2-carbonyl)-3-methylcyclobutyl)amino)pyrimidine-4-carboxylate,
(10) cis-6-chloro-5-((3-((S)-3-(3,5-difluorophenyl)isoxazolidine-2-carbonyl)-3-methylcyclobutyl)amino)pyrimidine-4-carboxamide,
(11) cis-6-((3-((S)-3-(3,5-difluorophenyl)isoxazolidine-2-carbonyl)-3-methylcyclobutyl)amino)-5-fluoropyrimidine-4-carboxamide,
(12) cis-2-((4-((S)-3-(4-chlorophenyl)isoxazolidine-2-carbonyl)cyclohexyl)amino)pyrimidine-4-carboxamide,
(13) cis-6-((4-((S)-3-(4-chlorophenyl)isoxazolidine-2-carbonyl)cyclohexyl)amino)pyrimidine-4-carboxamide,
(14) trans-2-(((4-((S)-3-(4-chlorophenyl)isoxazolidine-2-carbonyl)cyclohexyl)methyl)amino)pyrimidine-4-carbonitrile,
(15) trans-6-(((4-((S)-3-(4-chlorophenyl)isoxazolidine-2-carbonyl)cyclohexyl)methyl)amino)pyrimidine-4-carbonitrile,
(16) cis-2-((3-((S)-3-(3-fluorophenyl)isoxazolidine-2-carbonyl)cyclobutyl)amino)pyrimidine-4-carbonitrile,
(17) cis-6-((3-((S)-3-(3-fluorophenyl)isoxazolidine-2-carbonyl)cyclobutyl)amino)pyrimidine-4-carbonitrile,
(18) cis-6-((3-((S)-3-(3,5-difluorophenyl)isoxazolidine-2-carbonyl)cyclobutyl)amino)pyrimidine-4-carbonitrile,
(19) cis-2-((3-((S)-3-(3-cyano-5-fluorophenyl)isoxazolidine-2-carbonyl)cyclobutyl)amino)pyrimidine-4-carbonitrile,
(20) cis-6-((3-((S)-3-(3-cyano-5-fluorophenyl)isoxazolidine-2-carbonyl)cyclobutyl)amino)pyrimidine-4-carbonitrile,
(21) cis-2-((3-((S)-3-(3,4-difluorophenyl)isoxazolidine-2-carbonyl)cyclobutyl)amino)pyrimidine-4-carbonitrile,
(22) cis-6-((3-((S)-3-(3,4-difluorophenyl)isoxazolidine-2-carbonyl)cyclobutyl)amino)pyrimidine-4-carbonitrile,
(23) cis-6-((3-((S)-3-(4-chlorophenyl)isoxazolidine-2-carbonyl)cyclobutyl)amino)pyrimidine-4-carbonitrile,
(24) cis-2-((3-((S)-3-(4-chlorophenyl)isoxazolidine-2-carbonyl)cyclobutyl)amino)pyrimidine-4-carbonitrile,
(25) cis-3-((3-((S)-3-(4-chlorophenyl)isoxazolidine-2-carbonyl)cyclobutyl)amino)-5-fluorobenzonitrile,
(26) cis-3-fluoro-5-((3-((S)-3-(3-fluorophenyl)isoxazolidine-2-carbonyl)cyclobutyl)amino)benzonitrile,
(27) cis-3-((3-((S)-3-(3,4-difluorophenyl)isoxazolidine-2-carbonyl)cyclobutyl)amino)-5-fluorobenzonitrile,
(28) cis-6-((4-((S)-3-(3-cyano-5-fluorophenyl)isoxazolidine-2-carbonyl)cyclohexyl)amino)pyrimidine-4-carbonitrile,
(29) trans-6-(((4-((S)-3-(3-cyano-5-fluorophenyl)isoxazolidine-2-carbonyl)cyclohexyl)methyl)amino)pyrimidine-4-carboxamide,
(30) trans-6-(((4-((S)-3-(3-cyano-5-fluorophenyl)isoxazolidine-2-carbonyl)cyclohexyl)methyl)amino)pyrimidine-4-carbonitrile,
(31) trans-3-fluoro-5-((S)-2-(4-(((7-methyl-7H-pyrrolo[2,3-d]pyrimidin-4-yl)amino)methyl)cyclohexane-1-carbonyl)isoxazolidin-3-yl)benzonitrile,
(32) cis-5-((3-((S)-3-(3-cyano-5-fluorophenyl)isoxazolidine-2-carbonyl)cyclobutyl)amino)-2-fluorobenzonitrile,
(33) cis-2-fluoro-5-((3-((S)-3-(5-fluoropyridin-3-yl)isoxazolidine-2-carbonyl)cyclobutyl)amino)benzonitrile,
(34) trans-2-fluoro-5-(((4-((S)-3-(5-fluoropyridin-3-yl)isoxazolidine-2-carbonyl)cyclohexyl)methyl)amino)benzonitrile,
(35) trans-5-(((4-((S)-3-(3-cyano-5-fluorophenyl)isoxazolidine-2-carbonyl)cyclohexyl)methyl)amino)-2-fluorobenzonitrile,
(36) cis-3-fluoro-5-((3-((S)-3-(5-fluoropyridin-3-yl)isoxazolidine-2-carbonyl)cyclobutyl)amino)benzonitrile,
(37) cis-3-((S)-2-(3-((3-cyano-5-fluorophenyl)amino)cyclobutane-1-carbonyl)isoxazolidin-3-yl)-5-fluorobenzonitrile,
(38) trans-3-((S)-2-(4-(((3-cyano-5-fluorophenyl)amino)methyl)cyclohexane-1-carbonyl)isoxazolidin-3-yl)-5-fluorobenzonitrile,
(39) cis-3-((S)-2-(4-((3-cyano-5-fluorophenyl)amino)cyclohexane-1-carbonyl)isoxazolidin-3-yl)-5-fluorobenzonitrile,
(40) cis-3-((S)-2-(3-(1H-benzo[d]imidazol-1-yl)cyclobutane-1-carbonyl)isoxazolidin-3-yl)-5-fluorobenzonitrile,
(41) cis-(3-(1H-benzo[d]imidazol-1-yl)cyclobutyl)((S)-3-(3,5-difluorophenyl)isoxazolidin-2-yl)methanone,
(42) cis-(3-(1H-benzo[d]imidazol-1-yl)cyclobutyl)((S)-3-(5-fluoropyridin-3-yl)isoxazolidin-2-yl)methanone,
(43) trans-5-((4-((S)-3-(3,5-difluorophenyl)isoxazolidine-2-carbonyl)cyclohexyl)methoxy)-2-fluorobenzonitrile,
(44) trans-5-((4-((S)-3-(3-cyano-5-fluorophenyl)isoxazolidine-2-carbonyl)cyclohexyl)methoxy)-2-fluorobenzonitrile,
(45) trans-6-((4-((S)-3-(3,5-difluorophenyl)isoxazolidine-2-carbonyl)cyclohexyl)methoxy)pyrimidine-4-carbonitrile,
(46) trans-(4-(((6-chloropyrimidin-4-yl)oxy)methyl)cyclohexyl)((S)-3-(3,5-difluorophenyl)isoxazolidin-2-yl)methanone,
(47) trans-6-((4-((S)-3-(3,5-difluorophenyl)isoxazolidine-2-carbonyl)cyclohexyl)methoxy)pyrimidine-4-carboxamide,
(48) trans-3-((4-((S)-3-(3,5-difluorophenyl)isoxazolidine-2-carbonyl)cyclohexyl)methoxy)-5-fluorobenzonitrile,
(49) trans-3-((S)-2-(4-((3-cyano-5-fluorophenoxy)methyl)cyclohexane-1-carbonyl)isoxazolidin-3-yl)-5-fluorobenzonitrile,
(50) trans-3-(3-((S)-3-(3,5-difluorophenyl)isoxazolidine-2-carbonyl)cyclobutoxy)-5-fluorobenzonitrile,
(51) trans-5-(3-((S)-3-(3,5-difluorophenyl)isoxazolidine-2-carbonyl)cyclobutoxy)-2-fluorobenzonitrile,
(52) cis-5-((4-((S)-3-(3,5-difluorophenyl)isoxazolidine-2-carbonyl)cyclohexyl)methoxy)-2-fluorobenzonitrile,
(53) cis-3-((4-((S)-3-(3,5-difluorophenyl)isoxazolidine-2-carbonyl)cyclohexyl)methoxy)-5-fluorobenzonitrile,
(54) cis-6-((4-((S)-3-(3,5-difluorophenyl)isoxazolidine-2-carbonyl)cyclohexyl)methoxy)pyrimidine-4-carboxamide,
(55) cis-6-((4-((S)-3-(3,5-difluorophenyl)isoxazolidine-2-carbonyl)cyclohexyl)methoxy)pyrimidine-4-carbonitrile,
(56) cis-(4-(((6-chloropyrimidin-4-yl)oxy)methyl)cyclohexyl)((S)-3-(3,5-difluorophenyl)isoxazolidin-2-yl)methanone,
(57) trans-3-(4-chloro-3-((4-((S)-3-(3,5-difluorophenyl)isoxazolidine-2-carbonyl)cyclohexyl)methoxy)phenyl)-5,5-dimethyloxazolidine-2,4-dione,
(58) trans-1-(4-chloro-3-((4-((S)-3-(3,5-difluorophenyl)isoxazolidine-2-carbonyl)cyclohexyl)methoxy)phenyl)pyrrolidine-2,5-dione,
(59) trans-3-(3-((4-((S)-3-(3,5-difluorophenyl)isoxazolidine-2-carbonyl)cyclohexyl)methoxy)-4-fluorophenyl)-5,5-dimethyloxazolidine-2,4-dione,
(60) trans-1-(4-chloro-3-((4-((S)-3-(3,5-difluorophenyl)isoxazolidine-2-carbonyl)cyclohexyl)methoxy)phenyl)pyrrolidin-2-one,
(61) cis-3-(3-((S)-3-(3,5-difluorophenyl)isoxazolidine-2-carbonyl)cyclobutoxy)-5-fluorobenzonitrile,
(62) trans-((S)-3-(3,5-difluorophenyl)isoxazolidin-2-yl)(4-((4-(3,5-dimethyl-1H-pyrazol-1-yl)-2-fluorophenoxy)methyl)cyclohexyl)methanone,
(63) trans-3-(4-chloro-3-((4-((S)-3-(3,5-difluorophenyl)isoxazolidine-2-carbonyl)cyclohexyl)methoxy)phenyl)-1-methylimidazolidine-2,4-dione,
(64) trans-((S)-3-(3,5-difluorophenyl)isoxazolidin-2-yl)(4-((5-(3,5-dimethyl-4H-1,2,4-triazol-4-yl)-2-fluorophenoxy)methyl)cyclohexyl)methanone,
(65) cis-5-(3-((S)-3-(3,5-difluorophenyl)isoxazolidine-2-carbonyl)cyclobutoxy)-2-fluorobenzonitrile,
(66) trans-3-((4-((S)-3-(3,5-difluorophenyl)isoxazolidine-2-carbonyl)cyclohexyl)methoxy)benzenesulfonamide,
(67) trans-((S)-3-(3,5-difluorophenyl)isoxazolidin-2-yl)(4-((4-(3,5-dimethyl-4H-1,2,4-triazol-4-yl)-2-fluorophenoxy)methyl)cyclohexyl)methanone,
(68) trans-1-(4-((4-((S)-3-(3,5-difluorophenyl)isoxazolidine-2-carbonyl)cyclohexyl)methoxy)-3-fluorophenyl)-3-methyl-1,3-dihydro-2H-imidazol-2-one,
(69) cis-((S)-3-(3,5-difluorophenyl)isoxazolidin-2-yl)(3-(5-fluoro-1H-benzo[d]imidazol-1-yl)cyclobutyl)methanone,
(70) cis-3-fluoro-5-((S)-2-(3-(5-fluoro-1H-benzo[d]imidazol-1-yl)cyclobutane-1-carbonyl)isoxazolidin-3-yl)benzonitrile,
(71) trans-5-((S)-2-(4-((3-cyanophenoxy)methyl)cyclohexane-1-carbonyl)isoxazolidin-3-yl)nicotinonitrile,
(72) trans-5-((S)-2-(4-((3-cyano-4-fluorophenoxy)methyl)cyclohexane-1-carbonyl)isoxazolidin-3-yl)nicotinonitrile,
(73) trans-3-((4-((S)-3-(5-cyanopyridin-3-yl)isoxazolidine-2-carbonyl)cyclohexyl)methoxy)benzamide,
(74) trans-5-((4-((S)-3-(5-cyanopyridin-3-yl)isoxazolidine-2-carbonyl)cyclohexyl)methoxy)-2-fluorobenzamide,
(75) trans-2-chloro-5-((4-((S)-3-(5-cyanopyridin-3-yl)isoxazolidine-2-carbonyl)cyclohexyl)methoxy)benzamide,
(76) trans-3-((S)-2-(4-((4-(3,5-dimethyl-1H-pyrazol-1-yl)-2-fluorophenoxy)methyl)cyclohexane-1-carbonyl)isoxazolidin-3-yl)-5-fluorobenzonitrile,
(77) trans-3-((4-((S)-3-(3-cyano-5-fluorophenyl)isoxazolidine-2-carbonyl)cyclohexyl)methoxy)benzenesulfonamide,
(78) trans-3-fluoro-5-((S)-2-(4-((2-fluoro-4-(3-methyl-2-oxo-2,3-dihydro-1H-imidazol-1-yl)phenoxy)methyl)cyclohexane-1-carbonyl)isoxazolidin-3-yl)benzonitrile,
(79) trans-3-((S)-2-(4-((5-(5,5-dimethyl-2,4-dioxooxazolidin-3-yl)-2-fluorophenoxy)methyl)cyclohexane-1-carbonyl)isoxazolidin-3-yl)-5-fluorobenzonitrile,
(80) trans-3-((S)-2-(4-((2-chloro-5-(3-methyl-2,5-dioxoimidazolidin-1-yl)phenoxy)methyl)cyclohexane-1-carbonyl)isoxazolidin-3-yl)-5-fluorobenzonitrile,
(81) cis-3-((S)-2-(4-(5-(5,5-dimethyl-2,4-dioxooxazolidin-3-yl)-2-fluorophenoxy)cyclohexane-1-carbonyl)isoxazolidin-3-yl)-5-fluorobenzonitrile,
(82) cis-5-((4-((S)-3-(3-cyano-5-fluorophenyl)isoxazolidine-2-carbonyl)cyclohexyl)oxy)-2-fluorobenzonitrile,
(83) trans-5-((S)-2-(4-((3-cyano-5-fluorophenoxy)methyl)cyclohexane-1-carbonyl)isoxazolidin-3-yl)nicotinonitrile,
(84) trans-(3-(5-fluoro-1H-benzo[d]imidazol-1-yl)cyclobutyl)((S)-3-(3-fluorophenyl)isoxazolidin-2-yl)methanone,
(85) trans-5-((4-((S)-3-(3-cyano-5-fluorophenyl)isoxazolidine-2-carbonyl)cyclohexyl)methoxy)-2-methylbenzamide,
(86) trans-5-((4-((S)-3-(3,5-difluorophenyl)isoxazolidine-2-carbonyl)cyclohexyl)methoxy)-2-methylbenzamide,
(87) cis-((S)-3-(3,5-difluorophenyl)isoxazolidin-2-yl)(3-(6-fluoro-1H-benzo[d]imidazol-1-yl)cyclobutyl)methanone,
(88) trans-5-((S)-2-(4-((2-chloro-5-(3-methyl-2,5-dioxoimidazolidin-1-yl)phenoxy)methyl)cyclohexane-1-carbonyl)isoxazolidin-3-yl)nicotinonitrile,
(89) trans-5-((S)-2-(4-((2-fluoro-4-(3-methyl-2-oxo-2,3-dihydro-1H-imidazol-1-yl)phenoxy)methyl)cyclohexane-1-carbonyl)isoxazolidin-3-yl)nicotinonitrile,
(90) trans-5-((S)-2-(4-((4-(3,5-dimethyl-1H-pyrazol-1-yl)-2-fluorophenoxy)methyl)cyclohexane-1-carbonyl)isoxazolidin-3-yl)nicotinonitrile,
(91) trans-2-[[4-[(3S)-3-(3-cyano-5-fluoro-phenyl)isoxazolidine-2-carbonyl]cyclohexyl]methoxy]pyridine-4-carbonitrile,
(92) trans-5-((S)-2-(4-((5-(5,5-dimethyl-2,4-dioxooxazolidin-3-yl)-2-fluorophenoxy)methyl)cyclohexane-1-carbonyl)isoxazolidin-3-yl)nicotinonitrile,
(93) trans-4-[[4-[(3S)-3-(3-cyano-5-fluoro-phenyl)isoxazolidine-2-carbonyl]cyclohexyl]methoxy]pyridine-2-carbonitrile,
(94) trans-5-((S)-2-(4-(((4-cyanopyridin-2-yl)oxy)methyl)cyclohexane-1-carbonyl)isoxazolidin-3-yl)nicotinonitrile,
(95) trans-5-((S)-2-(4-(((5-cyanopyridin-2-yl)oxy)methyl)cyclohexane-1-carbonyl)isoxazolidin-3-yl)nicotinonitrile,
(96) cis-[(3S)-3-(3,5-difluorophenyl)isoxazolidin-2-yl]-[3-(5-fluoroindazol-1-yl)cyclobutyl]methanone,
(97) trans-[(3S)-3-(3,5-difluorophenyl)isoxazolidin-2-yl]-[3-(5-fluoroindazol-1-yl)cyclobutyl]methanone,
(98) trans-3-fluoro-5-[(3S)-2-[3-(5-fluoroindazol-1-yl)cyclobutanecarbonyl]isoxazolidin-3-yl]benzonitrile,
(99) trans-6-[[4-[(3S)-3-(5-cyano-3-pyridyl)isoxazolidine-2-carbonyl]cyclohexyl]methoxy]pyridine-3-carboxamide,
(100) trans-3-[[4-[(3S)-3-pyrazin-2-ylisoxazolidine-2-carbonyl]cyclohexyl]methoxy]benzamide,
(101) trans-2-fluoro-5-[[4-[(3S)-3-pyrazin-2-ylisoxazolidine-2-carbonyl]cyclohexyl]methoxy]benzamide,
(102) trans-6-[[4-[(3S)-3-pyrazin-2-ylisoxazolidine-2-carbonyl]cyclohexyl]methoxy]pyridine-3-carboxamide,
(103) trans-6-[[4-[(3S)-3-pyrazin-2-ylisoxazolidine-2-carbonyl]cyclohexyl]methoxy]pyridine-3-carbonitrile,
(104) trans-3-fluoro-5-[[4-[(3S)-3-pyrazin-2-ylisoxazolidine-2-carbonyl]cyclohexyl]methoxy]benzonitrile,
(105) trans-3-fluoro-5-[(3S)-2-[3-(5-fluoroindol-1-yl)cyclobutanecarbonyl]isoxazolidin-3-yl]benzonitrile,
(106) trans-[(3S)-3-(3,5-difluorophenyl)isoxazolidin-2-yl]-[3-(5-fluoroindol-1-yl)cyclobutyl]methanone,
(107) cis-3-fluoro-5-[(3S)-2-[3-(5-fluoroindol-1-yl)cyclobutanecarbonyl]isoxazolidin-3-yl]benzonitrile,
(108) cis-[(3S)-3-(3,5-difluorophenyl)isoxazolidin-2-yl]-[3-(5-fluoroindol-1-yl)cyclobutyl]methanone,
(109) cis-3-fluoro-5-[(3S)-2-[3-(5-fluoroindazol-2-yl)cyclobutanecarbonyl]isoxazolidin-3-yl]benzonitrile,
(110) cis-[(3S)-3-(3,5-difluorophenyl)isoxazolidin-2-yl]-[3-(5-fluoroindazol-2-yl)cyclobutyl]methanone,
(111) trans-4-fluoro-3-[[4-[(3S)-3-pyrazin-2-ylisoxazolidine-2-carbonyl]cyclohexyl]methoxy]benzamide,
(112) trans-3-fluoro-5-[[4-[(3S)-3-pyrazin-2-ylisoxazolidine-2-carbonyl]cyclohexyl]methoxy]benzamide,
(113) trans-3-fluoro-4-[[4-[(3S)-3-pyrazin-2-ylisoxazolidine-2-carbonyl]cyclohexyl]methoxy]benzamide,
(114) cis-2-[3-[(3S)-3-(3,5-difluorophenyl)isoxazolidine-2-carbonyl]cyclopentyl]pyridine-4-carboxamide,
(115) cis-6-[[3-[(3S)-3-(3-cyano-5-fluoro-phenyl)isoxazolidine-2-carbonyl]cyclopentyl]methoxy]pyrimidine-4-carboxamide,
(116) trans-6-[[3-[(3S)-3-(3-cyano-5-fluoro-phenyl)isoxazolidine-2-carbonyl]cyclopentyl]methoxy]pyrimidine-4-carboxamide stereoisomer 2,
(117) trans-6-[[3-[(3S)-3-(3-cyano-5-fluoro-phenyl)isoxazolidine-2-carbonyl]cyclopentyl]methoxy]pyrimidine-4-carboxamide stereoisomer 3,
(118) trans-6-[[3-[(3S)-3-(3,5-difluorophenyl)-1,2-oxazolidine-2-carbonyl]cyclopentyl]methoxy]pyrimidine-4-carboxamide stereoisomer 1,
(119) cis-6-[[3-[(3S)-3-(3,5-difluorophenyl)-1,2-oxazolidine-2-carbonyl]cyclopentyl]methoxy]pyrimidine-4-carboxamide stereoisomer 2,
(120) trans-6-[[3-[(3S)-3-(3,5-difluorophenyl)-1,2-oxazolidine-2-carbonyl]cyclopentyl]methoxy]pyrimidine-4-carboxamide stereoisomer 3,
(121) cis-6-[[3-[(3S)-3-(3,5-difluorophenyl)-1,2-oxazolidine-2-carbonyl]cyclopentyl]methoxy]pyrimidine-4-carboxamide stereoisomer 4,
(122) trans-6-[[4-[(3S)-3-(3,5-difluorophenyl)-1,2-oxazolidine-2-carbonyl]cycloheptyl]methoxy]pyrimidine-4-carboxamide stereoisomer 1,
(123) trans-6-[[4-[(3S)-3-(3,5-difluorophenyl)-1,2-oxazolidine-2-carbonyl]cycloheptyl]methoxy]pyrimidine-4-carboxamide stereoisomer 2,
(124) cis-6-[[4-[(3S)-3-(3,5-difluorophenyl)-1,2-oxazolidine-2-carbonyl]cycloheptyl]methoxy]pyrimidine-4-carboxamide stereoisomer 3,
(125) cis-6-[[4-[(3S)-3-(3,5-difluorophenyl)-1,2-oxazolidine-2-carbonyl]cycloheptyl]methoxy]pyrimidine-4-carboxamide stereoisomer 4,
(126) trans-6-[[4-[(3S)-3-(3-cyano-5-fluoro-phenyl)isoxazolidine-2-carbonyl]cycloheptyl]methoxy]pyrimidine-4-carboxamide stereoisomer 1,
(127) trans-6-[[4-[(3S)-3-(3-cyano-5-fluoro-phenyl)isoxazolidine-2-carbonyl]cycloheptyl]methoxy]pyrimidine-4-carboxamide stereoisomer 2,
(128) cis-6-[[4-[(3S)-3-(3-cyano-5-fluoro-phenyl)isoxazolidine-2-carbonyl]cycloheptyl]methoxy]pyrimidine-4-carboxamide,
(129) trans-3-[[4-[(3S)-3-(6-methoxypyrazin-2-yl)isoxazolidine-2-carbonyl]cyclohexyl]methoxy]benzamide,
(130) trans-3-[[4-[(3S)-3-(2-methylthiazol-4-yl)isoxazolidine-2-carbonyl]cyclohexyl]methoxy]benzamide, and
(131) trans-3-[[4-[(3S)-3-(2-pyridyl)isoxazolidine-2-carbonyl]cyclohexyl]methoxy]benzamide,
   or a pharmaceutically acceptable salt, solvate, or stereoisomer thereof.

According to one embodiment of the present disclosure, the compound of formula (1) is chosen from the group consisting of compounds (1), (6), (32), (35), (37), (38), (43), (44), (45), (46), (47), (48), (49), (51), (52), (53), (57), (59), (62), (63), (65), (66), (68), (69), (71), (72), (73), (76), (77), (78), (79), (83), (85), (86), (89), (90), (91), (93), (95), (104), (112), (122), (123), (127) and (129) or a pharmaceutically acceptable salt, solvate, or stereoisomer thereof.

According to another embodiment of the present disclosure, the compound of formula (1) is chosen from the group consisting of compounds (6), (35), (43), (51), (52), (53), (68), (71), (72), (79), (86), (89), (90), (93) and (123) or a pharmaceutically acceptable salt, solvate, or stereoisomer thereof.

Some compounds of the disclosure are described with their structure in Table 1, which is merely illustrative and does not limit the scope of the present disclosure.

NMR and LC/MS data in Table 1 were obtained according to methods which are detailed in the experimental part provided for the detailed examples synthesis.

TABLE 1
Structure and analytical characterization of compounds (1) to (131)

| Compound | | |
|---|---|---|
| No | Structure | Description |
| 1 | 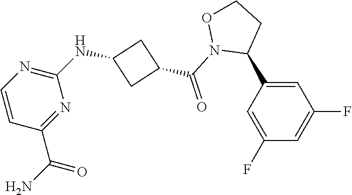 [SMILES: NC(=O)c1ccnc(N[C@H]2C[C@@H](C(=O)N3OCC[C@H]3c3cc(F)cc(F)c3)C2)n1] | 1H NMR (400.23 MHz, DMSO-d6) δ ppm 8.46 (br d, J = 4.03 Hz, 1 H), 8.06 (br s, 1 H), 7.73 (m, 2 H), 7.14 (m, 1 H), 7.05 (d, J = 4.77 Hz, 1 H), 7.00 (br d, J = 6.72 Hz, 2 H), 5.34 (dd, J = 8.62, 6.42 Hz, 1 H), 4.50 (br s, 1 H), 4.22 (m, 1 H), 3.79 (m, 1 H), 2.87 (m, 1 H), 2.33 (br s, 1 H), 2.20 (m, 3 H). LC/MS: m/z [M + H]⁺ 404.21, Rt (λ = 220 nm): 1.74 min (LC/MS method A), calc. m/z: 404.39 |

| | | |
|---|---|---|
| 2 | 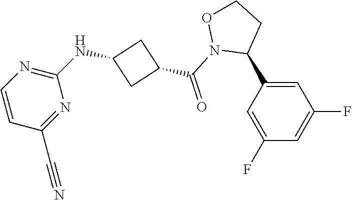 [SMILES: N#Cc1ccnc(N[C@H]2C[C@@H](C(=O)N3OCC[C@H]3c3cc(F)cc(F)c3)C2)n1] | 1H NMR (400.13 MHz, DMSO-d6) δ ppm 8.55 (br s, 1 H), 8.16 (br d, J = 7.34 Hz, 1 H), 7.13 (m, 1 H), 7.10 (d, J = 4.71 Hz, 1 H), 7.00 (br d, J = 6.53 Hz, 2 H), 5.33 (dd, J = 8.19, 6.43 Hz, 1 H), 4.28 (br s, 1 H), 4.21 (m, 1 H), 3.78 (m, 1 H), 3.21 (br s, 1 H), 2.87 (m, 1 H), 2.16 (m, 3 H), 1.24 (s, 1 H). LC/MS: m/z [M + H]⁺ 386.23, Rt (λ = 220 nm): 2.15 min (LC/MS method A), calc. m/z: 386.38 |

| | | |
|---|---|---|
| 3 | 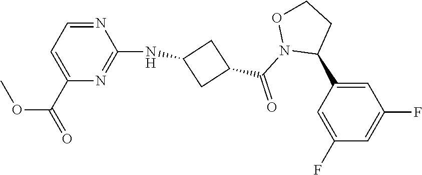 [SMILES: COC(=O)c1ccnc(N[C@H]2C[C@@H](C(=O)N3OCC[C@H]3c3cc(F)cc(F)c3)C2)n1] | 1H NMR (DMSO-d6) δ: 8.51 (br d, J = 3.9 Hz, 2H), 7.99 (br s, 1H), 6.97- 7.16 (m, 8H), 5.33 (dd, J = 8.7, 6.2 Hz, 2H), 4.34 (br d, J = 6.0 Hz, 2H), 4.21 (td, J = 7.7, 2.8 Hz, 2H), 3.85 (s, 6H), 3.75-3.82 (m, 2H), 3.11-3.27 (m, 2H), 2.87 (dddd, J = 12.2, 9.1, 6.5, 2.9 Hz, 2H), 2.45-2.47 (m, 1H), 2.06-2.27 (m, 6H), 1.13-1.34 (m, 1H). LC/MS: m/z [M + H]⁺ 419.25, Rt (λ = 220 nm): 2.04 min (LC/MS method |
| | | A), calc. m/z: 419.40 |

| | | |
|---|---|---|
| 4 | 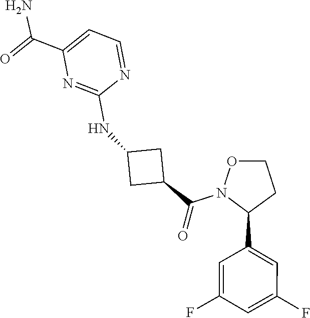 [SMILES: NC(=O)c1ccnc(N[C@H]2C[C@H](C(=O)N3OCC[C@H]3c3cc(F)cc(F)c3)C2)n1] | 1H NMR (400.13 MHz, DMSO-d6) δ ppm 8.48 (d, J = 4.77 Hz, 1 H), 7.75 (br s, 2 H), 7.14 (tt, J = 9.32, 9.32, 2.26, 2.26 Hz, 1 H), 7.04 (m, 3 H), 5.36 (m, 1 H), 4.46 (br d, J = 6.84 Hz, 1 H), 4.20 (td, J = 7.81, 7.81, 2.76 Hz, 1 H), 3.82 (m, 1 H), 2.88 (m, 1 H), 2.56 (br s, 1 H), 2.31 (m, 1 H), 2.19 (m, 2 H). LC/MS: m/z [M + H]⁺ 404.28, Rt (λ = 220 nm): 1.72 min (LC/MS method A), calc. m/z: 404.39 |

| | | |
|---|---|---|
| 5 | 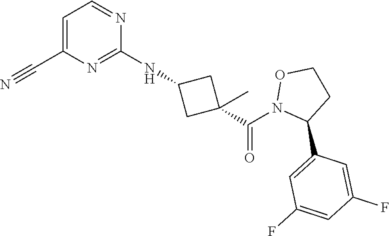 [SMILES: C[C@]1(C(=O)N2OCC[C@H]2c2cc(F)cc(F)c2)C[C@@H](Nc2nccc(C#N)n2)C1] | ¹H NMR (400 MHz, DMSO-d₆): δ ppm 8.54 (br s, 1 H), 8.07 (d, J = 7.09 Hz, 1 H), 7.14 (m, 1 H), 7.09 (d, J = 4.65 Hz, 1 H), 6.99 (br d, J = 6.48 Hz, 2 H), 5.32 (dd, J = 8.62, 6.66 Hz, 1 H), 4.34 (br s, 1 H), 4.23 (td, J = 7.67, 7.67, 2.75 Hz, 1 H), 3.84 (m, 1 H), 2.89 (m, 1 H), 2.43 (m, 1 H), 2.21 (m, 4 H), 1.45 (s, 3 H). LC/MS: m/z [M + H]⁺ 400.32, Rt (λ = 220 nm): 2.25 min (LC/MS method A), calc. m/z: 400.40 |

| | | |
|---|---|---|
| 6 | 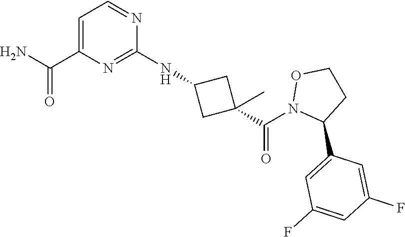 [SMILES: C[C@]1(C(=O)N2OCC[C@H]2c2cc(F)cc(F)c2)C[C@@H](Nc2nccc(C(N)=O)n2)C1] | ¹H NMR (400 MHz, DMSO-d₆): δ ppm 8.46 (br s, 1 H), 7.75 (br s, 1 H), 7.59 (br s, 1 H), 7.09 (m, 3 H), 6.99 (br d, J = 6.48 Hz, 2 H), 5.33 (dd, J = 8.50, 6.66 Hz, 1 H), 4.50 (br s, 1 H), 4.23 (td, J = 7.67, 2.63 Hz, 1 H), 3.85 (m, 1 H), 2.89 (m, 1 H), 2.43 (s, 1 H), 2.25 (m, 4 H), 1.46 (s, 3 H). LC/MS: m/z [M + H]⁺ 418.34, Rt (λ = 220 nm): 1.84 min (LC/MS method A), calc. m/z: 418.42. |

| | | |
|---|---|---|
| 7 | 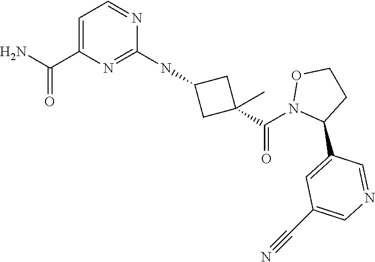 [SMILES: C[C@]1(C(=O)N2OCC[C@H]2c2cncc(C#N)c2)C[C@@H](Nc2nccc(C(N)=O)n2)C1] | 1H NMR (400.23 MHz, DMSO-d6) δ ppm 8.96 (d, J = 1.96 Hz, 1 H), 8.79 (d, J = 2.08 Hz, 1 H), 8.46 (br s, 1 H), 8.20 (t, J = 2.02, 2.02 Hz, 1 H), 8.05 (br s, 1 H), 7.75 (br s, 1 H), 7.56 (br s, 1 H), 7.05 (d, J = 4.77 Hz, 1 H), 5.40 (dd, J = 8.44, 6.97 Hz, 1 H), 4.58 (br s, 1 H), 4.28 (td, J = 7.67, 7.67, 2.75 Hz, 1 H), 3.90 (m, 1 H), 2.92 (dddd, J = 12.17, 9.08, 6.20, 2.87 Hz, 1 H), 2.44 (s, 1 H), 2.28 (m, 4 H), 1.47 (br s, 3 H). LC/MS: m/z [M + H]⁺ 408.4, Rt (λ = 220 nm): 1.37 min (LC/MS method A), calc. m/z: 408.43. |

| | | |
|---|---|---|
| 8 | 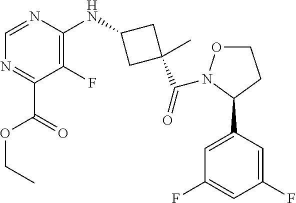 [SMILES: CCOC(=O)c1ncnc(N[C@H]2C[C@@](C)(C(=O)N3OCC[C@H]3c3cc(F)cc(F)c3)C2)c1F] | ¹H NMR (400 MHz, DMSO-d₆): δ ppm 8.29 (d, J = 1.96 Hz, 1 H), 8.22 (br d, J = 7.09 Hz, 1 H), 7.14 (m, 1 H), 7.00 (br d, J = 6.60 Hz, 2 H), 5.33 (m, 1 H), 4.59 (m, 1 H), 4.33 (q, J = 7.09 Hz, 2 H), 4.24 (m, 1 H), 3.84 (m, 1 H), 2.90 (m, 1 H), 2.57 (m, 1 H), 2.39 (m, 1 H), 2.26 (m, 3 H), 1.46 (s, 3 H), 1.30 (t, J = 7.09 Hz, 3 H). LC/MS: m/z [M + H]⁺ 465.38, Rt (λ = 220 nm): 2.15 min (LC/MS method A), calc. m/z: 465.45. |

| | | |
|---|---|---|
| 9 | 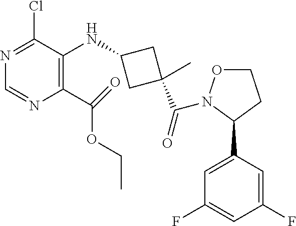 [SMILES: CCOC(=O)c1ncnc(Cl)c1N[C@H]1C[C@@](C)(C(=O)N2OCC[C@H]2c2cc(F)cc(F)c2)C1] | ¹H NMR (400 MHz, DMSO-d₆): δ ppm 8.33 (s, 1 H), 7.13 (m, 1 H), 6.99 (br d, J = 6.48 Hz, 2 H), 6.62 (d, J = 7.46 Hz, 1 H), 5.31 (dd, J = 8.50, 6.66 Hz, 1 H), 4.38 (q, J = 7.09 Hz, 2 H), 4.22 (m, 1 H), 4.02 (m, 1 H), 3.86 (m, 1 H), 2.89 (m, 1 H), 2.44 (m, 1 H), 2.30 (m, 3 H), 2.19 (m, 1 H), 1.35 (m, 6 H). LC/MS: m/z [M + H]⁺ 481.34, Rt (λ = 220 nm): 2.42 min (LC/MS method A), calc. m/z: 481.90. |
| 10 | 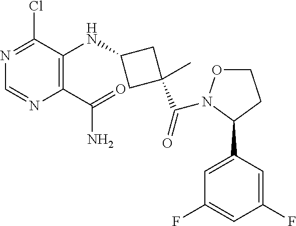 [SMILES: C[C@]1(C(=O)N2OCC[C@H]2c2cc(F)cc(F)c2)C[C@@H](Nc2c(Cl)ncnc2C(N)=O)C1] | ¹H NMR (400 MHz, DMSO-d₆): δ ppm 8.40 (br s, 1 H), 8.34 (m, 2 H), 7.97 (br s, 1 H), 7.13 (m, 1 H), 6.97 (m, 2 H), 5.32 (dd, J = 8.50, 6.79 Hz, 1 H), 4.39 (m, 1 H), 4.22 (m, 1 H), 3.88 (m, 1 H), 2.90 (m, 1 H), 2.42 (m, 1 H), 2.31 (m, 2 H), 2.22 (m, 1 H), 2.16 (m, 1 H), 1.43 (s, 3 H). LC/MS: m/z [M + H]⁺ 452.34, Rt (λ = 220 nm): 2.23 min (LC/MS method A), calc. m/z: 452.86. |

| | | |
|---|---|---|
| 11 | 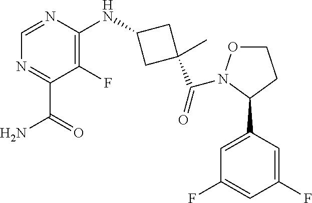 [SMILES: C[C@]1(C(=O)N2OCC[C@H]2c2cc(F)cc(F)c2)C[C@@H](Nc2ncnc(C(N)=O)c2F)C1] | ¹H NMR (400 MHz, DMSO-d₆): δ ppm 8.25 (d, J = 1.96 Hz, 1 H), 8.09 (br d, J = 6.85 Hz, 1 H), 7.94 (br s, 1 H), 7.70 (br s, 1 H), 7.14 (m, 1 H), 7.00 (m, 2 H), 5.33 (dd, J = 8.62, 6.66 Hz, 1 H), 4.59 (m, 1 H), 4.24 (td, J = 7.70, 2.81 Hz, 1 H), 3.85 (m, 1 H), 2.90 (m, 1 H), 2.57 (m, 1 H), 2.39 (m, 1 H), 2.24 (m, 3 H), 1.46 (s, 3 H). LC/MS: m/z [M + H]⁺ 436.31, Rt (λ = 220 nm): 1.83 min (LC/MS method A), calc. m/z: 436.41. |

| | | |
|---|---|---|
| 12 | 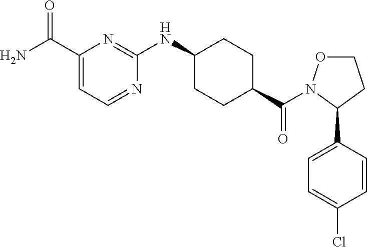 [SMILES: NC(=O)c1ccnc(N[C@H]2CC[C@@H](C(=O)N3OCC[C@H]3c3ccc(Cl)cc3)CC2)n1] | 1H NMR (600.05 MHz, DMSO-d6) δ ppm 8.47 (br s, 1 H), 7.86 (br s, 1 H), 7.73 (br s, 1 H), 7.40 (m(para), 2 H), 7.30 (m(para), J = 8.44 Hz, 2 H), 7.03 (d, J = 4.77 Hz, 1 H), 5.32 (m, 1 H), 4.24 (td, J = 7.66, 7.66, 2.84 Hz, 1 H), 4.01 (br s, 1 H), 3.86 (m, 4 H), 3.67 (br s, 28 H), 2.86 (m, 2 H), 2.15 (m, 1 H), 1.88 (br d, J = 4.22 Hz, 2 H), 1.79 (br d, J = 9.17 Hz, 1 H), 1.69 (m, 3 H), 1.61 (br s, 1 H), 1.52 (br s, 1 H). LC/MS: m/z [M + H]⁺ 430.36, Rt (λ = 220 nm): 2 min (LC/MS method A), calc. m/z: 430.91. |

| | | |
|---|---|---|
| 13 | 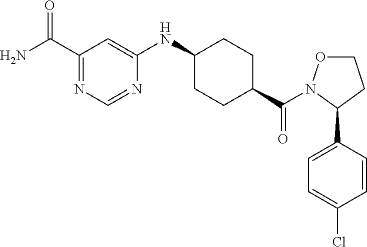 [SMILES: NC(=O)c1cc(N[C@H]2CC[C@@H](C(=O)N3OCC[C@H]3c3ccc(Cl)cc3)CC2)ncn1] | 1H NMR (600.05 MHz, DMSO-d6) δ ppm 8.53 (s, 1 H), 8.28 (br s, 1 H), 8.16 (br s, 1 H), 7.84 (br s, 1 H), 7.41 (m(para), 2 H), 7.30 (m(para), J = 8.44 Hz, 2 H), 7.17 (s, 1 H), 5.32 (m, 1 H), 4.25 (td, J = 7.66, 7.66, 2.84 Hz, 2 H), 4.14 (br s, 1 H), 3.87 (m, 7 H), 3.72 (br s, 11 H), 2.90 (br d, J = 3.12 Hz, 1 H), 2.87 (ddt, J = 11.99, 6.07, 2.98, 2.98 Hz, 2 H), 2.15 (m, 1 H), 1.86 (br s, 2 H), 1.78 (br s, 1 H), 1.73 (br d, J = 4.22 Hz, 1 H), 1.70 (br d, J = 13.02 Hz, 3 H), 1.53 (br s, 1 H). LC/MS: m/z [M + H]⁺ 430.36, Rt (λ = 220 nm): 1.86 min (LC/MS method |
| | | A), calc. m/z: 430.91 |

| | | |
|---|---|---|
| 14 | 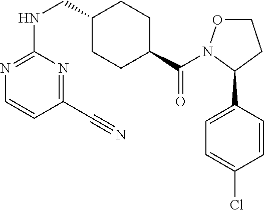 [SMILES: N#Cc1ccnc(NC[C@H]2CC[C@H](C(=O)N3OCC[C@H]3c3ccc(Cl)cc3)CC2)n1] | 1H NMR (600.05 MHz, DMSO-d6) δ ppm 8.55 (br s, 1 H), 8.51 (br s, 1 H), 7.88 (br s, 1 H), 7.40 (m(para), J = 8.44 Hz, 2 H), 7.28 (br m(para), J = 8.44 Hz, 2 H), 7.05 (br s, 1 H), 5.29 (br t, J = 6.60, 6.60 Hz, 1 H), 4.24 (m, 1 H), 3.84 (m, 1 H), 3.56 (br s, 13 H), 3.13 (br d, J = 17.24 Hz, 2 H), 2.85 (m, 1 H), 2.67 (br s, 1 H), 2.15 (m, 1 H), 1.87 (br d, J = 12.10 Hz, 1 H), 1.79 (br s, 2 H), 1.70 (br s, 1 H), 1.52 (br s, 1 H), 1.30 (br d, J = 10.45 Hz, 2 H), 1.01 (br s, 2 H). |
| | | LC/MS: m/z [M + H]⁺ 426.32, Rt ()= |
| | | 220 nm): 2.42 min (LC/MS method |
| | | A), calc. m/z: 426.92. |

| | | |
|---|---|---|
| 15 | 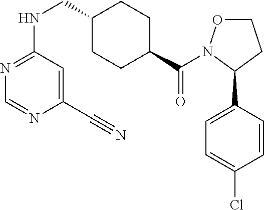 [SMILES: N#Cc1cc(NC[C@H]2CC[C@H](C(=O)N3OCC[C@H]3c3ccc(Cl)cc3)CC2)ncn1] | 1H NMR (600.05 MHz, DMSO-d6) δ ppm 8.48 (s, 1 H), 8.09 (br s, 1 H), 7.40 (d, J = 8.62 Hz, 2 H), 7.28 (d, J = 8.25 Hz, 2 H), 6.96 (s, 1 H), 5.29 (br s, 1 H), 4.24 (m, 1 H), 3.84 (m, 1 H), 3.21 (br t, J = 6.14, 6.14 Hz, 4 H), 2.85 (br dd, J = 6.60, 3.12 Hz, 1 H), 2.61 (d, J = 1.65 Hz, 1 H), 2.38 (m, 1 H), 2.15 (m, 1 H), 1.88 (br d, J = 11.19 Hz, 1 H), 1.78 (br s, 3 H), 1.52 (br s, 1 H), 1.30 (m, 2 H), 1.03 (m, 2 H). LC/MS: m/z [M + H]⁺ 426.32, Rt (λ = 220 nm): 2.23 min (LC/MS method |
| | | A), calc. m/z: 426.92. |

| | | |
|---|---|---|
| 16 | 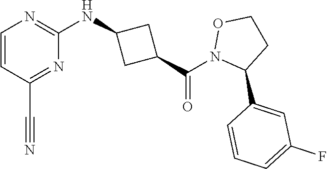 [SMILES: N#Cc1ccnc(N[C@H]2C[C@@H](C(=O)N3OCC[C@H]3c3cccc(F)c3)C2)n1] | 1H NMR (600.05 MHz, DMSO-d6) δ ppm 8.54 (br s, 1 H), 8.16 (br d, J = 7.33 Hz, 1 H), 7.40 (m, 1 H), 7.10 (m, 4 H), 5.32 (m, 1 H), 4.28 (br s, 1 H), 4.22 (br s, 1 H), 3.78 (q, J = 8.01, 8.01, 8.01 Hz, 1 H), 3.22 (br s, 1 H), 2.87 (m, 1 H), 2.38 (s, 1 H), 2.17 (m, 3 H). LC/MS: m/z [M + H]⁺ 368.29, Rt (λ = 220 nm): 2.04 min (LC/MS method A), calc. m/z: 368.38. |

| | | |
|---|---|---|
| 17 | 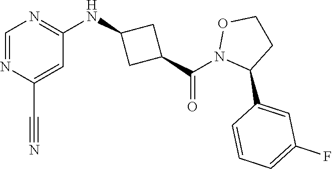 [SMILES: N#Cc1cc(N[C@H]2C[C@@H](C(=O)N3OCC[C@H]3c3cccc(F)c3)C2)ncn1] | 1H NMR (600.05 MHz, DMSO-d6) δ ppm 8.50 (s, 1 H), 8.42 (br d, J = 7.15 Hz, 1 H), 7.40 (m, 1 H), 7.11 (m, 3 H), 6.88 (s, 1 H), 5.33 (m, 1 H), 4.46 (br s, 1 H), 4.22 (m, 1 H), 3.79 (m, 3 H), 3.61 (br s, 18 H), 3.29 (br s, 1 H), 2.88 (m, 1 H), 2.61 (br s, 1 H), 2.17 (m, 2 H), 2.09 (br s, 1 H). LC/MS: m/z [M + H]⁺ 368.33, Rt (λ = 220 nm): 1.86 min (LC/MS method A), calc. m/z: 368.38. |

| | | |
|---|---|---|
| 18 | 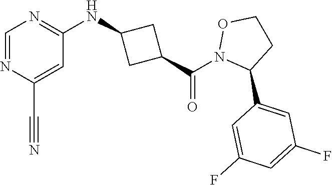 [SMILES: N#Cc1cc(N[C@H]2C[C@@H](C(=O)N3OCC[C@H]3c3cc(F)cc(F)c3)C2)ncn1] | 1H NMR (600.05 MHz, DMSO-d6) δ ppm 8.50 (s, 1 H), 8.41 (br d, J = 6.97 Hz, 1 H), 7.14 (br t, J = 9.26, 9.26 Hz, 1 H), 7.00 (br d, J = 6.97 Hz, 2 H), 6.88 (s, 1 H), 5.33 (m, 1 H), 4.46 (br d, J = 7.52 Hz, 1 H), 4.22 (m, 1 H), 3.79 (m, 1 H), 3.56 (br s, 24 H), 3.29 (br s, 1 H), 2.87 (m, 1 H), 2.61 (br s, 1 H), 2.17 (m, 2 H), 2.09 (br d, J = 9.17 Hz, 1 H). LC/MS: m/z [M + H]⁺ 386.31, Rt (λ = 220 nm): 1.95 min (LC/MS method A), calc. m/z: 386.38. |

| | | |
|---|---|---|
| 19 | 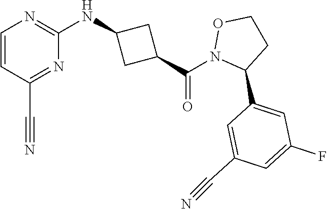 [SMILES: N#Cc1cc(F)cc([C@@H]2CCON2C(=O)[C@H]2C[C@@H](Nc3nccc(C#N)n3)C2)c1] | 1H NMR (600.05 MHz, DMSO-d6) δ ppm 8.54 (br s, 1 H), 8.16 (br d, J = 7.15 Hz, 1 H), 7.78 (s, 1 H), 7.77 (s, 1 H), 7.62 (s, 1 H), 7.50 (br d, J = 9.72 Hz, 1 H), 7.10 (d, J = 4.77 Hz, 1 H), 5.37 (dd, J = 8.62, 6.60 Hz, 1 H), 4.29 (br s, 1 H), 4.23 (m, 1 H), 3.80 (m, 1 H), 3.23 (br s, 1 H), 2.89 (dddd, J = 12.13, 9.10, 6.46, 2.93 Hz, 1 H), 2.38 (br d, J = 1.83 Hz, 1 H), 2.22 (m, 2 H), 2.11 (q, J = 9.66, 9.66, 9.66 Hz, 1 H). LC/MS: m/z [M + H]⁺ 393.33, Rt (λ = 220 nm): 1.99 min (LC/MS method |
| | | A), calc. m/z: 393.39. |

| | | |
|---|---|---|
| 20 | 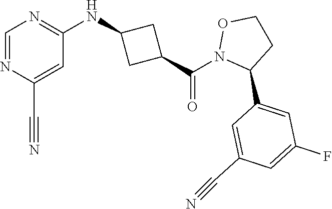 [SMILES: N#Cc1cc(F)cc([C@@H]2CCON2C(=O)[C@H]2C[C@@H](Nc3cc(C#N)ncn3)C2)c1] | 1H NMR (600.05 MHz, DMSO-d6) δ ppm 8.50 (s, 1 H), 8.41 (br d, J = 6.60 Hz, 1 H), 7.78 (br d, J = 8.44 Hz, 1 H), 7.62 (s, 1 H), 7.50 (br d, J = 9.17 Hz, 1 H), 6.88 (s, 1 H), 5.37 (m, 1 H), 4.46 (br d, J = 6.60 Hz, 1 H), 4.23 (m, 1 H), 3.81 (q, J = 8.01, 8.01, 8.01 Hz, 1 H), 2.89 (m, 1 H), 2.61 (br d, J = 1.83 Hz, 1 H), 2.39 (m, 1 H), 2.21 (m, 2 H), 2.09 (br d, J = 8.80 Hz, 1 H). LC/MS: m/z [M + H]⁺ 393.33, Rt (λ = 220 nm): 1.81 min (LC/MS method A), calc. m/z: 393.39. |

| | | |
|---|---|---|
| 21 | 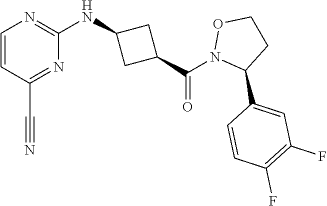 [SMILES: N#Cc1ccnc(N[C@H]2C[C@@H](C(=O)N3OCC[C@H]3c3ccc(F)c(F)c3)C2)n1] | 1H NMR (600.05 MHz, DMSO-d6) δ ppm 8.54 (br s, 1 H), 8.16 (br d, J = 7.15 Hz, 1 H), 7.41 (m, 1 H), 7.32 (t, J = 9.27, 9.27 Hz, 1 H), 7.15 (br s, 1 H), 7.10 (d, J = 4.58 Hz, 1 H), 5.31 (m, 1 H), 4.27 (br s, 1 H), 4.22 (m, 1 H), 3.78 (m, 1 H), 3.21 (br s, 1 H), 2.85 (m, 1 H), 2.39 (br d, J = 1.65 Hz, 1 H), 2.17 (m, 3 H). LC/MS: m/z [M + H]⁺ 386.3, Rt (λ = 220 nm): 2.11 min (LC/MS method A), calc. m/z: 386.38. |

| | | |
|---|---|---|
| 22 | 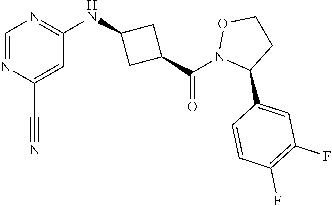 [SMILES: N#Cc1cc(N[C@H]2C[C@@H](C(=O)N3OCC[C@H]3c3ccc(F)c(F)c3)C2)ncn1] | 1H NMR (600.05 MHz, DMSO-d6) δ ppm 8.50 (s, 1 H), 8.41 (br d, J = 7.15 Hz, 1 H), 7.41 (m, 1 H), 7.32 (t, J = 9.34, 9.34 Hz, 1 H), 7.14 (br d, J = 8.44 Hz, 1 H), 6.88 (s, 1 H), 5.31 (m, 1 H), 4.45 (br d, J = 6.79 Hz, 1 H), 4.22 (m, 1 H), 3.79 (m, 1 H), 3.54 (br s, 19 H), 3.27 (br s, 1 H), 2.86 (dddd, J = 12.06, 9.08, 6.46, 3.03 Hz, 1 H), 2.17 (m, 2 H), 2.08 (br d, J = 9.35 Hz, 1 H). LC/MS: m/z [M + H]⁺ 386.32, Rt (λ = 220 nm): 1.93 min (LC/MS method A), calc. m/z: 386.38. |

| | | |
|---|---|---|
| 23 | 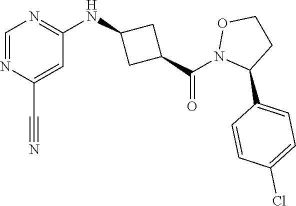 [SMILES: N#Cc1cc(N[C@H]2C[C@@H](C(=O)N3OCC[C@H]3c3ccc(Cl)cc3)C2)ncn1] | 1H NMR (600.05 MHz, DMSO-d6) δ ppm 8.50 (s, 1 H), 8.41 (br d, J = 6.79 Hz, 1 H), 7.41 (m(para), J = 8.44 Hz, 2 H), 7.30 (m(para), J = 8.25 Hz, 2 H), 6.88 (s, 1 H), 5.30 (br t, J = 7.24, 7.24 Hz, 1 H), 4.45 (br s, 1 H), 4.22 (br s, 1 H), 3.79 (m, 1 H), 2.86 (m, 1 H), 2.61 (s, 1 H), 2.39 (s, 1 H), 2.13 (m, 3 H). LC/MS: m/z [M + H]⁺ 384.27, Rt (λ = 220 nm): 2.03 min (LC/MS method A), calc. m/z: 384.84. |

| | | |
|---|---|---|
| 24 | 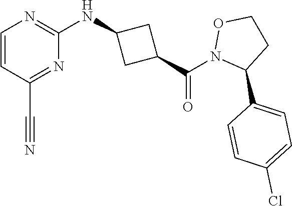 [SMILES: N#Cc1ccnc(N[C@H]2C[C@@H](C(=O)N3OCC[C@H]3c3ccc(Cl)cc3)C2)n1] | 1H NMR (DMSO-d6) δ: 8.51 (brs, 1H), 8.19 (br d, 1H), 7.41 (d, 2H), 7.31 (d, 2H), 7. 09 (m, 1H), 5.30 (dd, J = 8.7, 6.2 Hz, 2H), 4.30 (br m, 1H), 4.21 (m, 1H), 3.75 (m, 1H), 3.11-3.27 (m, 2H), 2.81 (m, 1H), 2.45-2.47 (m, 1H), 2.2-2.20 (m, 4H). LC/MS: m/z [M + H]⁺ 384.25, Rt (λ = 220 nm): 2.2 min (LC/MS method A), calc. m/z: 384.84. |

| | | |
|---|---|---|
| 25 | 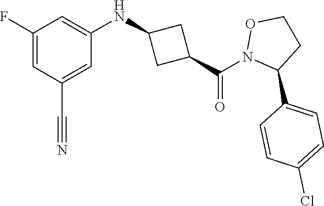 [SMILES: N#Cc1cc(F)cc(N[C@H]2C[C@@H](C(=O)N3OCC[C@H]3c3ccc(Cl)cc3)C2)c1] | ¹H NMR (600 MHz, DMSO-d₆): δ ppm 7.41 (d, J = 8.25 Hz, 2 H), 7.30 (d, J = 8.44 Hz, 2 H), 6.81 (m, 2 H), 6.71 (s, 1 H), 6.60 (br d, J = 12.10 Hz, 1 H), 5.30 (m, 1 H), 4.22 (br s, 1 H), 3.88 (br s, 1 H), 3.79 (m, 1 H), 3.28 (m, 1 H), 2.86 (m, 1 H), 2.60 (m, 2 H), 2.15 (m, 1 H), 2.02 (qm, 1 H), 1.95 (m, 1 H) LC/MS: m/z [M + H]⁺ 400.29, Rt (λ = 220 nm): 2.5 min (LC/MS method A), calc. m/z: 400.85. |

| | | |
|---|---|---|
| 26 | 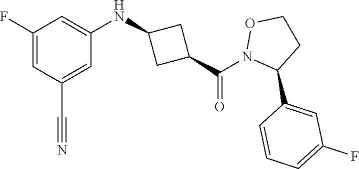 [SMILES: N#Cc1cc(F)cc(N[C@H]2C[C@@H](C(=O)N3OCC[C@H]3c3cccc(F)c3)C2)c1] | 1H NMR (600.05 MHz, DMSO-d6) δ ppm 7.39 (m, 1 H), 7.11 (m, 3 H), 6.82 (m, 1 H), 6.81 (br s, 1 H), 6.71 (s, 1 H), 6.60 (br d, J = 12.10 Hz, 1 H), 5.32 (m, 1 H), 4.22 (br s, 1 H), 3.88 (br s, 1 H), 3.80 (m, 1 H), 2.87 (dddd, J = 12.04, 9.06, 6.51, 3.03 Hz, 1 H), 2.61 (m, 2 H), 2.18 (m, 1 H), 2.04 (q, J = 9.90, 9.90, 9.90 Hz, 1 H), 1.95 (m, 1 H). LC/MS: m/z [M + H]⁺ 384.29, Rt (λ = 220 nm): 2.37 min (LC/MS method |
| | | A), calc. m/z: 384.40. |

| | | |
|---|---|---|
| 27 | 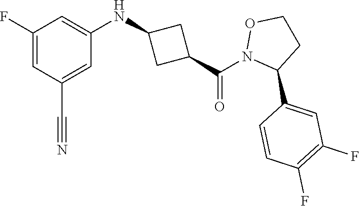 [SMILES: N#Cc1cc(F)cc(N[C@H]2C[C@@H](C(=O)N3OCC[C@H]3c3ccc(F)c(F)c3)C2)c1] | 1H NMR (600.05 MHz, DMSO-d6) δ ppm 7.41 (m, 1 H), 7.32 (t, J = 9.58, 9.58 Hz, 1 H), 7.14 (br s, 1 H), 6.82 (br d, J = 8.25 Hz, 2 H), 6.71 (s, 1 H), 6.60 (br d, J = 11.92 Hz, 1 H), 5.31 (m, 1 H), 4.22 (br s, 1 H), 3.89 (br d, J = 6.97 Hz, 1 H), 3.79 (m, 1 H), 3.28 (m, 2 H), 2.86 (dddd, J = 12.20, 9.08, 6.33, 2.84 Hz, 1 H), 2.61 (m, 2 H), 2.18 (m, 1 H), 2.03 (q, J = 9.66, 9.66, 9.66 Hz, 1 H), 1.95 (m, 1 H). LC/MS: m/z [M + H]⁺ 402.3, Rt (λ = 220 nm): 2.42 min (LC/MS method |
| | | A), calc. m/z: 402.39. |

| | | |
|---|---|---|
| 28 | 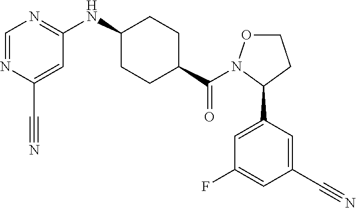 [SMILES: N#Cc1cc(F)cc([C@@H]2CCON2C(=O)[C@H]2CC[C@@H](Nc3cc(C#N)ncn3)CC2)c1] | ¹H NMR (600 MHz, DMSO-d₆): δ ppm 8.48 (s, 1 H), 8.03 (br d, J = 7.34 Hz, 1 H), 7.77 (d, J = 8.40 Hz, 1 H), 7.61 (s, 1 H), 7.48 (br d, J = 9.72 Hz, 1H), 7.00 (s, 1 H), 5.39 (dd, J = 8.44, 6.79 Hz, 1 H), 4.26 (td, J = 7.70, 7.70, 2.93 Hz, 1 H), 4.12 (br s, 1 H), 3.90 (m, 1 H), 2.90 (m, 2 H), 2.85 (br s, 1 H), 2.22 (m, 1 H), 1.81 (br s, 2 H), 1.76 (br d, J = 13.02 Hz, 2 H), 1.73 (br d, J = 8.25 Hz, 1 H), 1.68 (m, 2 H), 1.55 (br s, 1 H). LC/MS: m/z [M + H]⁺ 421.35, Rt (λ = 220 nm): 2.05 min (LC/MS method |
| | | A), calc. m/z: 421.45. |

| | | |
|---|---|---|
| 29 | 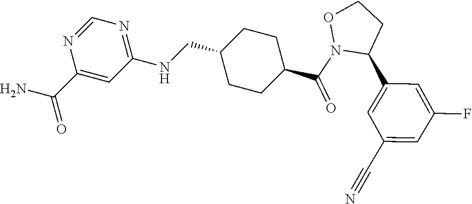 [SMILES: N#Cc1cc(F)cc([C@@H]2CCON2C(=O)[C@H]2CC[C@H](CNc3cc(C(N)=O)ncn3)CC2)c1] | 1H NMR (600.05 MHz, DMSO-d6) δ ppm 8.52 (s, 1 H), 8.35 (br s, 1 H), 8.18 (br s, 1 H), 7.84 (br s, 1 H), 7.77 (br d, J = 8.44 Hz, 1 H), 7.60 (s, 1 H), 7.47 (br d, J = 9.54 Hz, 1 H), 7.10 (s, 1 H), 5.37 (br t, J = 7.43, 7.43 Hz, 1 H), 4.26 (td, J = 7.61, 7.61, 2.75 Hz, 1 H), 3.87 (m, 2 H), 3.27 (br s, 2 H), 3.14 (br s, 1 H), 2.88 (dddd, J = 12.15, 9.17, 6.56, 3.12 Hz, 1 H), 2.70 (br s, 1 H), 2.22 (m, 1 H), 1.93 (br d, J = 12.47 Hz, 1 H), 1.81 (br s, 2 H), 1.74 (br d, J = 9.72 Hz, 1 H), 1.55 (brs, 1H), 1.32 |
| | | (m, 2H), 1.02 m, 2H). |
| | | LC/MS: m/z [M − H]⁻ 451.3, Rt (λ = 220 |
| | | nm): 1.6 min (LC/MS method A), calc. |
| | | m/z: 451.47. |

| | | |
|---|---|---|
| 30 | 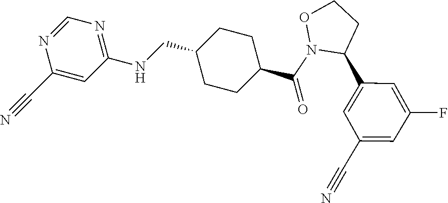 [SMILES: N#Cc1cc(F)cc([C@@H]2CCON2C(=O)[C@H]2CC[C@H](CNc3cc(C#N)ncn3)CC2)c1] | 1H NMR (600.05 MHz, DMSO-d6) δ ppm 8.48 (s, 1 H), 8.10 (m, 1 H), 7.76 (br d, J = 8.44 Hz, 1 H), 7.60 (s, 1 H), 7.47 (br d, J = 9.72 Hz, 1 H), 6.96 (s, 1 H), 5.36 (br t, J = 7.52, 7.52 Hz, 1 H), 4.26 (td, J = 7.61, 7.61, 2.75 Hz, 1 H), 3.87 (m, 1 H), 3.22 (br t, J = 5.78, 5.78 Hz, 2 H), 3.04 (br s, 1 H), 2.88 (dddd, J = 12.10, 9.12, 6.46, 3.12 Hz, 1 H), 2.69 (br s, 1 H), 2.22 (m, 1 H), 1.93 (br d, J = 13.02 Hz, 1 H), 1.80 (m, 2 H), 1.73 (br d, J = 11.00 Hz, 1 H), 1.53 (br s, 1 H), 1.32 (q, J = 12.0 Hz, 2H), 1.02 |
| | | m (1H). |
| | | LC/MS: m/z [M − H]⁻ 433.3, Rt (λ = 220 |
| | | nm): 1.96 min (LC/MS method A), |
| | | calc. m/z: 433.46. |

| | | |
|---|---|---|
| 31 | 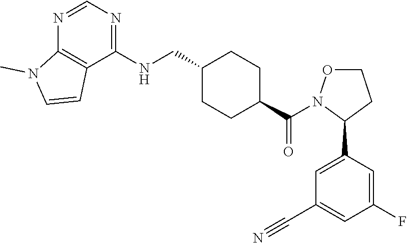 | ¹H NMR (600 MHz, DMSO-d₆): δ ppm 8.33 (s, 1 H), 7.77 (br d, J = 7.52 Hz, 1 H), 7.59 (s, 1 H), 7.47 (br d, J = 9.35 Hz, 1 H), 7.42 (br s, 1 H), 6.92 (br s, 1 H), 5.36 (br t, J = 7.24, 7.24 Hz, 1 H), 4.26 (td, J = 7.66, 7.66, 3.03 Hz, 1 H), 3.88 (m, 1 H), 3.80 (s, 3 H), 3.47 (2 H), 2.88 (m, 1 H), 2.73 (br s, 1 H), 2.61 (s, 1 H), 2.23 (m, 1 H), 1.95 (br d, J = 12.47 Hz, 1 H), 1.87 (br s, 2 H), 1.76 (br d, J = 10.82 Hz, 1 H), 1.67 (br s, 1 H), 1.35 (m, 2 H), 1.09 (m, 2 H). LC/MS: m/z [M − H + Formic acid]⁻ 507.3, Rt (λ = 220 nm): 1.37 min (LC/MS method A), calc. m/z: 507.54. |

| | | |
|---|---|---|
| 32 | 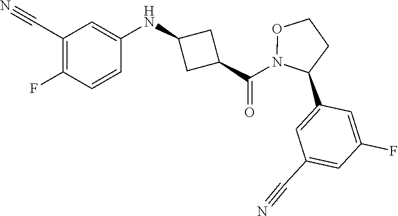 [SMILES: N#Cc1cc(F)cc([C@@H]2CCON2C(=O)[C@H]2C[C@@H](Nc3ccc(F)c(C#N)c3)C2)c1] | 1H NMR (600.05 MHz, DMSO-d6) δ ppm 7.77 (br d, J = 8.44 Hz, 1 H), 7.62 (s, 1 H), 7.50 (br d, J = 9.72 Hz, 1 H), 7.21 (t, J = 9.17, 9.17 Hz, 1 H), 6.86 (m, 1 H), 6.80 (dd, J = 4.95, 3.12 Hz, 1 H), 5.37 (dd, J = 8.44, 6.60 Hz, 1 H), 4.23 (m, 1 H), 3.82 (m, 2 H), 3.25 (br s, 2 H), 2.89 (m, 1 H), 2.60 (m, 2 H), 2.22 (m, 1 H), 2.04 (m, 1 H), 1.92 (q, J = 10.03, 10.03, 10.03 Hz, 1 H). LC/MS: m/z [M + H]⁺ 409.4, Rt (X = 220 nm): 2.26 min (LC/MS method A), calc. m/z: 409.41. |

| | | |
|---|---|---|
| 33 | 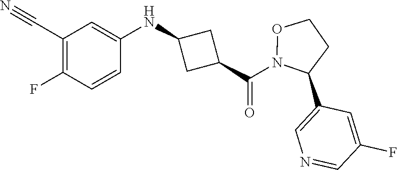 [SMILES: N#Cc1cc(N[C@H]2C[C@@H](C(=O)N3OCC[C@H]3c3cncc(F)c3)C2)ccc1F] | 1H NMR (600.05 MHz, DMSO-d6) δ ppm 8.50 (d, J = 2.57 Hz, 1 H), 8.41 (s, 1 H), 7.60 (br d, J = 10.09 Hz, 1 H), 7.21 (t, J = 9.08, 9.08 Hz, 1 H), 6.86 (m, 1 H), 6.80 (m, 1 H), 5.40 (m, 1 H), 4.25 (br s, 1 H), 3.83 (m, 2 H), 2.90 (m, 1 H), 2.61 (br d, J = 1.65 Hz, 2 H), 2.60 (br s, 1 H), 2.39 (br d, J = 1.65 Hz, 2 H), 2.27 (br d, J = 10.82 Hz, 1 H), 2.01 (br d, J = 9.54 Hz, 1 H), 1.93 (br d, J = 9.90 Hz, 1 H). |
| | | LC/MS: m/z [M + H]⁺ 385.3, Rt (λ = |
| | | 220 nm): 1.98 min (LC/MS method |
| | | A), calc. m/z: 385.39. |

| | | |
|---|---|---|
| 34 | 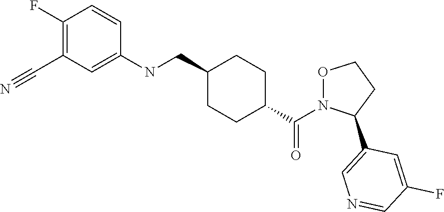 [SMILES: N#Cc1cc(NC[C@H]2CC[C@H](C(=O)N3OCC[C@H]3c3cncc(F)c3)CC2)ccc1F] | 1H NMR (600.05 MHz, DMSO-d6) δ ppm 8.49 (d, J = 2.57 Hz, 1 H), 8.40 (s, 1 H), 7.58 (br d, J = 9.90 Hz, 1 H), 7.19 (t, J = 9.17, 9.17 Hz, 1 H), 6.91 (m, 1 H), 6.84 (dd, J = 5.14, 2.93 Hz, 1 H), 5.40 (m, 1 H), 4.29 (td, J = 7.66, 7.66, 3.03 Hz, 1 H), 3.89 (m, 1 H), 2.89 (m, 2 H), 2.85 (d, J = 6.60 Hz, 2 H), 2.26 (m, 1 H), 1.88 (m, 3 H), 1.73 (br s, 1 H), 1.51 (br s, 1 H), 1.33 (m, 2 H), 1.04 (m, 2 H). LC/MS: m/z [M + H]⁺ 427.4, Rt (λ = 220 nm): 2.26 min (LC/MS method A), calc. m/z: 427.47. |

| | | |
|---|---|---|
| 35 | 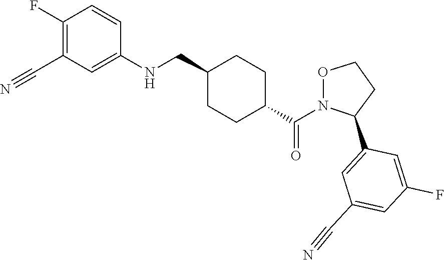 [SMILES: N#Cc1cc(F)cc([C@@H]2CCON2C(=O)[C@H]2CC[C@H](CNc3ccc(F)c(C#N)c3)CC2)c1] | ¹H NMR (600 MHz, DMSO-d₆): δ ppm 7.77 (d, J = 8.40 Hz, 1 H), 7.60 (s, 1 H), 7.47 (br d, J = 9.54 Hz, 1 H), 7.19 (t, J = 9.17, 9.17 Hz, 1 H), 6.91 (ddd, J = 9.17, 4.49, 3.03 Hz, 1 H), 6.84 (dd, J = 5.04, 3.03 Hz, 1 H), 6.07 (br s, 1 H), 5.37 (m, 1 H), 4.26 (td, J = 7.70, 7.70, 3.12 Hz, 1 H), 3.87 (m, 1 H), 2.89 (m, 1 H), 2.86 (br d, J = 6.60 Hz, 2 H), 2.72 (m, 1 H), 2.22 (m, 1 H), 1.93 (br d, J = 12.65 Hz, 1 H), 1.87 (br d, J = 8.25 Hz, 2 H), 1.74 (br d, J = 12.47 Hz, 1 H), 1.51 (m, 1 H), 1.33 (m, 2 H), 1.12 (m, 2 H). LC/MS: m/z [M + H]⁺ 451.4, Rt (λ = 220 nm): 2.51 min (LC/MS method |
| | | A), calc. m/z: 451.49. |

| | | |
|---|---|---|
| 36 | 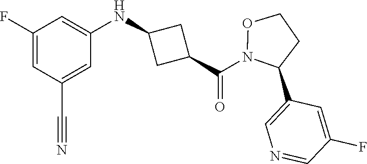 [SMILES: N#Cc1cc(F)cc(N[C@H]2C[C@@H](C(=O)N3OCC[C@H]3c3cncc(F)c3)C2)c1] | 1H NMR (400.23 MHz, DMSO-d6) δ ppm 8.50 (d, J = 2.69 Hz, 1 H), 8.41 (s, 1 H), 7.61 (br d, J = 9.78 Hz, 1 H), 6.81 (br d, J = 7.82 Hz, 2 H), 6.71 (s, 1 H), 6.60 (m, 1 H), 5.40 (dd, J = 8.62, 6.54 Hz, 1 H), 4.26 (td, J = 7.64, 7.64, 2.69 Hz, 1 H), 3.86 (td, J = 16.81, 16.81, 7.82 Hz, 2 H), 3.23 (br s, 2 H), 2.90 (dddd, J = 12.15, 9.12, 6.48, 2.93 Hz, 1 H), 2.63 (m, 2 H), 2.27 (m, 1 H), |
| | | 2.00 (m, 2 H). |
| | | LC/MS: m/z [M + H]⁺ 385.2, Rt (λ = |
| | | 220 nm): 2.06 min (LC/MS method |
| | | A), calc. m/z: 385.39. |

| | | |
|---|---|---|
| 37 | 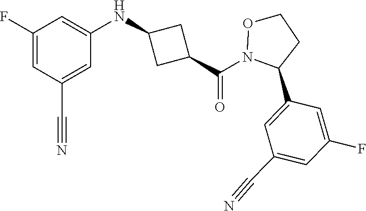 [SMILES: N#Cc1cc(F)cc(N[C@H]2C[C@@H](C(=O)N3OCC[C@H]3c3cc(F)cc(C#N)c3)C2)c1] | 1H NMR (400.23 MHz, DMSO-d6) δ ppm 7.77 (br d, J = 7.58 Hz, 1 H), 7.62 (s, 1 H), 7.50 (br d, J = 9.66 Hz, 1 H), 6.81 (br d, J = 7.70 Hz, 2 H), 6.71 (s, 1 H), 6.60 (m, 1 H), 5.37 (dd, J = 8.62, 6.42 Hz, 1 H), 4.23 (td, J = 7.70, 7.70, 2.57 Hz, 1 H), 3.89 (m, 1 H), 3.81 (m, 1 H), 3.24 (br s, 2 H), 2.89 (m, 1 H), 2.63 (m, 2 H), 2.22 (m, 1 H), 2.00 (m, 2 H). LC/MS: m/z [M + H]⁺ 409.2, Rt (λ = 220 nm): 2.33 min (LC/MS method A), calc. m/z: 409.41. |

| | | |
|---|---|---|
| 38 | 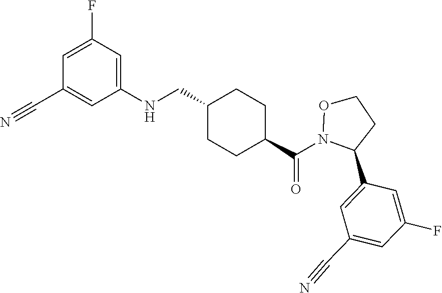 [SMILES: N#Cc1cc(F)cc(NC[C@H]2CC[C@H](C(=O)N3OCC[C@H]3c3cc(F)cc(C#N)c3)CC2)c1] | 1H NMR (400.23 MHz, DMSO-d6) δ ppm 7.76 (br d, J = 8.31 Hz, 1 H), 7.60 (s, 1 H), 7.47 (br d, J = 9.54 Hz, 1 H), 6.77 (s, 1 H), 6.76 (d, J = 10.21 Hz, 1 H), 6.66 (m, 1 H), 6.52 (br s, 1 H), 5.37 (m, 1 H), 4.26 (td, J = 7.64, 7.64, 2.93 Hz, 1 H), 3.87 (m, 1 H), 2.90 (br d, J = 6.60 Hz, 2 H), 2.86 (m, 1 H), 2.69 (m, 1 H), 2.22 (m, 1 H), 1.94 (br d, J = 12.23 Hz, 1 H), 1.86 (br d, J = 12.10 Hz, 2 H), 1.74 (br d, J = 11.62 Hz, 1 H), 1.51 (br s, 1 H), 1.34 (m, 2 H), 1.04 (qd, J = 12.82, 12.82, 12.82, 12.65 Hz, 2H). LC/MS: m/z [M + H]⁺ 451.3, Rt (λ = 220 nm): 2.56 min (LC/MS method A), calc. m/z: 451.49. |

| | | |
|---|---|---|
| 39 | 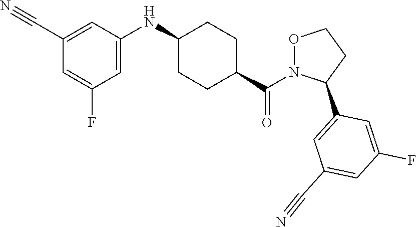 [SMILES: N#Cc1cc(F)cc(N[C@H]2CC[C@@H](C(=O)N3OCC[C@H]3c3cc(F)cc(C#N)c3)CC2)c1] | 1H NMR (400.23 MHz, DMSO-d6) δ ppm 7.77 (br d, J = 8.44 Hz, 1 H), 7.61 (s, 1 H), 7.47 (br d, J = 9.78 Hz, 1 H), 6.82 (s, 1 H), 6.72 (m, 2 H), 6.44 (br s, 1 H), 5.39 (m, 1 H), 4.26 (td, J = 7.67, 7.67, 3.00 Hz, 1 H), 3.89 (m, 1 H), 3.53 (br s, 2 H), 2.88 (m, 2 H), 2.33 (br d, J = 1.71 Hz, 1 H), 2.22 (m, 1 H), 1.83 (m, 1 H), 1.75 (br d, J = 8.93 Hz, 3 H), 1.65 (m, 3 H), 1.53 (br s, 1 H). LC/MS: m/z [M + H]⁺ 437.2, Rt (λ = 220 nm): 2.54 min (LC/MS method A), calc. m/z: 437.46. |

| | | |
|---|---|---|
| 40 | 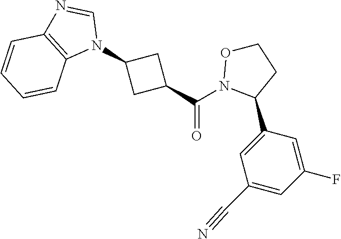 [SMILES: N#Cc1cc(F)cc([C@@H]2CCON2C(=O)[C@H]2C[C@@H](n3cnc4ccccc43)C2)c1] | 1H NMR (DMSO-d6) δ: 8.51 (br d, J = 3.9 Hz, 2H), 7.99 (br s, 1H), 6.97- 7.16 (m, 8H), 5.33 (dd, J = 8.7, 6.2 Hz, 2H), 4.34 (br d, J = 6.0 Hz, 2H), 4.21 (td, J = 7.7, 2.8 Hz, 2H), 3.85 (s, 6H), 3.75-3.82 (m, 2H), 3.11-3.27 (m, 2H), 2.87 (dddd, J = 12.2, 9.1, 6.5, 2.9 Hz, 2H), 2.45-2.47 (m, 1H), 2.06-2.27 (m, 6H), 1.13-1.34 (m, 1H). LC/MS: m/z [M + H]⁺ 391.2, Rt (λ = 220 nm): 1.38 min (LC/MS method A), calc. m/z: 391.42. |

| | | |
|---|---|---|
| 41 | 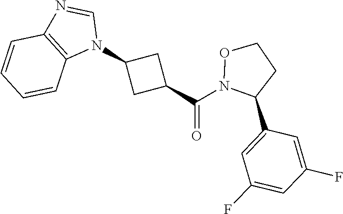 [SMILES: O=C([C@H]1C[C@@H](n2cnc3ccccc32)C1)N1OCC[C@H]1c1cc(F)cc(F)c1] | 1H NMR (DMSO-d6) δ: 9.42 (s, 1H), 7.91 (t, J = 4.9 Hz, 1H), 7.81 (t, J = 4.5 Hz, 1H), 7.50-7.57 (m, 2H), 7.13 (tt, J = 9.3, 2.2 Hz, 1H), 6.99-7.06 (m, 2H), 5.30-5.42 (m, 1H), 5.18 (quin, J = 8.3 Hz, 1H), 4.04-4.31 (m, 2H), 3.82-3.92 (m, 2H), 2.71-2.96 (m, 6H), 2.12-2.31 (m, 1H). LC/MS: m/z [M + H]⁺ 384.2, Rt (λ = 220 nm): 1.49 min (LC/MS method A), calc. m/z: 384.40. |

| | | |
|---|---|---|
| 42 | 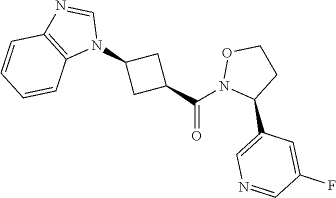 [SMILES: O=C([C@H]1C[C@@H](n2cnc3ccccc32)C1)N1OCC[C@H]1c1cncc(F)c1] | 1H NMR (400.23 MHz, DMSO-d6) δ ppm 8.49 (m, 1 H), 7.60 (m, 1 H), 4.30 (td, J = 7.67, 7.67, 2.75 Hz, 1 H), 3.94 (m, 4 H), 3.77 (m, 8 H), 3.53 (m, 1 H), 2.91 (m, 2 H), 2.79 (m, 1 H), 2.31 (m, 2H). LC/MS: m/z [M + H]⁺ 367.2, Rt (λ = 220 nm): 1.12 min (LC/MS method A), calc. m/z: 367.40. |

| | | |
|---|---|---|
| 43 | 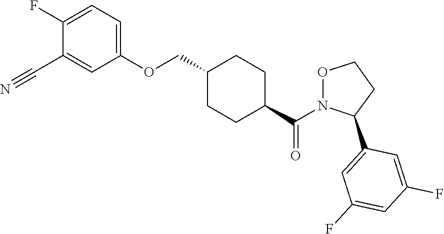 [SMILES: N#Cc1cc(OC[C@H]2CC[C@H](C(=O)N3OCC[C@H]3c3cc(F)cc(F)c3)CC2)ccc1F] | ¹H NMR (400 MHz, DMSO-d₆): δ ppm 7.49 (m, 1 H), 7.43 (t, J = 9.20, 9.20 Hz, 1 H), 7.34 (m, 1 H), 7.13 (m, 1 H), 6.98 (br d, J = 6.48 Hz, 2 H), 5.34 (m, 1 H), 4.25 (m, 1 H), 3.88 (m, 1 H), 2.87 (br dd, J = 6.66, 3.12 Hz, 1 H), 2.68 (m, 1 H), 2.19 (m, 1 H), 1.95 (br d, J = 12.23 Hz, 1 H), 1.88 (br s, 2 H), 1.73 (br s, 2 H), 1.38 (m, 2 H), 1.14 (br d, J = 12.72 Hz, 2 H). LC/MS: m/z [M + H]⁺ 445.2, Rt (λ = 220 nm): 2.72 min (LC/MS method A), calc. m/z: 445.45. |

| | | |
|---|---|---|
| 44 | 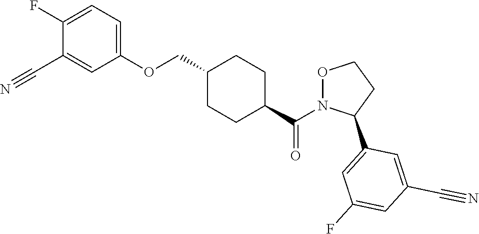 [SMILES: N#Cc1cc(F)cc([C@@H]2CCON2C(=O)[C@H]2CC[C@H](COc3ccc(F)c(C#N)c3)CC2)c1] | ¹H NMR (400 MHz, DMSO-d₆): δ ppm 7.78 (dd, J = 2.38, 1.28 Hz, 1 H), 7.60 (s, 1 H), 7.47 (m, 3 H), 7.35 (dd, J = 4.46, 3.24 Hz, 1 H), 5.38 (dd, J = 8.56, 6.48 Hz, 1 H), 4.27 (td, J = 7.73, 7.73, 2.87 Hz, 1 H), 3.89 (m, 1 H), 3.84 (d, J = 6.36 Hz, 2 H), 2.89 (m, 1 H), 2.73 (br t, J = 11.92, 11.92 Hz, 1 H), 2.22 (m, 1 H), 1.90 (m, 3 H), 1.75 (m, 2 H), 1.38 (m, 2 H), 1.15 (m, 2 H). LC/MS: m/z [M + H]⁺ 452.2, Rt (λ = 220 nm): 2.59 min (LC/MS method A), calc. m/z: 452.47. |

| | | |
|---|---|---|
| 45 | 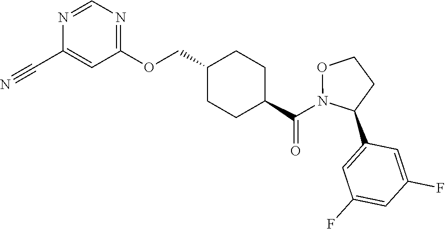 [SMILES: N#Cc1cc(OC[C@H]2CC[C@H](C(=O)N3OCC[C@H]3c3cc(F)cc(F)c3)CC2)ncn1] | ¹H NMR (400 MHz, DMSO-d₆): δ ppm 8.93 (d, J = 1.10 Hz, 1 H), 7.72 (d, J = 1.10 Hz, 1 H), 7.13 (m, 1 H), 6.98 (m, 2 H), 5.34 (dd, J = 8.62, 6.30 Hz, 1 H), 4.24 (m, 3 H), 3.87 (m, 1 H), 2.87 (m, 1 H), 2.69 (m, 1 H), 2.19 (m, 1 H), 1.95 (br d, J = 12.84 Hz, 1 H), 1.79 (m, 4 H), 1.37 (m, 2 H), 1.14 (m, 2 H). LC/MS: m/z [M + H]⁺ 429.2, Rt (λ = 220 nm): 2.46 min (LC/MS method A), calc. m/z: 429.44. |

| | | |
|---|---|---|
| 46 | 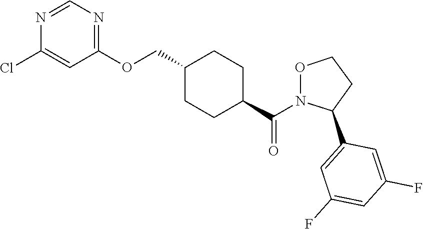 [SMILES: O=C([C@H]1CC[C@H](COc2cc(Cl)ncn2)CC1)N1OCC[C@H]1c1cc(F)cc(F)c1] | ¹H NMR (400 MHz, DMSO-d₆): δ ppm 8.66 (d, J = 0.73 Hz, 1 H), 7.18 (d, J = 0.73 Hz, 1 H), 7.13 (m, 1 H), 6.98 (m, 2 H), 5.34 (dd, J = 8.62, 6.42 Hz, 1 H), 4.24 (m, 1 H), 4.20 (d, J = 6.36 Hz, 2 H), 3.86 (m, 1 H), 2.87 (m, 1 H), 2.71 (m, 1 H), 2.18 (m, 1 H), 1.95 (m, 1 H), 1.86 (m, 2 H), 1.75 (m, 2 H), 1.37 (m 2 H), 1.14 (m, 2 H). LC/MS: m/z [M + H]⁺ 438.2, Rt (λ = 220 nm): 2.6 min (LC/MS method A), calc. m/z: 438.88. |

| | | |
|---|---|---|
| 47 | 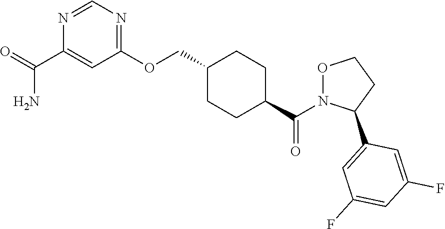 [SMILES: NC(=O)c1cc(OC[C@H]2CC[C@H](C(=O)N3OCC[C@H]3c3cc(F)cc(F)c3)CC2)ncn1] | 1H NMR (400.23 MHz, DMSO-d6) δ ppm 8.86 (d, J = 0.86 Hz, 1 H), 8.19 (br s, 1 H), 7.89 (br s, 1 H), 7.33 (s, 1 H), 7.12 (m, 1 H), 6.98 (br d, J = 6.60 Hz, 2 H), 5.34 (m, 1 H), 4.23 (br d, J = 6.48 Hz, 3 H), 3.86 (m, 1 H), 3.20 (br t, J = 6.11, 6.11 Hz, 1 H), 2.87 (m, 1 H), 2.69 (m, 1 H), 2.33 (s, 1 H), 2.19 (m, 1 H), 1.95 (br d, J = 12.72 Hz, 1 H), 1.86 (br s, 2 H), 1.77 (br s, 3 H), 1.37 (m, 3 H), 1.16 (m, 2 H). LC/MS: m/z [M + H]⁺ 447.2, Rt (λ = 220 nm): 2.12 min (LC/MS method A), calc. m/z: 447.46. |

| | | |
|---|---|---|
| 48 | 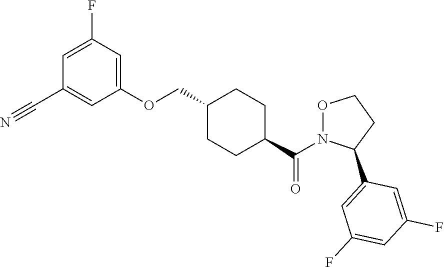 [SMILES: N#Cc1cc(F)cc(OC[C@H]2CC[C@H](C(=O)N3OCC[C@H]3c3cc(F)cc(F)c3)CC2)c1] | 1H NMR (400.23 MHz, DMSO-d6) δ ppm 7.38 (d, J = 7.91 Hz, 1 H), 7.33 (s, 1 H), 7.25 (dt, J = 11.03, 2.31, 2.31 Hz, 1 H), 7.13 (tt, J = 9.29, 9.29, 2.32, 2.32 Hz, 1 H), 6.98 (m, 2 H), 5.34 (dd, J = 8.62, 6.30 Hz, 1 H), 4.25 (td, J = 7.70, 7.70, 2.93 Hz, 1 H), 3.89 (d, J = 6.24 Hz, 2 H), 3.85 (m, 1 H), 2.87 (dddd, J = 12.09, 9.12, 6.48, 3.12 Hz, 1 H), 2.69 (m, 1 H), 2.19 (m, 1 H), 1.95 (br d, J = 12.59 Hz, 1 H), 1.88 (br d, J = 9.29 Hz, 2 H), 1.75 (br dd, J = 6.42, 2.87 Hz, 2 H), 1.39 (dtd, J = 12.81, 12.70, 12.70, 3.24 Hz, 2 H), 1.15 (m, 2H). LC/MS: m/z [M + H]⁺ 445.2, Rt (λ = |
| | | 220 nm): 2.76 min (LC/MS method |
| | | A), calc. m/z: 445.45. |

| | | |
|---|---|---|
| 49 | 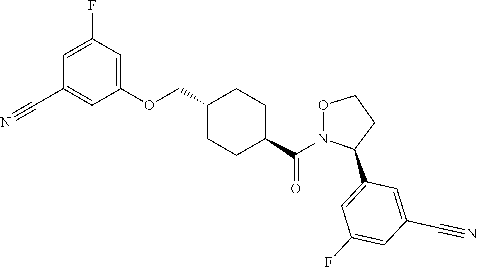 [SMILES: N#Cc1cc(F)cc(OC[C@H]2CC[C@H](C(=O)N3OCC[C@H]3c3cc(F)cc(C#N)c3)CC2)c1] | 1H NMR (400.23 MHz, DMSO-d6) δ ppm 7.77 (d, J = 7.98 Hz, 1 H), 7.60 (s, 1 H), 7.47 (d, J = 9.54 Hz, 1 H), 7.38 (d, J = 7.91 Hz, 1 H), 7.33 (s, 1 H), 7.25 (dt, J = 11.03, 2.31, 2.31 Hz, 1 H), 5.38 (dd, J = 8.56, 6.48 Hz, 1 H), 4.27 (td, J = 7.70, 7.70, 2.93 Hz, 1 H), 3.89 (d, J = 6.36 Hz, 3 H), 2.89 (dddd, J = 12.13, 9.14, 6.48, 3.06 Hz, 1 H), 2.73 (br t, J = 11.74, 11.74 Hz, 1 H), 2.22 (m, 1 H), 1.96 (br d, J = 12.84 Hz, 1 H), 1.88 (m, 2 H), 1.76 (br d, J = 11.62 Hz, 2 H), 1.38 (dtd, J = 12.84, 12.62, 12.62, 3.00 Hz, 2 H), 1.13 (m, 2H). LC/MS: m/z [M + H]⁺ 452.2, Rt (λ = 220 nm): 2.63 min (LC/MS method A), calc. m/z: 452.47. |

| | | |
|---|---|---|
| 50 | 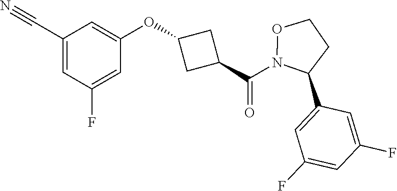 [SMILES: N#Cc1cc(F)cc(O[C@H]2C[C@H](C(=O)N3OCC[C@H]3c3cc(F)cc(F)c3)C2)c1] | 1H NMR (400.23 MHz, DMSO-d6) δ ppm 7.40 (d, J = 8.47 Hz, 1 H), 7.15 (m, 3 H), 7.04 (m, 2 H), 5.36 (dd, J = 8.68, 6.36 Hz, 1 H), 4.85 (qd, J = 6.09, 6.09, 6.09, 5.93 Hz, 1 H), 4.20 (td, J = 7.73, 7.73, 2.87 Hz, 1 H), 3.86 (m, 1 H), 3.56 (br s, 1 H), 2.89 (dddd, J = 12.10, 9.11, 6.48, 3.00 Hz, 1 H), 2.75 (m, 2 H), 2.36 (m, 2 H), 2.20 (m, 1 H). LC/MS: m/z [M + H]⁺ 403.2, Rt (λ = 220 nm): 2.55 min (LC/MS method |
| | | A), calc. m/z: 403.37. |

| | | |
|---|---|---|
| 51 | 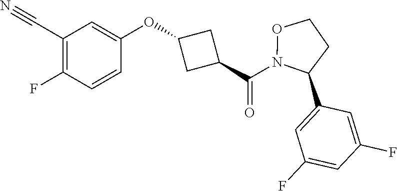 [SMILES: N#Cc1cc(O[C@H]2C[C@H](C(=O)N3OCC[C@H]3c3cc(F)cc(F)c3)C2)ccc1F] | ¹H NMR (400 MHz, DMSO-d₆): δ ppm 7.44 (t, J = 9.05, 9.05 Hz, 1 H), 7.36 (t, J = 4.37, 4.37 Hz, 1 H), 7.25 (dt, J = 9.23, 3.76, 3.76 Hz, 1 H), 7.14 (tt, J = 9.35, 9.35, 2.32, 2.32 Hz, 1 H), 7.04 (m, 2 H), 5.36 (dd, J = 8.68, 6.36 Hz, 1 H), 4.79 (m, 1 H), 4.20 (td, J = 7.73, 7.73, 2.87 Hz, 1 H), 3.85 (m, 1 H), 3.55 (br s, 1 H), 2.89 (m, 1 H), 2.72 (m, 2 H), 2.37 (m, 3 H). LC/MS: m/z [M + H]⁺ 403.2, Rt (λ = 220 nm): 2.51 min (LC/MS method |
| | | A), calc. m/z: 403.37. |

| | | |
|---|---|---|
| 52 | 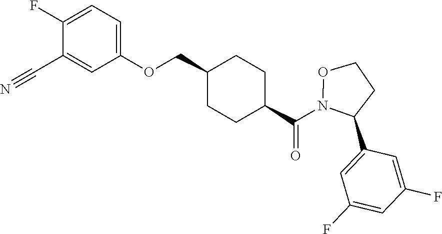 [SMILES: N#Cc1cc(OC[C@H]2CC[C@@H](C(=O)N3OCC[C@H]3c3cc(F)cc(F)c3)CC2)ccc1F] | ¹H NMR (400 MHz, DMSO-d₆): δ ppm 7.52 (dd, J = 5.07, 3.12 Hz, 1 H), 7.37 (m, 2 H), 7.12 (tt, J = 9.29, 9.29, 2.32, 2.32 Hz, 1 H), 6.97 (m, 2 H), 5.34 (dd, J = 8.62, 6.42 Hz, 1 H), 4.23 (td, J = 7.70, 7.70, 2.93 Hz, 1 H), 3.88 (m, 3 H), 2.87 (m, 2 H), 2.17 (m, 1 H), 1.94 (m, 1 H), 1.81 (m, 1 H), 1.65 (m, 4 H), 1.49 (m, 2 H). LC/MS: m/z [M + H]⁺ 445.2, Rt (λ = 220 nm): 2.79 min (LC/MS method A), calc. m/z: 445.45. |

| | | |
|---|---|---|
| 53 | 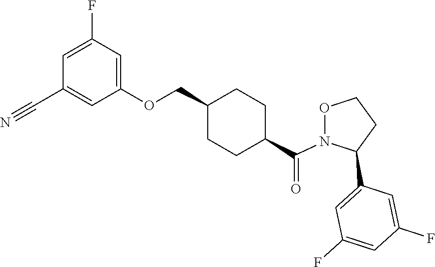 [SMILES: N#Cc1cc(F)cc(OC[C@H]2CC[C@@H](C(=O)N3OCC[C@H]3c3cc(F)cc(F)c3)CC2)c1] | 1H NMR (400.23 MHz, DMSO-d6) δ ppm 7.35 (d, J = 2.20 Hz, 2 H), 7.26 (br m, 1 H), 7.12 (m, 1 H), 6.97 (br d, J = 6.36 Hz, 2 H), 5.35 (s, 1 H), 3.96 (d, J = 6.85 Hz, 2 H), 2.87 (brd, J = 3.30 Hz, 1 H), 2.85 (br s, 1 H), 2.67 (d, J = 1.71 Hz, 1 H), 2.33 (d, J = 1.83 Hz, 3 H), 1.68 (br d, J = 6.97 Hz, 5 H), 1.50 (br s, 3 H). LC/MS: m/z [M + H]⁺ 445.2, Rt (λ = 220 nm): 2.84 min (LC/MS method A), calc. m/z: 445.45. |

| | | |
|---|---|---|
| 54 | 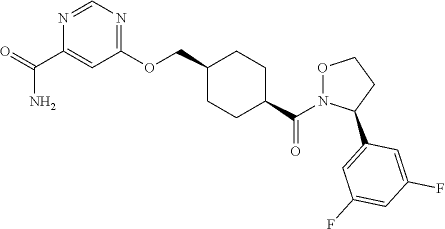 [SMILES: NC(=O)c1cc(OC[C@H]2CC[C@@H](C(=O)N3OCC[C@H]3c3cc(F)cc(F)c3)CC2)ncn1] | 1H NMR (DMSO-d6) δ: 9.42 (s, 1H), 7.91 (t, J = 4.9 Hz, 1H), 7.81 (t, J = 4.5 Hz, 1H), 7.50-7.57 (m, 2H), 7.13 (tt, J = 9.3, 2.2 Hz, 1H), 6.99-7.06 (m, 2H), 5.30-5.42 (m, 1H), 5.18 (quin, J = 8.3 Hz, 1H), 4.04-4.31 (m, 2H), 3.82-3.92 (m, 2H), 2.71-2.96 (m, 6H), 2.12-2.31 (m, 1H). LC/MS: m/z [M + H]⁺ 447.2, Rt (λ = 220 nm): 2.18 min (LC/MS method A), calc. m/z: 447.46. |

| | | |
|---|---|---|
| 55 | 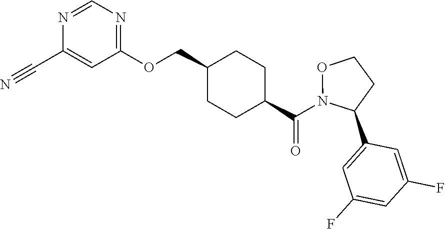 [SMILES: N#Cc1cc(OC[C@H]2CC[C@@H](C(=O)N3OCC[C@H]3c3cc(F)cc(F)c3)CC2)ncn1] | 1H NMR (DMSO-d6) δ: 8.93 (d, J = 1.0 Hz, 1H), 7.71 (d, J = 1.1 Hz, 1H), 7.13 (t, J = 8.9 Hz, 1H), 6.99 (s, 1H), 6.97 (s, 1H), 5.35 (dd, J = 8.7, 6.4 Hz, 1H), 4.21-4.38 (m, 3H), 3.83-3.90 (m, 1H), 2.83-2.93 (m, 2H), 2.12-2.23 (m, 1H), 2.00 (br d, J = 3.9 Hz, 1H), 1.75- 1.86 (m, 1H), 1.45-1.73 (m, 7H), 0.01 (s, 1H). LC/MS: m/z [M + H]⁺ 429.2, Rt (λ = 220 nm): 2.52 min (LC/MS method A), calc. m/z: 429.44. |

| | | |
|---|---|---|
| 56 | 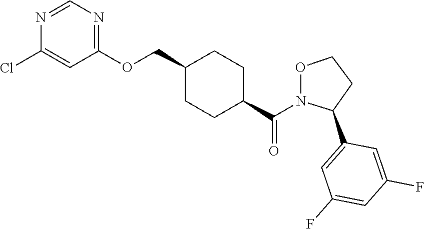 [SMILES: O=C([C@H]1CC[C@@H](COc2cc(Cl)ncn2)CC1)N1OCC[C@H]1c1cc(F)cc(F)c1] | 1H NMR (DMSO-d6) δ: 8.65 (s, 1H), 7.16 (d, J = 0.6 Hz, 1H), 7.13 (tt, J = 9.3, 2.4 Hz, 1H), 6.94-7.01 (m, 2H), 5.34 (dd, J = 8.6, 6.4 Hz, 1H), 4.20-4.33 (m, 3H), 3.82-3.89 (m, 1H), 2.82-2.92 (m, 2H), 2.12-2.22 (m, 1H), 1.97 (br d, J = 3.7 Hz, 1H), 1.74-1.86 (m, 1H), 1.43-1.72 (m, 7H), 0.00 (s, 1H). LC/MS: m/z [M + H]⁺ 438.2, Rt (λ = 220 nm): 2.66 min (LC/MS method A), calc. m/z: 438.88. |

| | | |
|---|---|---|
| 57 | 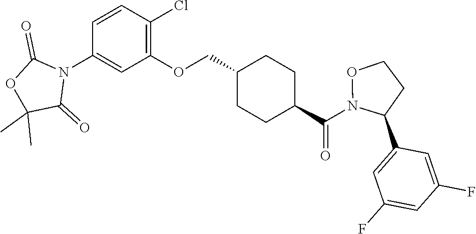 [SMILES: CC1(C)OC(=O)N(c2ccc(Cl)c(OC[C@H]3CC[C@H](C(=O)N4OCC[C@H]4c4cc(F)cc(F)c4)CC3)c2)C1=O] | 1H NMR (400.23 MHz, DMSO-d6) δ ppm 7.58 (d, J = 8.44 Hz, 1 H), 7.34 (d, J = 2.20 Hz, 1 H), 7.10 (m, 2 H), 6.98 (br d, J = 6.48 Hz, 2 H), 5.34 (m, 1 H), 4.25 (td, J = 7.76, 7.76, 2.81 Hz, 1 H), 3.86 (br d, J = 5.99 Hz, 3 H), 2.87 (m, 1 H), 2.73 (s, 1 H), 2.67 (br d, J = 1.71 Hz, 1 H), 2.19 (m, 1 H), 1.95 (m, 3 H), 1.78 (br d, J = 14.43 Hz, 2 H), 1.64 (s, 6 H), 1.41 (m, 2 H), 1.19 (m, 2 H). LC/MS: m/z [M + H]⁺ 563.3, Rt (λ = 220 nm): 2.76 min (LC/MS method A), calc. m/z: 564.00. |

| | | |
|---|---|---|
| 58 | 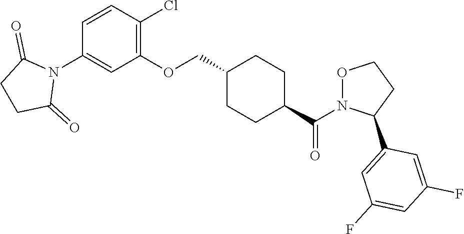 [SMILES: O=C1CCC(=O)N1c1ccc(Cl)c(OC[C@H]2CC[C@H](C(=O)N3OCC[C@H]3c3cc(F)cc(F)c3)CC2)c1] | 1H NMR (400.23 MHz, DMSO-d6) δ ppm 7.53 (d, J = 8.44 Hz, 1 H), 7.13 (tt, J = 9.29, 9.29, 2.26, 2.26 Hz, 1 H), 7.05 (d, J = 1.96 Hz, 1 H), 6.98 (br d, J = 6.48 Hz, 2 H), 6.85 (dd, J = 8.44, 2.08 Hz, 1 H), 5.34 (dd, J = 8.50, 6.54 Hz, 1 H), 4.25 (td, J = 7.76, 7.76, 2.93 Hz, 1 H), 3.84 (m, 3 H), 2.87 (m, 1 H), 2.77 (s, 4 H), 2.67 (br d, J = 1.83 Hz, 1 H), 2.19 (m, 1 H), 1.94 (m, 3 H), 1.79 (br s, 2 H), 1.40 (m, 2 H), 1.19 (m, 2 H). LC/MS: m/z [M + H]⁺ 533.3, Rt (λ = 220 nm): 2.47 min (LC/MS method A), calc. m/z: 533.97. |

| | | |
|---|---|---|
| 59 | 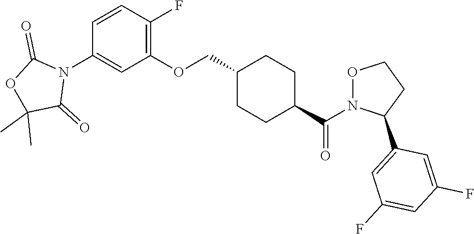 [SMILES: CC1(C)OC(=O)N(c2ccc(F)c(OC[C@H]3CC[C@H](C(=O)N4OCC[C@H]4c4cc(F)cc(F)c4)CC3)c2)C1=O] | 1H NMR (400.23 MHz, DMSO-d6) δ ppm 7.36 (m, 2 H), 7.09 (m, 2 H), 6.98 (br d, J = 6.60 Hz, 2 H), 5.34 (m, 1 H), 4.25 (td, J = 7.76, 7.76, 2.81 Hz, 1 H), 3.88 (m, 1 H), 3.85 (d, J = 6.36 Hz, 2 H), 2.87 (m, 1 H), 2.73 (br s, 1 H), 2.19 (m, 1 H), 1.98 (br s, 1 H), 1.92 (br d, J = 17.73 Hz, 2 H), 1.77 (br d, J = 14.43 Hz, 2 H), 1.63 (s, 6 H), 1.40 (m, 2 H), 1.16 (m, 2 H). LC/MS: m/z [M + H]⁺ 547.3, Rt (λ = 220 nm): 2.64 min (LC/MS method A), calc. m/z: 547.54. |

| | | |
|---|---|---|
| 60 | 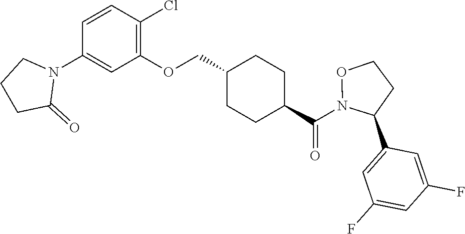 [SMILES: O=C1CCCN1c1ccc(Cl)c(OC[C@H]2CC[C@H](C(=O)N3OCC[C@H]3c3cc(F)cc(F)c3)CC2)c1] | 1H NMR (400.23 MHz, DMSO-d6) δ ppm 7.57 (d, J = 2.32 Hz, 1 H), 7.38 (d, J = 8.80 Hz, 1 H), 7.13 (m, 2 H), 6.98 (br d, J = 6.48 Hz, 2 H), 5.34 (dd, J = 8.68, 6.36 Hz, 1 H), 4.25 (td, J = 7.64, 7.64, 2.93 Hz, 1 H), 3.85 (m, 5 H), 2.87 (dddd, J = 12.10, 9.08, 6.33, 2.93 Hz, 1 H), 2.70 (m, 1 H), 2.19 (m, 1 H), 2.05 (quin, J = 7.52, 7.52, 7.52, 7.52 Hz, 2 H), 1.94 (m, 3 H), 1.77 (br d, J = 11.37 Hz, 2 H), 1.40 (m, 2 H), 1.19 (m, 2 H). LC/MS: m/z [M + H]⁺ 519.3, Rt (λ = 220 nm): 2.66 min (LC/MS method A), calc. m/z: 519.99. |

| | | |
|---|---|---|
| 61 | 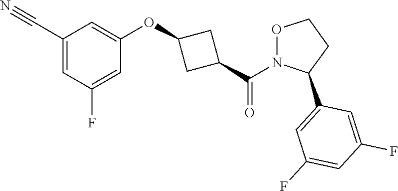 [SMILES: N#Cc1cc(F)cc(O[C@H]2C[C@@H](C(=O)N3OCC[C@H]3c3cc(F)cc(F)c3)C2)c1] | ¹H NMR (400 MHz, DMSO-d₆): δ ppm 7.40 (br d, J = 8.44 Hz, 1 H), 7.25 (s, 1 H), 7.15 (m, 2 H), 7.00 (m, J = 6.60 Hz, 2 H), 5.34 (dd, J = 8.56, 6.36 Hz, 1 H), 4.82 (br t, J = 7.09 Hz, 1 H), 4.22 (td, J = 7.76, 2.69 Hz, 1 H), 3.82 (m, 1 H), 3.20 (br s, 1 H), 2.88 (m, 1 H), 2.75 (m, 2 H), 2.20 (m, 3 H). LC/MS: m/z [M + H]⁺ 403.2, Rt (λ = 220 nm): 2.48 min (LC/MS method A), calc. m/z: 403.37. |

| | | |
|---|---|---|
| 62 | 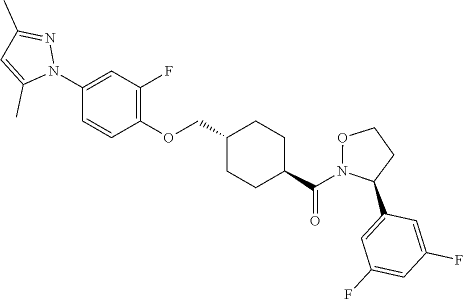 [SMILES: Cc1cc(C)n(-c2ccc(OC[C@H]3CC[C@H](C(=O)N4OCC[C@H]4c4cc(F)cc(F)c4)CC3)c(F)c2)n1] | 1H NMR (400.23 MHz, DMSO-d6) δ ppm 7.39 (d, J = 2.20 Hz, 1 H), 7.36 (s, 1 H), 7.24 (d, J = 5.87 Hz, 1 H), 7.23 (s, 1 H), 7.13 (t, J = 9.32, 9.32 Hz, 1 H), 6.98 (br d, J = 6.60 Hz, 2 H), 6.03 (s, 1 H), 5.35 (m, 1 H), 4.25 (td, J = 7.67, 7.67, 2.87 Hz, 1 H), 3.93 (d, J = 6.36 Hz, 2 H), 3.87 (m, 1 H), 2.88 (m, 1 H), 2.73 (br s, 1 H), 2.67 (br s, 1 H), 2.33 (s, 1 H), 2.20 (m, 7 H), 1.93 (m, 3 H), 1.77 (br d, J = 11.37 Hz, 2 H), 1.41 (m, 2 H), 1.18 (m, 2 H). LC/MS: m/z [M + H]⁺ 514.4, Rt (λ = 220 nm): 2.79 min (LC/MS method A), calc. m/z: 514.56. |

| | | |
|---|---|---|
| 63 | 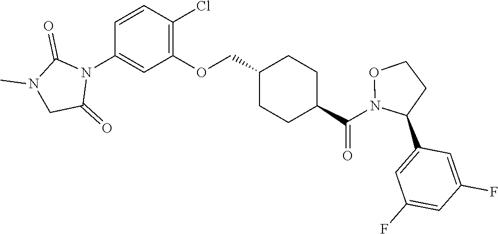 [SMILES: CN1CC(=O)N(c2ccc(Cl)c(OC[C@H]3CC[C@H](C(=O)N4OCC[C@H]4c4cc(F)cc(F)c4)CC3)c2)C1=O] | 1H NMR (400.23 MHz, DMSO-d6) δ ppm 7.52 (d, J = 8.44 Hz, 1 H), 7.13 (m, 2 H), 6.98 (br d, J = 6.60 Hz, 2 H), 6.95 (d, J = 8.40 Hz, 1 H), 5.34 (m, 1 H), 4.25 (td, J = 7.70, 7.70, 2.93 Hz, 1 H), 4.10 (s, 2 H), 3.85 (m, 3 H), 2.93 (s, 3 H), 2.87 (m, 1 H), 2.71 (m, 1 H), 2.19 (m, 1 H), 1.93 (m, 3 H), 1.79 (br s, 2 H), 1.40 (m, 2 H), 1.19 (m, 2 H). LC/MS: m/z [M + H]⁺ 548.3, Rt (λ = 220 nm): 2.45 min (LC/MS method A), calc. m/z: 548.99. |

| | | |
|---|---|---|
| 64 | 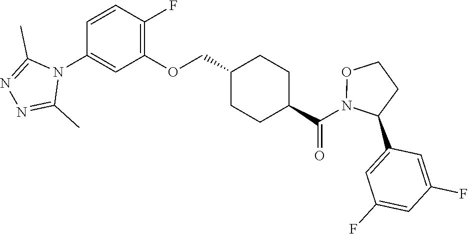 [SMILES: Cc1nnc(C)n1-c1ccc(F)c(OC[C@H]2CC[C@H](C(=O)N3OCC[C@H]3c3cc(F)cc(F)c3)CC2)c1] | 1H NMR (400.23 MHz, DMSO-d6) δ ppm 7.48 (t, J = 10.00, 10.00 Hz, 1 H), 7.43 (d, J = 7.07 Hz, 1 H), 7.13 (m, 2 H), 6.98 (br d, J = 6.72 Hz, 2 H), 5.34 (m, 1 H), 4.25 (td, J = 7.64, 7.64, 2.81 Hz, 1 H), 3.89 (m, 3 H), 2.87 (m, 1 H), 2.70 (m, 1 H), 2.35 (br d, J = 18.83 Hz, 1 H), 2.27 (s, 6 H), 2.19 (m, 1 H), 1.96 (br d, J = 12.23 Hz, 1 H), 1.90 (br d, J = 10.51 Hz, 2 H), 1.78 (br s, 2 H), 1.40 (m, 2 H), 1.15 (m, 3 H). LC/MS: m/z [M + H]⁺ 515.4, Rt (λ = 220 nm): 2.22 min (LC/MS method A), calc. m/z: 515.55. |

| | | |
|---|---|---|
| 65 | 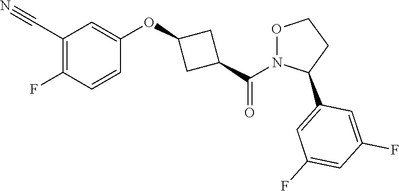 [SMILES: N#Cc1cc(O[C@H]2C[C@@H](C(=O)N3OCC[C@H]3c3cc(F)cc(F)c3)C2)ccc1F] | 1H NMR (400.23 MHz, DMSO-d6) δ ppm 7.42 (m, 2 H), 7.27 (dt, J = 9.14, 3.87, 3.87 Hz, 1 H), 7.13 (m, 1 H), 7.00 (br d, J = 6.60 Hz, 2 H), 5.33 (dd, J = 8.62, 6.42 Hz, 1 H), 4.75 (quin, J = 7.15, 7.15, 7.15, 7.15 Hz, 1 H), 4.22 (td, J = 7.70, 7.70, 2.81 Hz, 1 H), 3.82 (m, 1 H), 3.20 (br s, 1 H), 2.88 (m, 1 H), 2.73 (m, 2 H), 2.19 (m, 3 H), 2.07 (s, 1 H), 1.22 (m, 1 H). LC/MS: m/z [M + H]⁺ 403.2, Rt (λ = 220 nm): 2.44 min (LC/MS method |
| | | A), calc. m/z: 403.37. |

| | | |
|---|---|---|
| 66 | 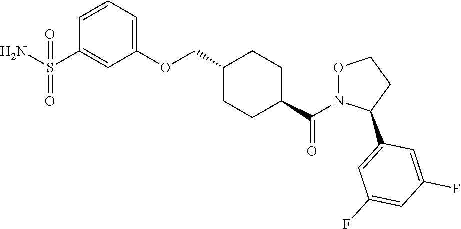 [SMILES: NS(=O)(=O)c1cccc(OC[C@H]2CC[C@H](C(=O)N3OCC[C@H]3c3cc(F)cc(F)c3)CC2)c1] | 1H NMR (400.23 MHz, DMSO-d6) δ ppm 7.46 (m, 1 H), 7.34 (m, 4 H), 7.14 (m, 2 H), 6.98 (br d, J = 6.60 Hz, 2 H), 5.35 (m, 1 H), 4.25 (td, J = 7.73, 7.73, 3.00 Hz, 1 H), 3.88 (m, 1 H), 3.85 (br d, J = 6.24 Hz, 2 H), 2.87 (dddd, J = 12.18, 9.12, 6.51, 3.00 Hz, 1 H), 2.73 (br s, 1 H), 2.67 (br s, 1 H), 2.19 (m, 1 H), 1.98 (br s, 1 H), 1.92 (br d, J = 13.33 Hz, 2 H), 1.76 (br d, J = 6.48 Hz, 2 H), 1.40 (m, 2 H), 1.17 (m, 2 H). LC/MS: m/z [M + H]⁺ 481.3, Rt (λ = 220 nm): 2.24 min (LC/MS method A), calc. m/z: 481.53. |

| | | |
|---|---|---|
| 67 | 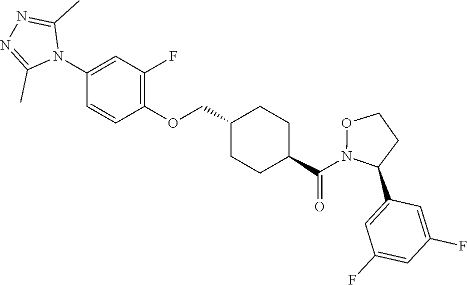 [SMILES: Cc1nnc(C)n1-c1ccc(OC[C@H]2CC[C@H](C(=O)N3OCC[C@H]3c3cc(F)cc(F)c3)CC2)c(F)c1] | 1H NMR (400.23 MHz, DMSO-d6) δ ppm 7.55 (dd, J = 11.61, 2.45 Hz, 1 H), 7.35 (m, 2 H), 7.13 (tt, J = 9.29, 9.29, 2.26, 2.26 Hz, 1 H), 6.98 (br d, J = 6.48 Hz, 2 H), 5.35 (m, 1 H), 4.25 (td, J = 7.64, 7.64, 2.93 Hz, 1 H), 3.97 (d, J = 6.11 Hz, 2 H), 3.87 (m, 1 H), 2.88 (dddd, J = 12.12, 9.12, 6.45, 3.12 Hz, 4 H), 2.73 (br s, 1 H), 2.22 (s, 7 H), 1.93 (m, 3 H), 1.78 (br d, J = 12.10 Hz, 2 H), 1.42 (m, 2 H), 1.18 (m, 2 H). LC/MS: m/z [M + H]⁺ 515.4, Rt (λ = 220 nm): 2.16 min (LC/MS method A), calc. m/z: 515.57. |

| | | |
|---|---|---|
| 68 | 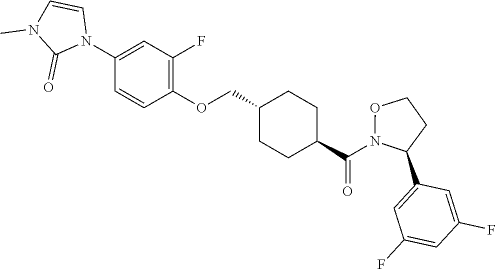 [SMILES: Cn1ccn(-c2ccc(OC[C@H]3CC[C@H](C(=O)N4OCC[C@H]4c4cc(F)cc(F)c4)CC3)c(F)c2)c1=O] | 1H NMR (400.23 MHz, DMSO-d6) δ ppm 7.67 (dd, J = 13.14, 2.63 Hz, 1 H), 7.46 (d, J = 9.02 Hz, 1 H), 7.16 (m, 2 H), 7.01 (s, 1 H), 6.98 (br d, J = 6.60 Hz, 2 H), 6.71 (d, J = 3.06 Hz, 1 H), 5.34 (dd, J = 8.56, 6.24 Hz, 1 H), 4.25 (td, J = 7.67, 7.67, 3.00 Hz, 1 H), 3.88 (d, J = 6.36 Hz, 2 H), 3.85 (m, 1 H), 3.18 (s, 4 H), 2.87 (dddd, J = 12.13, 9.11, 6.45, 3.12 Hz, 1 H), 2.71 (m, 1 H), 2.19 (m, 1 H), 1.92 (m, 3 H), 1.76 (br dd, J = 6.60, 2.81 Hz, 2 H), 1.39 (m, 2 H), 1.17 (m, 3 H). LC/MS: m/z [M + H]⁺ 516.4, Rt (λ = 220 nm): 2.39 min (LC/MS method A), calc. m/z: 516.53. |

| | | |
|---|---|---|
| 69 | 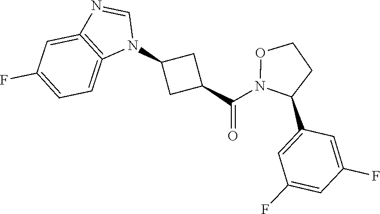 | 1H NMR (400.23 MHz, DMSO-d6) δ ppm 9.02 (s, 1 H), 7.84 (dd, J = 8.99, 4.59 Hz, 1 H), 7.60 (dd, J = 9.23, 2.38 Hz, 1 H), 7.32 (td, J = 9.29, 9.29, 2.32 Hz, 1 H), 7.14 (m, 1 H), 7.03 (br d, J = 6.72 Hz, 2 H), 5.37 (m, 1 H), 5.11 (quin, J = 8.38, 8.38, 8.38, 8.38 Hz, 1 H), 4.25 (m, 1 H), 3.87 (br dd, J = 16.44, 7.64 Hz, 5 H), 2.88 (m, 4 H), 2.73 (m, 1 H), 2.22 (m, 1 H). LC/MS: m/z [M + H]⁺ 402.2, Rt (λ = 220 nm): 1.73 min (LC/MS method A), calc. m/z: 402.39. |

| | | |
|---|---|---|
| 70 | 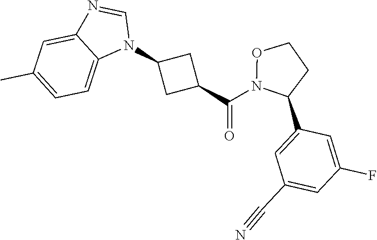 | 1H NMR (400.23 MHz, DMSO-d6) δ ppm 8.89 (br s, 1 H), 7.80 (m, 2 H), 7.66 (s, 1 H), 7.56 (m, 2 H), 7.29 (td, J = 9.26, 9.26, 2.63 Hz, 1 H), 5.42 (m, 1 H), 5.09 (br t, J = 8.50, 8.50 Hz, 1 H), 4.26 (m, 1 H), 3.89 (m, 1 H), 2.89 (m, 6 H), 2.71 (m, 1 H), 2.33 (m, 1 H), 2.25 (m, 1 H). LC/MS: m/z [M + H]⁺ 409.2, Rt (λ = 220 nm): 1.6 min (LC/MS method A), calc. m/z: 409.41. |

| | | |
|---|---|---|
| 71 | 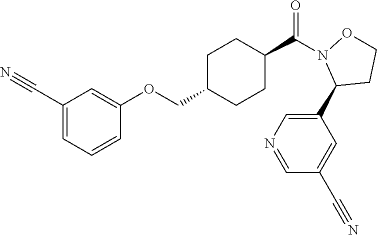 [SMILES: N#Cc1cccc(OC[C@H]2CC[C@H](C(=O)N3OCC[C@H]3c3cncc(C#N)c3)CC2)c1] | ¹H NMR (400 MHz, DMSO-d₆): δ ppm 8.94 (d, J = 1.96 Hz, 1 H), 8.78 (d, J = 2.20 Hz, 1 H), 8.20 (t, J = 2.02, 2.02 Hz, 1 H), 7.42 (m, 3 H), 7.29 (d, J = 8.01 Hz, 1 H), 5.42 (dd, J = 8.62, 6.30 Hz, 1 H), 4.30 (td, J = 7.67, 7.67, 3.12 Hz, 1 H), 3.92 (td, J = 8.59, 8.59, 6.66 Hz, 1 H), 3.86 (d, J = 6.36 Hz, 2 H), 2.91 (dddd, J = 12.12, 9.15, 6.42, 3.24 Hz, 1 H), 2.71 (m, 1 H), 2.30 (m, 1 H), 1.95 (br d, J = 12.35 Hz, 1 H), 1.89 (br d, J = 13.20 Hz, 2 H), 1.76 (m, 2 H), 1.38 (m, 2 H), 1.15 (m, 2 H). LC/MS: m/z [M + H]⁺ 417.2, Rt (λ = 220 nm): 2.24 min (LC/MS method |
| | | A), calc. m/z: 417.48. |

| | | |
|---|---|---|
| 72 | 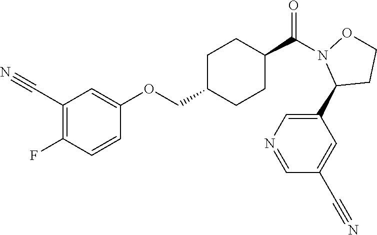 [SMILES: N#Cc1cncc([C@@H]2CCON2C(=O)[C@H]2CC[C@H](COc3ccc(F)c(C#N)c3)CC2)c1] | 1H NMR (DMSO-d6) δ: 7.71 (d, J = 8.6 Hz, 1H), 7.55 (s, 1H), 7.42 (br d, J = 9.5 Hz, 1H), 7.30-7.35 (m, 1H), 7.17 (s, 1H), 7.18 (d, J = 5.8 Hz, 2H), 5.98 (s, 1H), 5.30-5.36 (m, 1H), 4.21 (td, J = 7.6, 3.1 Hz, 1H), 3.80-3.89 (m, 3H), 2.84 (dtt, J = 12.1, 6.2, 3.1 Hz, 1H), 2.62-2.72 (m, 1H), 2.11-2.22 (m, 4H), 2.10 (s, 3H), 1.94 (br s, 1H), 1.86 (br d, J = 12.8 Hz, 2H), 1.72 (br d, J = 9.2 Hz, 2H), 1.28-1.40 (m, 2H), 1.03-1.20 (m, 2H), -0.06 (s, 1H). LC/MS: m/z [M + H]⁺ 435.23, Rt (λ = 220 nm): 2.29 min (LC/MS method A), calc. m/z: 435.47. |

| | | |
|---|---|---|
| 73 | 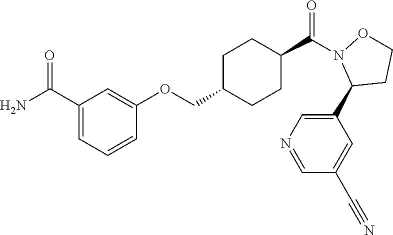 [SMILES: N#Cc1cncc([C@@H]2CCON2C(=O)[C@H]2CC[C@H](COc3cccc(C(N)=O)c3)CC2)c1] | 1H NMR (400.23 MHz, DMSO-d6) δ ppm 8.94 (d, J = 1.96 Hz, 1 H), 8.78 (d, J = 2.08 Hz, 1 H), 8.20 (t, J = 1.96, 1.96 Hz, 1 H), 7.93 (br s, 1 H), 7.44 (s, 1 H), 7.42 (m, 2 H), 7.33 (m, 2 H), 7.07 (dd, J = 8.13, 1.77 Hz, 1 H), 5.42 (dd, J = 8.50, 6.54 Hz, 1 H), 4.30 (td, J = 7.70, 7.70, 2.93 Hz, 1 H), 3.93 (m, 1 H), 3.83 (d, J = 6.24 Hz, 2 H), 2.91 (dddd, J = 12.17, 9.17, 6.42, 3.06 Hz, 1 H), 2.74 (br t, J = 11.92, 11.92 Hz, 1 H), 2.30 (m, 1 H), 1.93 (m, 3 H), 1.77 (m, 2 H), 1.39 (m, 2 H), 1.16 (m, 2 H). LC/MS: m/z [M + H]⁺ 435.3, Rt (X = 220 nm): 1.73 min (LC/MS method |
| | | A), calc. m/z: 435.50. |

| | | |
|---|---|---|
| 74 | 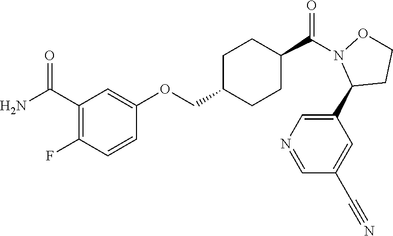 [SMILES: N#Cc1cncc([C@@H]2CCON2C(=O)[C@H]2CC[C@H](COc3ccc(F)c(C(N)=O)c3)CC2)c1] | 1H NMR (400.23 MHz, DMSO-d6) δ ppm 8.94 (d, J = 1.96 Hz, 1 H), 8.78 (d, J = 2.08 Hz, 1 H), 8.20 (t, J = 2.02, 2.02 Hz, 1 H), 7.64 (br s, 1 H), 7.61 (br s, 1 H), 7.16 (m, 2 H), 7.05 (m, 1 H), 5.42 (dd, J = 8.56, 6.48 Hz, 1 H), 4.30 (td, J = 7.70, 7.70, 3.06 Hz, 1 H), 3.92 (m, 1 H), 3.80 (d, J = 6.24 Hz, 2 H), 2.90 (dddd, J = 12.10, 9.17, 6.54, 3.12 Hz, 1 H), 2.73 (br t, J = 11.80, 11.80 Hz, 1 H), 2.30 (m, 1 H), 1.91 (m, 3 H), 1.75 (m, 2 H), 1.38 (m, 2 H), 1.15 (m, 2 H). LC/MS: m/z [M + H]⁺ 453.3, Rt (X = 220 nm): 1.8 min (LC/MS method A), calc. m/z: 453.49. |

| | | |
|---|---|---|
| 75 | 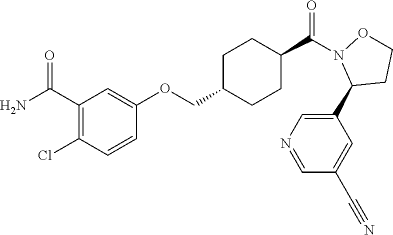 [SMILES: N#Cc1cncc([C@@H]2CCON2C(=O)[C@H]2CC[C@H](COc3ccc(Cl)c(C(N)=O)c3)CC2)c1] | 1H NMR (400.23 MHz, DMSO-d6) δ ppm 8.94 (d, J = 1.96 Hz, 1 H), 8.78 (d, J = 2.20 Hz, 1 H), 8.20 (t, J = 1.96, 1.96 Hz, 1 H), 7.81 (s, 1 H), 7.54 (s, 1 H), 7.34 (d, J = 8.93 Hz, 1 H), 6.98 (d, J = 9.73 Hz, 1 H), 6.97 (s, 1 H), 5.42 (dd, J = 8.50, 6.54 Hz, 1 H), 4.30 (td, J = 7.73, 7.73, 3.12 Hz, 1 H), 3.92 (m, 1 H), 3.81 (d, J = 6.24 Hz, 2 H), 2.90 (m, 1 H), 2.72 (br t, J = 11.86, 11.86 Hz, 1 H), 2.30 (m, 1 H), 1.95 (br d, J = 12.35 Hz, 1 H), 1.88 (br d, J = 12.47 Hz, 2 H), 1.76 (m, 2 H), 1.38 (m, 2 H), 1.13 (m, 2 H). LC/MS: m/z [M + H]⁺ 469.3, Rt (λ = |
| | | 220 nm): 1.83 min (LC/MS method |
| | | A), calc. m/z: 469.94. |

| | | |
|---|---|---|
| 76 | 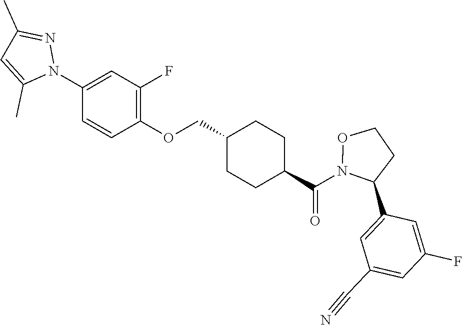 [SMILES: Cc1cc(C)n(-c2ccc(OC[C@H]3CC[C@H](C(=O)N4OCC[C@H]4c4cc(F)cc(C#N)c4)CC3)c(F)c2)n1] | 1H NMR (DMSO-d6) δ: 7.71 (d, J = 8.6 Hz, 1H), 7.55 (s, 1H), 7.42 (br d, J = 9.5 Hz, 1H), 7.30-7.35 (m, 1H), 7.17 (s, 1H), 7.18 (d, J = 5.8 Hz, 2H), 5.98 (s, 1H), 5.30-5.36 (m, 1H), 4.21 (td, J = 7.6, 3.1 Hz, 1H), 3.80-3.89 (m, 3H), 2.84 (dtt, J = 12.1, 6.2, 3.1 Hz, 1H), 2.62-2.72 (m, 1H), 2.11-2.22 (m, 4H), 2.10 (s, 3H), 1.94 (br s, 1H), 1.86 (br d, J = 12.8 Hz, 2H), 1.72 (br d, J = 9.2 Hz, 2H), 1.28-1.40 (m, 2H), 1.03-1.20 (m, 2H), -0.06 (s, 1H). LC/MS: m/z [M + H]⁺ 521.4, Rt (λ = 220 nm): 2.66 min (LC/MS method A), calc. m/z: 521.58. |

| | | |
|---|---|---|
| 77 | 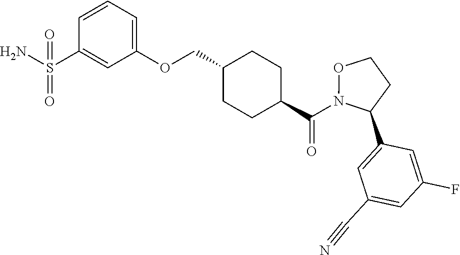 [SMILES: N#Cc1cc(F)cc([C@@H]2CCON2C(=O)[C@H]2CC[C@H](COc3cccc(S(N)(=O)=O)c3)CC2)c1] | 1H NMR (DMSO-d6) δ: 7.71 (d, J = 8.6 Hz, 1H), 7.55 (s, 1H), 7.42 (br d, J = 9.5 Hz, 1H), 7.30-7.35 (m, 1H), 7.17 (s, 1H), 7.18 (d, J = 5.8 Hz, 2H), 5.98 (s, 1H), 5.30-5.36 (m, 1H), 4.21 (td, J = 7.6, 3.1 Hz, 1H), 3.80-3.89 (m, 3H), 2.84 (dtt, J = 12.1, 6.2, 3.1 Hz, 1H), 2.62-2.72 (m, 1H), 2.11-2.22 (m, 4H), 2.10 (s, 3H), 1.94 (br s, 1H), 1.86 (br d, J = 12.8 Hz, 2H), 1.72 (br d, J = 9.2 Hz, 2H), 1.28-1.40 (m, 2H), 1.03-1.20 (m, 2H), -0.06 (s, 1H). LC/MS: m/z [M + H]⁺ 488.3, Rt (λ = 220 nm): 2.1 min (LC/MS method A), calc. m/z: 488.55. |

| | | |
|---|---|---|
| 78 | 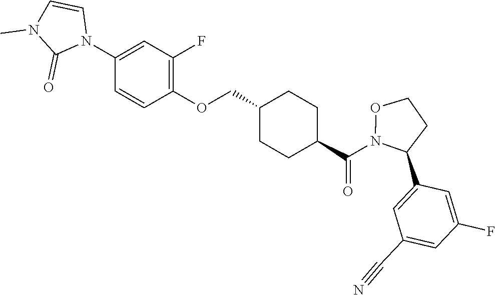 [SMILES: Cn1ccn(-c2ccc(OC[C@H]3CC[C@H](C(=O)N4OCC[C@H]4c4cc(F)cc(C#N)c4)CC3)c(F)c2)c1=O] | 1H NMR (DMSO-d6) δ: 7.72 (br d, J = 8.7 Hz, 1H), 7.62 (dd, J = 13.1, 2.6 Hz, 1H), 7.56 (s, 1H), 7.42 (br t, J = 8.6 Hz, 2H), 7.17 (t, J = 9.2 Hz, 1H), 6.96 (d, J = 3.2 Hz, 1H), 6.67 (d, J = 3.1 Hz, 1H), 5.31-5.37 (m, 1H), 4.20-4.26 (m, 1H), 3.78-3.90 (m, 3H), 3.13 (s, 3H), 2.84 (br dd, J = 6.4, 3.1 Hz, 1H), 2.64- 2.73 (m, 1H), 2.14-2.23 (m, 1H), 1.81-1.97 (m, 3H), 1.72 (br s, 2H), 1.28-1.40 (m, 2H), 1.00-1.24 (m, 3H), −0.04 (s, 2H). LC/MS: m/z [M + H]⁺ 523.3, Rt (λ = 220 nm): 2.26 min (LC/MS method A), calc. m/z: 523.55. |

| | | |
|---|---|---|
| 79 | 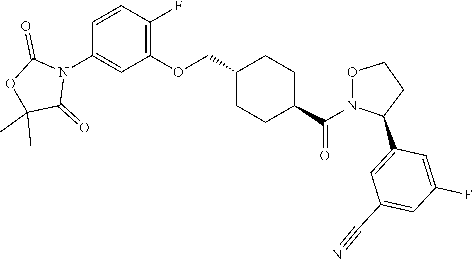 [SMILES: CC1(C)OC(=O)N(c2ccc(F)c(OC[C@H]3CC[C@H](C(=O)N4OCC[C@H]4c4cc(F)cc(C#N)c4)CC3)c2)C1=O] | 1H NMR (DMSO-d6) δ: 7.69-7.74 (m, 1H), 7.55 (s, 1H), 7.42 (br d, J = 9.5 Hz, 1H), 7.28-7.35 (m, 2H), 6.98-7.03 (m, 1H), 5.33 (dd, J = 8.5, 6.7 Hz, 1H), 4.21 (td, J = 7.8, 2.9 Hz, 1H), 3.74- 3.88 (m, 3H), 2.84 (dddd, J = 12.2, 9.2, 6.6, 3.1 Hz, 1H), 2.62-2.72 (m, 1H), 2.12-2.22 (m, 1H), 1.86 (br d, J = 13.0 Hz, 2H), 1.72 (br d, J = 12.7 Hz, 2H), 1.58 (s, 6H), 1.25-1.43 (m, 2H), 1.00- 1.22 (m, 2H), -0.05 (s, 2H). LC/MS: m/z [M + H]⁺ 554.3, Rt (λ = 220 nm): 2.51 min (LC/MS method A), calc. m/z: 554.56. |

| | | |
|---|---|---|
| 80 | 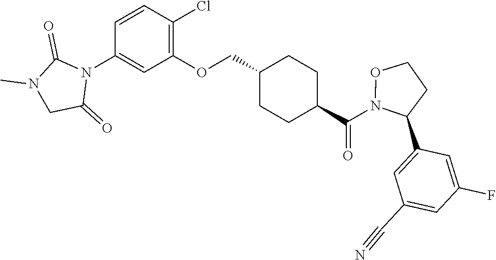 [SMILES: CN1CC(=O)N(c2ccc(Cl)c(OC[C@H]3CC[C@H](C(=O)N4OCC[C@H]4c4cc(F)cc(C#N)c4)CC3)c2)C1=O] | 1H NMR (DMSO-d6) δ: 7.84 (d, J = 8.6 Hz, 1H), 7.68 (s, 1H), 7.52-7.62 (m, 1H), 7.21 (d, J = 2.2 Hz, 1H), 7.02 (dd, J = 8.5, 2.1 Hz, 1H), 5.45 (dd, J = 8.4, 6.5 Hz, 1H), 5.22-5.30 (m, 1H), 4.79-5.00 (m, 1H), 4.34 (td, J = 7.7, 2.9 Hz, 1H), 4.17 (s, 1H), 3.88- 4.00 (m, 2H), 3.00 (s, 2H), 2.92-2.99 (m, 1H), 2.75-2.85 (m, 1H), 2.25-2.34 (m, 1H), 1.94-2.08 (m, 2H), 1.85 (br d, J = 11.7 Hz, 1H), 1.44 (d, J = 6.2 Hz, 5H), 1.17-1.36 (m, 5H). LC/MS: m/z [M + H]⁺ 555.3, Rt (λ = 220 nm): 2.32 min (LC/MS method A), calc. m/z: 556.01. |

| | | |
|---|---|---|
| 81 | 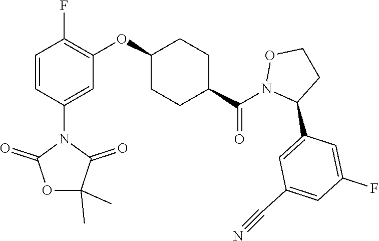 [SMILES: CC1(C)OC(=O)N(c2ccc(F)c(O[C@H]3CC[C@@H](C(=O)N4OCC[C@H]4c4cc(F)cc(C#N)c4)CC3)c2)C1=O] | 1H NMR (400.23 MHz, DMSO-d6) δ ppm 7.62 (s, 1 H), 7.39 (m, 1 H), 7.37 (m, 2 H), 2.67 (d, J = 1.96 Hz, 2 H), 2.33 (s, 4 H), 1.73 (m, 7 H), 1.63 (s, 10 H). LC/MS: m/z [M + H]⁺ 540.3, Rt (λ = 220 nm): 2.43 min (LC/MS method A), calc. m/z: 540.54. |

| | | |
|---|---|---|
| 82 | 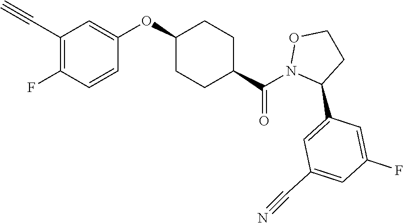 [SMILES: C#Cc1cc(O[C@H]2CC[C@@H](C(=O)N3OCC[C@H]3c3cc(F)cc(C#N)c3)CC2)ccc1F] | 1H NMR (400.23 MHz, DMSO-d6) δ ppm 7.77 (d, J = 7.96 Hz, 1 H), 7.61 (s, 1 H), 7.47 (m, 3 H), 7.35 (m, 1 H), 5.38 (dd, J = 8.62, 6.66 Hz, 1 H), 4.64 (br s, 1 H), 4.27 (td, J = 7.73, 7.73, 2.87 Hz, 1 H), 3.90 (m, 1 H), 2.88 (m, 2 H), 2.22 (m, 1 H), 1.91 (m, 2 H), 1.71 (m, 5 H), 1.52 (br d, J = 11.00 Hz, 1 H), 1.22 (br dd, J = 6.30, 4.10 Hz, 1 H). LC/MS: m/z [M + H]⁺ 438.2, Rt (λ = 220 nm): 2.45 min (LC/MS method A), calc. m/z: 438.45. |

| | | |
|---|---|---|
| 83 | 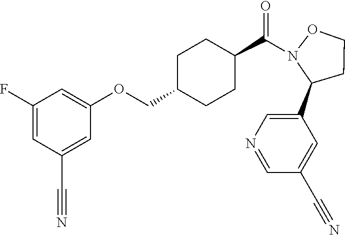 [SMILES: N#Cc1cc(F)cc(OC[C@H]2CC[C@H](C(=O)N3OCC[C@H]3c3cncc(C#N)c3)CC2)c1] | 1H NMR (400 MHz, DMSO-d6): δ ppm 8.94 (d, J = 1.96 Hz, 1 H), 8.78 (d, J = 2.20 Hz, 1 H), 8.20 (t, J = 1.96, 1.96 Hz, 1 H), 7.38 (d, J = 8.52 Hz, 1 H), 7.33 (s, 1 H), 7.25 (dt, J = 11.13, 2.32, 2.32 Hz, 1 H), 5.42 (dd, J = 8.44, 6.48 Hz, 1 H), 4.30 (td, J = 7.76, 7.76, 3.18 Hz, 1 H), 3.90 (m, 3 H), 2.90 (dddd, J = 12.15, 9.15, 6.39, 3.12 Hz, 1 H), 2.73 (br t, J = 11.68, 11.68 Hz, 1 H), 2.30 (m, 1 H), 1.95 (br d, J = 13.08 Hz, 1 H), 1.88 (br d, J = 11.86 Hz, 2 H), 1.76 (m, 2 H), 1.38 (m, 2 H), 1.13 (m, 2 H). LC/MS: m/z [M + H]⁺ 435.2, Rt (λ = |
| | | 220 nm): 2.34 min (LC/MS method |
| | | A), calc. m/z: 435.47. |

| | | |
|---|---|---|
| 84 | 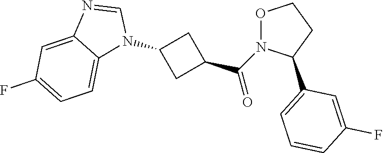 [SMILES: O=C([C@H]1C[C@H](n2cnc3cc(F)ccc32)C1)N1OCC[C@H]1c1cccc(F)c1] | 1H NMR (400.23 MHz, DMSO-d6) δ ppm 9.11 (br s, 1 H), 7.81 (dd, J = 8.99, 4.58 Hz, 1 H), 7.61 (dd, J = 9.23, 2.38 Hz, 1 H), 7.42 (m, 1 H), 7.30 (td, J = 9.29, 9.29, 2.45 Hz, 1 H), 7.15 (m, 3 H), 5.41 (dd, J = 8.62, 6.30 Hz, 1 H), 5.15 (quin, J = 7.98, 7.98, 7.98, 7.98 Hz, 1 H), 4.24 (td, J = 7.70, 7.70, 2.93 Hz, 1 H), 3.90 (m, 3 H), 3.86 (br s, 2 H), 2.91 (m, 5 H), 2.23 (m, 1 H). |
| | | LC/MS: m/z [M + H]⁺ 384.2, Rt (λ = |
| | | 220 nm): 1.67 min (LC/MS method |
| | | A), calc. m/z: 384.40. |

| | | |
|---|---|---|
| 85 | 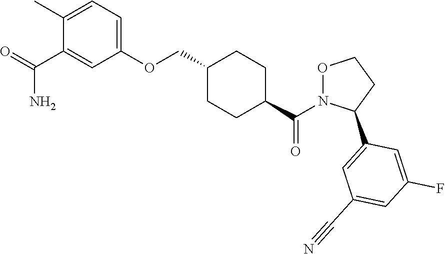 [SMILES: Cc1ccc(OC[C@H]2CC[C@H](C(=O)N3OCC[C@H]3c3cc(F)cc(C#N)c3)CC2)cc1C(N)=O] | 1H NMR (400.23 MHz, DMSO-d6) δ ppm 7.77 (d, J = 8.64 Hz, 1 H), 7.66 (br s, 1 H), 7.60 (s, 1 H), 7.48 (br d, J = 9.54 Hz, 1 H), 7.29 (br s, 1 H), 7.11 (d, J = 8.31 Hz, 1 H), 6.90 (s, 1 H), 6.87 (d, J = 8.63 Hz, 1 H), 5.38 (dd, J = 8.68, 6.36 Hz, 1 H), 4.27 (td, J = 7.73, 7.73, 3.12 Hz, 1 H), 3.89 (m, 1 H), 3.78 (d, J = 6.36 Hz, 2 H), 2.89 (m, 1 H), 2.73 (br t, J = 11.80, 11.80 Hz, 1 H), 2.27 (s, 3 H), 2.22 (m, 1 H), 1.91 (m, 3 H), 1.75 (m, 2 H), 1.39 (m, 2 H), 1.14 (m, 2 H). LC/MS: m/z [M + H]⁺ 466.3, Rt (λ = 220 nm): 2.12 min (LC/MS method A), calc. m/z: 466.52. |

| | | |
|---|---|---|
| 86 | 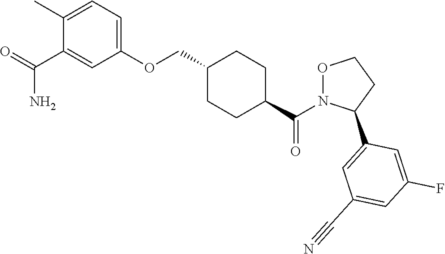 | 1H NMR (400.23 MHz, DMSO-d6) δ ppm 7.66 (br s, 1 H), 7.29 (br s, 1 H), 7.12 (m, 2 H), 6.98 (m, 2 H), 6.89 (m, 2 H), 5.34 (dd, J = 8.50, 6.30 Hz, 1 H), 4.25 (td, J = 7.73, 7.73, 2.87 Hz, 1 H), 3.86 (m, 1 H), 3.78 (d, J = 6.36 Hz, 2 H), 2.87 (dddd, J = 12.06, 9.12, 6.51, 3.12 Hz, 1 H), 2.69 (m, 1 H), 2.27 (s, 3 H), 2.19 (m, 1 H), 1.92 (m, 3 H), 1.75 (m, 2 H), 1.39 (m, 2 H), 1.14 (m, 2 H). LC/MS: m/z [M + H]⁺ 459.3, Rt (λ = 220 nm): 2.25 min (LC/MS method A), calc. m/z: 459.51. |

| | | |
|---|---|---|
| 87 | 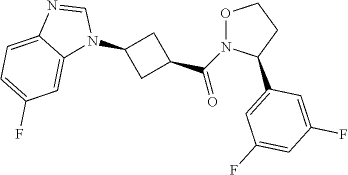 [SMILES: O=C([C@H]1C[C@@H](n2cnc3ccc(F)cc32)C1)N1OCC[C@H]1c1cc(F)cc(F)c1] | 1H NMR (400.23 MHz, DMSO-d6) δ ppm 9.23 (s, 1 H), 7.81 (m, 2 H), 7.35 (td, J = 9.32, 9.32, 2.38 Hz, 1 H), 7.14 (tt, J = 9.31, 9.31, 2.31, 2.31 Hz, 1 H), 7.04 (m, 2 H), 5.38 (br dd, J = 8.19, 6.72 Hz, 1 H), 5.10 (dt, J = 16.72, 8.45, 8.45 Hz, 1 H), 4.69 (br s, 3 H), 4.26 (td, J = 7.64, 7.64, 2.69 Hz, 1 H), 3.88 (m, 1 H), 2.89 (m, 4 H), 2.74 (m, 1 H), 2.22 (m, 1 H). LC/MS: m/z [M + H]⁺ 402.2, Rt (λ = |
| | | 220 nm): 1.76 min (LC/MS method |
| | | A), calc. m/z: 402.39. |

| | | |
|---|---|---|
| 88 | 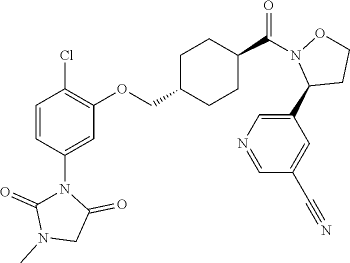 [SMILES: CN1CC(=O)N(c2ccc(Cl)c(OC[C@H]3CC[C@H](C(=O)N4OCC[C@H]4c4cncc(C#N)c4)CC3)c2)C1=O] | 1H NMR (400.23 MHz, DMSO-d6) δ ppm 8.94 (d, J = 1.83 Hz, 1 H), 8.78 (d, J = 2.08 Hz, 1 H), 8.20 (t, J = 1.90, 1.90 Hz, 1 H), 7.52 (d, J = 8.44 Hz, 1 H), 7.39 (m, 1 H), 7.14 (d, J = 2.08 Hz, 1 H), 6.95 (dd, J = 8.44, 2.08 Hz, 1 H), 5.42 (m, 1 H), 4.30 (td, J = 7.67, 7.67, 3.00 Hz, 1 H), 4.10 (s, 1 H), 3.87 (m, 3 H), 2.93 (s, 2 H), 2.89 (m, 1 H), 2.69 (m, 2 H), 2.30 (m, 1 H), 1.93 (m, 3 H), 1.79 (br d, J = 10.03 Hz, 2 H), 1.39 (m, 2 H), 1.18 (m, 2 H). LC/MS: m/z [M + H]⁺ 538.3, Rt (λ = 220 nm): 2.02 min (LC/MS method A), calc. m/z: 539.00. |

| | | |
|---|---|---|
| 89 | 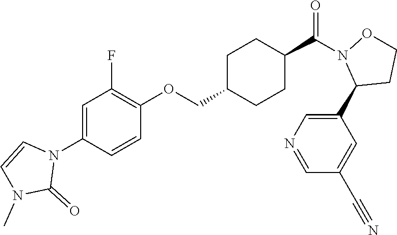 [SMILES: Cn1ccn(-c2ccc(OC[C@H]3CC[C@H](C(=O)N4OCC[C@H]4c4cncc(C#N)c4)CC3)c(F)c2)c1=O] | 1H NMR (400.23 MHz, DMSO-d6) δ ppm 8.94 (d, J = 1.83 Hz, 1 H), 8.78 (d, J = 2.08 Hz, 1 H), 8.20 (t, J = 1.90, 1.90 Hz, 1 H), 7.67 (dd, J = 13.08, 2.57 Hz, 1 H), 7.46 (d, J = 9.05 Hz, 1 H), 7.21 (t, J = 9.29, 9.29 Hz, 1 H), 7.01 (d, J = 3.06 Hz, 1 H), 6.71 (d, J = 3.06 Hz, 1 H), 5.42 (dd, J = 8.50, 6.54 Hz, 1 H), 4.30 (td, J = 7.73, 7.73, 3.00 Hz, 1 H), 3.90 (m, 3 H), 3.18 (s, 3 H), 2.91 (m, 1 H), 2.71 (m, 1 H), 2.30 (m, 1 H), 1.97 (br s, 1 H), 1.90 (br d, J = 13.45 Hz, 2 H), 1.79 (m, 1 H), 1.76 (m, 1 H), 1.37 (q, J = 12.63 Hz, 2H), 1.15 (m, 2H). LC/MS: m/z [M + H]⁺ 506.3, Rt (λ = |
| | | 220 nm): 1.95 min (LC/MS method |
| | | A), calc. m/z: 506.55. |

| | | |
|---|---|---|
| 90 | 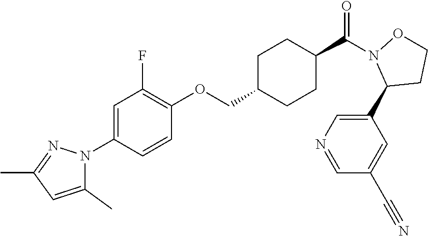 [SMILES: Cc1cc(C)n(-c2ccc(OC[C@H]3CC[C@H](C(=O)N4OCC[C@H]4c4cncc(C#N)c4)CC3)c(F)c2)n1] | 1H NMR (400.23 MHz, DMSO-d6) δ ppm 8.94 (d, J = 1.83 Hz, 1 H), 8.78 (d, J = 2.08 Hz, 1 H), 8.20 (t, J = 1.90, 1.90 Hz, 1 H), 7.38 (m, 1 H), 7.24 (d, J = 5.74 Hz, 1 H), 7.23 (s, 1 H), 6.03 (s, 1 H), 5.43 (dd, J = 8.44, 6.60 Hz, 1 H), 4.30 (td, J = 7.67, 7.67, 3.00 Hz, 1 H), 3.93 (m, 3 H), 2.91 (dddd, J = 12.09, 9.12, 6.42, 3.18 Hz, 1 H), 2.74 (br t, J = 11.92, 11.92 Hz, 1 H), 2.30 (m, 1 H), 2.26 (s, 3 H), 2.16 (s, 3 H), 1.98 (br s, 1 H), 1.92 (br d, J = 14.06 Hz, 2 H), 1.79 (br d, J = 9.78 Hz, 2 H), 1.39 (m 2H), 1.17 (m, 2H). LC/MS: m/z [M + H]⁺ 504.4, Rt (λ = |
| | | 220 nm): 2.38 min (LC/MS method |
| | | A), calc. m/z: 504.58. |

| | | |
|---|---|---|
| 91 | 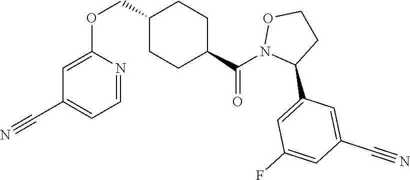 [SMILES: N#Cc1cc(F)cc([C@@H]2CCON2C(=O)[C@H]2CC[C@H](COc3cc(C#N)ccn3)CC2)c1] | LC/MS: m/z [M + H]⁺ 435.1, Rt (λ = 220 nm): 1.014 min (LC/MS method B), calc. m/z: 435.19. |

| | | |
|---|---|---|
| 92 | 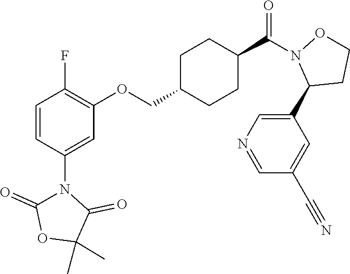 [SMILES: CC1(C)OC(=O)N(c2ccc(F)c(OC[C@H]3CC[C@H](C(=O)N4OCC[C@H]4c4cncc(C#N)c4)CC3)c2)C1=O] | 1H NMR (400.23 MHz, DMSO-d6) δ ppm 8.94 (d, J = 1.96 Hz, 1 H), 8.78 (d, J = 2.20 Hz, 1 H), 8.20 (t, J = 1.96, 1.96 Hz, 1 H), 7.36 (m, 2 H), 7.06 (m, 1 H), 5.42 (dd, J = 8.62, 6.42 Hz, 1 H), 4.30 (td, J = 7.70, 7.70, 3.06 Hz, 1 H), 3.92 (m, 1 H), 3.85 (d, J = 6.36 Hz, 2 H), 3.23 (m, 1 H), 2.91 (dddd, J = 12.13, 9.17, 6.45, 3.06 Hz, 1 H), 2.72 (m, 1 H), 2.30 (m, 1 H), 1.97 (br s, 1 H), 1.91 (br d, J = 14.55 Hz, 3 H), 1.80 (m, 1 H), 1.77 (br s, 1 H), 1.63 (s, 6 H), 1.39 (m, 2 H), 1.15 (m, 2 H). LC/MS: m/z [M + H]⁺ 537.3, Rt (λ = 220 nm): 2.24 min (LC/MS method A), calc. m/z: 537.56. |

| | | |
|---|---|---|
| 93 | 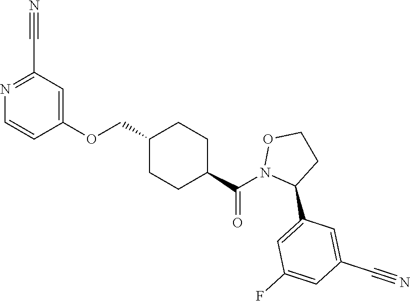 [SMILES: N#Cc1cc(F)cc([C@@H]2CCON2C(=O)[C@H]2CC[C@H](COc3ccnc(C#N)c3)CC2)c1] | LC/MS: m/z [M + H]⁺ 435.3, Rt (λ = 220 nm): 0.941 min (LC/MS method B), calc. m/z: 435.19. |

| | | |
|---|---|---|
| 94 | 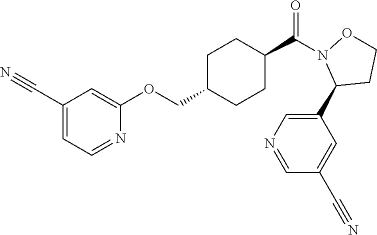 [SMILES: N#Cc1cncc([C@@H]2CCON2C(=O)[C@H]2CC[C@H](COc3cc(C#N)ccn3)CC2)c1] | 1H NMR (400.23 MHz, DMSO-d6) δ ppm 8.94 (d, J = 1.96 Hz, 1 H), 8.78 (d, J = 2.08 Hz, 1 H), 8.38 (dd, J = 5.01, 0.73 Hz, 1 H), 8.19 (t, J = 1.96, 1.96 Hz, 1 H), 7.38 (d, J = 5.86 Hz, 1 H), 7.37 (s, 1 H), 5.42 (dd, J = 8.50, 6.54 Hz, 1 H), 4.30 (td, J = 7.73, 7.73, 3.00 Hz, 1 H), 4.13 (d, J = 6.48 Hz, 2 H), 3.92 (m, 1 H), 2.90 (dddd, J = 12.13, 9.14, 6.42, 3.12 Hz, 1 H), 2.72 (br t, J = 11.92, 11.92 Hz, 1 H), 2.30 (m, 1 H), 1.94 (br d, J = 12.23 Hz, 1 H), 1.86 (br d, J = 12.35 Hz, 2 H), 1.77 (br d, J = 11.62 Hz, 2 H), 1.37 ( m, 2H), 1.13 (m, 2H). |
| | | LC/MS: m/z [M + H]⁺ 418.2, Rt (λ = |
| | | 220 nm): 2.13 min (LC/MS method |
| | | A), calc. m/z: 418.47. |

| | | |
|---|---|---|
| 95 | 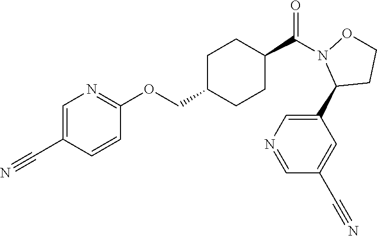 [SMILES: N#Cc1ccc(OC[C@H]2CC[C@H](C(=O)N3OCC[C@H]3c3cncc(C#N)c3)CC2)nc1] | 1H NMR (400.23 MHz, DMSO-d6) δ ppm 8.94 (d, J = 1.96 Hz, 1 H), 8.77 (d, J = 1.83 Hz, 1 H), 8.67 (d, J = 1.83 Hz, 1 H), 8.16 (m, 2 H), 7.01 (d, J = 8.80 Hz, 1 H), 5.41 (br t, J = 7.46, 7.46 Hz, 1 H), 4.29 (m, 1 H), 4.17 (d, J = 6.48 Hz, 1 H), 3.91 (m, 1 H), 2.90 (m, 1 H), 2.69 (m, 1 H), 2.30 (m, 1 H), 1.93 (m, 1 H), 1.85 (br dd, J = 8.31, 3.30 Hz, 2 H), 1.76 (m, 2 H), 1.59 (m, 1 H), 1.35 (m, 2 H), 1.12 (m, 2 H). LC/MS: m/z [M + H]⁺ 418.2, Rt (λ = 220 nm): 2.07 min (LC/MS method A), calc. m/z: 418.47. |

| | | |
|---|---|---|
| 96 | 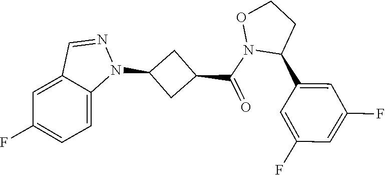 [SMILES: O=C([C@H]1C[C@@H](n2ncc3cc(F)ccc32)C1)N1OCC[C@H]1c1cc(F)cc(F)c1] | 1H NMR (400.23 MHz, DMSO-d6) δ ppm 8.09 (s, 1 H), 7.75 (dd, J = 9.11, 4.22 Hz, 1 H), 7.54 (dd, J = 9.17, 2.20 Hz, 1 H), 7.30 (td, J = 9.14, 9.14, 2.51 Hz, 1 H), 7.15 (tt, J = 9.35, 9.35, 2.32, 2.32 Hz, 1 H), 7.03 (m, 2 H), 5.38 (dd, J = 8.62, 6.30 Hz, 1 H), 5.29 (quin, J = 8.34, 8.34, 8.34, 8.34 Hz, 1 H), 4.24 (td, J = 7.70, 7.70, 2.81 Hz, 1 H), 3.84 (m, 1 H), 2.88 (m, 2 H), 2.72 (m, 3 H), 2.21 (m, 1 H). |
| | | LC/MS: m/z [M + H]⁺ 402.2, Rt (λ = |
| | | 220 nm): 2.43 min (LC/MS method |
| | | A), calc. m/z: 402.14. |

| | | |
|---|---|---|
| 97 | 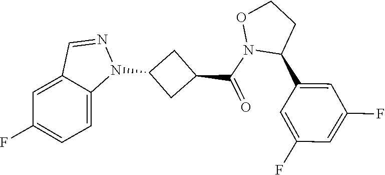 [SMILES: O=C([C@H]1C[C@H](n2ncc3cc(F)ccc32)C1)N1OCC[C@H]1c1cc(F)cc(F)c1] | 1H NMR (400.23 MHz, DMSO-d6) δ ppm 8.15 (s, 1 H), 7.70 (dd, J = 9.11, 4.22 Hz, 1 H), 7.54 (dd, J = 9.17, 2.20 Hz, 1 H), 7.26 (td, J = 9.14, 9.14, 2.51 Hz, 1 H), 7.15 (tt, J = 9.29, 9.29, 2.32, 2.32 Hz, 1 H), 7.06 (m, 2 H), 5.41 (dd, J = 8.68, 6.24 Hz, 1 H), 5.31 (quin, J = 7.49, 7.49, 7.49, 7.49 Hz, 1 H), 4.23 (td, J = 7.73, 7.73, 3.00 Hz, 1 H), 3.87 (ddd, J = 9.38, 7.98, 6.72 Hz, 1 H), 3.68 (br s, 1 H), 2.86 (m, 5 H), |
| | | 2.22 (m, 1 H). |
| | | LC/MS: m/z [M + H]⁺ 402.2, Rt (λ = |
| | | 220 nm): 2.49 min (LC/MS method |
| | | A), calc. m/z: 402.14. |

| | | |
|---|---|---|
| 98 | 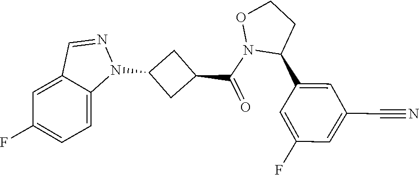 [SMILES: N#Cc1cc(F)cc([C@@H]2CCON2C(=O)[C@H]2C[C@H](n3ncc4cc(F)ccc43)C2)c1] | 1H NMR (400.23 MHz, DMSO-d6) δ ppm 8.15 (s, 1 H), 7.79 (d, J = 8.64 Hz, 1 H), 7.69 (m, 1 H), 7.69 (s, 1 H), 7.55 (t, J = 8.80, 8.80 Hz, 2 H), 7.26 (td, J = 9.14, 9.14, 2.51 Hz, 1 H), 5.45 (dd, J = 8.68, 6.36 Hz, 1 H), 5.31 (quin, J = 7.61, 7.61, 7.61, 7.61 Hz, 1 H), 4.24 (td, J = 7.70, 7.70, 2.93 Hz, 1 H), 3.89 (ddd, J = 9.35, 8.01, 6.60 Hz, 1 H), 3.69 (br s, 2 H), 2.93 (m, 1 H), 2.84 (m, 4 H), 2.25 (m, 1 H). LC/MS: m/z [M + H]⁺ 409.2, Rt (λ = 220 nm): 2.36 min (LC/MS method A), calc. m/z: 409.15. |

| | | |
|---|---|---|
| 99 | 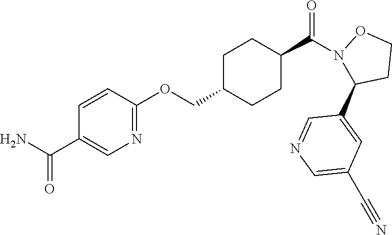 [SMILES: N#Cc1cncc([C@@H]2CCON2C(=O)[C@H]2CC[C@H](COc3ccc(C(N)=O)cn3)CC2)c1] | 1H NMR (400.23 MHz, DMSO-d6) δ ppm 8.94 (d, J = 1.83 Hz, 1 H), 8.78 (d, J = 2.08 Hz, 1 H), 8.65 (d, J = 2.08 Hz, 1 H), 8.19 (s, 1 H), 8.11 (dd, J = 8.68, 2.45 Hz, 1 H), 6.86 (d, J = 8.68 Hz, 1 H), 5.41 (s, 1 H), 4.30 (br d, J = 3.18 Hz, 1 H), 4.13 (d, J = 6.48 Hz, 2 H), 3.92 (br d, J = 9.41 Hz, 1 H), 3.51 (br s, 3 H), 2.67 (br d, J = 1.83 Hz, 7 H), 1.87 (br d, J = 12.35 Hz, 3 H), 1.37 (br d, J = 12.96 Hz, 1 H). LC/MS: m/z [M + H]⁺ 436.2, Rt (λ = 220 nm): 1.58 min (LC/MS method A), calc. m/z: 436.20. |
| 100 | 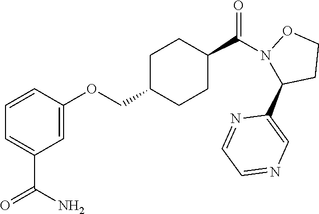 [SMILES: NC(=O)c1cccc(OC[C@H]2CC[C@H](C(=O)N3OCC[C@H]3c3cnccn3)CC2)c1] | 1H NMR (400.23 MHz, DMSO-d6) δ ppm 8.62 (m, 1 H), 8.58 (dd, J = 7.27, 1.90 Hz, 2 H), 7.93 (br s, 1 H), 7.37 (m, 4 H), 7.07 (dd, J = 8.13, 1.77 Hz, 1 H), 5.42 (dd, J = 8.93, 6.11 Hz, 1 H), 4.32 (td, J = 7.58, 7.58, 3.42 Hz, 1 H), 3.96 (m, 1 H), 3.83 (d, J = 4.65 Hz, 1 H), 3.82 (s, 1 H), 2.83 (m, 1 H), 2.70 (m, 1 H), 2.33 (m, 1 H), 1.91 (br d, J = 10.64 Hz, 3 H), 1.75 (br d, J = 9.05 Hz, 2 H), 1.39 (m, 2 H), 1.14 (m, 2 H). LC/MS: m/z [M + H]⁺ 411.2, Rt (λ = 220 nm): 1.6 min (LC/MS method A), |
| | | calc. m/z: 411.20. |
| 101 | 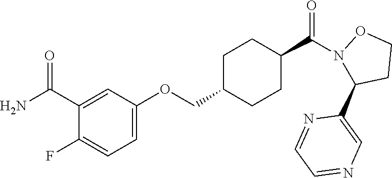 [SMILES: NC(=O)c1cc(OC[C@H]2CC[C@H](C(=O)N3OCC[C@H]3c3cnccn3)CC2)ccc1F] | 1H NMR (400.23 MHz, DMSO-d6) δ ppm 8.62 (m, 1 H), 8.58 (dd, J = 6.72, 1.96 Hz, 2 H), 7.64 (br s, 1 H), 7.61 (br s, 1 H), 7.15 (m, 2 H), 7.05 (m, 1 H), 5.42 (dd, J = 8.86, 6.05 Hz, 1 H), 4.31 (td, J = 7.61, 7.61, 3.48 Hz, 1 H), 3.96 (m, 1 H), 3.79 (d, J = 6.24 Hz, 2 H), 3.51 (br s, 2 H), 3.20 (br d, J = 6.36 Hz, 1 H), 2.83 (m, 1 H), 2.69 (m, 1 H), 1.90 (m, 3 H), 1.74 (br d, J = 12.23 Hz, 2 H), 1.72 (br s, 1 H), 1.37 (m, 3 H), |
| | | 1.13 (m, 2 H). |
| | | LC/MS: m/z [M + H]⁺ 429.2, Rt (λ = |
| | | 220 nm): 1.66 min (LC/MS method |
| | | A), calc. m/z: 429.19. |
| 102 | 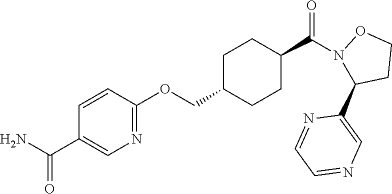 [SMILES: NC(=O)c1ccc(OC[C@H]2CC[C@H](C(=O)N3OCC[C@H]3c3cnccn3)CC2)nc1] | ″1H NMR (400.23 MHz, DMSO-d6) δ ppm 8.61 (m, 4 H), 8.11 (dd, J = 8.68, 2.45 Hz, 1 H), 7.94 (br s, 1 H), 7.36 (br s, 1 H), 6.86 (d, J = 8.68 Hz, 1 H), 5.42 (dd, J = 8.68, 6.11 Hz, 1 H), 4.31 (td, J = 7.52, 7.52, 3.30 Hz, 1 H), 4.13 (d, J = 6.48 Hz, 2 H), 3.95 (m, 1 H), 3.51 (br s, 1 H), 2.83 (m, 1 H), 2.69 (m, 1 H), 1.88 (m, 3 H), 1.75 (br s, 2 H), 1.36 (br t, J = 12.78, 12.78 Hz, 2 H), 1.13 (m, 2 H). LC/MS: m/z [M + H]⁺ 412.2, Rt (λ = |
| | | 220 nm): 1.43 min (LC/MS method |
| | | A), calc. m/z: 412.20. |
| 103 | 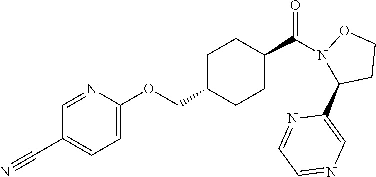 [SMILES: N#Cc1ccc(OC[C@H]2CC[C@H](C(=O)N3OCC[C@H]3c3cnccn3)CC2)nc1] | 1H NMR (400.23 MHz, DMSO-d6) δ ppm 8.61 (m, 3 H), 8.14 (dd, J = 8.74, 2.38 Hz, 1 H), 7.01 (d, J = 8.68 Hz, 1 H), 5.41 (dd, J = 8.68, 6.24 Hz, 1 H), 4.31 (td, J = 7.64, 7.64, 3.55 Hz, 1 H), 4.16 (d, J = 6.48 Hz, 2 H), 3.95 (m, 1 H), 2.82 (m, 1 H), 2.69 (m, 1 H), 1.87 (m, 3 H), 1.76 (br s, 2 H), 1.36 (m, 2 H), 1.13 (m, 2 H). LC/MS: m/z [M + H]⁺ 394.2, Rt (λ = 220 nm): 1.91 min (LC/MS method |
| | | A), calc. m/z: 394.19. |
| 104 | 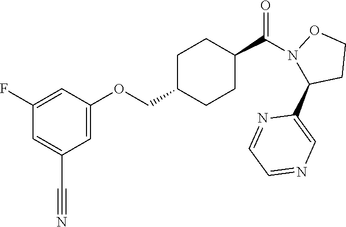 [SMILES: N#Cc1cc(F)cc(OC[C@H]2CC[C@H](C(=O)N3OCC[C@H]3c3cnccn3)CC2)c1] | 1H NMR (400.23 MHz, DMSO-d6) δ ppm 8.60 (m, 3 H), 7.38 (br d, J = 8.31 Hz, 1 H), 7.33 (s, 1 H), 7.25 (m, 1 H), 5.42 (dd, J = 8.80, 6.11 Hz, 1 H), 4.31 (td, J = 7.55, 7.55, 3.48 Hz, 1 H), 3.95 (m, 1 H), 3.88 (d, J = 6.36 Hz, 2 H), 2.83 (m, 1 H), 2.71 (br t, J = 12.65, 12.65 Hz, 1 H), 1.88 (m, 3 H), 1.76 (m, 2 H), 1.37 (br t, J = 12.65, 12.65 Hz, 2 H), 1.13 (m, 2 H). LC/MS: m/z [M + H]⁺ 411.2, Rt (λ = 220 nm): 2.2 min (LC/MS method A), calc. m/z: 411.18. |
| 105 | 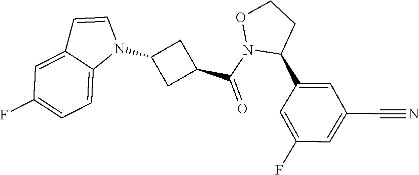 [SMILES: N#Cc1cc(F)cc([C@@H]2CCON2C(=O)[C@H]2C[C@H](n3ccc4cc(F)ccc43)C2)c1] | 1H NMR (400.23 MHz, DMSO-d6) δ ppm 7.73 (m, 3 H), 7.57 (br d, J = 9.66 Hz, 1 H), 7.44 (dd, J = 8.93, 4.40 Hz, 1 H), 7.30 (dd, J = 9.90, 2.45 Hz, 1 H), 6.95 (td, J = 9.20, 9.20, 2.51 Hz, 1 H), 6.49 (d, J = 3.06 Hz, 1 H), 5.46 (dd, J = 8.62, 6.54 Hz, 1 H), 5.03 (quin, J = 7.89, 7.89, 7.89, 7.89 Hz, 1 H), 4.24 (td, J = 7.73, 7.73, 2.87 Hz, 1 H), 3.90 (m, 1 H), 3.61 (br s, 1 H), 2.93 (m, 1 H), 2.81 (m, 3 H), 2.68 (m, 1 H), |
| | | 2.26 (m, 1 H). |
| | | LC/MS: m/z [M + H]⁺ 408.2, Rt (λ = |
| | | 220 nm): 2.52 min (LC/MS method |
| | | A), calc. m/z: 408.15. |
| 106 | 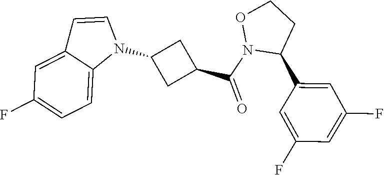 [SMILES: O=C([C@H]1C[C@H](n2ccc3cc(F)ccc32)C1)N1OCC[C@H]1c1cc(F)cc(F)c1] | 1H NMR (400.23 MHz, DMSO-d6) δ ppm 7.74 (d, J = 3.18 Hz, 1 H), 7.44 (dd, J = 8.93, 4.40 Hz, 1 H), 7.30 (dd, J = 9.90, 2.45 Hz, 1 H), 7.15 (m, 1 H), 7.06 (br d, J = 6.72 Hz, 2 H), 6.95 (td, J = 9.20, 9.20, 2.51 Hz, 1 H), 6.49 (d, J = 3.06 Hz, 1 H), 5.42 (dd, J = 8.50, 6.42 Hz, 1 H), 5.04 (dq, J = 8.19, 7.95, 7.95, 7.95 Hz, 1 H), 4.23 (td, J = 7.61, 7.61, 2.75 Hz, 1 H), 3.88 (m, 1 H), 3.60 (br s, 1 H), 2.91 (m, 1 H), 2.79 |
| | | (m, 3 H), 2.67 (m, 1 H), 2.22 (m, 1 |
| | | H). |
| | | LC/MS: m/z [M + H]⁺ 401.2, Rt (λ = |
| | | 220 nm): 2.65 min (LC/MS method |
| | | A), calc. m/z: 401.15. |
| 107 | 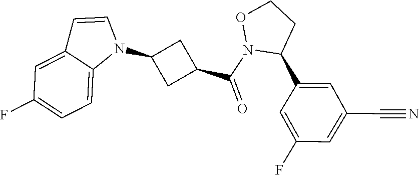 [SMILES: N#Cc1cc(F)cc([C@@H]2CCON2C(=O)[C@H]2C[C@@H](n3ccc4cc(F)ccc43)C2)c1] | 1H NMR (400.23 MHz, DMSO-d6) δ ppm 7.78 (br d, J = 7.58 Hz, 1 H), 7.66 (s, 1 H), 7.57 (d, J = 3.18 Hz, 1 H), 7.53 (m, 2 H), 7.30 (dd, J = 9.84, 2.51 Hz, 1 H), 6.97 (td, J = 9.20, 9.20, 2.51 Hz, 1 H), 6.46 (d, J = 3.06 Hz, 1 H), 5.42 (dd, J = 8.44, 6.60 Hz, 1 H), 5.00 (m, 1 H), 4.26 (m, 1 H), 3.87 (m, 1 H), 2.92 (m, 1 H), 2.76 (td, J = 7.49, 7.49, 3.48 Hz, 2 H), 2.65 (m, 1 H), 2.56 (m, 1 H), 2.25 (m, 1 H). |
| | | LC/MS: m/z [M + H]⁺ 408.2, Rt (λ = |
| | | 220 nm): 2.49 min (LC/MS method |
| | | A), calc. m/z: 408.15. |
| 108 | 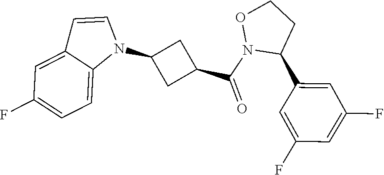 [SMILES: O=C([C@H]1C[C@@H](n2ccc3cc(F)ccc32)C1)N1OCC[C@H]1c1cc(F)cc(F)c1] | 1H NMR (400.23 MHz, DMSO-d6) δ ppm 7.57 (d, J = 3.18 Hz, 1 H), 7.52 (dd, J = 8.99, 4.46 Hz, 1 H), 7.30 (dd, J = 9.90, 2.45 Hz, 1 H), 7.14 (br t, J = 9.35, 9.35 Hz, 1 H), 7.04 (br d, J = 6.72 Hz, 2 H), 6.97 (t, J = 9.35, 9.35 Hz, 1 H), 6.46 (d, J = 3.06 Hz, 1 H), 5.38 (m, 1 H), 5.00 (quin, J = 8.28, 8.28, 8.28, 8.28 Hz, 1 H), 4.25 (m, 1 H), 3.86 (m, 1 H), 2.90 (m, 1 H), 2.77 (m, 1 H), 2.73 (br s, 1 H), 2.61 (m, 2 |
| | | H), 2.21 (m, 1 H). |
| | | LC/MS: m/z [M + H]⁺ 404.2, Rt (λ = |
| | | 220 nm): 2.62 min (LC/MS method |
| | | A), calc. m/z: 401.15. |
| 109 | 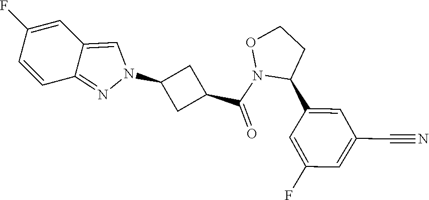 [SMILES: N#Cc1cc(F)cc([C@@H]2CCON2C(=O)[C@H]2C[C@@H](n3cc4cc(F)ccc4n3)C2)c1] | 1H NMR (400.23 MHz, DMSO-d6) δ ppm 8.40 (s, 1 H), 7.79 (br d, J = 8.44 Hz, 1 H), 7.69 (dd, J = 9.48, 4.83 Hz, 1 H), 7.66 (s, 1 H), 7.54 (br d, J = 9.66 Hz, 1 H), 7.41 (dd, J = 9.60, 2.26 Hz, 1 H), 7.14 (td, J = 9.38, 9.38, 2.51 Hz, 1 H), 5.42 (dd, J = 8.56, 6.60 Hz, 1 H), 5.18 (quin, J = 8.22, 8.22, 8.22, 8.22 Hz, 1 H), 4.26 (td, J = 7.70, 7.70, 2.69 Hz, 1 H), 3.87 (m, 1 H), 2.90 (m, 2 H), 2.76 (m, 2 H), 2.25 (m, 1 H). LC/MS: m/z [M + H]⁺ 409.2, Rt (λ = 220 nm): 2.16 min (LC/MS method |
| | | A), calc. m/z: 409.15. |
| 110 | 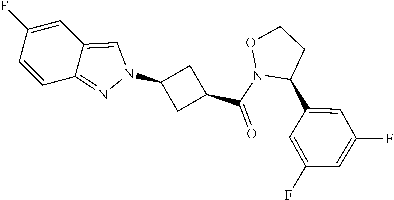 [SMILES: O=C([C@H]1C[C@@H](n2cc3cc(F)ccc3n2)C1)N1OCC[C@H]1c1cc(F)cc(F)c1] | 1H NMR (400.23 MHz, DMSO-d6) δ ppm 8.40 (s, 1 H), 7.69 (dd, J = 9.29, 4.77 Hz, 1 H), 7.40 (dd, J = 9.54, 2.32 Hz, 1 H), 7.14 (m, 2 H), 7.03 (br d, J = 6.60 Hz, 2 H), 5.38 (dd, J = 8.50, 6.54 Hz, 1 H), 5.18 (br t, J = 8.25, 8.25 Hz, 1 H), 4.24 (m, 1 H), 3.85 (m, 2 H), 2.89 (m, 2 H), 2.76 (m, 3 H), 2.21 (m, 1 H). LC/MS: m/z [M + H]⁺ 402.2, Rt (λ = 220 nm): 2.29 min (LC/MS method A), calc. m/z: 402.14. |
| 111 | 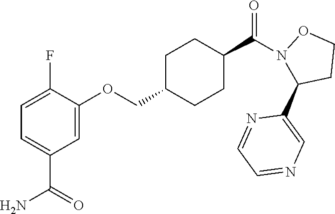 [SMILES: NC(=O)c1ccc(F)c(OC[C@H]2CC[C@H](C(=O)N3OCC[C@H]3c3cnccn3)CC2)c1] | 1H NMR (400.23 MHz, DMSO-d6) δ ppm 8.59 (m, 3 H), 7.98 (br s, 1 H), 7.63 (dd, J = 8.38, 1.77 Hz, 1 H), 7.48 (m, 1 H), 7.38 (br s, 1 H), 7.27 (dd, J = 11.06, 8.38 Hz, 1 H), 5.42 (dd, J = 8.50, 6.05 Hz, 1 H), 4.32 (td, J = 7.58, 7.58, 3.42 Hz, 1 H), 3.93 (m, 3 H), 2.83 (m, 1 H), 2.69 (m, 1 H), 1.91 (br d, J = 10.64 Hz, 3 H), 1.78 (br s, 2 H), 1.39 (m, 2 H), 1.15 (br t, J = 12.53, 12.53 Hz, 2 H). LC/MS: m/z [M + H]⁺ 429.2, Rt (λ = 220 nm): 1.64 min (LC/MS method |
| | | A), calc. m/z: 429.19. |
| 112 | 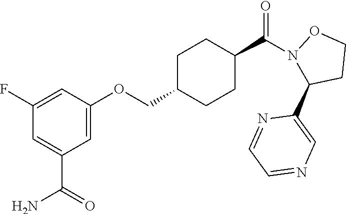 [SMILES: NC(=O)c1cc(F)cc(OC[C@H]2CC[C@H](C(=O)N3OCC[C@H]3c3cnccn3)CC2)c1] | 1H NMR (400.23 MHz, DMSO-d6) δ ppm 8.59 (m, 3 H), 8.01 (br s, 1 H), 7.48 (br s, 1 H), 7.29 (s, 1 H), 7.22 (br d, J = 8.93 Hz, 1 H), 6.99 (m, 1 H), 5.42 (m, 1 H), 4.31 (dt, J = 7.70, 3.85, 3.85 Hz, 1 H), 3.96 (q, J = 7.66, 7.66, 7.66 Hz, 1 H), 3.85 (d, J = 6.36 Hz, 2 H), 2.83 (m, 1 H), 2.69 (br d, J = 18.10 Hz, 2 H), 1.90 (br s, 3 H), 1.77 (br s, 2 H), 1.38 (br t, J = 12.90, 12.90 Hz, 2 H), 1.16 (m, 2 H). LC/MS: m/z [M + H]⁺ 429.2, Rt (λ = 220 nm): 1.72 min (LC/MS method A), calc. m/z: 429.19. |
| 113 | 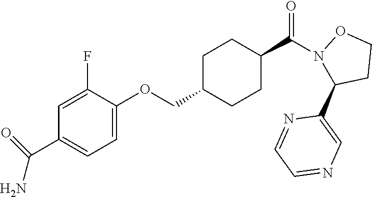 [SMILES: NC(=O)c1ccc(OC[C@H]2CC[C@H](C(=O)N3OCC[C@H]3c3cnccn3)CC2)c(F)c1] | LC/MS: m/z [M + H]⁺ 429.2, Rt (λ = 220 nm): 1.58 min (LC/MS method A), calc. m/z: 429.19. |
| 114 | 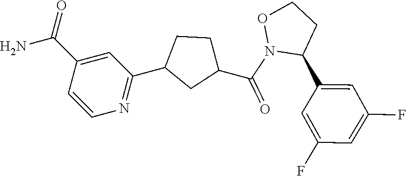 [SMILES: NC(=O)c1ccnc(C2CCC(C(=O)N3OCC[C@H]3c3cc(F)cc(F)c3)C2)c1] | Stereoisomer 2 ¹H NMR (400 MHz, CDCl₃): δ ppm 8.67 (d, J = 5.1 Hz, 1 H), 7.65 (s, 1 H), 7.48 (dd, J = 1.5, 5.1 Hz, 1 H), 6.84 (dd, J = 1.9, 7.9 Hz, 2 H), 6.70 (tt, J = 2.1, 8.8 Hz, 1 H), 6.45-6.10 (m, 1 H), 5.84-5.52 (m, 1 H), 5.39 (dd, J = 6.2, 8.8 Hz, 1 H), 4.27 (dt, J = 3.3, 7.8 Hz, 1 H), 3.94-3.85 (m, 1 H), 3.52- 3.33 (m, 2 H), 2.87 (dddd, J = 3.3, 6.4, 9.2, 12.3 Hz, 1 H), 2.60-2.47 (m, 1 H), |
| | | 2.40-2.28 (m, 1 H), 2.25-2.14 (m, 2 |
| | | H), 2.13-2.05 (m, 1 H), 2.05-1.93 (m, |
| | | 2 H). |
| | | SFC (Column: Chiralpak AD-3 |
| | | 50 × 4.6 mm I.D., 3 μm; mobile phase: |
| | | CO₂ and MeOH (0.05% DEA), |
| | | gradient: 5%-40% MeOH (0.05% |
| | | DEA); flow rate: 3 ml/min; column |
| | | temperature: 35 °C; 100 bar): Rt = |
| | | 2.69 min (100%). |
| | | LC/MS: m/z [M + H]⁺ 402.2, Rt (λ = |
| | | 220 nm): 0.777 min (LC/MS method |
| | | B), calc. m/z: 402.41. |
| 115 | 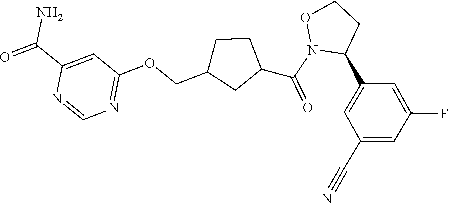 [SMILES: N#Cc1cc(F)cc([C@@H]2CCON2C(=O)C2CCC(COc3cc(C(N)=O)ncn3)C2)c1] | Stereoisomer 1 ¹H NMR (400 MHz, CDCl₃): δ ppm 8.74 (s, 1 H), 7.76 (br s, 1 H), 7.50 (s, 1 H), 7.42 (s, 1 H), 7.29-7.25 (m, 2 H), 5.85 (br s, 1 H), 5.41 (dd, J = 6.6, 8.7 Hz, 1 H), 4.36 (d, J = 7.1 Hz, 2 H), 4.28 (dt, J = 2.8, 7.9 Hz, 1 H), 3.92-3.84 (m, 1 H), 3.30 (quin, J = 8.3 Hz, 1 H), 2.96- 2.83 (m, 1 H), 2.56-2.46 (m, 1 H), 2.38-2.21 (m, 2 H), 1.94-1.88 (m, 3 H), 1.71-1.58 (m, 2 H). SFC (Column: Chiralpak AS-3 50 × 4.6 mm I.D., 3 μm; mobile phase: |
| | | CO₂ and MeOH (0.05% DEA), |
| | | gradient: 5%-40% MeOH (0.05% |
| | | DEA); flow rate: 3 ml/min; column |
| | | temperature: 35 °C; 100 bar): Rt = |
| | | 1.79 min (100%). |
| | | LC/MS: m/z [M + H]⁺ 440.1, Rt (λ = |
| | | 220 nm): 0.631 min (LC/MS method |
| | | B), calc. m/z: 440.18. |
| 116 | 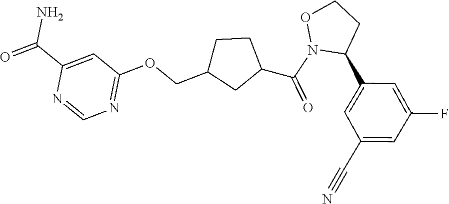 [SMILES: N#Cc1cc(F)cc([C@@H]2CCON2C(=O)C2CCC(COc3cc(C(N)=O)ncn3)C2)c1] | Stereoisomer 2 ¹H NMR (400 MHz, CDCl₃): δ ppm 8.76 (d, J = 1.0 Hz, 1 H), 7.76 (br s, 1 H), 7.53 (d, J = 1.0 Hz, 1 H), 7.42 (s, 1H), 7.27 (s, 2 H), 5.67 (br s, 1 H), 5.41 (dd, J = 6.3, 8.7 Hz, 1 H), 4.37- 4.32 (m, 2 H), 4.28 (dt, J = 2.6, 8.0 Hz, 1 H), 3.88 (ddd, J = 6.6, 8.2, 9.5 Hz, 1 H), 3.34 (quin, J = 7.9 Hz, 1 H), 2.90 (dddd, J = 2.9, 6.4, 9.2, 12.3 Hz, 1 H), 2.65-2.56 (m, 1 H), 2.36-2.25 (m, 1 H), 2.14 (ddd, J = 7.2, 8.4, 13.4 Hz, 1 H), 2.06-1.97 (m, 2 H), 1.90-1.81 (m, |
| | | 2 H), 1.52-1.44 (m, 1 H). |
| | | SFC (Column: Chiralpak AS-3 |
| | | 50 × 4.6 mm I.D., 3 μm; mobile phase: |
| | | CO₂ and MeOH (0.05% DEA), |
| | | gradient: 5%-40% MeOH (0.05% |
| | | DEA); flow rate: 3 ml/min; column |
| | | temperature: 35 °C; 100 bar): Rt = |
| | | 1.66 min (99.9%). |
| | | LC/MS: m/z [M + H]⁺ 440.1, Rt (λ = |
| | | 220 nm): 0.629 min (LC/MS method |
| | | B), calc. m/z: 440.18. |
| 117 | 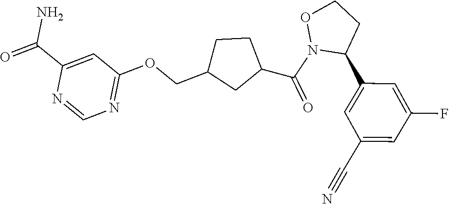 [SMILES: N#Cc1cc(F)cc([C@@H]2CCON2C(=O)C2CCC(COc3cc(C(N)=O)ncn3)C2)c1] | Stereoisomer 3 ¹H NMR (400 MHz, CDCl₃): δ ppm 8.76 (d, J = 0.6 Hz, 1 H), 7.82-7.71 (m, 1 H), 7.53 (d, J = 0.6 Hz, 1 H), 7.42 (s, 1 H), 7.28-7.26 (m, 2 H), 5.70 (br s, 1 H), 5.41 (dd, J = 6.4, 8.7 Hz, 1 H), 4.39-4.25 (m, 3 H), 3.93-3.84 (m, 1 H), 3.34 (quin, J = 7.9 Hz, 1 H), 2.90 (dddd, J = 3.0, 6.5, 9.2, 12.3 Hz, 1 H), 2.60 (td, J = 7.6, 14.7 Hz, 1 H), 2.36- 2.25 (m, 1 H), 2.18-2.00 (m, 3 H), 1.96-1.85 (m, 1 H), 1.71 (ddd, J = 7.0, 8.9, 13.2 Hz, 1 H), 1.52-1.42 (m, 1 H). |
| | | SFC (Column: Chiralpak AS-3 |
| | | 50 × 4.6 mm I.D., 3 μm; mobile phase: |
| | | CO₂ and MeOH (0.05% DEA), |
| | | gradient: 5%-40% MeOH (0.05% |
| | | DEA); flow rate: 3 ml/min; column |
| | | temperature: 35 °C; 100 bar): Rt = |
| | | 2.12 min (100%). |
| | | LC/MS: m/z [M + H]⁺ 440.1, Rt (λ = |
| | | 220 nm): 0.633 min (LC/MS method |
| | | B), calc. m/z: 440.18. |
| 118 | 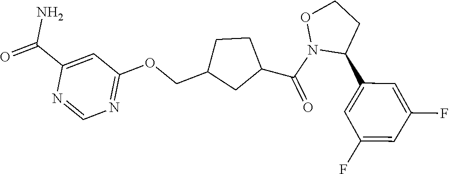 [SMILES: NC(=O)c1cc(OCC2CCC(C(=O)N3OCC[C@H]3c3cc(F)cc(F)c3)C2)ncn1] | Stereoisomer 1 ¹H NMR (400 MHz, CDCl₃): δ ppm 8.75 (s, 1 H), 7.76 (br s, 1 H), 7.53 (s, 1 H), 6.89-6.80 (m, 2 H), 6.70 (tt, J = 2.2, 8.8 Hz, 1 H), 5.72 (br s, 1 H), 5.37 (dd, J = 6.2, 8.7 Hz, 1 H), 4.34 (d, J = 6.7 Hz, 2 H), 4.25 (dt, J = 3.4, 7.8 Hz, 1 H), 3.95-3.80 (m, 1 H), 3.39- 3.27 (m, 1 H), 2.90-2.77 (m, 1 H), 2.60 (td, J = 7.5, 14.7 Hz, 1 H), 2.37- 2.25 (m, 1 H), 2.20-2.10 (m, 1 H), |
| | | 2.08-1.93 (m, 2 H), 1.92-1.78 (m, 2 |
| | | H), 1.47 (qd, J = 8.6, 12.3 Hz, 1 H). |
| | | SFC (Column: Chiralpak AS-3 |
| | | 50 × 4.6 mm I.D., 3 μm; mobile phase: |
| | | CO₂ and EtOH (0.05% DEA), |
| | | gradient: 5%-40% EtOH (0.05% |
| | | DEA); flow rate: 3 ml/min; column |
| | | temperature: 35 °C; 100 bar): Rt = |
| | | 1.59 min (100%). |
| | | LC/MS: m/z [M + H]⁺ 433.1, Rt (λ = |
| | | 220 nm): 0.651 min (LC/MS method |
| | | B), calc. m/z: 433.43. |
| 119 | 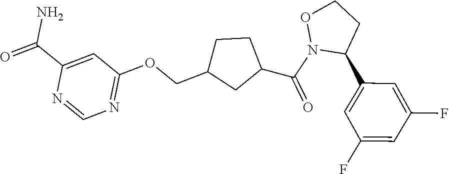 [SMILES: NC(=O)c1cc(OCC2CCC(C(=O)N3OCC[C@H]3c3cc(F)cc(F)c3)C2)ncn1] | Stereoisomer 2 ¹H NMR (400 MHz, CDCl₃): δ ppm 8.74 (s, 1 H), 7.76 (br s, 1 H), 7.50 (s, 1 H), 6.88-6.79 (m, 2 H), 6.69 (tt, J = 2.2, 8.8 Hz, 1 H), 5.73 (br d, J = 1.5 Hz, 1 H), 5.37 (dd, J = 6.2, 8.7 Hz, 1 H), 4.36 (d, J = 7.2 Hz, 2 H), 4.25 (dt, J = 3.2, 7.8 Hz, 1 H), 3.92-3.81 (m, 1 H), 3.39-3.22 (m, 1 H), 2.90-2.78 (m, 1 H), 2.56-2.43 (m, 1 H), 2.38-2.20 (m, 2 H), 1.99-1.84 (m, 3 H), 1.68 (td, |
| | | J = 9.4, 12.8 Hz, 1 H), 1.61-1.52 (m, 1 |
| | | H). |
| | | SFC (Column: Chiralpak AS-3 |
| | | 50 × 4.6 mm I.D., 3 μm; mobile phase: |
| | | CO₂ and EtOH (0.05% DEA), |
| | | gradient: 5%-40% EtOH (0.05% |
| | | DEA); flow rate: 3 ml/min; column |
| | | temperature: 35 °C; 100 bar): Rt = |
| | | 1.68 min (99.5%). |
| | | LC/MS: m/z [M + H]⁺ 433.1, Rt (λ = |
| | | 220 nm): 0.654 min (LC/MS method |
| | | B), calc. m/z: 433.43. |
| 120 | 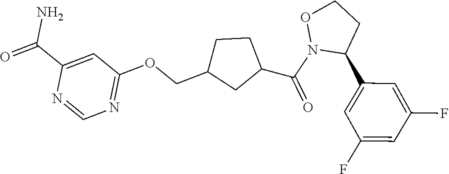 [SMILES: NC(=O)c1cc(OCC2CCC(C(=O)N3OCC[C@H]3c3cc(F)cc(F)c3)C2)ncn1] | Stereoisomer 3 ¹H NMR (400 MHz, CDCl₃): δ ppm 8.75 (s, 1 H), 7.77 (br s, 1 H), 7.52 (s, 1 H), 6.92-6.80 (m, 2 H), 6.70 (tt, J = 2.2, 8.8 Hz, 1 H), 5.78 (br s, 1 H), 5.37 (dd, J = 6.1, 8.7 Hz, 1 H), 4.33 (dq, J = 6.8, 10.4 Hz, 2 H), 4.24 (dt, J = 3.2, 7.8 Hz, 1 H), 3.95-3.79 (m, 1 H), 3.40-3.24 (m, 1 H), 2.85 (dddd, J = 3.2, 6.4, 9.2, 12.2 Hz, 1 H), 2.67- 2.52 (m, 1 H), 2.39-2.25 (m, 1 H), |
| | | 2.18-2.06 (m, 2 H), 2.06-1.97 (m, 1 |
| | | H), 1.96-1.86 (m, 1 H), 1.75-1.65 (m, |
| | | 1 H), 1.46 (qd, J = 8.3, 12.2 Hz, 1 H). |
| | | SFC (Column: Chiralpak AS-3 |
| | | 50 × 4.6 mm I.D., 3 μm; mobile phase: |
| | | CO₂ and EtOH (0.05% DEA), |
| | | gradient: 5%-40% EtOH (0.05% |
| | | DEA); flow rate: 3 ml/min; column |
| | | temperature: 35 °C; 100 bar): Rt = |
| | | 2.08 min (100%). |
| | | LC/MS: m/z [M + H]⁺ 433.1, Rt (λ = |
| | | 220 nm): 0.652 min (LC/MS method |
| | | B), calc. m/z: 433.43. |
| 121 | 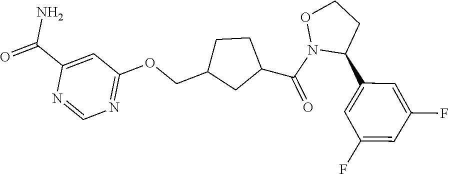 [SMILES: NC(=O)c1cc(OCC2CCC(C(=O)N3OCC[C@H]3c3cc(F)cc(F)c3)C2)ncn1] | Stereoisomer 4 ¹H NMR (400 MHz, CDCl₃): δ ppm 8.74 (d, J = 0.9 Hz, 1 H), 7.76 (br s, 1 H), 7.51 (d, J = 0.9 Hz, 1 H), 6.90-6.79 (m, 2 H), 6.70 (tt, J = 2.2, 8.8 Hz, 1 H), 5.78 (br s, 1 H), 5.37 (dd, J = 6.2, 8.7 Hz, 1 H), 4.44-4.30 (m, 2 H), 4.25 (dt, J = 3.2, 7.8 Hz, 1 H), 3.92-3.80 (m, 1 H), 3.30 (br t, J = 8.0 Hz, 1 H), 2.84 (dddd, J = 3.2, 6.5, 9.2, 12.2 Hz, 1 H), 2.57-2.42 (m, 1 H), 2.39-2.25 (m, 1 |
| | | H), 2.17-1.83 (m, 5 H), 1.61-1.51 (m, |
| | | 1 H). |
| | | SFC (Column: Chiralpak AS-3 |
| | | 50 × 4.6 mm I.D., 3 μm; mobile phase: |
| | | CO₂ and EtOH (0.05% DEA), |
| | | gradient: 5%-40% EtOH (0.05% |
| | | DEA); flow rate: 3 ml/min; column |
| | | temperature: 35 °C; 100 bar): Rt = |
| | | 2.36 min (100%). |
| | | LC/MS: m/z [M + H]⁺ 433.1, Rt (λ = |
| | | 220 nm): 0.668 min (LC/MS method |
| | | B), calc. m/z: 433.43. |
| 122 | 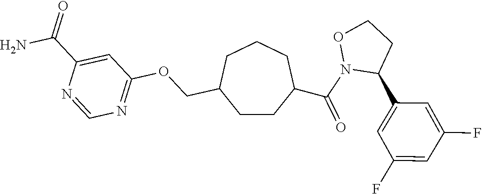 [SMILES: NC(=O)c1cc(OCC2CCCC(C(=O)N3OCC[C@H]3c3cc(F)cc(F)c3)CC2)ncn1] | Stereoisomer 1 ¹H NMR (400 MHz, DMSO-d₆): δ ppm 8.85 (d, J = 0.9 Hz, 1 H), 8.21 (br s, 1H), 7.91 (br s, 1 H), 7.31 (d, J = 0.9 Hz, 1 H), 7.13 (tt, J = 2.2, 9.3 Hz, 1 H), 6.98 (br d, J = 6.6 Hz, 2 H), 5.34-5.29 (m, 1 H), 4.26-4.22 (m, 1 H), 4.19 (br d, J = 6.8 Hz, 2 H), 3.89-3.82 (m, 1 H), 2.94-2.82 (m, 2 H), 2.22-2.13 (m, 1 H), 2.04-1.95 (m, 1 H), 1.85-1.72 (m, 6 H), 1.55-1.45 (m, 2 H), 1.40-1.30 (m, 1 H), 1.24-1.15 (m, 1 H). |
| | | SFC (Column: Chiralpak AS-3 |
| | | 50 × 4.6 mm I.D., 3 μm; mobile phase: |
| | | CO₂ and IPA (0.05% DEA), gradient: |
| | | 5%-40% IPA (0.05% DEA); flow rate: |
| | | 3 ml/min; column temperature: 35 °C; |
| | | 100 bar): Rt = 2.13 min (100%). |
| | | LC/MS: m/z [M + H]⁺ 461.00, Rt (λ = |
| | | 220 nm): 0.683 min (LC/MS method |
| | | B), calc. m/z: 461.48. |
| 123 | 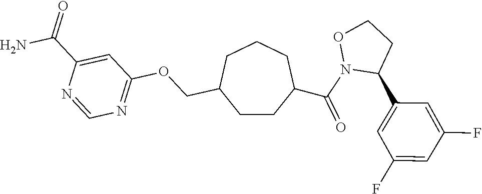 [SMILES: NC(=O)c1cc(OCC2CCCC(C(=O)N3OCC[C@H]3c3cc(F)cc(F)c3)CC2)ncn1] | Stereoisomer 2 ¹H NMR (400 MHz, DMSO-d₆): δ ppm 8.86 (d, J = 1.0 Hz, 1 H), 8.21 (br s, 1 H), 7.91 (br s, 1 H), 7.32 (d, J = 0.9 Hz, 1 H), 7.13 (tt, J = 2.2, 9.3 Hz, 1 H), 6.98 (br d, J = 6.5 Hz, 2 H), 5.34-5.29 (m, 1 H), 4.27-4.23 (m, 1 H), 4.20 (br d, J = 6.6 Hz, 2 H), 3.89-3.82 (m, 1 H), 2.91-2.83 (m, 2 H), 2.21-2.15 (m, 1 H), 1.97-1.92 (m, 2 H), 1.81-1.73 (m, 2 H), 1.62 (br d, J = 2.8 Hz, 3 H), 1.59- 1.47 (m, 2 H), 1.36-1.25 (m, 2 H). |
| | | SFC (Column: Chiralpak AS-3 |
| | | 50 × 4.6 mm I.D., 3 μm; mobile phase: |
| | | CO₂ and IPA (0.05% DEA), gradient: |
| | | 5%-40% IPA (0.05% DEA); flow rate: |
| | | 3 ml/min; column temperature: 35 °C; |
| | | 100 bar): Rt = 2.09 min (100%). |
| | | LC/MS: m/z [M + H]⁺ 461.00, Rt (λ = |
| | | 220 nm): 0.684 min (LC/MS method |
| | | B), calc. m/z: 461.48. |
| 124 | 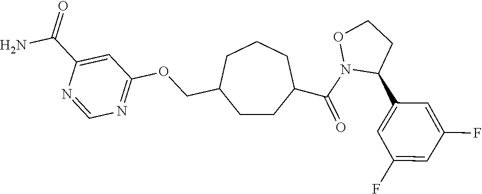 [SMILES: NC(=O)c1cc(OCC2CCCC(C(=O)N3OCC[C@H]3c3cc(F)cc(F)c3)CC2)ncn1] | Stereoisomer 3 ¹H NMR (400 MHz, DMSO-d₆): δ ppm 8.85 (d, J = 1.0 Hz, 1 H), 8.20 (br s, 1H), 7.90 (br s, 1 H), 7.32 (d, J = 1.0 Hz, 1 H), 7.13 (tt, J = 2.4, 9.3 Hz, 1 H), 7.00-6.95 (m, 2 H), 5.32 (dd, J = 6.2, 8.4 Hz, 1 H), 4.26-4.22 (m, 1 H), 4.20 (dd, J = 1.4, 6.5 Hz, 2 H), 3.89-3.83 (m, 1 H), 2.92-2.82 (m, 2 H), 2.22- 2.13 (m, 1 H), 2.00-1.94 (m, 1 H), 1.92-1.85 (m, 2 H), 1.81-1.73 (m, 2 H), 1.66-1.59 (m, 3 H), 1.56-1.45 (m, |
| | | 1 H), 1.40-1.30 (m, 1 H), 1.28-1.20 |
| | | (m, 1 H). |
| | | SFC (Column: Chiralpak AS-3 |
| | | 50 × 4.6 mm I.D., 3 μm; mobile phase: |
| | | CO₂ and IPA (0.05% DEA), gradient: |
| | | 5%-40% IPA (0.05% DEA); flow rate: |
| | | 3 ml/min; column temperature: 35 °C; |
| | | 100 bar): Rt = 2.39 min (100%). |
| | | LC/MS: m/z [M + H]⁺ 461.1, Rt (λ = |
| | | 220 nm): 0.884 min (LC/MS method |
| | | B), calc. m/z: 461.48. |
| 125 | 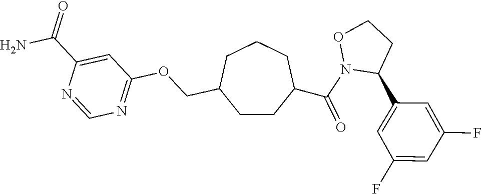 [SMILES: NC(=O)c1cc(OCC2CCCC(C(=O)N3OCC[C@H]3c3cc(F)cc(F)c3)CC2)ncn1] | Stereoisomer 4 ¹H NMR (400 MHz, DMSO-d₆): δ ppm 8.86 (d, J = 0.9 Hz, 1 H), 8.20 (br s, 1 H), 7.90 (br s, 1 H), 7.31 (d, J = 0.9 Hz, 1 H), 7.13 (tt, J = 2.3, 9.2 Hz, 1 H), 7.00-6.95 (m, 2 H), 5.32 (dd, J = 6.6, 8.4 Hz, 1 H), 4.26-4.22 (m, 1 H), 4.20 (d, J = 6.7 Hz, 2 H), 3.90-3.82 (m, 1 H), 2.94-2.82 (m, 2 H), 2.22-2.13 (m, 1 H), 1.98 (br dd, J = 3.7, 8.1 Hz, 2 H), 1.87-1.82 (m, 2 H), 1.74-1.65 (m, 3 H), 1.57-1.37 (m, 3 H), 1.23-1.15 (m, 1 H). |
| | | SFC (Column: Chiralpak AS-3 |
| | | 50 × 4.6 mm I.D., 3 μm; mobile phase: |
| | | CO₂ and IPA (0.05% DEA), gradient: |
| | | 5%-40% IPA (0.05% DEA); flow rate: |
| | | 3 ml/min; column temperature: 35 °C; |
| | | 100 bar): Rt = 2.57 min (99.5%). |
| | | LC/MS: m/z [M + H]⁺ 461.1, Rt (λ = |
| | | 220 nm): 0.89 min (LC/MS method |
| | | B), calc. m/z: 461.48. |
| 126 | 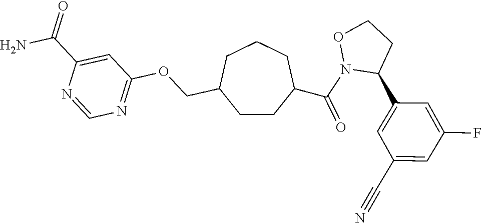 [SMILES: N#Cc1cc(F)cc([C@@H]2CCON2C(=O)C2CCCC(COc3cc(C(N)=O)ncn3)CC2)c1] | Stereoisomer 1 ¹H NMR (400 MHz, DMSO-d₆): δ ppm 8.85 (s, 1 H), 8.21 (br s, 1 H), 7.91 (br s, 1 H), 7.77 (br d, J = 8.4 Hz, 1 H), 7.60 (s, 1 H), 7.47 (br d, J = 9.5 Hz, 1 H), 7.31 (d, J = 0.6 Hz, 1 H), 5.35 (dd, J = 6.7, 8.3 Hz, 1 H), 4.25 (dt, J = 2.9, 7.8 Hz, 1 H), 4.19 (d, J = 6.7 Hz, 2 H), 3.91-3.84 (m, 1 H), 2.94-2.83 (m, 2 H), 2.26-2.16 (m, 1 H), 1.99 (br d, J = 3.1 Hz, 1 H), 1.85-1.73 (m, 6 H), 1.55-1.45 (m, 2 H), 1.35 (br d, J = 12.2 Hz, 1 H), 1.23-1.17 (m, 1 H). SFC (Column: Chiralpak IG-3 50 × 4.6 |
| | | mm I.D., 3 μm; mobile phase: 40% |
| | | IPA + ACN (0.05% DEA) in CO₂; flow |
| | | rate: 3 ml/min; column temperature: |
| | | 35 °C; 100 bar): Rt = 1.80 min |
| | | (99.8%). |
| | | LC/MS: m/z [M + H]⁺ 468.3, Rt (λ = |
| | | 220 nm): 0.83 min (LC/MS method |
| | | B), calc. m/z: 468.21. |
| 127 | 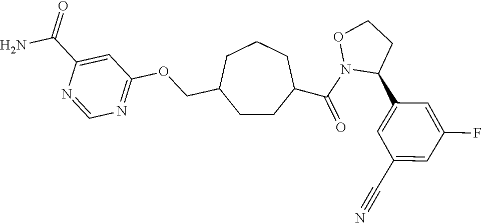 [SMILES: N#Cc1cc(F)cc([C@@H]2CCON2C(=O)C2CCCC(COc3cc(C(N)=O)ncn3)CC2)c1] | Stereoisomer 2 ¹H NMR (400 MHz, DMSO-d₆): δ ppm 8.86 (d, J = 0.6 Hz, 1 H), 8.21 (br s, 1 H), 7.91 (br s, 1 H), 7.77 (br d, J = 7.7 Hz, 1 H), 7.61 (s, 1 H), 7.48 (br d, J = 9.4 Hz, 1 H), 7.32 (d, J = 0.7 Hz, 1 H), 5.36 (dd, J = 6.8, 8.3 Hz, 1 H), 4.26 (dt, J = 2.8, 7.6 Hz, 1 H), 4.20 (d, J = 6.5 Hz, 2 H), 3.91-3.84 (m, 1 H), 2.92- 2.84 (m, 2 H), 2.26-2.17 (m, 1 H), 2.00-1.88 (m, 3 H), 1.80-1.69 (m, 2 H), 1.62 (br s, 3 H), 1.57-1.48 (m, 1 H), 1.36-1.23 (m, 2 H). SFC Column: Chiralpak IG-3 50 × 4.6 |
| | | mm I.D., 3 μm; mobile phase: 40% |
| | | IPA + CAN (0.05% DEA) in CO₂; flow |
| | | rate: 3 ml/min; column temperature: |
| | | 35 °C; 100 bar): Rt = 2.09 min |
| | | (98.6%). |
| | | LC/MS: m/z [M + H]⁺ 468.3, Rt (λ = |
| | | 220 nm): 0.84 min (LC/MS method |
| | | B), calc. m/z: 468.21. |
| 128 | 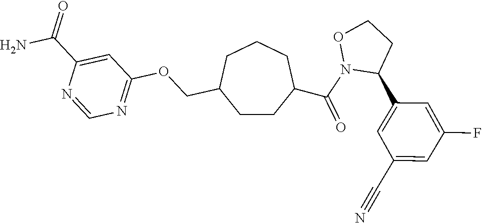 [SMILES: N#Cc1cc(F)cc([C@@H]2CCON2C(=O)C2CCCC(COc3cc(C(N)=O)ncn3)CC2)c1] | Stereoisomer 3 ¹H NMR (400 MHz, DMSO-d₆): δ ppm 8.86 (d, J = 0.9 Hz, 1 H), 8.20 (br s, 1 H), 7.90 (br s, 1 H), 7.77 (br d, J = 7.6 Hz, 1 H), 7.60 (s, 1 H), 7.47 (br d, J = 9.7 Hz, 1 H), 7.32 (d, J = 0.9 Hz, 1 H), 5.36 (dd, J = 6.5, 8.6 Hz, 1 H), 4.25 (dt, J = 3.0, 7.7 Hz, 1 H), 4.20 (dd, J = 1.8, 6.5 Hz, 2 H), 3.92-3.85 (m, 1 H), 2.92-2.84 (m, 2 H), 2.26-2.17 (m, 1 H), 1.96 (br dd, J = 4.4, 9.0 Hz, 1 H), 1.91-1.85 (m, 2 H), 1.81-1.73 (m, 2 H), 1.66-1.59 (m, 3 H), 1.55-1.45 (m, 1 H), 1.38-1.31 (m, 1 H), 1.27-1.18 (m, 1 H). |
| | | SFC (Column: Chiralpak IG-3 50 × 4.6 |
| | | mm I.D., 3 μm; mobile phase: |
| | | 40% IPA + ACN (0.05% DEA) in CO₂; |
| | | flow rate: 3 ml/min; column |
| | | temperature: 35 °C; 100 bar): Rt = |
| | | 2.44 min (100%). |
| | | LC/MS: m/z [M + H]⁺ 468.1, Rt (λ = |
| | | 220 nm): 0.86 min (LC/MS method |
| | | B), calc. m/z: 468.21. |
| 129 | 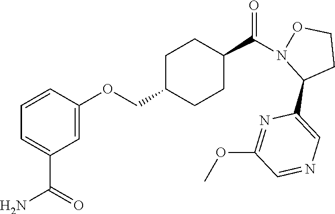 [SMILES: COc1cncc([C@@H]2CCON2C(=O)[C@H]2CC[C@H](COc3cccc(C(N)=O)c3)CC2)n1] | 1H NMR (400.23 MHz, DMSO-d6) δ ppm 8.22 (s, 1 H), 8.13 (s, 1 H), 7.93 (br s, 1 H), 7.38 (m, 5 H), 7.17 (m, 1 H), 7.07 (dd, J = 7.95, 1.83 Hz, 1 H), 5.34 (dd, J = 8.93, 5.50 Hz, 1 H), 4.31 (td, J = 7.58, 7.58, 4.28 Hz, 1 H), 4.09 (br s, 1 H), 3.98 (m, 1 H), 3.91 (s, 4 H), 3.83 (br d, J = 6.24 Hz, 3 H), 3.72 (br s, 1 H), 3.55 (br s, 27 H), 2.77 (m, 2 H), 2.43 (m, 1 H), 1.91 (br d, J = 11.74 Hz, 4 H), 1.75 (br s, 2 H), 1.38 (m, 3 H), 1.15 (m, 3 H). LC/MS: m/z [M + H]⁺ 441.2, Rt (λ = |
| | | 220 nm): 1.85 min (LC/MS method |
| | | A), calc. m/z: 441.21. |
| 130 | 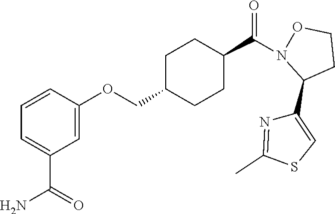 [SMILES: Cc1nc([C@@H]2CCON2C(=O)[C@H]2CC[C@H](COc3cccc(C(N)=O)c3)CC2)cs1] | 1H NMR (400.23 MHz, DMSO-d6) δ ppm 7.93 (br s, 1 H), 7.44 (s, 1 H), 7.42 (br d, J = 5.75 Hz, 2 H), 7.33 (t, J = 7.82, 7.82 Hz, 2 H), 7.20 (s, 1 H), 7.07 (dd, J = 8.01, 1.90 Hz, 1 H), 5.38 (dd, J = 8.50, 5.81 Hz, 1 H), 4.24 (td, J = 7.55, 7.55, 3.97 Hz, 1 H), 3.84 (m, 9 H), 3.74 (br s, 20 H), 2.70 (m, 2 H), 2.63 (s, 4 H), 2.40 (m, 1 H), 1.90 (br d, J = 11.86 Hz, 3 H), 1.74 (br s, 2 H), 1.39 (m, 2 H), 1.14 (m, 2 H). LC/MS: m/z [M + H]⁺ 430.2, Rt (λ = 220 nm): 1.79 min (LC/MS method |
| | | A), calc. m/z: 430.18. |
| 131 | 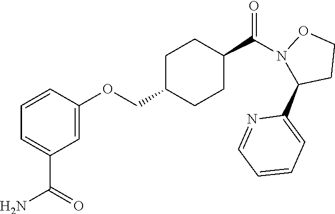 [SMILES: NC(=O)c1cccc(OC[C@H]2CC[C@H](C(=O)N3OCC[C@H]3c3ccccn3)CC2)c1] | 1H NMR (400.23 MHz, DMSO-d6) δ ppm 8.55 (d, J = 4.28 Hz, 1 H), 7.93 (br s, 1 H), 7.82 (td, J = 7.70, 7.70, 1.59 Hz, 1 H), 7.44 (s, 1 H), 7.42 (d, J = 4.93 Hz, 2 H), 7.33 (m, 4 H), 7.07 (dd, J = 7.95, 1.96 Hz, 1 H), 5.36 (dd, J = 8.80, 5.99 Hz, 1 H), 4.26 (td, J = 7.70, 7.70, 3.30 Hz, 1 H), 3.90 (br s, 1 H), 3.82 (m, 26 H), 3.51 (br s, 1 H), 2.81 (m, 2 H), 2.43 (m, 1 H), 1.91 (br d, J = 12.35 Hz, 3 H), 1.75 (br d, J = 2.93 Hz, 2 H), 1.40 (m, 2 H), 1.15 (m, 2 H). |
| | | LC/MS: m/z [M + H]⁺ 410.2, Rt (λ = |
| | | 220 nm): 1.63 min (LC/MS method |
| | | A), calc. m/z: 410.21. |

### Process of Making

The compounds of formula (1) may be prepared using the methods disclosed herein and routine modifications thereof, which will be apparent given the disclosure herein and methods well known in the art.

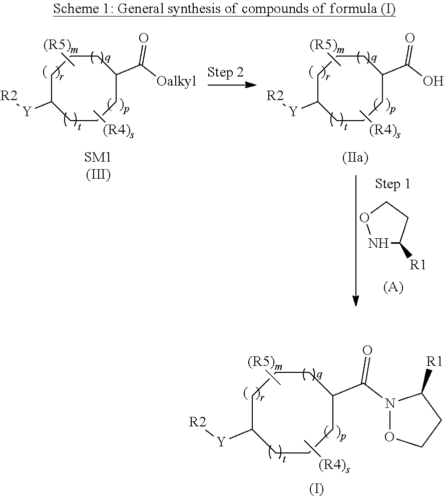

According to a first Synthetic Method (SM1), compounds of formula (I) can be obtained from an alkyl ester of formula (III). Step 2 may consist in hydrolysis of the alkyl ester of formula (III) to form the compound of formula (IIa) and can be achieved under well-known conditions using e.g. lithium hydroxide or sodium hydroxide in water or in a solvent mixture, such as THF/water or THF/water/MeOH. Step 2 is in particular illustrated in step 4 of example 1. Step 1 is an amide coupling between an appropriate substituted isoxazolidine compound of formula (A) and the compound of formula (IIa). It can be achieved by standard acid activation methods using for example ethyl cyanohydroxyiminoacetate (Oxyma), acid chloride, HOBt, HATU, HBTU, PyBOP or 1-propanephosphonic anhydride (T₃P) under basic conditions, e.g. in presence of triethylamine, diisopropylethylamine or the like, in aprotic solvents like DMF, DMSO, acetonitrile, or the like to form compounds of formula (1). Step 1 is in particular illustrated in step 3a of example 3.1, in step 3 of example 5 and in step 5 of example 1.

Substituted isoxazolidines of formula (A) can be synthesized by known procedures from the literature, e.g. WO2017096301, WO2019130230 and WO2021245070.

Compounds of formula (IIb) can be synthesized by known procedures from the literature or as described herein.

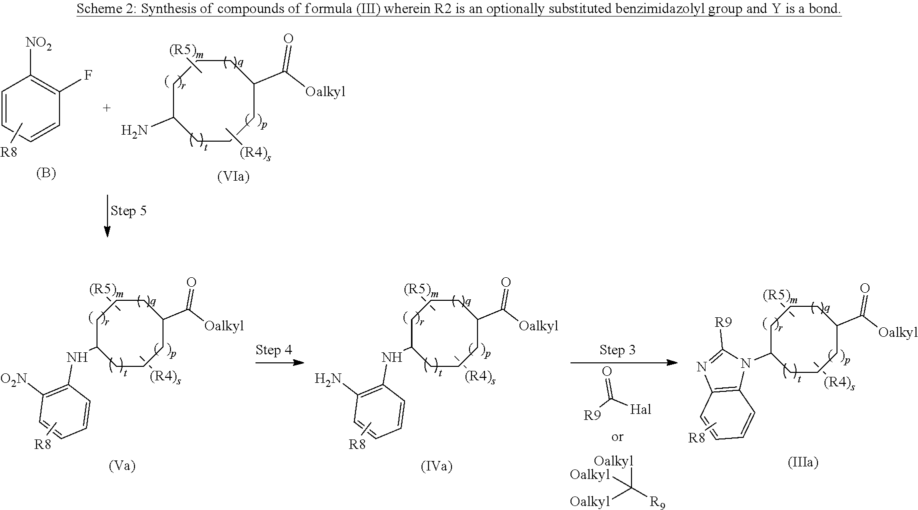

Compounds of formula (1) with R2 representing an optionally substituted imidazolyl group and Y is a bond can be synthesized starting from optionally substituted ortho fluoro nitrobenzene (B) and a compound of formula (IVa). In Scheme 2, R4, R5, R6, m, s, p, q, r and t are as defined in formula (I), o is 0, 1, 2 or 3, R8 either represents a hydrogen atom or a R6 group and R9 represents a hydrogen atom or a group selected from halogen, cyano, —OH, a (C₁-C₄)-alkyl group, —CF₃, —C(O)NH₂, a —C(O)NH—(C₁-C₄)-alkyl group, —C(O)OH, a —C(O)O—(C₁-C₄)-alkyl group, —SO₂NH₂, a (C₁-C₄)-alkoxy group.

Step 5 is a S_{N}Ar reaction implemented in the presence of a base, such as diisopropylethylamine, in an appropriate solvent, such as acetonitrile. The obtained nitro aniline compound (Va) can be subsequently transformed (Step 4) into the corresponding amino aniline compound (IVa), which can be achieved by e.g. palladium catalyzed reduction under hydrogen atmosphere in an appropriate solvent, such as methanol. Step 3 allows obtaining compound (IIIa) via condensation with appropriate ortho esters or acid halides bearing a R9 radical, for example acetyl chloride or trimethyl orthoformate. This type of reactions can be run in solvents such as methanol or dioxane, respectively.

Optionally substituted ortho fluoro nitrobenzene (B) can be synthesized by known procedures from the literature or as described herein.

Compounds of formula (VIa) can be synthesized by known procedures from the literature or as described herein.

A synthesis of compound of formula (IIIa) via Scheme 2 may be illustrated in the example 1 hereafter.

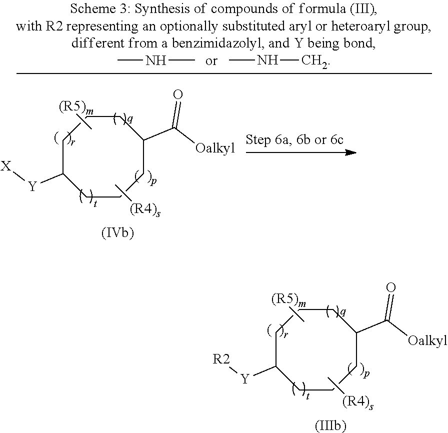

Compounds of the general formula (IIIb), where R2 is as defined in formula (I), can be synthesized starting from compound (IVb).

In the case where Y is —NH— or —NH—CH₂—, compound (IIIb) can be obtained from compound (IVb), wherein X is a hydrogen atom, via step 6a. Step 6a may be a coupling reaction implemented in the presence of a metal catalyst, in particular copper catalyst, such as copper(I) iodide, copper(I) bromide or copper(I) oxide, and a base such as Cs₂CO₃, K₃PO₄ or sodium acetate, in a polar solvent, such as DMF, DMSO, DMA (dimethylacetamide) or NMP (N-Methyl-2-pyrrolidone). Step 6a is illustrated in particular in examples 2.1 and 2.2.

In a case where Y is —NH— or —NH—CH₂, compound (IIIb) can be obtained from compound (IVb) wherein X is a hydrogen atom, via step 6b. Step 6b may be a S_{N}Ar reaction implemented in the presence of a base, such as diisopropylethylamine or K₂CO₃, in an appropriate solvent, such as acetonitrile and DMSO. Step 6b is illustrated in particular in examples 2.3, 2.4, 2.5 and 3.1.

In the case where Y is a bond, compound (IIIb) can be obtained from compound (IVb) wherein X is a leaving group, via step 6c. In the case where R2 represents an optionally substituted heteroaryl group containing a —NH group, compound (IIIb) can be obtained from compound (IVb) wherein X is a leaving group via alkylation Step 6c. Compound (IVb) where X is a leaving group in particular a halide or a alkylsulfonate, such as mesylate, tosylate or nosylate, may be reacted with an appropriate heteroaromatic compound containing a —NH group in inert solvents, as for example DMF or THF in the presence of bases such as Cs₂CO₃ or K₂CO₃. Step 6c is illustrated in particular in examples 4.1 and 4.2.

Compounds of formula (IVb) can be synthesized by known procedures from the literature or as described herein.

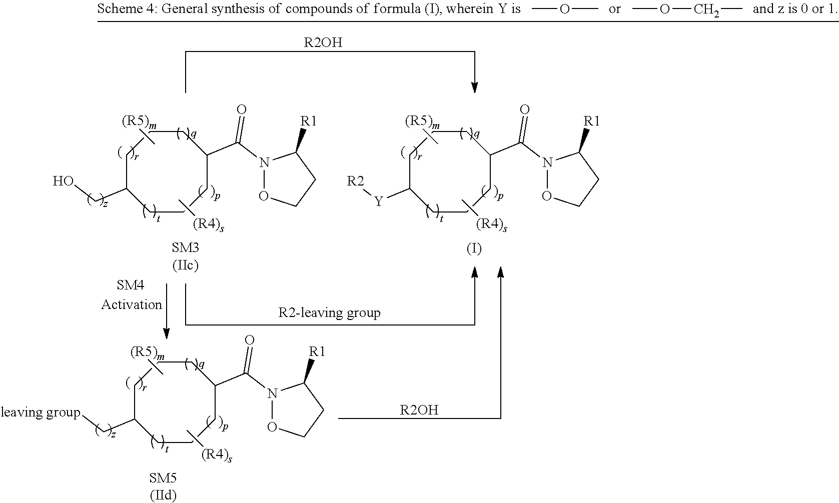

According to a third Synthetic Method (SM3), represented in scheme 4 by an upper arrow, compounds of formula (I) can be obtained from an amide of formula ((Ic). The step may consist in Mitsunobu reaction under well-known conditions. It can be achieved by using compound of formula (IIc) and a compound of formula R2OH, R2 being as defined in formula (I) and in particular an aryl group optionally substituted, in the presence of an azodicarboxylate reactant, for example DEAD, DIAD, ADDP (1,1′-(azodicarbonyl)dipiperidine), TMAD (N,N,N′,N′-tetramethyldicarboxamide), DCAD (di-4-chlorobenzyl azodicarboxylate), DNAD (di-4-nitrobenzyl azodicarboxylate), with a phosphine, for example triphenylphosphine, PH₃, DPPE (1,2-Bis(diphenylphosphino)ethane), PPh₂Py (diphenyl-2-pyridylphosphine), Tris-DAP (Tris(dimethylamino)phosphine), in an aprotic solvent like THF, CH₂Cl₂ or Et₂O or the like to form compounds of formula (I). SM3 route is in particular illustrated in step 3a of example 9, in steps 2a and 2b of example 12.2, and step 2 of example 12.6.

According to a fourth Synthetic Method (SM4), represented in scheme 4 by a middle arrow, compounds of formula (I) can be obtained from an amide of formula (IIc). The step may consist in a nucleophilic substitution, in particular an aromatic nucleophilic substitution under well-known conditions. It can be achieved by using a compound of formula R2-leaving group, R2 being as defined in formula (I) and in particular being an aryl group optionally substituted or the like, the leaving group being an halogen, for example a chlorine atom, or a cyano, in the presence of a base, for example diisopropylethylamine, K₂CO₃, NaH, KH, K₃PO₄, Na₂CO₃, tBuOK, in an aprotic solvent like THF, acetonitrile, DMF, DMA, DMSO, NMP, dioxane, or the like to form compounds of formula (I). SM4 route is in particular illustrated in steps 2a and 2b of example 12.1.

According to a third Synthetic Method (SM5), represented in scheme 4 by a lower arrow, compounds of formula (I) can be obtained from an amide of formula (IId) comprising a leaving group. As an example, compound of formula (IId) may be obtained from compound of formula (IIc) by an activation of hydroxyl group to form a leaving group, represented in scheme 4 by a dotted arrow. Said activation may be performed by a well-known nucleophilic substitution of the hydroxyl group of a compound of formula (IIc) on a sulfonyl chloride, in particular a halide, in particular an iodide, or an alkylsulfonyl chloride, such as mesyl chloride, arylsulfonyl chloride, such as tosyl chloride or 4-nitrobenzenesulfonyl chloride, in the presence of a base, in particular an amine base, such as triethylamine or diisopropylethylamine, in an aprotic solvent like CH₂Cl₂, ethyl acetate, THF or DMF, to form a compound of formula (IId). Activation step is in particular illustrated in step 4a of example 9.

The step to form compounds of formula (I) using SM5 may consist in in a nucleophilic substitution under well-known conditions. It can be achieved by using compound of formula (IId) and R2-OH compound, R2 being as defined in formula (I) and in particular being an aryl group optionally substituted or the like, in the presence of a base, for example, Cs₂CO₃, NaH, Potassium tert-butoxide, and the like, in an aprotic solvent like DMF, acetonitrile, DMSO, NMP or THF, or the like, to form compounds of formula (I). SM5 route is in particular illustrated in steps 5a and 5b of example 9.

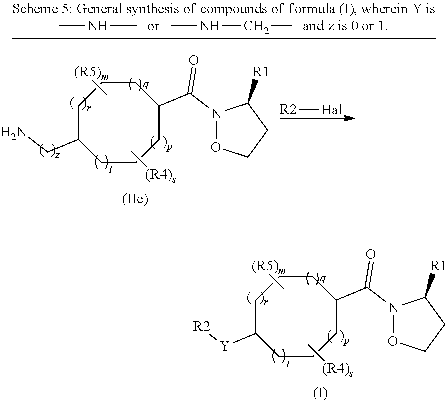

Compounds of the general formula (I) where R2 represents an optionally substituted phenyl or heteroaryl group can be synthesized starting from compound (IIe) via an aromatic nucleophilic substitution, in particular an aromatic nucleophilic substitution (S_{N}Ar). It can be achieved by reacting compound (IIe) with a compound of formula R2-Hal, R2 being as defined in formula (I) and in particular being an aryl or heteroaryl group substituted by a Hal leaving group, Hal being a halogen, for example a chlorine atom, in the presence of a base, for example diisopropylethylamine, K₂CO₃ NaH, KH, K₃PO₄, Na₂CO₃, or tBuOK, in an aprotic solvent like THF, DMSO, acetonitrile, DMF, NMP or DMA, or the like to form compounds of formula (1). Such route is in particular illustrated in example step 2 of 11.2, in step 2b of example 11.9 and in step 3 of example 11.10.

Functional groups like acids, esters, amides, nitriles, halogens in compounds can be transformed (functional group interconversion) into other functional groups with standard methods like esterification, saponification, halogenation, Suzuki reaction to yield further compounds of formula (1).

The synthesis of typical compounds described herein may be accomplished as described in the following examples. If available, reagents may be purchased commercially, e.g., from Sigma Aldrich or other chemical suppliers. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedure.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in Wuts, P. G. M., Greene, T. W., & Greene, T. W. (2006), Greene's protective groups in organic synthesis, Hoboken, N.J., Wiley-Interscience, and references cited therein.

Furthermore, the compounds of this disclosure may contain one or more chiral centers. Accordingly, if desired, such compounds can be prepared or isolated as pure stereoisomers, i.e., as individual enantiomers or diastereomers or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents, and the like.

The starting materials for the following reactions are generally known compounds or can be prepared by known procedures or obvious modifications thereof. For example, many of the starting materials are available from commercial suppliers such as Aldrich Chemical Co. (Milwaukee, Wisconsin, USA), Bachem (Torrance, California, USA), Emka-Chemce or Sigma (St. Louis, Missouri, USA). Others may be prepared by procedures or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989) Organic Reactions, Volumes 1-40 (John Wiley, and Sons, 1991), March's Advanced Organic Chemistry, (John Wiley, and Sons, 5^{th} Edition, 2001), and Larock's Comprehensive Organic Transformations (VCH Publishers Inc., 1989). The terms “solvent,” “inert organic solvent” or “inert solvent” refer to a solvent inert under the conditions of the reaction being described in conjunction therewith (including, for example, benzene, toluene, acetonitrile, tetrahydrofuran (“THF”), dimethylformamide (“DMF”), chloroform, methylene chloride (or dichloromethane, “DCM”), diethyl ether, methanol, pyridine and the like). Unless specified to the contrary, the solvents used in the reactions of the present disclosure are inert organic solvents, and the reactions are carried out under an inert gas, preferably argon.

### Pharmaceutical Compositions

The compounds provided herein is usually administered in the form of pharmaceutical compositions. Thus, provided herein are also pharmaceutical compositions that contain one or more of the compounds described herein or a pharmaceutically acceptable salt, tautomer, stereoisomer, mixture of stereoisomers, prodrug, or deuterated analog thereof and one or more pharmaceutically acceptable vehicles selected from carriers, adjuvants and excipients.

According to another aspect, pharmaceutical compositions are disclosed that include a compound described herein as an active ingredient. The compounds of the disclosure will normally, but not necessarily, be formulated into pharmaceutical compositions prior to administration to a patient. These pharmaceutical compositions comprise an effective dose of at least one compound of the disclosure as defined herein, or a pharmaceutically acceptable salt thereof, and one or more pharmaceutically acceptable excipients.

The said excipients are selected, in accordance with the pharmaceutical form and method of administration desired, from the customary excipients, which are known to a person skilled in the art.

In these pharmaceutical compositions for oral, sublingual, subcutaneous, intramuscular, intravenous, topical, local, intra-tracheal, intranasal, transdermal or rectal administration, the active ingredient is a compound of formula (I) or its salt or solvate where appropriate may be administered in a unit administration form, in a mixture with conventional pharmaceutical excipients, to a subject like human beings for the prophylaxis or treatment of a disease or disorder or condition that is at least partly mediated by receptor-interacting protein kinase 1 and in particular acute and chronic neurodegenerative diseases like Parkinson's disease, Alzheimer's disease, amyotrophic lateral sclerosis (ALS) or multiple sclerosis (MS).

### Unit Administration

The unit administration forms appropriate include oral forms such as tablets, soft or hard gel capsules, powders, granules and oral solutions or suspensions, sublingual, buccal, intratracheal, intra-ocular and intranasal administration forms, intra-muscular or intravenous administration, rectal administration forms and implants.

When prepared in unit administration form, the pharmaceutical compositions of the disclosure typically contain from 1 mg to 1000 mg of the active ingredient. The amount of active ingredient that is combined with one or more excipients to produce a single unit administration form will necessarily vary depending upon the host treated and the particular route of administration. For example, a formulation intended for oral administration to humans will generally contain, for example, from 0.5 mg to 0.5 g of active agent compounded with an appropriate and convenient amount of excipients which may vary from about 5 to about 98 percent by weight of the total composition.

As an example, a unit administration form of a herein described compound in tablet form may comprise the following components:

| Compound | 50.0 | mg |
|---|---|---|
| Mannitol | 223.75 | mg |
| Sodium croscarmellose | 6.0 | mg |
| Corn starch | 15.0 | mg |
| Hydroxypropylmethylcellulose | 2.25 | mg |
| Magnesium stearate | 3.0 | mg |

In using a compound of the disclosure for therapeutic or prophylactic purposes it will generally be administered so that a daily dose in the range, for example, from 0.1 mg/kg to 75 mg/kg body weight is received, given if required in divided doses.

In general, lower doses will be administered when a parenteral route is employed. Thus, for example, for intravenous or intraperitoneal administration, a dose in the range, for example, from 0.1 mg/kg to 30 mg/kg body weight will generally be used. Oral administration may also be suitable, particularly in tablet form. Typically, unit dosage forms will contain about from 0.5 mg to 0.5 g of a compound of this disclosure.

There may be particular cases in which higher or lower dosages are appropriate; such dosages do not depart from the scope of the disclosure. According to usual practice, the dosage that is appropriate for each patient is determined by the doctor according to the mode of administration and the weight and response of the said patient.

### Methods of Treatment

In other embodiments, provided herein is a method of treating a receptor-interacting protein kinase 1-mediated disease or disorder. The method includes administering a therapeutically effective amount of a compound or pharmaceutical composition as described herein to a subject in need thereof. In some embodiments, the receptor-interacting protein kinase 1-mediated disease or disorder is Parkinson's disease, Alzheimer's disease, amyotrophic lateral sclerosis (ALS) or multiple sclerosis (MS).

The receptor-interacting protein kinase 1 inhibitors of the present disclosure are therefore useful for treating diseases and conditions mediated by receptor-interacting protein kinase 1, including but not limited to neurodegenerative diseases, central nervous system (CNS) diseases.

### Neurodegenerative and CNS Diseases

The receptor-interacting protein kinase 1 inhibitors described herein may also be used to treat neurodegenerative diseases. Neurodegenerative diseases can affect many of the body's activities, such as balance, movement, talking, breathing, and heart function. Neurodegenerative diseases can be genetic or caused by medical conditions such as alcoholism, tumors, strokes, toxins, chemicals, and viruses. Non-limiting examples of neurodegenerative diseases include Alzheimer's disease, amyotrophic lateral sclerosis (ALS) and Parkinson's disease

In certain embodiments, the compounds and compositions of the present disclosure are useful for treating Alzheimer's disease. In certain embodiments, the compounds and compositions of the present disclosure are useful for treating Parkinson's disease. In certain embodiments, the compounds and compositions of the present disclosure are useful for treating amyotrophic lateral sclerosis (ALS).

More generally, the receptor-interacting protein kinase 1 inhibitors described herein can be used to preserve neuron viability and promote axon growth and nerve functions within the central nervous system (CNS). Accordingly, the compounds may be used to reduce or even reverse the loss of cognitive, motor, and sensory functions associated with a CNS disease or disorder, by preserving neuron viability and/or promoting axon regeneration and/or nerve functions.

If desired, the effective daily dose of the active compound may be administered as two, three, four, five, six or more sub-doses administered separately at appropriate intervals throughout the day, optionally, in unit dosage forms. In certain embodiments, the present disclosure relates to compounds for inhibiting cell death, wherein the compounds are represented by formula (I). In certain embodiments, the compounds of the present disclosure are inhibitors of cell death. In any event, the compounds of the present disclosure in another embodiment exert their effect on inhibiting cell death at a concentration less than about 50 micromolar, more in another embodiment at a concentration less than about 10 micromolar and most in another embodiment at a concentration less than 1 micromolar. The compounds of the disclosure can be tested in standard animal models of stroke and standard protocols such as described by Hara, H., et al. Proc. Natl. Acad. Sci. USA, 1997.94(5): 2007-12.

When the compounds of the present disclosure are administered as pharmaceuticals, to humans and animals, they can be given per se or as a pharmaceutical composition containing, for example, 0.1% to 99.5% (more in another embodiment, 0.5% to 90%) of active ingredient in combination with a pharmaceutically acceptable carrier.

The compounds of the present disclosure or the compositions thereof may be administered once, twice, three or four times daily. Also, administration or treatment with the compounds may be continued for a number of days; for example, commonly treatment would continue for at least 7 days, 14 days or 28 days, for one cycle of treatment. Treatment cycles are well known and are frequently alternated with resting periods of about 1 to 28 days, commonly about 7 days or about 14 days, between cycles. The treatment cycles, in certain embodiments, may also be continuous.

When administered orally, the total daily dosage for a human subject may be between 1 mg and 1,000 mg, between about 1,000-2,000 mg/day, between about 10-500 mg/day, between about 50-300 mg/day, between about 75-200 mg/day or between about 100-150 mg/day.

The daily dosage may also be described as a total amount of a compound described herein administered per dose or per day. Daily dosage of a compound may be between about 1 mg and 4,000 mg, between about 2,000 to 4,000 mg/day, between about 1 to 2,000 mg/day, between about 1 to 1,000 mg/day, between about 10 to 500 mg/day, between about 20 to 500 mg/day, between about 50 to 300 mg/day, between about 75 to 200 mg/day or between about 15 to 150 mg/day. In certain embodiments, the method comprises administering to the subject an initial daily dose of about 1 to 800 mg of a compound described herein and increasing the dose by increments until clinical efficacy is achieved. Increments of about 5, 10, 25, 50 or 100 mg can be used to increase the dose. The dosage can be increased daily, every other day, twice per week or once per week.

In certain embodiments, a compound or pharmaceutical preparation is administered orally. In certain embodiments, the compound or pharmaceutical preparation is administered intravenously. Alternative routes of administration include sublingual, intramuscular and transdermal administrations.

The preparations of the present disclosure may be given orally, parenterally, topically or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. In certain embodiments, the administration is oral.

### EXAMPLES

The examples which follow describe the preparation of certain compounds. These examples are not limitative and merely illustrative. Numerous modifications and alternative compositions, methods, and systems may be devised by those skilled in the art without departing from the spirit and scope of the present disclosure.

### Abbreviations:

| | |
|---|---|
| ACN | Acetonitrile |
| ADP | Adenosindiphosphate |
| ATP | Adenosintriphosphate |
| BOC | Tertiar butyl-oxy-carbonyl |
| BPD | 4,4,4′,4′,5,5,5′,5′-octamethyl-2,2′-bi- |
| | 1,3,2-dioxaborolane |
| br | Broad |
| CDI | Carbonyldiimidazole |
| CMC | Carboxy-methyl-cellulose |
| CRC | Concentration response curve |
| d | Dublett |
| DBU | 1,8-Diazabicyclo[5.4.0]undec-7-en |
| DCM | Dichloromethane |
| dd | Dublett of dubletts |
| DEA | Diethylamine |
| DIAD | Diisopropyl azodicarboxylate |
| DMAP | Dimethylaminopyridine |
| DME | Dimethoxyethane |
| DMF | Dimethylformamide |
| DMSO | Dimethylsulfoxide |
| DPPF | 1,1′-Bis(diphenylphosphino)ferrocen |
| dt | Dublett of tripletts |
| dtbbpy | 4,4′-di-tert-butyl-2,2′-bipyridine |
| EA | Ethyl acetate |
| eq. | Equivalent |
| ES | Electro spray |
| EtOH | Ethanol |
| FBS | Fetal bovine serum |
| HATU | 1-[Bis(dimethylamino)methylene]-1H- |
| | 1,2,3-triazolo[4,5-b]pyridinium 3-oxide |
| | hexafluorophosphate |
| HBTU | 2-(1H-Benzotriazole-1-yl)-1, 1,3,3- |
| | tetramethyluronium hexafluorophosphate |
| HOBt | 1-Hydroxybenzotriazole |
| HPLC | High pressure liquid chromatography |
| hr / hrs | Hour/hours |
| Hz | Hertz |
| IL6 | Interleukin 6 |
| IPA | Isopropylalcohol |
| Ir[dF | [4,4′-Bis(1,1-dimethylethyl)-2,2′- |
| (CF₃)ppy]₂ | bipyridine-N1, N1′]bis[3,5-difluoro-2- |
| (dtbpy)(PF₆) | [5-(trifluoromethyl)-2-pyridinyl-N]phenyl- |
| | C]Iridium(III) hexafluorophosphate, |
| | [Ir{dF(CF₃)ppy}₂(dtbpy)]PF₆ |
| [Ir(dF | [(4,4′-Di-tert-butyl-2,2′-bipyridine)-bis-(5- |
| (Me)ppy)₂ | methyl-2-(5-fluoro-phenyl)-pyridine)- |
| (dtbbpy)]PF₆ | iridium(III)]hexafluorophosphate |
| LC/MS | Liquid chromatography mass spectrum |
| m | Multiplett |
| m/z | Mass per charge |
| MeOH | Methanol |
| MHz | Mega Hertz |
| min | Minute(s) |
| MTBE | 2-Methoxy-2-methylpropan |
| N | Normal |
| n-BuOH | n-Butanol |
| NBS | N-bromosuccinimide |
| NMR | Nuclear magnetic resonance |
| Oxyma | Ethyl cyanohydroxyiminoacetate |
| PBS | Phosphate buffered saline |
| PG | Protecting group |
| PyBOP | Benzotriazol-1- |
| | yloxytripyrrolidinophosphonium |
| | hexafluorophosphate |
| q | Quadruplett |
| quin | Quintuplett |
| rH | Relative humidity |
| rt | Room temperature |
| Rt | Retention time |
| S | Singulett |
| S-PHOS | 2-Dicyclohexylphosphino-2′,6′- |
| | dimethoxybiphenyl |
| SFC | Supercritical fluid chromatography |
| SNAr | Nucleophilic Aromatic Substitution |
| t | Triplett |
| T3P | Propanephosphonic acid anhydride |
| TBAF | Tetra-n-butylammonium fluoride |
| td | Triplett of dubletts |
| TDAE | Tetrakis(dimethylamino)ethylene |
| TEA | Triethyl amine |
| TFA | Trifluoroacetic acid |
| TFAA | Trifluoroacetic acid anhydride |
| THF | Tetrahydrofurane |
| TLC | Thin layer chromatography |
| TNF | Tumor Necrosis Factor |
| TTMSS | tris(trimethylsilyl)silane |
| UV | Ultraviolet |
| X-PHOS | 2-Dicyclohexylphosphin-2′,4′,6′- |
| | triisopropylbiphenyl |

### Silica Gel Chromatography

Silica gel chromatography was performed using CombiFlash® Rf (Teledyne ISCO), Biotage Isolera One automated flash purification system or two Büchi systems (C-660, C-605, C-620, C-635 combination and C-660, C-605, C-615, C-630 combination) with pre-packed cartridges.

### Preparative Reversed-Phase HPLC

For preparative reversed-phase HPLC an Agilent 1200 preparative HPLC machine, Gilson equipment (GX-271 liquid handler, 331/332-pump, UV/VIS-155) or a Waters Autopurification LC Prep System was used.

### Preparative RP-LC

Reversed phase liquid chromatography was performed with a Biotage equipment using 018 columns and a water (0.1% formic acid)/acetonitrile gradient.

### NMR

400 MHz: ¹H NMR spectra were recorded on a Bruker AVANCE II 400 spectrometer operating at a proton frequency of 400.23 MHz. The instrument was equipped with a 5 mm BBI room temperature probe head. Alternatively, a Bruker AVANCE III HD 400 MHz, or a Bruker AVANCE NEO 400 MHz was used.

600 MHz: ¹H NMR spectra were recorded on a Bruker AVANCE III 600 spectrometer operating at a proton frequency of 600.05 MHz. The instrument was equipped with a 5 mm BBI room temperature probe head.

### Analytical LC/MS Equipment for Method A

Retention time and mass detection were done on a Waters Acquity UHPLC system coupled with a Waters SQD mass detector. The injection volume was 1.0 μl. Molecular weights are given in gram per mol [g/mol], detected masses in mass per charge [m/z].

### Analytical LC/MS Equipment for Method B and Method C

For retention time and mass detection a LC/MS-system from Agilent (LC 1200 Series/MS 6120 quadrupole LC/MS, LC 1260 infinity/MS 6120 quadrupole LC/MS or LC 1260 Infinity II/MSD Infinity Lab) was used. Molecular weights are given in gram per mol [g/mol], detected masses in mass per charge [m/z].

### LC/MS-Method A

Gradient: 98% H₂O (0.05% formic acid)/2% acetonitrile (0.035% formic acid) for 0.2 min, then from 98% H₂O (0.05% formic acid) to 98% acetonitrile (0.035% formic acid) in 3.6 min, then 98% acetonitrile (0.035% formic acid) for 0.5 min, flow rate: 1.0 ml/min, column: 2.1×50 mm Waters ACQUITY UPLC BEH C18, 1.7 μm, 55° C. UV data: retention time ad λ=220 nm given in min MS data: ES+ ionisation, m/z given as [M+H]⁺ unless otherwise noted.

### LC/MS-Method B

Gradient: From 95% H₂O (0.0375% TFA)/5% acetonitrile (0.01875% TFA) to 5% H₂O (0.0375% TFA)/95% acetonitrile (0.01875% TFA) in 0.8 min, flow rate: 1.5 ml/min, column: Kinetex EVO C18 2.1×30 mm, 5 μm, 50° C. UV data: retention time ad λ=220 nm given in min MS data: ES+ ionisation, m/z given as [M+H]⁺ unless otherwise noted.

### LC/MS-Method C

Gradient: From 100% H₂O (0.0375% TFA)/0% acetonitrile (0.01875% TFA) to 60% H₂O (0.0375% TFA)/40% acetonitrile (0.01875% TFA) in 0.8 min, flow rate: 1.5 ml/min, column: Kinetex EVO C18 2.1×30 mm, 5 μm, 50° C. UV data: retention time ad λ=220 nm given in min MS data: ES+ ionisation, m/z given as [M+H]⁺ unless otherwise noted.

### Salts

In compounds described as HCl-, TFA- or as another salt the exact amount of the respective salt is usually not determined. Therefore, the amount of the salt can range from as low as 0.01 eq. up to 5.0 eq. depending on the chemical structure (e.g. number of basic centres).

### Chiral Purity

Compounds are drawn and named as a single enantiomer, if the enantiomeric ratio exceeded 90:10. For enantiomeric ratios below 90:10 the racemic form is used.

### Example 1: Synthesis of Compounds (40), (41), (42), (69), (70), (84) and (87)

### Example 1.0

STEP 1: Synthesis of methyl trans-4-[(2-nitroanilino)methyl]cyclohexanecarboxylate

To a stirred suspension of 1-fluoro-2-nitrobenzene (749 μl, 7.09 mmol) and methyl 4-(aminomethyl)cyclohexanecarboxylate hydrochloride (1.50 g, 7.09 mmol) in CH₃cN (22 ml) Net/Pr₂ was added (3.71 ml, 21.26 mmol) at rt. The solution was heated at reflux for 2 hrs. Volatile components were removed under reduced pressure and the resulting residue was partioned between EA and water. The aqueous layer was extracted with EA, the combined organic layers were dried over Na₂SO₄, filtered and concentrated to afford crude 4-[(2-nitroanilino)methyl]cyclohexanecarboxylate, which was purified by column chromatography (SiO₂; EA/heptane gradient), (860 mg, 2.95 mmol, 41% yield).

¹H NMR (600 MHz, DMSO-d₆): δ ppm 8.18 (br t, J=5.41 Hz, 1H), 8.06 (m, 1H), 7.52 (t, J=7.84 Hz, 1H), 7.07 (d, J=8.44 Hz, 1H), 6.67 (t, J=7.85 Hz, 1H), 3.58 (s, 3H), 3.27 (m, 2H), 2.27 (m, 1H), 1.93 (m, 2H), 1.82 (m, 2H), 1.63 (m, 1H), 1.32 (m, 2H), 1.06 (m, 2H).

STEP 2: Synthesis of methyl trans-4-[(2-aminoanilino)methyl]cyclohexanecarboxylate

A stirred suspension of methyl trans-4-(((2-nitrophenyl)amino)methyl)cyclohexane-1-carboxylate (862 mg, 2.95 mmol) in MeOH (70 ml) and Pd/C (10%, 54% water, 470 mg, 442 μmol) in a round bottom flask was evacuated and backfilled with H₂ at 0° C. This process was repeated 3 times. The suspension was vigorously stirred under a H₂ atmosphere (H₂ ballon) at rt for 2 hrs. The suspension was filtered, the filter cake rinsed with MeOH and the filtrate concentrated under reduced pressure. The title compound was obtained as a yellow solid and used in the next reaction without further purification (695 mg, 2.65 mmol, 90% yield).

¹H NMR (400 MHz, DMSO-d₆): δ ppm 6.49 (m, 2H), 6.38 (m, 2H), 4.47 (s, 2H), 4.32 (t, J=5.62 Hz, 1H), 3.58 (s, 3H), 2.86 (t, J=6.11 Hz, 2H), 2.27 (m, 1H), 1.92 (m, 4H), 1.55 (m, 1H), 1.31 (m, 2H), 1.00 (m, 2H)

STEP 3: Synthesis of methyl trans-4-(99enzimidazole-1-ylmethyl)cyclohexanecarboxylate

To a stirred solution of methyl trans-4-(((2-aminophenyl)amino)methyl)cyclohexane-1-carboxylate (694 mg, 2.65 mmol) and trimethyl orthoformate (15 ml, 137 mmol) in MeOH (15 ml) conc. HCl (1.54 ml) was added at rt. The solution was stirred at rt for 1 hr. Volatile components were removed under reduced pressure and the resulting residue partitioned between saturated aqueous NaHCO₃ and EA. The aqueous layer was extracted with EA, the combined organic layers were dried over Na₂SO₄, filtered and concentrated to afford methyl trans-4-benzimidazole-1-ylmethyl)cyclohexanecarboxylate, which was used in the next reaction without further purification (700 mg, 2.57 mmol, 97% yield).

¹H NMR (400 MHz, DMSO-d₆): δ ppm 8.18 (s, 1H), 7.64 (m, 2H), 7.22 (m, 2H), 4.10 (d, J=7.09 Hz, 2H), 3.56 (s, 3H), 2.24 (m, 1H), 1.85 (m, 3H), 1.58 (m, 2H), 1.25 (m, 2H), 1.07 (m, 2H).

STEP 4: Synthesis of trans-4-(benzimidazol-1-ylmethyl)cyclohexanecarboxylic acid—Hydrolysis as illustrated in step 2 of Scheme 1.

To a solution of methyl trans-4-(benzimidazol-1-ylmethyl)cyclohexanecarboxylate (700 mg, 2.57 mmol) in MeOH (12 ml) and THF (12 ml) a solution of lithium hydroxide (185 mg, 7.71 mmol) in water (12 ml) was added. The resulting solution was stirred at rt for 3 hrs. Volatile components were removed under reduced pressure and the remaining aqueous solution acidified with 1 N HCl (10.0 ml, 10.0 mmol). The obtained solution was lyophilized. The title compound was obtained as a white solid (2×LiCl mixture), which was used in the next reaction without further purification (1.05 g, quant.).

¹H NMR (400 MHz, DMSO-d₆): δ ppm 12.04 (br s, 1H), 9.14 (s, 1H), 7.90 (d, J=7.21 Hz, 1H), 7.80 (d, J=7.27 Hz, 1H), 7.48 (m, 2H), 4.27 (d, J=7.21 Hz, 2H), 2.14 (m, 1H), 1.91 (m, 3H), 1.61 (m, 2H), 1.24 (m, 2H), 1.09 (m, 2H).

STEP 5: Synthesis of trans-1-((4-((S)-3-(3-cyano-5-fluorophenyl)isoxazolidine-2-carbonyl)cyclohexyl)methyl)-6-fluoro-1H-benzo[d]imidazole-5-carbonitrile

To a stirred solution of trans-4-[(5-cyano-6-fluoro-benzimidazol-1-yl)methyl]cyclohexane carboxylic acid (100 mg, 331 μmol), Net/Pr₂ (174 μl, 995 μmol) and 3-fluoro-5-[(3S)-isoxazolidin-3-yl]benzonitrile hydrochloride (98.7 mg, 431 μmol) in DMF (2 ml) HATU (151 mg, 398 μmol) was added at rt. Stirring at rt was continued for 2 hrs. The reaction mixture was filtered and subjected to preparative reversed phase HPLC (70 mg, 147 μmol, 44% yield).

### Example 1.1: Synthesis of Compounds (40), (41) and (42)

STEP 1: Synthesis of methyl cis-3-(2-nitroanilino)cyclobutanecarboxylate—Compound responding to formula (Va) as defined in Scheme 2 is performed in a similar way as in Example 1.0, but using methyl cis-3-(amino)cyclobutanecarboxylate instead of methyl 4-(aminomethyl)cyclohexanecarboxylate.

STEP 2: Synthesis of methyl cis-3-(2-aminoanilino)cyclobutanecarboxylate—Compound responding to formula (VIa) as defined in Scheme 2 is performed in a similar way as in Example 1.0. but starting from cis-3-(2-nitroanilino)cyclobutanecarboxylate

STEP 3: Synthesis of methyl cis-3-(benzimidazol-1-yl)cyclobutanecarboxylate—Compound responding to formula (VIa) as defined in Scheme 2 is performed in a similar way as in Example 1.0, but starting from methyl cis-3-(2-aminoanilino)cyclobutanecarboxylate

STEP 4: Synthesis of cis-3-(benzimidazol-1-yl)cyclobutanecarboxylic acid—Compound responding to formula (IIa) in Scheme 1 is performed in a similar way as in Example 1.0, but stering from methyl cis-3-(benzimidazol-1-yl)cyclobutanecarboxylate

STEP 5a: Synthesis of cis-3-((S)-2-(3-(1H-benzo[d]imidazol-1-l)cyclobutane-1-carbonyl)isoxazolidin-3-yl)-5-fluorobenzonitrile (Compound (40)) is performed as in STEP 5 of Example 1.0, but starting from cis-3-(benzimidazol-1-yl)cyclobutanecarboxylic acid instead of trans-4-[(5-cyano-6-fluoro-benzimidazol-1-yl)methyl]cyclohexanecarboxylic acid.

STEP 5b: Synthesis of cis-(3-(1H-benzo[d]imidazol-1-yl)cyclobutyl)((S)-3-(3,5-difluorophenyl)isoxazolidin-2-yl)methanone (Compound (41)) is performed in a similar way as in STEP 5 of Example 1.0 but starting from cis-3-(benzimidazol-1-yl)cyclobutanecarboxylic acid and (S)-3-(3,5-difluorophenyl)isoxazolidine.

STEP 5c: Synthesis of cis-(3-(1H-benzo[d]imidazol-1-yl)cyclobutyl)((S)-3-(5-fluoropyridin-3-yl)isoxazolidin-2-yl)methanone (Compound (42)) is performed in a similar way as in STEP 5 of Example 1.0 but starting from cis-3-(benzimidazol-1-yl)cyclobutanecarboxylic acid and (S)-3-(5-fluoropyridin-3-yl)isoxazolidine.

### Example 1.2: Synthesis of Compounds (69) and (70)

STEP 1: Synthesis of methyl cis-3-(4-fluoro-2-nitro-anilino)cyclobutanecarboxylate—Compound responding to formula (Va) as defined in Scheme 2—is performed in a similar way as detailed in STEP 1 of Example 1.0.

STEP 2: Synthesis of methyl cis-3-(2-amino-4-fluoro-anilino)cyclobutanecarboxylate—Compound responding to formula (IVa) as defined in Scheme 2—is performed in a similar way as detailed in STEP 2 of Example 1.0. but starting from methyl cis-3-(4-fluoro-2-nitro-anilino)cyclobutanecarboxylate

STEP 3: Synthesis of methyl cis-3-(5-fluorobenzimidazol-1-yl)cyclobutanecarboxylate—Corresponding to compound (IIIa) in Scheme 2—is performed in a similar way as detailed in STEP 3 of Example 1.0, but starting from methyl cis-3-(2-amino-4-fluoro-anilino)cyclobutanecarboxylate

STEP 4: Synthesis of cis-3-(5-fluorobenzimidazol-1-yl)cyclobutanecarboxylic acid is performed in a similar way as detailed in STEP 4 of Example 1.0—step 2 of scheme 1, but starting from methyl cis-3-(5-fluorobenzimidazol-1-yl)cyclobutanecarboxylate

STEP 5a: Synthesis of cis-((S)-3-(3,5-difluorophenyl)isoxazolidin-2-yl)(3-(5-fluoro-1H-benzo[d]imidazol-1-yl)cyclobutyl)methanone (Compound (69)) is performed in a similar way as in STEP 5 of Example 1.0 but starting from cis-3-(5-fluorobenzimidazol-1-yl)cyclobutanecarboxylic acid and (S)-3-(3,5-difluorophenyl)isoxazolidine.

STEP 5b: Synthesis of cis-3-fluoro-5-((S)-2-(3-(5-fluoro-1H-benzo[d]imidazol-1-yl)cyclobutane-1-carbonyl)isoxazolidin-3-yl)benzonitrile (Compound (70)) is performed in a similar way as in STEP 5a of Example 1.0 but starting from cis-3-(5-fluorobenzimidazol-1-yl)cyclobutanecarboxylic acid and 3-fluoro-5-[(3S)-isoxazolidin-3-yl]benzonitrile hydrochloride.

### Example 1.3: Synthesis of Compound (84)

STEP 1: Synthesis of methyl trans-3-(4-fluoro-2-nitro-anilino)cyclobutanecarboxylate—Compound responding to formula (Va) as defined in Scheme 2—is performed in a similar way as detailed in STEP 1 of Example 1.0 but starting from methyl trans-3-(amino)cyclobutanecarboxylate.

STEP 2: Synthesis of methyl trans-3-(2-amino-4-fluoro-anilino)cyclobutanecarboxylate—Compound responding to formula (VIa) as defined in Scheme 2—is performed in a similar way as detailed in STEP 2 of Example 1.0, but starting from methyl trans-3-(4-fluoro-2-nitro-anilino)cyclobutanecarboxylate

STEP 3: Synthesis of methyl trans-3-(5-fluorobenzimidazol-1-yl)cyclobutanecarboxylate—Corresponding to compound (IIIa) in Scheme 2—is performed in a similar way as detailed in STEP 3 of Example 1.0., but starting from trans-3-(2-amino-4-fluoro-anilino)cyclobutanecarboxylate

STEP 4: Synthesis of trans-3-(5-fluorobenzimidazol-1-yl)cyclobutanecarboxylic acid is performed in a similar way as detailed in STEP 4 of Example 1.0—Compound responding to formula (IIa) in Scheme 1, but starting from methyl trans-3-(5-fluorobenzimidazol-1-yl)cyclobutanecarboxylate

STEP 5: Synthesis of trans-(3-(5-fluoro-1H-benzo[d]imidazol-1-yl)cyclobutyl)((S)-3-(3-fluorophenyl)isoxazolidin-2-yl)methanone (Compound (84)) is performed in a similar way as in STEP 5 of Example 1.0. but starting from trans-3-(5-fluorobenzimidazol-1-yl)cyclobutanecarboxylic acid

### Example 1.4: Synthesis of Compound (87)

STEP 1: Synthesis of methyl cis-3-(5-fluoro-2-nitro-anilino)cyclobutanecarboxylate—Compound responding to formula (Va) as defined in Scheme 2—is performed in a similar way as detailed in STEP 1 of Example 1.1 but starting from 1,3-difluoro-4-nitrobenzene.

STEP 2: Synthesis of methyl cis-3-(2-amino-5-fluoro-anilino)cyclobutanecarboxylate—Compound responding to formula (VIa) as defined in Scheme 2—is performed in a similar way as detailed in STEP 2 of Example 1.1, but starting from methyl cis-3-(5-fluoro-2-nitro-anilino)cyclobutanecarboxylate

STEP 3: Synthesis of methyl cis-3-(6-fluorobenzimidazol-1-yl)cyclobutanecarboxylate—Corresponding to compound (IIIa) in Scheme 2—is performed in a similar way as detailed in STEP 3 of Example 1.1, but starting from methyl cis-3-(2-amino-5-fluoro-anilino)cyclobutanecarboxylate

STEP 4: Synthesis of cis-3-(6-fluorobenzimidazol-1-yl)cyclobutanecarboxylic acid is performed in a similar way as detailed in STEP 4 of Example 1.1, but starting from methyl cis-3-(6-fluorobenzimidazol-1-yl)cyclobutanecarboxylate

STEP 5: Synthesis of cis-((S)-3-(3,5-difluorophenyl)isoxazolidin-2-yl)(3-(6-fluoro-1H-benzo[d]imidazol-1-yl)cyclobutyl)methanone (Compound (87)) is performed in a similar way as in STEP 5b of Example 1.1, but starting from cis-3-(6-fluorobenzimidazol-1-yl)cyclobutanecarboxylic acid

### Example 2: Synthesis of Compounds (32), (33), (34), (35), (25), (26), (27), (36), (37), (38), (39)

### Example 2.1: Synthesis of Compounds (32) and (33)

STEP 1: Synthesis of methyl cis-3-(3-cyano-4-fluoro-anilino)cyclobutanecarboxylate—reaction under step 6a of scheme 3

To a stirred suspension of cis-3-aminocyclobutane-1-carboxylate hydrochloride (361.4 mg, 2.18 mmol), Cs₂CO₃ (1.29 g, 3.97 mmol), CuI (75.6 mg, 396.8 μmol) and 2-(2-methylpropanoyl)cyclohexanone (276 μl, 1.59 mmol) in DMF (4.06 ml, 5.11 mmol) 2-fluoro-5-iodo-benzonitrile (500 mg, 1.98 mmol) was added at rt. Stirring was continued for 5 hrs, quenched with water and the aqueous layer extracted with EA, the combined organic layers were dried over Na₂SO₄, filtered and concentrated to afford the crude title compound, which was purified by column chromatography (SiO₂; EA/heptane gradient) (183 mg, 735 μmol, 37% yield).

¹H NMR (600 MHz, DMSO-d₆): δ ppm 7.21 (t, J=9.17 Hz, 1H), 6.86 (m, 1H), 6.80 (dd, J=5.04, 3.03 Hz, 1H), 6.40 (d, J=7.15 Hz, 1H), 3.79 (m, 1H), 3.60 (s, 3H), 2.86 (m, 1H), 2.62 (m, 2H), 1.96 (m, 2H).

STEP 2: Synthesis of cis-3-(3-cyano-4-fluoro-anilino)cyclobutanecarboxylic acid is performed in a similar way as detailed in STEP 4 of Example 1.1, but starting from methyl cis-3-(3-cyano-4-fluoro-anilino)cyclobutanecarboxylate

STEP 4a: Synthesis of cis-5-((3-((S)-3-(3-cyano-5-fluorophenyl)isoxazolidine-2-carbonyl)cyclobutyl)amino)-2-fluorobenzonitrile (Compound (32)) is performed in a similar way as in STEP 5a of Example 1.1., but starting from cis-3-(3-cyano-4-fluoro-anilino)cyclobutanecarboxylic acid

STEP 4b: Synthesis of cis-2-fluoro-5-((3-((S)-3-(5-fluoropyridin-3-yl)isoxazolidine-2-carbonyl)cyclobutyl)amino)benzonitrile (Compound (33)) is performed in a similar way as in STEP 5b of Example 1.1, but starting from cis-3-(3-cyano-4-fluoro-anilino)cyclobutanecarboxylic acid

### Example 2.2: Synthesis of Compounds (34) and (35)

STEP 1: Synthesis of methyl trans-4-[(3-cyano-4-fluoro-anilino)methyl]cyclohexanecarboxylate is performed in a similar way as in STEP 1 of example 2.1, but starting from trans-4-[aminomethyl]cyclohexanecarboxylate

STEP 2: Synthesis of trans-4-[(3-cyano-4-fluoro-anilino)methyl]cyclohexanecarboxylic acid is performed in a similar way as in STEP 1 of example 2.1 but starting from methyl trans-4-[(3-cyano-4-fluoro-anilino)methyl]cyclohexanecarboxylate

STEP 4a: Synthesis of trans-2-fluoro-5-(((4-((S)-3-(5-fluoropyridin-3-yl)isoxazolidine-2-carbonyl)cyclohexyl)methyl)amino)benzonitrile (Compound (34)) is performed in a similar way as in STEP 5c of Example 1 but starting from trans-4-[(3-cyano-4-fluoro-anilino)methyl]cyclohexanecarboxylic acid.

STEP 4b: Synthesis of trans-5-(((4-((S)-3-(3-cyano-5-fluorophenyl)isoxazolidine-2-carbonyl)cyclohexyl)methyl)amino)-2-fluorobenzonitrile (Compound (35)) is performed in a similar way as in STEP 5a of Example 1.1 but starting from trans-4-[(3-cyano-4-fluoro-anilino)methyl]cyclohexanecarboxylic acid

### Example 2.3: Synthesis of Compound (38)

STEP 1: Synthesis of methyl trans-4-[(3-cyano-5-fluoro-anilino)methyl]cyclohexanecarboxylate—reaction under step 6b of scheme 3

To a stirred suspension of 3,5-difluorobenzonitrile (140 mg, 1.01 mmol) and methyl trans-4-(aminomethyl)cyclohexane-1-carboxylate hydrochloride (313.6 mg, 1.51 mmol) in DMSO (2 ml) K₂CO₃ (281.0 mg, 2.01 mmol) was added and the resulting suspension was heated to 80° C. for 16 hrs. The mixture was allowed to reach rt and poured on water (50 ml). The suspension was stirred for 10 min, filtered and the filter cake washed with little water. The solid was dried under reduced pressure. The title compound was obtained as a white solid, which was used in the next reaction without further purification (34 mg, 117 μmol, 12% yield).

¹H NMR (400 MHz, DMSO-d₆): δ ppm 6.76 (s, 1H), 6.76 (d, J=11.17 Hz, 1H), 6.65 (m, 1H), 6.52 (br t, J=5.50 Hz, 1H), 3.58 (s, 3H), 2.89 (t, J=6.14 Hz, 2H), 2.26 (m, 1H), 1.92 (br d, J=10.45 Hz, 2H), 1.84 (br d, J=10.45 Hz, 2H), 1.49 (m, 1H), 1.31 (m, 2H), 0.99 (m, 2H).

STEP 2: Synthesis of trans-4-[(3-cyano-5-fluoro-anilino)methyl]cyclohexanecarboxylic acid is performed in a similar way as detailed in STEP 4 of Example 1.1, but starting from methyl trans-4-[(3-cyano-5-fluoro-anilino)methyl]cyclohexanecarboxylate

STEP 4a: Synthesis of trans-3-((S)-2-(4-(((3-cyano-5-fluorophenyl)amino)methyl)cyclohexane-1-carbonyl)isoxazolidin-3-yl)-5-fluorobenzonitrile (Compound (38)) is performed in a similar way as in STEP 5a of Example 1.1, but starting from trans-4-[(3-cyano-5-fluoro-anilino)methyl]cyclohexanecarboxylic acid

### Example 2.4: Synthesis of Intermediates for Compounds (36) and (37)

STEP 1: Synthesis of methyl cis-3-(3-cyano-5-fluoro-anilino)cyclobutanecarboxylate is performed in a similar way as in STEP 1 of example 2.3, but starting from cis-3-aminocyclobutane-1-carboxylate

STEP 2: Synthesis of cis-3-(3-cyano-5-fluoro-anilino)cyclobutanecarboxylic acid is performed in a similar way as detailed in STEP 4 of Example 1.1 but starting from methyl cis-3-(3-cyano-5-fluoro-anilino)cyclobutanecarboxylate

### Example 2.5: Synthesis of Compounds (39)

STEP 1: Synthesis of methyl cis-4-(3-cyano-5-fluoro-anilino)cyclohexanecarboxylate is performed in a similar way as in STEP 1 of example 2.3, but starting from cis-4-aminocyclohexanecarboxylate

STEP 2: Synthesis of cis-4-(3-cyano-5-fluoro-anilino)cyclohexanecarboxylic acid is performed in a similar way as detailed in STEP 4 of Example 1.1—compound responding to formula (IIa) in scheme 1, but starting from methyl cis-4-(3-cyano-5-fluoro-anilino)cyclohexanecarboxylate

STEP 4: Synthesis of cis-3-((S)-2-(4-((3-cyano-5-fluorophenyl)amino)cyclohexane-1-carbonyl)isoxazolidin-3-yl)-5-fluorobenzonitrile (Compound (39)) is performed in a similar way as in STEP 5a of Example 1.1.

### Example 3: Synthesis of Compounds (5), (6), (7), (8), (9), (10), (11)

### Synthesis of methyl cis-3-amino-1-methylcyclobutane-1-carboxylate trifluoroacetate

To a stirred solution of methyl cis-3-(tert-butoxycarbonylamino)-1-methyl-cyclobutanecarboxylate (300 mg, 1.23 mmol) in CH₂Cl₂ (9.0 ml) TFA (2.50 ml) was added and stirring was continued for 2 hrs at rt. The solution was concentrated under reduced pressure. The resulting crude material was redissolved in CH₃CN and water, and lyophilized (374 mg, 1.39 mmol, quant. Yield).

¹H NMR (400 MHz, DMSO-d₆): δ ppm 7.96 (br s, 3H), 3.78 (m, 1H), 3.64 (s, 3H), 2.44 (m, 2H), 2.14 (m, 2H), 1.36 (s, 3H).

(cis-3-amino-1-methyl-cyclobutyl)-[(3S)-3-(3,5-difluorophenyl)isoxazolidin-2-yl]methanone trifluoroacetate is synthesized similarly but starting from (3S)-3-(3,5-difluorophenyl)isoxazolidine.

Synthesis of cis-3-(tert-butoxycarbonylamino)-1-methyl-cyclobutanecarboxylic acid is performed in a similar way as detailed in STEP 4 of Example 1.1—Compound responding to formula (IIa) in Scheme 1, but starting from methyl cis-3-(tert-butoxycarbonylamino)-1-methyl-cyclobutanecarboxylilate

### Example 3.1: Synthesis of Compounds (5), (6) and (7)

STEP 1: Synthesis of methyl cis-3-[(4-cyanopyrimidin-2-yl)amino]-1-methyl-cyclobutanecarboxylate—Compound responding to formula (III) in Scheme 1—reaction under step 6b of scheme 3

To a stirred suspension of 2-chloropyrimidine-4-carbonitrile (215 mg, 1.51 mmol) and methyl cis-3-amino-1-methylcyclobutane-1-carboxylate-trifluoroacetate (353 mg, 1.37 mmol) in CH₃CN (3.5 ml) in a micro wave vial, Net/Pr₂ was added (957 μl, 5.49 mmol) at rt. The vial was capped and the solution was heated to 80° C. in a micro wave reactor for 2 hrs. Volatile components were removed under reduced pressure and the resulting residue purified by column chromatography (SiO₂; CH₂Cl₂/EtOH gradient). The obtained product was dissolved in CH₃CN/water and lyophilized (221 mg, 900 μmol, 65% yield).

STEP 2: Synthesis of cis-3-[(4-cyanopyrimidin-2-yl)amino]-1-methyl-cyclobutanecarboxylic acid trifluoroacetate and cis-3-[(4-carbamoylpyrimidin-2-yl)amino]-1-methyl-cyclobutanecarboxylic acid trifluoroacetate—Compound responding to formula (IIa) in Scheme 1

To a solution of methyl cis-3-[(4-cyanopyrimidin-2-yl)amino]-1-methyl-cyclobutane-carboxylate (220 mg, 893 μmol) in THF (15 ml) a solution of lithium hydroxide (44 mg, 1.80 mmol) in water (1.5 ml) was added. The resulting solution was stirred at rt for 4 hrs. Volatile components were removed under reduced pressure and the remaining aqueous solution acidified with 1 N HCl. The aqueous layer was extracted with CH₂Cl₂, the combined organic layers were dried over Na₂SO₄, filtered and concentrated to afford a mixture of cis-3-[(4-cyanopyrimidin-2-yl)amino]-1-methyl-cyclobutanecarboxylic acid and cis-3-[(4-carbamoy-lpyrimidin-2-yl)amino]-1-methyl-cyclobutanecarboxylic acid (158 mg). 100 mg of the mixture were subjected to preparative reversed phase chromatography to give Cis-3-[(4-cyanopyrimidin-2-yl)amino]-1-methyl-cyclobutanecarboxylic acid trifluoro acetate (37 mg, 107 μmol, 12% yield) and cis-3-[(4-carbamoylpyrimidin-2-yl)amino]-1-methyl-cyclobutanecarboxylic acid trifluoro acetate (45 mg, 123 μmol, 14% yield). The remaining 58 mg were used in the next reaction without further purification.

Cis-3-[(4-cyanopyrimidin-2-yl)amino]-1-methyl-cyclobutanecarboxylic acid trifluoro acetate: ¹H NMR (400 MHz, DMSO-d₆): δ ppm 12.18 (brs, 1H), 8.55 (brs, 1H), 8.14 (br d, J=6.97 Hz, 1H), 7.10 (d, J=4.65 Hz, 1H), 4.35 (br s, 1H), 2.35 (m, 2H), 2.17 (m, 2H), 1.37 (s, 3H).

Cis-3-[(4-carbamoylpyrimidin-2-yl)amino]-1-methyl-cyclobutanecarboxylic acid trifluoro acetate: ¹H NMR (400 MHz, DMSO-d₆): δ ppm 12.18 (br s, 1H), 8.46 (br d, J=3.91 Hz, 1H), 8.08 (br s, 1H), 7.74 (br s, 1H), 7.64 (br s, 1H), 7.05 (d, J=4.77 Hz, 1H), 4.57 (br s, 1H), 2.36 (m, 2H), 2.18 (m, 2H), 1.40 (s, 3H).

STEP 3a: Synthesis of cis-2-((3-((S)-3-(3,5-difluorophenyl)isoxazolidine-2-carbonyl)-3-methylcyclobutyl)amino)pyrimidine-4-carbonitrile (Compound (5)) and cis-2-((3-((S)-3-(3,5-difluorophenyl)isoxazolidine-2-carbonyl)-3-methylcyclobutyl)amino)pyrimidine-4-carboxamide (Compound (6))

A mixture of cis-3-[(4-cyanopyrimidin-2-yl)amino]-1-methyl-cyclobutanecarboxylic acid and cis-3-[(4-carbamoylpyrimidin-2-yl)amino]-1-methyl-cyclobutanecarboxylic acid (35 mg) was dissolved in DMF (4.5 ml). With stirring iPrNEt₂ (100 μl, 0.58 mmol) and (S)-3-(3,5-difluorophenyl)isoxazolidine (30 mg, 160 μmol), dissolved in dry DMF (0.5 ml), were added followed by HATU (113 mg, 290 μmol) 15 min later. After stirring for 1 hr the mixture stood overnight. Then the mixture was directly purified by preperative reversed phase chromatography.

STEP 3b: Synthesis of cis-2-((3-((S)-3-(5-cyanopyridin-3-yl)isoxazolidine-2-carbonyl)-3-methylcyclobutyl)amino)pyrimidine-4-carboxamide (Compound (7)) is performed similarly as STEP 3a but starting from (S)-3-(5-cyanopyridin-3-yl)isoxazolidine.

### Example 3.2: Synthesis of Compounds (8) and (9)

To a stirred suspension of ethyl 6-chloro-5-fluoro-pyrimidine-4-carboxylate (25 mg, 134 μmol) and (cis-3-amino-1-methyl-cyclobutyl)-[(3S)-3-(3,5-difluorophenyl) isoxazolidin-2-yl]methanone trifluoroacetate (55 mg, 134 μmol) in CH₃CN (1.5 ml) in a micro wave vial Net/Pr₂ was added (75 μl, 428 μmol) at rt. The vial was capped and the solution was heated to 80° C. in a micro wave reactor for 30 min. Volatile components were removed under reduced pressure and the resulting residue purified by column chromatography (SiO₂; heptane/EA gradient) to give compound (8) (30 mg, 65 μmol, 53% yield) and compound (9) (20 mg, 42 μmol, 34% yield).

### Example 3.3: Synthesis of Compounds (1), (3) and (4)

cis-2-((3-((S)-3-(3,5-difluorophenyl)isoxazolidine-2-carbonyl)cyclobutyl)amino)pyrimidine-4-carboxamide (Compound (1)), methyl cis-2-((3-((S)-3-(3,5-difluorophenyl)isoxazolidine-2-carbonyl)cyclobutyl)amino)pyrimidine-4-carboxylate (Compound (3)) and trans-2-((3-((S)-3-(3,5-difluorophenyl)isoxazolidine-2-carbonyl)cyclobutyl)amino)pyrimidine-4-carboxamide (Compound (4)) are synthesised similarly to compounds (8) and (9) in Example 3.2 but starting respectively from (cis-3-amino-cyclobutyl)-[(3S)-3-(3,5-difluorophenyl) isoxazolidin-2-yl]methanone and (trans-3-amino-cyclobutyl)-[(3S)-3-(3,5-difluorophenyl) isoxazolidin-2-yl]methanone.

### Example 3.4: Synthesis of cis-6-chloro-5-((3-((S)-3-(3,5-difluorophenyl)isoxazolidine-2-carbonyl)-3-methylcyclobutyl)amino)pyrimidine-4-carboxamide (Compound (10))

Ethyl 6-chloro-5-[[cis-3-[(3S)-3-(3,5-difluorophenyl)isoxazolidine-2-carbonyl]-3-methyl-cyclobutyl]amino]pyrimidine-4-carboxylate (3 mg, 10 μmol) was dissolved in 7 M NH₃ solution in methanol (0.3 ml). After stirring for 1 hr the solvent mixture was removed in vacuo. The residue was lyophilized to afford the title compound in quantitative yield.

### Example 3.5: Synthesis of cis-6-((3-((S)-3-(3,5-difluorophenyl)isoxazolidine-2-carbonyl)-3-methylcyclobutyl)amino)-5-fluoropyrimidine-4-carboxamide (Compound (11))

Ethyl 6-[[cis-3-[(3S)-3-(3,5-difluorophenyl)isoxazolidine-2-carbonyl]-3-methyl-cyclobutyl]amino]-5-fluoro-pyrimidine-4-carboxylate (26 mg, 60 μmol) was dissolved in 7 M NH₃ solution in methanol (2.3 ml). After stirring for 1 hr the solvent mixture was removed in vacuo. The residue was purified by reversed phase chromatography to give 15.9 mg (36.5 μmol, 61% yield) of the title compound after lyophilisation.

### Example 4: Synthesis of Compounds (96), (97), (98), (109), (110)

### Example 4.0: Synthesis of methyl trans-4-(methylsulfonyloxymethyl)cyclohexanecarboxylate

To a stirred solution of methyl trans-4-(hydroxymethyl)cyclohexanecarboxylate (4 g, 22.5 mmol) and Net₃ (8.79 ml, 63.1 mmol) in THF (100 ml) methansulfonyl chloride (2.67 ml, 33.8 mmol) was added dropwise at 0° C. The resulting suspension was stirred at rt for 2 hrs. The reaction mixture was filtered, the filter cake rinsed with THF and the filtrate concentrated under reduced pressure. The title compound was obtained as pale yellow solid, which was used in the next reaction without further purification (5.64 g, 22.5 mmol, quant. Yield).

¹H NMR (400 MHz, CDCl₃): δ ppm 4.05 (d, J=7.21 Hz, 2H), 3.67 (s, 3H), 3.01 (s, 3H) 2.28 (m, 1H), 2.05 (m, 2H), 1.89 (m, 2H), 1.75 (m, 1H), 1.42 (m, 2H), 1.09 (m, 2H)

Synthesis of methyl 3-methylsulfonyloxycyclobutanecarboxylate is performed in a similar way as detailed for methyl trans-4-(methylsulfonyloxymethyl)cyclohexanecarboxylate in Example 4.0, but starting from methyl 3-hydroxycyclobutanecarboxylate

### Example 4.1: Synthesis of Compounds (96), (97), (98), (109) and (110)

STEP 1: Synthesis of methyl trans-3-(5-fluoroindazol-1-yl)cyclobutanecarboxylate, methyl trans-3-(5-fluoroindazol-2-yl)cyclobutanecarboxylate, methyl cis-3-(5-fluoroindazol-1-yl)cyclobutanecarboxylate and methyl cis-3-(5-fluoroindazol-2-yl)cyclobutanecarboxylate—reaction under step 6c of scheme 3

A solution of methyl 3-methylsulfonyloxycyclobutanecarboxylate (3.21 g, 15.43 mmol, 1.4 eq.), 5-fluoro-1H-indazole (1.5 g, 11.02 mmol, 1 eq.) and Cs₂CO₃ (7.18 g, 22.04 mmol, 2 eq.) in DMF (10 ml) was stirred at 80° C. for 16 hrs. Saturated NH₄Cl solution was added at 25° C. to adjust pH=7-8 and extracted with EA 150 ml (50 ml×3). The combined organic layers were washed with saturated brine 100 ml (50 ml×2), dried over absolute Na₂SO₄, filtered and concentrated under reduced pressure to give a residue, which was purified by prep-HPLC (column: Phenomenex luna C18 (250*70 mm, 10 μm); mobile phase: [water (0.1% TFA)-ACN]; ACN 32%-62%, 25 min) to give the title compound methyl trans-3-(5-fluoroindazol-1-yl)cyclobutanecarboxylate (0.413 g, 1.66 mmol, 15% yield) as a yellow solid, methyl trans-3-(5-fluoroindazol-2-yl)cyclobutanecarboxylate (0.95 g, 3.83 mmol, 35% yield) as a yellow oil, methyl cis-3-(5-fluoroindazol-1-yl)cyclobutanecarboxylate (0.218 g, 878.14 μmol, 8% yield) as a yellow solid and methyl cis-3-(5-fluoroindazol-2-yl)cyclobutanecarboxylate (0.44 g, 1.77 mmol, 16% yield) as a yellow oil.

Methyl trans-3-(5-fluoroindazol-1-yl)cyclobutanecarboxylate: ¹H NMR (400 MHz, CDCl₃): δ ppm 8.03 (s, 1H), 7.71 (dd, J=4.6, 9.3 Hz, 1H), 7.24 (dd, J=2.1, 9.0 Hz, 1H), 5.04 (t, J=8.3 Hz, 1H), 3.77 (s, 3H), 3.15-3.03 (m, 1H), 3.03-2.85 (m, 4H).

Methyl trans-3-(5-fluoroindazol-2-yl)cyclobutanecarboxylate: ¹H NMR (400 MHz, CDCl₃): δ ppm 7.92 (s, 1H), 7.72 (dd, J=4.6, 9.3 Hz, 1H), 7.23 (dd, J=2.0, 9.0 Hz, 1H), 7.12 (dt, J=2.4, 9.3 Hz, 1H), 5.32 (t, J=8.1 Hz, 1H), 3.80 (s, 3H), 3.40-3.27 (m, 1H), 3.17-3.03 (m, 2H), 2.94-2.81 (m, 2H).

Methyl cis-3-(5-fluoroindazol-1-yl)cyclobutanecarboxylate: ¹H NMR (400 MHz, CDCl₃): δ ppm 7.91 (s, 1H), 7.39 (dd, J=4.1, 9.1 Hz, 1H), 7.27 (dd, J=2.3, 8.6 Hz, 1H), 7.09 (dt, J=2.4, 9.0 Hz, 1H), 4.99-4.83 (m, 1H), 3.68 (s, 3H), 3.09-2.91 (m, 3H), 2.80-2.66 (m, 2H).

Methyl cis-3-(5-fluoroindazol-2-yl)cyclobutanecarboxylate: ¹H NMR (400 MHz, CDCl₃): δ ppm 8.03 (s, 1H), 7.44-7.32 (m, 2H), 7.17 (dt, J=2.4, 9.0 Hz, 1H), 5.37 (quin, J=8.0 Hz, 1H), 3.81 (s, 3H), 3.37-3.24 (m, 1H), 3.16-3.00 (m, 2H), 2.90-2.73 (m, 2H).

STEP 2: Synthesis of cis-3-(5-fluoroindazol-2-yl)cyclobutanecarboxylic acid, cis-3-(5-fluoroindazol-1-yl)cyclobutanecarboxylic acid and trans-3-(5-fluoroindazol-1-yl)cyclobutanecarboxylic acid are performed in a similar way as detailed in STEP 4 of Example 1.1 from their respective methylesters—compounds responding to formula (IIa) in scheme 1

STEP 3a: Synthesis of cis-[(3S)-3-(3,5-difluorophenyl)isoxazolidin-2-yl]-[3-(5-fluoroindazol-1-yl)cyclobutyl]methanone (Compound (96)) is performed in a similar way as in STEP 5b of Example 1.1.

STEP 3b: Synthesis of trans-[(3S)-3-(3,5-difluorophenyl)isoxazolidin-2-yl]-[3-(5-fluoroindazol-1-yl)cyclobutyl]methanone (Compound (97)) is performed in a similar way as in STEP 5b of Example 1.1.

STEP 3c: Synthesis of trans-3-fluoro-5-[(3S)-2-[3-(5-fluoroindazol-1-yl)cyclobutanecarbonyl]isoxazolidin-3-yl]benzonitrile (Compound (98)) is performed in a similar way as in STEP 5a of Example 1.1.

STEP 3d: Synthesis of cis-3-fluoro-5-[(3S)-2-[3-(5-fluoroindazol-2-yl)cyclobutanecarbonyl]isoxazolidin-3-yl]benzonitrile (Compound (109)) is performed in a similar way as in STEP 5a of Example 1.1.

STEP 3e: Synthesis of cis-[(3S)-3-(3,5-difluorophenyl)isoxazolidin-2-yl]-[3-(5-fluoroindazol-2-yl)cyclobutyl]methanone (Compound (110)) is performed in a similar way as in STEP 5b of Example 1.1.

### Example 4.2: Synthesis of Compounds (105), (106), (107) and (108)

STEP 1: Synthesis of methyl 3-(5-fluoroindol-1-yl)cyclobutanecarboxylate—compound responding to formula (III) in scheme 1—reaction under step 6c of scheme 3

To a solution of 5-fluoro-1H-indole (1.5 g, 11.10 mmol, 1 eq.) in DMF (15 ml) was added Cs₂CO₃ (7.23 g, 22.20 mmol, 2 eq.) and methyl 3-methylsulfonyloxycyclobutane-carboxylate (3.47 g, 16.65 mmol, 1.5 eq.). The mixture was stirred at 80° C. for 12 hrs. The reaction mixture was adjusted to pH 3-4 with 1 N HCl. The precipitate was collected by filtration, dried under reduced pressure to give the desired compound. The aqueous phase was extracted with EA (100 ml×2), dried over Na₂SO₄, filtered and concentrated under reduced pressure to give a residue. The crude product was purified by reversed-phase HPLC (0.1% formic acid condition) to give methyl 3-(5-fluoroindol-1-yl)cyclobutanecarboxylate (823 mg, 3.33 mmol, 30% yield).

STEP 2: Synthesis trans-3-(5-fluoroindol-1-yl)cyclobutanecarboxylic acid and cis-3-(5-fluoroindol-1-yl)cyclobutanecarboxylic acid—compounds responding to formula (IIa) in scheme 1

To a solution of methyl 3-(5-fluoroindol-1-yl)cyclobutanecarboxylate (1.71 g, 6.92 mmol, 1 eq.) in THF (9 ml) and water (9 ml) was added LiOH·H₂O (290.18 mg, 6.92 mmol, 1 eq.). The mixture was stirred at 25° C. for 1 hr. Several new peaks were shown on LC/MS and ~81% of desired compound was detected. The reaction mixture was concentrated. The crude product was purified by reversed-phase HPLC (0.1% formic acid condition) and by prep-HPLC (column: Phenomenex luna C18 150*40 mm, 15 μm; mobile phase: [water (0.225% formic acid)-ACN]; gradient: 38%-48% ACN in 10 min) to give a cis/trans mixture of 3-(5-fluoroindol-1-yl)cyclobutanecarboxylic acid (1.3 g, 76% yield) as a white solid.

Cis/trans mixture of 3-(5-fluoroindol-1-yl)cyclobutanecarboxylic acid (1.54 g, 6.58 mmol, 1 eq.) was separated by SFC (column: DAICEL CHIRALPAK AD (250*30 mm, 10 μm); mobile phase: 20% MeOH [+0.1% NH₃H₂O] in supercritical CO₂, single batch cycle process with 6.1 min lag time between two sequential injections; total duration 400 min) to give trans-3-(5-fluoroindol-1-yl)cyclobutanecarboxylic acid (375 mg, >99.9% e.e.) as yellow solid and cis-3-(5-fluoroindol-1-yl)cyclobutanecarboxylic acid (1.1 g, >99.9% e.e.) As yellow oil.

Trans-3-(5-fluoroindol-1-yl)cyclobutanecarboxylic acid: ¹H NMR (400 MHz, DMSO-d₆): δ ppm 7.70 (d, J=3.2 Hz, 1H), 7.43 (dd, J=4.5, 8.9 Hz, 1H), 7.30 (dd, J=2.6, 9.9 Hz, 1H), 6.95 (dt, J=2.6, 9.2 Hz, 1H), 6.47 (d, J=3.1 Hz, 1H), 5.10 (quin, J=8.3 Hz, 1H), 3.11-3.01 (m, 1H), 2.77-2.59 (m, 4H).

Cis-3-(5-fluoroindol-1-yl)cyclobutanecarboxylic acid: ¹H NMR (400 MHz, DMSO-d₆): δ ppm 7.57 (d, J=3.2 Hz, 1H), 7.53 (dd, J=4.5, 9.0 Hz, 1H), 7.30 (dd, J=2.5, 9.8 Hz, 1H), 6.97 (dt, J=2.6, 9.2 Hz, 1H), 6.45 (d, J=3.2 Hz, 1H), 4.96-4.78 (m, 1H), 4.03 (q, J=7.2 Hz, 1H), 2.96-2.79 (m, 1H), 2.76-2.65 (m, 2H), 2.57-2.51 (m, 2H).

STEP 3a: Synthesis of trans-3-fluoro-5-[(3S)-2-[3-(5-fluoroindol-1-yl)cyclobutanecarbonyl]isoxazolidin-3-yl]benzonitrile (Compound (105)) is performed in a similar way as in STEP 5a of Example 1.1.

STEP 3b: Synthesis of trans-[(3S)-3-(3,5-difluorophenyl)isoxazolidin-2-yl]-[3-(5-fluoroindol-1-yl)cyclobutyl]methanone (Compound (106)) is performed in a similar way as in STEP 5b of Example 1.1.

STEP 3c: Synthesis of cis-3-fluoro-5-[(3S)-2-[3-(5-fluoroindol-1-yl)cyclobutanecarbonyl]isoxazolidin-3-yl]benzonitrile (Compound (107)) is performed in a similar way as in STEP 5a of Example 1.1.

STEP 3d: Synthesis of cis-[(3S)-3-(3,5-difluorophenyl)isoxazolidin-2-yl]-[3-(5-fluoroindol-1-yl)cyclobutyl]methanone (Compound (108)) is performed in a similar way as in STEP 5b of Example 1.1.

### Example 5: Synthesis of Compound (114)

STEP 1: Synthesis of methyl cis-3-(4-carbamoyl-2-pyridyl)cyclopentanecarboxylate—compound responding to formula (III) in scheme 1

### Step a: Synthesis of 2-bromopyridine-4-carboxamide

A mixture of 2-bromopyridine-4-carboxylic acid (8.6 g, 42.57 mmol, 1 eq.), NH₄Cl (3.42 g, 63.86 mmol, 1.5 eq.), Net/Pr₂ (22.01 g, 170.29 mmol, 29.66 ml, 4 eq.) and CDI (8.28 g, 51.09 mmol, 1.2 eq.) in DMF (80 ml) was degassed, purged with N₂ for 3 times and stirred at 20° C. for 3 hrs under N₂ atmosphere. The mixture was diluted with water (500 ml), extracted with EA (300 ml×4), dried over Na₂SO₄, filtered and concentrated. The residue was triturated with DCM (30 ml) to give the title compound (5.7 g, 67% yield) as white solid.

¹H NMR (400 MHz, DMSO-d₆): δ ppm 8.53 (d, J=5.0 Hz, 1H), 8.31 (br s, 1H), 8.00 (s, 1H), 7.91-7.76 (m, 2H).

### Step b: Synthesis of methyl 3-(trifluoromethylsulfonyloxy)cyclopent-2-ene-1-carboxylate

To a solution of methyl 3-oxocyclopentanecarboxylate (19 g, 133.66 mmol, 1 eq.) and Net/Pr₂ (25.91 g, 200.49 mmol, 34.92 ml, 1.5 eq.) in toluene (500 ml) Tf₂O (56.57 g, 200.49 mmol, 33.08 ml, 1.5 eq.) was added dropwise at 45° C. After addition, the mixture was stirred at 45° C. for 1 hr. The mixture was diluted with water (300 ml) and extracted with EA (300 ml×2). The combined organic layers were dried over Na₂SO₄, filtered, concentrated under reduced pressure and purified by column chromatography (SiO₂, petroleum ether/EA=10/1 to 3/1) to give the title compound (29.3 g, 80% yield) as a black oil.

### Step c: Synthesis of methyl 3-(4,4,5,5-tetramethyl-1,3,2-dioxaborolan-2-yl)cyclopent-2-ene-1-carboxylate

A mixture of methyl 3-(trifluoromethylsulfonyloxy)cyclopent-2-ene-1-carboxylate (29 g, 105.76 mmol, 1 eq.), KOAc (10.38 g, 105.76 mmol, 1 eq.), BPD (26.86 g, 105.76 mmol, 1 eq.), KOAc (10.38 g, 105.76 mmol, 1 eq.) and DPPF (2.93 g, 5.29 mmol, 0.05 eq.) in dioxane (300 ml) was degassed and purged with N₂ for 3 times and stirred at 20° C. 0.5 hrs. Pd(dppf)Cl₂·CH₂Cl₂ (4.32 g, 5.29 mmol, 0.05 eq.) was added and the mixture was stirred at 90° C. under N₂ atmosphere for 12 hrs. The mixture was concentrated and diluted with water (200 ml), extracted with EA (150 ml×3), dried over Na₂SO₄, filtered, concentrated under reduced pressure and purified by flash silica gel chromatography (petroleum ether/EA=5/1) to give the title compound (25 g, 94% yield) as a yellow oil.

¹H NMR: (400 MHz, CDCl₃): δ ppm 6.52-6.36 (m, 1H), 3.69 (d, J=1.0 Hz, 3H), 3.67-3.60 (m, 1H), 3.20-3.08 (m, 1H), 2.87-2.70 (m, 2H), 2.68-2.55 (m, 1H), 2.53-2.40 (m, 1H), 2.21-2.11 (m, 1H), 1.27 (s, 12H).

### Step d: Synthesis of methyl 3-(4-carbamoyl-2-pyridyl)cyclopent-2-ene-1-carboxylate

A mixture of methyl 3-(4,4,5,5-tetramethyl-1,3,2-dioxaborolan-2-yl)cyclopent-2-ene-1-carboxylate (4.52 g, 17.91 mmol, 1.2 eq.), 2-bromopyridine-4-carboxamide (3 g, 14.92 mmol, 1 eq.), Na₂CO₃ (3.16 g, 29.85 mmol, 2 eq.), X-PHOS (1.07 g, 2.24 mmol, 0.15 eq.) and Pd(PPh₃)₄ (1.72 g, 1.49 mmol, 0.1 eq.) in dioxane (40 ml) and water (10 ml) was degassed and purged with N₂ for 3 times and stirred at 80° C. under N₂ atmosphere for 12 hrs. The mixture was cooled to 20° C. and diluted with EA (300 ml), washed with water (50 ml×2) dried over Na₂SO₄, filtered, concentrated and purified by prep. RP-LC (column: Welch Ultimate XB C18, 120 Å, I.D. 72×300 mm, 20-40 μm, mobile phase: water (0.1% formic acid) and ACN; gradient: 10%-35% ACN in 30 min; 35% ACN for 5 min; flow: 200 ml/min) to give the title compound (1.1 g, 30% yield) as a yellow solid.

LC/MS: m/z 247.2 [M+H]⁺

### Step e: Synthesis of methyl cis-3-(4-carbamoyl-2-pyridyl)cyclopentanecarboxylate

To a solution of methyl 3-(4-carbamoyl-2-pyridyl)cyclopent-2-ene-1-carboxylate (1.1 g, 4.47 mmol, 1 eq.) in MeOH (10 ml) Pd/C (13 mg, 4.47 mmol, 10% purity, 1.00 eq.) was added under N₂ atmosphere. The suspension was degassed and purged with H₂ for 3 times and stirred under H₂ (15 Psi) at 20° C. for 1 hr. The reaction mixture was filtered, concentrated under reduced pressure and purified by column chromatography (SiO₂, DCM:MeOH=10:1) to give the title compound (1 g, 88% yield) as yellow solid.

LC/MS: m/z 249.1 [M+H]⁺

¹H NMR (400 MHz, DMSO-d₆): δ ppm 8.61 (d, J=5.0 Hz, 1H), 8.21 (br s, 1H), 7.66 (s, 2H), 7.58 (dd, J=1.4, 5.1 Hz, 1H), 3.65-3.59 (m, 3H), 3.17 (d, J=5.3 Hz, 1H), 3.05-2.90 (m, 1H), 2.35-2.24 (m, 1H), 2.14-1.92 (m, 4H), 1.89-1.76 (m, 1H).

STEP 2: Synthesis of cis-3-(4-carbamoyl-2-pyridyl)cyclopentanecarboxylic acid is performed in a similar way as detailed in STEP 4 of Example 1.1—compounds responding to formula (IIa) in scheme 1, starting from methyl cis-3-(4-carbamoyl-2-pyridyl)cyclopentanecarboxylilate

STEP 3: Synthesis of cis-2-[3-[(3S)-3-(3,5-difluorophenyl)isoxazolidine-2-carbonyl]cyclopentyl]pyridine-4-carboxamide (Compound (114)).

To a solution of cis-3-(4-carbamoyl-2-pyridyl)cyclopentanecarboxylic acid (200.00 mg, 853.79 μmol, 1 eq.), Net/Pr₂ (331.03 mg, 2.56 mmol, 446.13 μl, 3 eq.) and T₃P (651.98 mg, 1.02 mmol, 609.33 μl, 50% purity, 1.2 eq.) in DMF (2 ml) (3S)-3-(3,5-difluorophenyl)isoxazolidine (198.69 mg, 896.48 μmol, 1.05 eq., HCl) was added. The mixture was stirred at 20° C. for 3 hrs, diluted with water (20 ml), extracted with EA (10 ml×3), washed with brine (10 ml), dried over Na₂SO₄, filtered, concentrated under reduced pressure to give the title compound (150 mg, crude) as a yellow oil.

Racemic mixture of cis-2-[3-[(3S)-3-(3,5-difluorophenyl)isoxazolidine-2-carbonyl]cyclopentyl]pyridine-4-carboxamide (150 mg) was separated by SFC (column: DAICEL CHIRALPAK AD, 250×30 mm, 10 μm; mobile phase: 45% MeOH (0.1% NH₃·H₂O) in supercritical CO₂, single batch cycle process with 5.2 min lag time between two sequential injections; total duration 30 min) to give cis-2-[3-[(3S)-3-(3,5-difluorophenyl)isoxazolidine-2-carbonyl]cyclopentyl]pyridine-4-carboxamide stereoisomer 1 (peak 1, 77 mg) and compound 114 stereoisomer 2 (peak 2, 42 mg) as off-white solid.

Cis-2-[3-[(3S)-3-(3,5-difluorophenyl)isoxazolidine-2-carbonyl]cyclopentyl]pyridine-4-carboxamide Stereisomer 1, peak 1:

LC/MS: m/z 402.2 [M+H]⁺, Rt=0.788 min (LC/MS method B)

SFC (Column: Chiralpak AD-3 50×4.6 mm I.D., 3 μm; mobile phase: CO₂ and MeOH (0.05% DEA), gradient: 5%-40% MeOH (0.05% DEA); flow rate: 3 ml/min; column temperature: 35° C.; 100 bar): Rt=1.93 min (100%).

¹H NMR (400 MHz, CDCl₃): δ ppm 8.67 (d, J=5.0 Hz, 1H), 7.69 (s, 1H), 7.48 (dd, J=1.5, 5.0 Hz, 1H), 6.90-6.81 (m, 2H), 6.71 (tt, J=2.1, 8.9 Hz, 1H), 6.27 (br dd, J=4.0, 6.9 Hz, 1H), 5.82-5.51 (m, 1H), 5.39 (dd, J=6.1, 8.8 Hz, 1H), 4.26 (dt, J=3.1, 7.9 Hz, 1H), 3.94-3.83 (m, 1H), 3.51-3.35 (m, 2H), 2.86 (dddd, J=3.0, 6.5, 9.2, 12.3 Hz, 1H), 2.44-2.27 (m, 2H), 2.24-2.09 (m, 4H), 2.04-1.91 (m, 1H).

### Example 6: Synthesis of Compounds (122), (123), (124), (125), (126), (127), (128)

### Example 6.0: Synthesis of trans-4-[(6-carbamoyl-5-fluoro-benzimidazol-1-yl)methyl]cyclohexane carboxylic acid

To a solution of trans-4-[(6-cyano-5-fluoro-benzimidazol-1-yl)methyl]cyclohexane carboxylic acid (100 mg, 332 μmol) in THF (4 ml) hydrogen peroxide (50%, 40 μl, 663 μmol) was added at rt followed by a solution of lithium hydroxide (19 mg, 797 μmol) in water (4 ml). The resulting solution was stirred at rt for 30 min after which another equivalent of hydrogen peroxide was added. Volatile components were removed under reduced pressure and the remaining aqueous solution diluted with approx. 3 ml water and acidified with 1 N HCl (796 μl, 796 μmol). The obtained suspension was stirred for 10 min and filtered. The title compound was obtained as a white solid, which was used in the next reaction without further purification (76 mg, 72%).

### Example 6.1

STEP 1: Synthesis of methyl 4-[(6-cyanopyrimidin-4-yl)oxymethyl]cycloheptanecarboxylate—compound responding to formula (III) in scheme 1

### Step a: Synthesis of 2-(3-oxo-3-phenyl-propyl)cyclopentanone

To a solution of 3-(dimethylamino)-1-phenyl-propan-1-one hydrochloride (15.00 g, 70.19 mmol, 1 eq.) in dioxane (150 ml) 4-(cyclopenten-1-yl)morpholine (10.75 g, 70.19 mmol, 11.23 ml, 1 eq.) was added and the mixture was stirred at 110° C. for 12 hrs. The reaction mixture was concentrated and diluted with EA (150 ml), washed with 1 N HCl (30 ml), the aqueous layer was extracted with EA (20 ml×3), combined organic layers were washed with brine (100 ml), dried over anhydrous Na₂SO₄, filtered and concentrated under reduced pressure. The residue was purified by column chromatography (SiO₂, petroleum ether/EA=10/1) to give the title compound (8 g, 53% yield) as a white solid.

LC/MS: m/z 217.1 [M+H]⁺

¹H NMR (400 MHz, CDC₃): δ ppm 7.98 (d, J=7.3 Hz, 2H), 7.64-7.52 (m, 1H), 7.52-7.43 (m, 2H), 3.14 (ddd, J=4.7, 6.6, 8.0 Hz, 2H), 2.41-2.08 (m, 5H), 2.04-1.98 (m, 1H), 1.90-1.75 (m, 2H), 1.68-1.52 (m, 2H).

### Step b: Synthesis of 4-phenylcyclohept-3-ene-1-carboxylic acid

To a solution of 2-(3-oxo-3-phenyl-propyl)cyclopentanone (8 g, 36.99 mmol, 1 eq.) in AcOH (65 ml) and HCl (12 M, 15 ml, 4.87 eq.) was added and the mixture was stirred at 110° C. for 4 hrs. The mixture was concentrated and poured into 1 N NaOH (10 ml), washed with DCM (10 ml), the pH was adjusted to pH 1 by addition of 1 N HCl and extracted with EA (30 ml×2) to give the title compound (6.2 g, crude) as yellow oil.

LC/MS: m/z 217.2 [M+H]⁺

¹H NMR (400 MHz, DMSO-d₆): δ ppm 12.05-11.98 (m, 1H), 7.32-7.29 (m, 4H), 7.27-7.23 (m, 1H), 6.24-5.84 (m, 1H), 2.71-2.62 (m, 2H), 2.36-2.30 (m, 2H), 2.23 (s, 1H), 2.15-1.79 (m, 6H), 1.63-1.42 (m, 2H).

### Step c: Synthesis of methyl 4-phenylcyclohept-3-ene-1-carboxylate

To a solution of 4-phenylcyclohept-3-ene-1-carboxylic acid (6.2 g, 28.67 mmol, 1 eq.) in MeOH (60 ml) H₂SO₄ (562.33 mg, 5.73 mmol, 305.61 μl, 0.2 eq.) was added and the mixture was stirred at 70° C. for 12 hrs. The mixture was concentrated, diluted with EA (50 ml), washed with water (20 ml×2), dried over anhydrous Na₂SO₄, filtered and concentrated to give the title compound (6 g, crude) as a yellow oil.

LC/MS: m/z 231.2 [M+H]⁺

### Step d: Synthesis of 4-methoxycarbonylcycloheptanecarboxylic acid

To a solution of methyl 4-phenylcyclohept-3-ene-1-carboxylate (5.8 g, 24.97 mmol, 1 eq.) and NaIO₄ (96.12 g, 449.39 mmol, 24.90 ml, 18 eq.) in ACN (60 ml) and water (120 ml) CCl₄ (60 ml) and RuCl₃ (103.57 mg, 499.32 μmol, 33.30 μl, 0.02 eq.) were added and the mixture was stirred at 20° C. for 12 hrs. The mixture was filtered and concentrated, the pH was adjusted to pH 8 by saturated sodium bicarbonate solution, washed with DCM (40 ml), acidified with 1 N HCl to pH 1, extracted with EA (80 ml×2), dried over Na₂SO₄, filtered and concentrated to give the title compound (3 g, crude) as a yellow oil.

¹H NMR (400 MHz, CD₃OD): δ ppm 4.88 (br s, 3H), 2.64-2.41 (m, 2H), 2.10-2.05 (m, 1H), 2.00-1.81 (m, 5H), 1.79-1.58 (m, 4H), 1.53-1.38 (m, 1H).

### Step e: Synthesis of methyl 4-(hydroxymethyl)cycloheptanecarboxylate

To a solution of 4-methoxycarbonylcycloheptanecarboxylic acid (3 g, 14.98 mmol, 1 eq.) in THF (30 ml) BH₃·Me₂S (10 M, 1.95 ml, 1.3 eq.) was added at 0° C. under N₂ atmosphere and the mixture was stirred at 20° C. for 2 hrs. The reaction mixture was concentrated under reduced pressure and purified by column chromatography (SiO₂, DCM/MeOH=5/1) to give the title compound (1.8 g, 65% yield) as yellow oil.

¹H NMR (400 MHz, CD₃OD): δ ppm 3.64 (s, 3H), 3.38-3.32 (m, 1H), 2.61-2.43 (m, 1H), 2.07-2.02 (m, 1H), 2.01-1.93 (m, 1H), 1.93-1.80 (m, 3H), 1.79-1.67 (m, 2H), 1.67-1.52 (m, 3H), 1.49-1.34 (m, 1H), 1.32-1.17 (m, 1H), 1.16-1.00 (m, 1H).

### Step f: Synthesis of methyl 4-[(6-chloropyrimidin-4-yl)oxymethyl]cycloheptanecarboxylate—

To a solution of 4,6-dichloropyrimidine (1.12 g, 7.52 mmol, 1 eq.) and methyl 4-(hydroxymethyl)cycloheptanecarboxylate (1.4 g, 7.52 mmol, 1 eq.) in DMF (14 ml) NaH (450.97 mg, 11.28 mmol, 60% purity, 1.5 eq.) was added at 0° C. and the mixture was stirred at 20° C. for 1 hr. The reaction mixture was diluted with water (100 ml), extracted with EA (30 ml×3), washed with brine (50 ml), dried over anhydrous Na₂SO₄, filtered, concentrated under reduced pressure and purified by column chromatography (SiO₂, DCM/MeOH=5/1) to give the title compound (1.5 g, 64% yield) as a yellow oil.

LC/MS: m/z 299.1 [M+H]⁺

¹H NMR (400 MHz, CDCl₃): δ ppm 8.56 (s, 1H), 6.77 (s, 1H), 4.24-4.15 (m, 2H), 3.68 (s, 3H), 2.61-2.45 (m, 1H), 2.13-1.97 (m, 2H), 1.96-1.84 (m, 3H), 1.82-1.73 (m, 1H), 1.72-1.60 (m, 2H), 1.56-1.49 (m, 1H), 1.48-1.31 (m, 1H), 1.30-1.16 (m, 1H).

### Step g: Synthesis of methyl 4-[(6-cyanopyrimidin-4-yl)oxymethyl]cycloheptanecarboxylate—compound responding to formula (III) in scheme 1

A mixture of methyl 4-[(6-chloropyrimidin-4-yl)oxymethyl]cycloheptanecarboxylate (1.5 g, 5.02 mmol, 1 eq.), Zn(CN)₂ (2.36 g, 20.08 mmol, 1.27 ml, 4 eq.) and Pd(PPh₃)₄ (1.16 g, 1.00 mmol, 0.2 eq.) in DMF (15 ml) was purged with N₂ for 3 times and stirred at 100° C. under N₂ atmosphere for 16 hrs. The reaction was diluted with water (200 ml), extracted with EA (80 ml×3), washed with brine (100 ml×2), dried over anhydrous Na₂SO₄, filtered, concentrated in vacuo and purified by flash silica gel chromatography (SiO₂, DCM:MeOH=10:1) to give the title compound (1.2 g, 79% yield) as a yellow solid.

LC/MS: m/z 290.1 [M+H]⁺

¹H NMR (400 MHz, CDCl₃): δ ppm 8.82 (s, 1H), 7.09 (s, 1H), 4.23 (br d, J=6.6 Hz, 2H), 3.68 (s, 3H), 2.63-2.46 (m, 1H), 2.14-1.98 (m, 2H), 1.98-1.84 (m, 3H), 1.83-1.73 (m, 1H), 1.73-1.62 (m, 2H), 1.49-1.31 (m, 1H), 1.30-1.18 (m, 1H).

STEP 2: Synthesis of 4-[(6-carbamoylpyrimidin-4-yl)oxymethyl]cycloheptanecarboxylic acid—compound responding to formula (IIa) in scheme 1 is performed in a similar way as in Example 6.1

STEP 3a: Synthesis of a diastereomeric mixture of 6-[[4-[(3S)-3-(3,5-difluorophenyl)isoxazolidine-2-carbonyl]cycloheptyl]methoxy]pyrimidine-4-carboxamide is performed in a similar way as in STEP 5b of Example 1.1, but starting from (3S)-3-(3,5-difluorophenyl)isoxazolidine.

The diastereomeric mixture of 6-[[4-[(3S)-3-(3,5-difluorophenyl)isoxazolidine-2-carbonyl]cycloheptyl]methoxy]pyrimidine-4-carboxamide was separated by SFC (column: DAICEL CHIRALPAK AD, 250×30 mm, 10 μm, mobile phase: 60% IPA (0.1% NH₃·H₂O) in CO₂, single batch cycle process with 4.3 min lag time between two sequential injections, total duration 30 min; column: DAICEL CHIRALCEL OJ, 250×30 mm, 10 μm, mobile phase: 25% MeOH (0.1% NH₃·H₂O) in CO₂, single batch cycle process with 2.4 min lag time between two sequential injections, total duration 30 min; and column: DAICEL CHIRALPAK AD, 250×30 mm, 10 μm, mobile phase: 55% IPA (0.1% NH₃·H₂O) in CO₂, single batch cycle process with 3.0 min lag time between two sequential injections, total duration 30 min) to give compound (122) stereoisomer 1 (peak 1, 47 mg, 102.07 μmol, 12% yield), compound (123) stereoisomer 2 (peak 2, 71 mg, 154.19 μmol, 19% yield), compound (124) stereoisomer 3 (peak 3, 73 mg, 158.53 μmol, 19% yield) and compound (125) stereoisomer 4 (peak 4, 75 mg, 162.88 μmol, 20% yield) as white solid.

STEP 3b: Synthesis of a diastereomeric mixture of 6-[[4-[3-(3-cyano-5-fluoro-phenyl)isoxazolidine-2-carbonyl]cycloheptyl]-methoxy]pyrimidine-4-carboxamide is performed in a similar way as in STEP 5a of Example 1.1.

The diastereomeric mixture of 6-[[4-[3-(3-cyano-5-fluoro-phenyl)isoxazolidine-2-carbonyl]cycloheptyl]-methoxy]pyrimidine-4-carboxamide was separated by SFC (column: DAICEL CHIRALPAK AD, 250×30 mm, 10 μm, mobile phase: 70% IPA (0.1% NH₃·H₂O) in CO₂, single batch cycle process with 6.5 min lag time between two sequential injections, total duration 30 min) to give a mixture of compound (126) stereoisomer 1 and compound (127) stereoisomer 2 (peak 1 and peak 2, 80 mg) as a white solid, compound (128) stereoisomer 3 (peak 3, 50 mg, 106.95 μmol, 18% yield) as a white solid and cis-6-[[4-[(3S)-3-(3-cyano-5-fluoro-phenyl)isoxazolidine-2-carbonyl]cycloheptyl]methoxy]pyrimidine-4-carboxamide stereoisomer 4 (peak 4, 55 mg, 117.65 μmol, 20% yield) as a yellow solid. Then the mixture of peak 1 and peak 2 (80 mg) was separated by SFC (column: DAICEL CHIRALCEL OJ, 250×30 mm, 10 μm, mobile phase: 30% MeOH (0.1% NH₃·H₂O) in CO₂, single batch cycle process with 2.8 min lag time between two sequential injections; total duration 25 min) to give compound (126) stereoisomer 1 (peak 1, 30 mg, 64.17 μmol, 11% yield) as a white solid and compound (127) stereoisomer 2 (peak 2, 39 mg, 83.42 μmol, 14% yield) as a white solid.

### Example 7: Synthesis of Compounds (85), (86)

STEP 1: Synthesis of Synthesis of methyl (1R,4R)-4-(iodomethyl)cyclohexane-1-carboxylate

To a solution of methyl (1R,4R)-4-(hydroxymethyl)cyclohexane-1-carboxylate (25 g, 145.16 mmol, 1 eq), imidazole (14.82 g, 217.74 mmol, 1.5 eq) and I₂ (55.27 g, 217.74 mmol, 43.86 mL, 1.5 eq) in DCM (250 mL) was added a solution of PPh₃ (57.11 g, 217.74 mmol, 1.5 eq) under N₂. The reaction mixture was stirred at 25° C. for 4 hours. TLC indicated no starting material was remained, and one major new spot with larger polarity was detected. The reaction mixture was poured into H₂O (1000 mL) and extracted with EA (200 mL*3). The combined organic layer was washed with brine (200 mL), dried over Na₂SO₄, filtered and concentrated. The residue was purified by column chromatography (SiO₂, Petroleum ether/Ethyl acetate=100/1 to 10/1). Methyl (1r,4r)-4-(iodomethyl)cyclohexane-1-carboxylate (28 g, 97.26 mmol, 67.00% yield, 98% purity) was obtained as a yellow oil.

¹H NMR (400 MHz, CHLOROFORM-d) 5=3.67 (s, 3H), 3.11 (d, J=6.4 Hz, 2H).

STEP 2: Synthesis of methyl (1r,4r)-4-((3-carbamoyl-4-methylphenoxy)methyl)cyclohexane-1-carboxylate—compound responding to formula (III) in scheme 1

To a solution of 5-hydroxy-2-methylbenzamide (200 mg, 1.32 mmol) in DMF (3.7 mL), NaH (60% in mineral oil, 32 mg, 1.32 mmol) was added, and the mixture stirred for 10 min at rt. Then methyl (1r,4r)-4-(iodomethyl)cyclohexane-1-carboxylate (0.75 g, 2.65 mmol) in DMF (3.7 mL) was added and the mixture stirred at rt for 1 h. An additional equivalent of NaH (60% in mineral oil, 32 mg, 1.32 mmol) was added and the mixture stirred at 50° C. for 3 h and then at rt overnight. The mixture was diluted with EA and washed with 0.1 M NaOH and the organic layer dried and concentrated. The residue was purified by column chromatography on silica gel to give methyl (1R,4R)-4-((3-carbamoyl-4-methylphenoxy)methyl)cyclohexane-1-carboxylate (90 mg, 22%).

LCMS, m/z 306.2[M+H]+; RT 1.88 min (Method A).

¹H NMR (DMSO-d6, 400 MHz) δ ppm 4.63 (s, 2H), 3.60 (s, 3H), 3.58 (s, 1H), 3.20 (d, J=6.24 Hz, 1H), 2.24 (m, 3H), 2.04 (m, 2H), 1.91 (m, 3H), 1.40 (m, 3H), 1.18 (m, 1H), 1.04 (m, 1H)

STEP 3: Synthesis of trans-4-[(3-carbamoyl-4-methyl-phenoxy)methyl]cyclohexanecarboxylic acid is performed in a similar way as detailed in STEP 4 of Example 1.1, but starting from methyl (1r,4r)-4-((3-carbamoyl-4-methylphenoxy)methyl)cyclohexane-1-carboxylate

LCMS, m/z 292.2 [M+H]+; RT 1.50 min (Method A).

¹H NMR (DMSO-d6, 400 MHz) δ ppm 12.00 (br s, 1H), 7.65 (br s, 1H), 7.29 (br s, 1H), 7.10 (d, J=8.31 Hz, 1H), 6.89 (s, 1H), 6.87 (d, J=8.67 Hz, 1H), 3.77 (d, J=6.36 Hz, 2H), 2.27 (s, 3H), 2.16 (m, 1H), 1.90 (m, 4H), 1.70 (m, 1H), 1.32 (m, 2H), 1.07 (dtd, J=12.75, 12.58, 12.58, 3.06 Hz, 2H).

STEP 4a: Synthesis of trans-5-((4-((S)-3-(3-cyano-5-fluorophenyl)isoxazolidine-2-carbonyl)cyclohexyl)methoxy)-2-methylbenzamide (Compound (85)) is performed in a similar way as in STEP 5a of Example 1.1 but starting from (1r,4r)-4-((3-carbamoyl-4-methylphenoxy)methyl)cyclohexane-1-carboxylic acid.

STEP 4b: Synthesis of trans-5-((4-((S)-3-(3,5-difluorophenyl)isoxazolidine-2-carbonyl)cyclohexyl)methoxy)-2-methylbenzamide (Compound (86)) is performed in a similar way as in STEP 5a of Example 1.1. but starting from (S)-3-(3,5-difluorophenyl)isoxazolidine.

### Example 8: Synthesis of Compounds (115), (116), (117), (118), (119), (120) and (121)

STEP 1: Synthesis of 3-[(6-carbamoylpyrimidin-4-yl)oxymethyl]cyclopentanecarboxylic acid

### Step a: Synthesis of dimethyl trans-cyclopentane-1,3-dicarboxylate

A solution of trans-cyclopentane-1,3-dicarboxylate (9.5 g, 60.07 mmol, 1 eq.) and H₂SO₄ (5.89 g, 60.07 mmol, 3.20 ml, 1 eq.) in MeOH (95 ml) was stirred at 80° C. for 3 hrs. The reaction mixture was concentrated under reduced pressure to remove solvent, NaHCO₃ was added to adjust to pH=7-8 and extracted with EA (100 ml×3). The combined organic layers were washed with brine (100 ml×2), dried over Na₂SO₄, filtered and concentrated under reduced pressure to give the title compound (9.8 g, 52.63 mmol, 88% yield) as a colorless oil.

¹H NMR (400 MHz, CDCl₃): δ ppm 3.61 (s, 6H), 2.81-2.64 (m, 2H), 2.17 (td, J=7.9, 13.0 Hz, 1H), 2.09-1.97 (m, 1H), 1.95-1.79 (m, 4H).

### Step b: Synthesis of trans-3-ethoxycarbonylcyclopentanecarboxylic acid

A mixture of dimethyl trans-cyclopentane-1,3-dicarboxylate (5.8 g, 31.15 mmol, 1 eq.) in EtOH (60 ml) and NaOH (1.25 g, 31.15 mmol, 1 eq.) was stirred at 80° C. for 5 hrs. The reaction mixture was concentrated under reduced pressure to remove solvent, adjusted to pH 5-6 with 1 M HCl and extracted with EA (100 ml×2). The aqueous phase was adjusted to pH 5-6 with 1 M HCl, extracted with EA (50 ml×3) and washed with brine (50 ml×2). The combined organic layers were dried over anhydrous Na₂SO₄, filtered and concentrated under reduced pressure to give the title compound (3.9 g, crude) as a colorless oil.

### Step c: Synthesis of ethyl trans-3-(hydroxymethyl)cyclopentanecarboxylate

A solution of trans-3-ethoxycarbonylcyclopentanecarboxylic acid (3.9 g, 20.94 mmol, 1 eq.) in THF (40 ml) and BH₃·Me₂S (10 M, 3.14 ml, 1.5 eq.) was stirred at 0° C. for 5 hrs. Water (5 ml) and HCl (5 ml) was added, the mixture was stirred for 10 min and extracted with EA (50 ml×2). The combined organic layers were separated, washed with brine (50 ml×2), dried over anhydrous Na₂SO₄, filtered, concentrated under reduced pressure and purified by column chromatography (SiO₂, petroleum ether/EA=5/1 to 4/1) to give the title compound (2.7 g, 15.68 mmol, 75% yield) as a colorless oil.

¹H NMR (400 MHz, CDCl₃): δ ppm 4.14 (q, J=7.1 Hz, 2H), 3.63-3.57 (m, 1H), 3.57-3.49 (m, 1H), 2.88-2.69 (m, 1H), 2.24-2.03 (m, 2H), 1.99-1.87 (m, 2H), 1.86-1.69 (m, 2H), 1.65-1.40 (m, 2H), 1.29-1.24 (m, 3H).

### Step d: Synthesis of ethyl trans-3-[(6-chloropyrimidin-4-yl)oxymethyl]cyclopentanecarboxylate—compound responding to formula (III) in scheme 1

A solution of ethyl trans-3-(hydroxymethyl)cyclopentanecarboxylate (1.6 g, 9.29 mmol, 1 eq.), 4,6-dichloropyrimidine (1.38 g, 9.29 mmol, 1 eq.) in DMF (16 ml) was stirred at 0° C. for 5 min. Then NaH (445.90 mg, 11.15 mmol, 60% purity, 1.2 eq.) was added and the mixture was stirred at 0° C. under N₂ atmosphere for 2 hrs. The reaction mixture was quenched by addition of saturated NH₄Cl (20 ml) at 0° C. and extracted with EA (30 ml×3). The combined organic layers were dried over anhydrous Na₂SO₄, filtered, concentrated under reduced pressure and purified by column chromatography (SiO₂, petroleum ether/EA=20/1) to give the title compound (1.71 g, 6.01 mmol, 65% yield) as a colorless oil.

LC/MS: m/z 285.1 [M+H]⁺

¹H NMR (400 MHz, CDCl₃): δ ppm 8.57 (s, 1H), 6.84-6.69 (m, 1H), 4.40-4.32 (m, 1H), 4.31-4.25 (m, 1H), 4.15 (q, J=7.1 Hz, 2H), 2.93-2.77 (m, 1H), 2.62-2.36 (m, 1H), 2.24-2.08 (m, 1H), 2.02-1.88 (m, 2H), 1.67-1.55 (m, 2H), 1.28 (dt, J=1.4, 7.1 Hz, 3H).

### Step e: Synthesis of ethyl trans-3-[(6-cyanopyrimidin-4-yl)oxymethyl]cyclopentanecarboxylate

A solution of ethyl trans-3-[(6-chloropyrimidin-4-yl)oxymethyl]cyclopentanecarboxylate (1.8 g, 6.32 mmol, 1 eq.), Pd(PPh₃)₄ (1.46 g, 1.26 mmol, 0.2 eq.), Zn(CN)₂ (2.23 g, 18.96 mmol, 1.20 ml, 3 eq.) in DMF (18 ml) was degassed and purged with N₂ for 3 times, and stirred at 100° C. under N₂ atmosphere for 15 hrs. The reaction mixture was filtered, concentrated under reduced pressure and purified by prep-TLC (SiO₂, petroleum ether/EA=5/1) to give the title compound (1.1 g, 4.00 mmol, 63% yield) as a yellow oil.

LC/MS: m/z 276.2 [M+H]⁺

¹H NMR (400 MHz, CDCl₃): δ ppm 8.83 (d, J=0.7 Hz, 1H), 7.15-7.05 (m, 1H), 4.45-4.28 (m, 2H), 4.15 (q, J=7.1 Hz, 2H), 2.96-2.79 (m, 1H), 2.66-2.37 (m, 1H), 2.25-2.11 (m, 1H), 2.06-1.85 (m, 3H), 1.76-1.52 (m, 2H), 1.28 (dt, J=1.3, 7.1 Hz, 3H).

### Step f: Synthesis of 3-[(6-carbamoylpyrimidin-4-yl)oxymethyl]cyclopentanecarboxylic acid

To a mixture of ethyl trans-3-[(6-cyanopyrimidin-4-yl)oxymethyl]cyclopentanecarboxylate (1.3 g, 4.25 mmol, 90% purity, 1 eq.) in THF (13 ml) a mixture of H₂O₂ (963.59 mg, 8.50 mmol, 816.60 μl, 30% purity, 2 eq.) and LiOH·H₂O (1 M, 4.25 ml, 1 eq.) was added, degassed and purged with N₂ for 3 times and stirred at 25° C. for 30 min. LiOH·H₂O (1 M, 1.49 ml, 0.35 eq.) was added and the mixture was stirred at 25° C. under N₂ atmosphere for 12 hrs. The reaction mixture was quenched by addition of Na₂SO₃ solution, adjusted to pH 5-6 with 1 M HCl and filtered. The precipitate was triturated with petroleum ether/EA=1/1 at 25° C. for 30 min to give the title compound (0.57 g, 2.15 mmol, 51% yield) as a white solid. Analytical data showed that cis-trans-isomerization took place.

LC/MS: m/z 266.2 [M+H]⁺

SFC (Column: Chiralpak IG-3 50×4.6 mm I.D., 3 μm; gradient elution: IPA (0.05% DEA) in CO₂ from 5% to 40%, flow rate: 3 ml/min; column temperature: 35° C., 100 bar): Rt=1.97 min (peak 1, 28.4%), 2.02 min (peak 2, 22.0%), 2.12 min (peak 3, 18.0%) and 2.20 min (peak 4, 31.6%).

¹H NMR (400 MHz, DMSO-d₆): δ ppm 12.09 (br s, 1H), 8.86 (d, J=0.7 Hz, 1H), 8.23 (br s, 1H), 7.93 (br s, 1H), 7.37-7.25 (m, 1H), 4.35-4.27 (m, 1H), 4.39-4.18 (m, 1H), 2.86-2.66 (m, 1H), 2.43-2.30 (m, 1H), 2.12-1.88 (m, 2H), 1.87-1.73 (m, 2H), 1.59-1.31 (m, 2H).

STEP 2a: Synthesis of a diastereomeric mixture of 6-[[3-[(3S)-3-(3-cyano-5-fluoro-phenyl)isoxazolidine-2-carbonyl]cyclopentyl]methoxy]-pyrimidine-4-carboxamide is performed in a similar way as in STEP 5a of Example 1.1, but starting from (3S)-3-(3-cyano-5-fluoro-phenyl)isoxazolidine

Diastereomeric mixture of 6-[[3-[(3S)-3-(3-cyano-5-fluoro-phenyl)isoxazolidine-2-carbonyl]cyclopentyl]methoxy]-pyrimidine-4-carboxamide was separated by SFC (condition: column: DAICEL CHIRALPAK AD, 250×30 mm, 10 μm; mobile phase: 55% EtOH (0.1% NH₃·H₂O) in CO₂, single batch cycle process with 3.5 min lag time between two sequential injections; total duration 70 min; and column: DAICEL CHIRALPAK AS, 250×30 mm, 10 μm; mobile phase: 65% MeOH (0.1% NH₃·H₂O) in CO₂, single batch cycle process with 4.4 min lag time between two sequential injections; total duration 25 min) to give compound (115) stereoisomer 1 (peak 1, 96 mg, 204.98 μmol, 18% yield), compound (116) stereoisomer 2 (peak 2, 57 mg, 126.36 μmol, 11% yield), compound (117) stereoisomer 3 (peak 3, 41 mg, 89.95 μmol, 8% yield) and cis-6-[[3-[(3S)-3-(3-cyano-5-fluoro-phenyl)isoxazolidine-2-carbonyl]cyclopentyl]methoxy]pyrimidine-4-carboxamide stereoisomer 4 (peak 4, 80 mg, 180.85 μmol, 16% yield) all as yellow solids.

Cis-6-[[3-[(3S)-3-(3-cyano-5-fluoro-phenyl)isoxazolidine-2-carbonyl]cyclopentyl]methoxy]pyrimidine-4-carboxamide Stereisomer 4 (peak 4):

LC/MS: m/z 440.3 [M+H]⁺;

SFC (Column: Chiralpak AS-3 50×4.6 mm I.D., 3 μm; mobile phase: CO₂ and MeOH (0.05% DEA), gradient: 5%-40% MeOH (0.05% DEA); flow rate: 3 ml/min; column temp: 35° C.; 100 bar): Rt=2.40 min (100%);

¹H NMR (400 MHz, CDCl₃): δ ppm 8.74 (d, J=1.0 Hz, 1H), 7.76 (br s, 1H), 7.51 (d, J=1.0 Hz, 1H), 7.42 (s, 1H), 7.29-7.26 (m, 2H), 5.72 (br s, 1H), 5.41 (dd, J=6.4, 8.7 Hz, 1H), 4.41-4.27 (m, 3H), 3.87 (ddd, J=6.7, 8.2, 9.6 Hz, 1H), 3.31 (quin, J=8.3 Hz, 1H), 2.90 (dddd, J=2.9, 6.5, 9.2, 12.3 Hz, 1H), 2.51 (td, J=7.8, 16.0 Hz, 1H), 2.36-2.24 (m, 1H), 2.14-1.90 (m, 4H), 1.65-1.57 (m, 2H).

STEP 2b: Synthesis of a diastereomeric mixture of 6-[[3-[(3S)-3-(3,5-difluorophenyl)isoxazolidine-2-carbonyl]cyclopentyl]methoxy]pyrimidine-4-carboxamide is performed in a similar way as in STEP 5a of Example 1.1 but starting from (3S)-3-(3,5-difluorophenyl)isoxazolidine

Diastereomeric mixture of 6-[[3-[(3S)-3-(3,5-difluorophenyl)isoxazolidine-2-carbonyl]cyclopentyl]methoxy]pyrimidine-4-carboxamide was separated by SFC (condition: column: DAICEL CHIRALPAK AS, 250×30 mm, 10 μm; mobile phase: 70% EtOH (0.1% NH₃·H₂O) in CO₂; single batch cycle process with 10.0 min lag time between two sequential injections; total duration 50 min; column: DAICEL CHIRALPAK AS, 250×30 mm, 10 μm; mobile phase: 40% EtOH (0.1% NH₃·H₂O) in CO₂, single batch cycle process with 2.1 min lag time between two sequential injections; total duration 40 min and column: DAICEL CHIRALPAK AS, 250×30 mm, 10 μm; mobile phase: 65% MeOH (0.1% NH₃·H₂O) in CO₂, single batch cycle process with 5.2 min lag time between two sequential injections; total duration 40 min) to give compound (118) stereoisomer 1 (peak 1, 44 mg, 99.15 μmol, 6% yield), compound (119) stereoisomer 2 (peak 2, 164 mg, 377.58 μmol, 22% yield), compound (120) stereoisomer 3 (peak 3, 37 mg, 84.75 μmol, 5% yield) as a yellow solid and compound (121) stereoisomer 4 (peak 4, 154 mg, 356.14 μmol, 20% yield) as a white solid.

### Example 9: Synthesis of Compounds (71), (72), (73), (74), (75), (83), (88), (89), (90), (92), (94), (95), (99), (100), (101), (102), (103), (104), (111), (112), (113), (129), (130) and (131)

### Synthesis of trans-4-[[tert-butyl(dimethyl)silyl]oxymethyl]cyclohexanecarboxylic acid

To a mixture of trans-4-(hydroxymethyl)cyclohexanecarboxylic acid (1.00 g, 6.32 mmol) and DMF (40 ml) was added TBDMS-Cl (2.10 mg, 13.9 mmol) and TEA (2.4 ml, 17.2 mmol). The mixture was stirred for 3 hrs at rt. The mixture was diluted with water. HCl (aqueous, 1 M) was added until a slightly acidic pH was reached. The mixture was extracted with MTBE (2×). The combined organic layers were washed with brine, dried (Na₂SO₄), filtered and concentrated. The residue (2.9 g) was dissolved in DMF/water (20:1, 35 ml) and heated to 70° C. for 2 hrs. At rt, the mixture was diluted with MTBE. The organic layer was separated, washed with NH₄Cl (saturated aqueous solution) and brine, dried (Na₂SO₄), filtered and concentrated. The crude was purified by column chromatography (SiO₂; EA/heptane gradient 1/4 to 1/2) to afford the title compound (1.22 g, 4.48 mmol, 71% yield).

¹H NMR (400 MHz, DMSO-d₆): δ ppm 11.95 (br s, 1H), 3.32 (m, 2H), 2.07 (tt, J=12.12, 12.12, 3.53, 3.53 Hz, 1H), 1.87 (m, 2H), 1.72 (m, 2H), 1.29 (m, 3H), 0.91 (m, 2H), 0.85 (s, 9H), 0.00 (s, 6H).

STEP 1a: Synthesis of 5-[(3S)-2-[trans-4-[[tert-butyl(dimethyl)silyl]oxymethyl]cyclohexanecarbonyl]isoxazolidin-3-yl]pyridine-3-carbonitrile

To a mixture of trans-4-[[tert-butyl(dimethyl)silyl]oxymethyl]cyclohexanecarboxylic acid (500 mg, 1.84 mmol) and DMF (20 ml) was added at 0° C. Oxyma (770 mg, 5.2 mmol) and NaHCO₃ (1.43 g, 17.02 mmol). The mixture was stirred for 1 hr at rt. 5-[(3S)-isoxazolidin-3-yl]pyridine-3-carbonitrile (320 mg, 1.83 mmol) in DMF (10 ml) was added. The mixture was warmed to 40° C. for 2 hrs. At rt, the mixture was diluted with EA and washed with water. The organic layer was separated, washed with NH₄Cl (saturated aqueous solution), NaHCO₃ (saturated aqueous solution) and brine, dried (Na₂SO₄), filtered and concentrated. The crude title compound (770 mg, 1.79 mmol, 98% crude yield) was used directly in the next step.

STEP 1b: [trans-4-[[tert-butyl(dimethyl)silyl]oxymethyl]cyclohexyl]-[(3S)-3-pyrazin-2-ylisoxazolidin-2-yl]methanone, [trans-4-[[tert-butyl(dimethyl)silyl]oxymethyl]cyclohexyl]-[(3S)-3-(6-methoxypyrazin-2-yl)isoxazolidin-2-yl]methanone, [trans-4-[[tert-butyl(dimethyl)silyl]oxymethyl]cyclohexyl]-[(3S)-3-(2-methylthiazol-4-yl)isoxazolidin-2-yl]methanone and [trans-4-[[tert-butyl(dimethyl)silyl]oxymethyl]cyclohexyl]-[(3S)-3-(2-pyridyl)isoxazolidin-2-yl]methanone is performed as detailed in STEP 1a of Example 9.

STEP 2a: Synthesis of 5-((S)-2-(trans-4-(hydroxymethyl)cyclohexane-1-carbonyl)isoxazolidin-3-yl)nicotinonitrile—compound responding to formula (IIc) in scheme 4

To a mixture of 5-[(3S)-2-[trans-4-[[tert-butyl(dimethyl)silyl]oxymethyl]cyclohexane-carbonyl]isoxazolidin-3-yl]pyridine-3-carbonitrile (770 mg, 1.79 mmol) and DCM (30 ml) was added TFA (2 ml). The mixture was stirred for 15 min. The reaction was quenched by addition of NaHCO₃ (saturated aqueous solution) and diluted with EA. The organic layer was separated, washed with brine, dried (Na₂SO₄), filtered and concentrated. The crude was purified by preparative HPLC to afford the title compound (340 mg, 1.08 mmol, 60% yield).

¹H NMR (400 MHz, DMSO-d₆): δ ppm 8.94 (d, J=1.83 Hz, 1H), 8.77 (d, J=2.08 Hz, 1H), 8.19 (t, J=1.96, 1.96 Hz, 1H), 5.41 (dd, J=8.62, 6.54 Hz, 1H), 4.36 (br s, 1H), 4.29 (td, J=7.70, 7.70, 3.06 Hz, 1H), 3.91 (m, 1H), 3.21 (br d, J=5.50 Hz, 2H), 2.89 (m, 1H), 2.66 (br t, J=11.92, 11.92 Hz, 1H), 2.29 (m, 1H), 1.90 (br d, J=11.74 Hz, 1H), 1.76 (m, 3H), 1.31 (m, 3H), 0.94 (m, 2H).

STEP 2b: [trans-4-(hydroxymethyl)cyclohexyl]-[(3S)-3-pyrazin-2-ylisoxazolidin-2-yl]methanone, [trans-4-(hydroxymethyl)cyclohexyl]-[(3S)-3-(6-methoxypyrazin-2-yl)isoxazolidin-2-yl]methanone, [trans-4-(hydroxymethyl)cyclohexyl]-[(3S)-3-(2-pyridyl)isoxazolidin-2-yl]methanone and [trans-4-(hydroxymethyl)cyclohexyl]-[(3S)-3-(2-methylthiazol-4-yl)isoxazolidin-2-yl]methanone are synthesized in a similar way as in STEP 2a of Example 9.

STEP 3a: Synthesis of trans-5-((S)-2-(4-((3-cyanophenoxy)methyl)cyclohexane-1-carbonyl)isoxazolidin-3-yl)nicotinonitrile (Compound (71)—according to route SM3 in scheme 4

To a mixture of 5-((S)-2-(trans-4-(hydroxymethyl)cyclohexane-1-carbonyl) isoxazolidin-3-yl)nicotinonitrile (50 mg, 159 μmol), 3-hydroxybenzonitrile (40 mg, 336 μmol) and THF (2 ml) were added PPh₃ (polymer-bound, 100 mg) and DIAD (70 μl, 0.36 mmol). The mixture was stirred for 2 hrs at rt, diluted with THF (15 ml) and filtered. The filtrate was concentrated and purified by preparative HPLC to afford the title compound (20 mg, 48.0 μmol, 30% yield).

STEP 3b: Synthesis of trans-5-((S)-2-(4-((3-cyano-4-fluorophenoxy)methyl)cyclohexane-1-carbonyl)isoxazolidin-3-yl)nicotinonitrile (Compound (72)), trans-3-((4-((S)-3-(5-cyanopyridin-3-yl)isoxazolidine-2-carbonyl)cyclohexyl)methoxy)benzamide (Compound (73)), trans-5-((4-((S)-3-(5-cyanopyridin-3-yl)isoxazolidine-2-carbonyl)cyclohexyl)methoxy)-2-fluorobenzamide (Compound (74)) and trans-2-chloro-5-((4-((S)-3-(5-cyanopyridin-3-yl)isoxazolidine-2-carbonyl)cyclohexyl)methoxy)benzamide (Compound (75)) are performed in a similar way as detailed in STEP 3a of Example 9 but starting respectively from 3-hydroxy-5 fluorobenzonitrile, 3-hydroxybenzamide, 5-hydroxy-2-fluorobenzamide and 2-chloro-5-hydroxybenzamide.

STEP 4a: Synthesis of [trans-4-[(3S)-3-(5-cyano-3-pyridyl)isoxazolidine-2-carbonyl]cyclohexyl]methyl 4-nitrobenzenesulfonate—responding to compound of formula (lid) in scheme 4—activation step before route SM5 of scheme 4

A mixture of trans-5-[(3S)-2-[4-(hydroxymethyl)cyclohexanecarbonyl]isoxazolidin-3-yl]pyridine-3-carbonitrile (50 mg, 159 μmol) and DCM (2 ml) was added TEA (40 μl, 287 μmol) and 4-nitrobenzenesulfonyl chloride (50 mg, 221 μmol). The mixture was stirred for 14 hrs, diluted with EA and washed with water. The organic layer was concentrated, redissolved in EA and filtered over a short pad of silica. The filtrate was concentrated to afford the title compound (53 mg, 106 μmol, 67% yield).

¹H NMR (400 MHz, DMSO-d₆): δ ppm 8.93 (d, J=1.96 Hz, 1H), 8.76 (d, J=2.20 Hz, 1H), 8.46 (d, J=8.06 Hz, 2H), 8.19 (m, 3H), 5.39 (m, 1H), 4.27 (td, J=7.64, 7.64, 2.93 Hz, 1H), 3.99 (d, J=5.99 Hz, 2H), 3.90 (m, 1H), 2.89 (dddd, J=12.13, 9.14, 6.42, 3.12 Hz, 1H), 2.64 (m, 1H), 2.28 (m, 1H), 1.88 (br d, J=11.98 Hz, 1H), 1.68 (br d, J=12.10 Hz, 3H), 1.63 (br d, J=3.30 Hz, 1H), 1.29 (m, 2H), 1.00 (m, 2H).

STEP 4b: [trans-4-[(3S)-3-pyrazin-2-ylisoxazolidine-2-carbonyl]cyclohexyl]methyl-4-nitrobenzenesulfonate, [trans-4-[(3S)-3-(6-methoxypyrazin-2-yl)isoxazolidine-2-carbonyl]cyclohexyl]methyl-4-nitrobenzenesulfonate, [trans-4-[(3S)-3-(2-pyridyl)isoxazolidine-2-carbonyl]cyclohexyl]methyl-4-nitrobenzenesulfonate and [trans-4-[(3S)-3-(2-methylthiazol-4-yl)isoxazolidine-2-carbonyl]cyclohexyl]methyl-4-nitrobenzenesulfonate were synthesized in a similar way as in STEP 4a of Example 9.

STEP 5a: Synthesis of (Compound (83))—route SM5 of scheme 4

A mixture of [trans-4-[(3S)-3-(5-cyano-3-pyridyl)isoxazolidine-2-carbonyl]cyclohexyl]methyl 4-nitrobenzenesulfonate (23 mg, 46.0 μmol), 3-fluoro-5-hydroxy-benzo-nitrile (10 mg, 73 μmol) and DMF (2 ml) was added Cs₂CO₃ (40 mg, 123 μmol). The mixture was stirred for 2.5 hrs at 50° C. At rt, the mixture was filtered and the filtrate purified by preparative HPLC to afford the title compound (12 mg, 27.6 μmol, 60% yield).

STEP 5b: Synthesis of trans-5-((S)-2-(4-((2-chloro-5-(3-methyl-2,5-dioxoimidazolidin-1-yl)phenoxy)methyl)cyclohexane-1-carbonyl)isoxazolidin-3-yl)nicotinonitrile (88) is performed in a similar way as in STEP 5a of Example 9 but starting respectively from 2-chloro-5-(3-methyl-2,5-dioxoimidazolidin-1-yl)phenol.

STEP 5c: Synthesis of trans-5-((S)-2-(4-((2-fluoro-4-(3-methyl-2-oxo-2,3-dihydro-1H-imidazol-1-yl)phenoxy)methyl)cyclohexane-1-carbonyl)isoxazolidin-3-yl)nicotinonitrile (89) is performed in a similar way as in STEP 5a of Example 9 but starting respectively from 2-fluoro-4-(3-methyl-2-oxo-2,3-dihydro-1H-imidazol-1-yl)phenol.

STEP 5d: Synthesis of trans-5-((S)-2-(4-((4-(3,5-dimethyl-1H-pyrazol-1-yl)-2-fluorophenoxy)methyl)cyclohexane-1-carbonyl)isoxazolidin-3-yl)nicotinonitrile (90) is performed in a similar way as in STEP 5a of Example 9 but starting respectively from 4-(3,5-dimethyl-1H-pyrazol-1-yl)-2-fluorophenol.

STEP 5e: Synthesis of trans-5-((S)-2-(4-((5-(5,5-dimethyl-2,4-dioxooxazolidin-3-yl)-2-fluorophenoxy)methyl)cyclohexane-1-carbonyl)isoxazolidin-3-yl)nicotinonitrile (92) is performed in a similar way as in STEP 5^{a} of Example 9 but starting respectively from 5-(5,5-dimethyl-2,4-dioxooxazolidin-3-yl)-2-fluorophenol.

STEP 5f: Synthesis of trans-5-((S)-2-(4-(((4-cyanopyridin-2-yl)oxy)methyl)cyclohexane-1-carbonyl)isoxazolidin-3-yl)nicotinonitrile (94) is performed in a similar way as in STEP 5a of Example 9 but starting respectively from 2-hydroxypyridine-4-carbonitrile.

STEP 5g: Synthesis of trans-5-((S)-2-(4-(((5-cyanopyridin-2-yl)oxy)methyl)cyclohexane-1-carbonyl)isoxazolidin-3-yl)nicotinonitrile (95) is performed in a similar way as in STEP 5a of Example 9 but starting respectively from 3-cyano-6-hydroxypyridine.

STEP 5h: Synthesis of trans-6-[[4-[(3S)-3-(5-cyano-3-pyridyl)isoxazolidine-2-carbonyl]cyclohexyl]methoxy]pyridine-3-carboxamide (99) is performed in a similar way as in STEP 5a of Example 9 but starting respectively from 6-hydroxypyridine-3-carboxamide.

STEP 5i: Synthesis of trans-3-[[4-[(3S)-3-pyrazin-2-ylisoxazolidine-2-carbonyl]cyclohexyl]methoxy]benzamide (100) is performed in a similar way as in STEP 5a of Example 9 but starting respectively from 3-hydroxybenzamide.

STEP 5j: Synthesis of trans-2-fluoro-5-[[4-[(3S)-3-pyrazin-2-ylisoxazolidine-2-carbonyl]cyclohexyl]methoxy]benzamide (101) is performed in a similar way as in STEP 5a of Example 9 but starting respectively from 2-fluoro-5-hydroxybenzamide.

STEP 5k: Synthesis of trans-6-[[4-[(3S)-3-pyrazin-2-ylisoxazolidine-2-carbonyl]cyclohexyl]methoxy]pyridine-3-carboxamide (102), is performed in a similar way as in STEP 5a of Example 9 but starting respectively from from 6-hydroxypyridine-3-carboxamide.

STEP 5l: Synthesis of trans-6-[[4-[(3S)-3-pyrazin-2-ylisoxazolidine-2-carbonyl]cyclohexyl]methoxy]pyridine-3-carbonitrile (103) is performed in a similar way as in STEP 5^{a} of Example 9 but starting respectively from 6-hydroxynicotinonitrile.

STEP 5m: Synthesis of trans-3-fluoro-5-[[4-[(3S)-3-pyrazin-2-ylisoxazolidine-2-carbonyl]cyclohexyl]methoxy]benzonitrile (104) is performed in a similar way as in STEP 5a of Example 9 but starting respectively from 3-fluoro-5-hydroxybenzonitrile.

STEP 5n: Synthesis of trans-4-fluoro-3-[[4-[(3S)-3-pyrazin-2-ylisoxazolidine-2-carbonyl]cyclohexyl]methoxy]benzamide (111) is performed in a similar way as in STEP 5a of Example 9 but starting respectively from 4-fluoro-3-hydroxybenzamide.

STEP 5o: Synthesis of trans-3-fluoro-5-[[4-[(3S)-3-pyrazin-2-ylisoxazolidine-2-carbonyl]cyclohexyl]methoxy]benzamide (112) is performed in a similar way as in STEP 5a of Example 9 but starting respectively from 3-fluoro-5-hydroxybenzamide.

STEP 5p: Synthesis of trans-3-fluoro-4-[[4-[(3S)-3-pyrazin-2-ylisoxazolidine-2-carbonyl]cyclohexyl]methoxy]benzamide (113) is performed in a similar way as in STEP 5a of Example 9 but starting respectively from 3-fluoro-4-hydroxybenzamide.

STEP 5q: Synthesis of trans-3-[[4-[(3S)-3-(6-methoxypyrazin-2-yl)isoxazolidine-2-carbonyl]cyclohexyl]methoxy]benzamide (129) is performed in a similar way as in STEP 5a of Example 9 but starting respectively from 3-hydroxybenzamide.

STEP 5r: Synthesis of trans-3-[[4-[(3S)-3-(2-methylthiazol-4-yl)isoxazolidine-2-carbonyl]cyclohexyl]methoxy]benzamide (130) is performed in a similar way as in STEP 5a of Example 9 but starting respectively from 3-hydroxybenzamide.

STEP 5s: Synthesis of trans-3-[[4-[(3S)-3-(2-pyridyl)isoxazolidine-2-carbonyl]cyclohexyl]methoxy]benzamide (131) is performed in a similar way as in STEP 5a of Example 9 but starting respectively from 3-hydroxybenzamide.

### Example 10: Synthesis of Compounds (93)

### Example 10.1: Synthesis of Compound (93)

STEP 1: Synthesis of methyl trans-4-[(2-cyano-4-pyridyl)oxymethyl]cyclohexanecarboxylate

To a solution of 4-nitropyridine-2-carbonitrile (300 mg, 2.01 mmol, 1 eq) in DMF (3 mL) was added NaH (120.71 mg, 3.02 mmol, 60% purity, 1.5 eq) and methyl (1r,4r)-4-(hydroxymethyl)cyclohexane-1-carboxylate (381.16 mg, 2.21 mmol, 1.1 eq). The mixture was stirred at 25° C. for 1 hr. TLC indicated no starting material remained, and a new spot was detected. The reaction mixture was concentrated under reduced pressure to remove DMF. The residue was diluted with H₂O 20 mL and extracted with EA 20 mL (20 mL*2), dried over Na₂SO₄, filtered and concentrated under reduced pressure to give a residue. The residue was purified by column chromatography (SiO2, Petroleum ether/Ethyl acetate=1/1 to 5/1) to give methyl 4-[(2-cyano-4-pyridyl)oxymethyl]cyclohexanecarboxylate (532 mg, 1.51 mmol, 75.19% yield, 78% purity) as a white solid.

LC/MS m/z 275.2 [M+H]+; RT 0.905 min (Method B).

¹H NMR (CDCl₃; 400 MHz) δ 8.50 (d, J=5.9 Hz, 1H), 7.21 (d, J=2.5 Hz, 1H), 6.98 (dd, J=2.5, 5.9 Hz, 1H), 3.86 (d, J=6.3 Hz, 2H), 3.69 (s, 3H), 2.36-2.26 (m, 1H), 2.17-2.04 (m, 3H), 2.03-1.93 (m, 3H), 1.91-1.79 (m, 2H), 1.57-1.42 (m, 4H), 1.20-1.08 (m, 4H).

STEP 2: Synthesis of 4-[(2-cyano-4-pyridyl)oxymethyl]cyclohexanecarboxylic acid

To a solution of methyl 4-[(2-cyano-4-pyridyl)oxymethyl]cyclohexanecarboxylate (732 mg, 2.67 mmol, 1 eq) in THF (7 mL) was added LiOH·H₂O (1 M, 3.20 mL, 1.2 eq). The mixture was stirred at 25° C. for 1 hr. LC-MS showed no starting material remained. Several new peaks were shown on LC-MS and desired compound was detected. The reaction mixture was concentrated. The residue was purified by prep-HPLC (column: Phenomenex Synergi C18 150*25 mm*10 um; mobile phase: [water (0.225% FA)-ACN]; B %: 23%-56%, 10 min) to give 4-[(2-cyano-4-pyridyl)oxymethyl]cyclohexanecarboxylic acid (218 mg, 837.53 umol, 31.39% yield, 100% purity) as a white solid.

LC/MS m/z 261.2 [M+H]+; RT 0.814 min (Method B).

¹H NMR (CDCl₃; 400 MHz) δ 8.51 (d, J=5.7 Hz, 1H), 7.21 (d, J=2.3 Hz, 1H), 6.99 (dd, J=2.5, 5.8 Hz, 1H), 3.87 (d, J=6.2 Hz, 2H), 2.36 (tt, J=3.5, 12.2 Hz, 1H), 2.20-2.09 (m, 2H), 2.05-1.95 (m, 2H), 1.91-1.81 (m, 1H), 1.54-1.47 (m, 2H), 1.23-1.10 (m, 2H).

STEP 3: Synthesis of (trans-4-[[4-[(3S)-3-(3-cyano-5-fluoro-phenyl)isoxazolidine-2-carbonyl]cyclohexyl]methoxy]pyridine-2-carbonitrile (Compound (93)) is performed in a similar way as in STEP 5a of Example 1.1, but starting from 4-[(2-cyano-4-pyridyl)oxymethyl]cyclohexanecarboxylic acid

### Example 11: Synthesis of Compounds (12), (13), (14), (15), (16), (17), (18), (19), (20), (21), (22), (23), (24), (28), (29) and (30)

### Example 11.1: Synthesis of Compounds (12) and (13)

STEP 1: Synthesis of cis-(4-aminocyclohexyl)-[(3S)-3-(4-chlorophenyl)isoxazolidin-2-yl]methanone hydrochloric acid salt—responding to compound of formula (IIe) in scheme 5

To a stirred solution of tert-butyl N-[cis-4-[(3S)-3-(4-chlorophenyl)isoxazolidine-2-carbonyl]cyclohexyl]carbamate (260 mg, 635.8 μmol) in CH₂Cl₂ (2.7 ml) TFA (1.35 ml, 17.5 mmol) was added and stirring was continued for 1 hr at rt. The solution was diluted with toluene and concentrated under reduced pressure. The resulting crude material was redissolved in toluene and concentrated again. Finally the obtained material was dissolved in aqueous 0.1 N HCl and lyophilized. The title compound was obtained as a pale yellow oil, which was used in the next reaction without further purification (230 mg, 660 μmol, quant. Yield).

¹H NMR (600 MHz, DMSO-d₆): δ ppm 7.87 (br s, 3H), 7.41 (d, J=8.44 Hz, 2H), 7.30 (d, J=8.44 Hz, 2H), 5.31 (m, 1H), 4.24 (m, 1H), 3.88 (m, 1H), 3.85 (s, 1H), 3.12 (m, 1H), 2.86 (m, 2H), 2.14 (m, 1H), 1.95-1.70 (m, 5H), 1.51 (m, 2H).

STEP 2: Synthesis of compounds cis-2-((4-((S)-3-(4-chlorophenyl)isoxazolidine-2-carbonyl)cyclohexyl)amino)pyrimidine-4-carboxamide (12) is performed in a similar way as for STEP 2 of Example 11.2 but starting respectively from 2-chloropyrimidine-4-carboxamide.

STEP 2a: Synthesis of cis-6-((4-((S)-3-(4-chlorophenyl)isoxazolidine-2-carbonyl)cyclohexyl)amino)pyrimidine-4-carboxamide (13) is performed in a similar way as for STEP 2 of Example 11.2 but starting respectively from 6-chloropyrimidine-4-carboxamide.

### Example 11.2: Synthesis of Compound (28)

STEP 1: Synthesis of cis-3-[(3S)-2-(4-aminocyclohexanecarbonyl)isoxazolidin-3-yl]-5-fluoro-benzonitrile hydrochloride is performed as in STEP 1 of Example 11.1—responding to compound of formula (lie) in scheme 5, but starting from tert-butyl N-[cis-4-[(3S)-3-(3-cyano-5-fluorophenyl)isoxazolidine-2-carbonyl]cyclohexyl]carbamate

STEP 2: Synthesis of cis-6-((4-((S)-3-(3-cyano-5-fluorophenyl)isoxazolidine-2-carbonyl)cyclohexyl)amino)pyrimidine-4-carbonitrile Compound (28)—route of scheme 5

To a stirred solution of 6-chloropyrimidine-4-carbonitrile (28 mg, 198 μmol) and cis-3-[(3S)-2-(4-aminocyclohexanecarbonyl)isoxazolidin-3-yl]-5-fluoro-benzonitrile hydrochloride (70 mg, 198 μmol) in CH₃CN (2 ml) Net/Pr₂ was added (75 μl, 428 μmol) at rt. The solution was heated to reflux for 2.5 hrs. Volatile components were removed under reduced pressure, the obtained residue dissolved in DMF, filtered and subjected to preparative reversed phase HPLC (33 mg, 79 μmol, 40% yield).

### Example 11.3: Synthesis of Compounds (14) and (15)

STEP 1: Synthesis of trans-[4-(aminomethyl)cyclohexyl]-[(3S)-3-(4-chlorophenyl)isoxazolidin-2-yl]methanone hydrochloride is performed as in STEP 1 of Example 11.1—responding to compound of formula (IIe) in scheme 5, but starting from tert-butyl N-[trans-4-[(3S)-3-(4-chlorophenyl)isoxazolidine-2-carbonyl]cyclohexyl]carbamate

STEP 2: Synthesis of trans-2-(((4-((S)-3-(4-chlorophenyl)isoxazolidine-2-carbonyl)cyclohexyl)methyl)amino)pyrimidine-4-carbonitrile (Compound (14)) is performed in a similar way as for STEP 2 of Example 11.2 but starting respectively from 2-chloropyrimidine-4-carbonitrile.

STEP 2a: Synthesis of trans-6-(((4-((S)-3-(4-chlorophenyl)isoxazolidine-2-carbonyl)cyclohexyl)methyl)amino)pyrimidine-4-carbonitrile (Compound (15)) is performed in a similar way as for STEP 2 of Example 11.2 but starting respectively from 6-chloropyrimidine-4-carbonitrile.

### Example 11.4: Synthesis of Compounds (16) and (17)

STEP 1: Synthesis of cis-(3-aminocyclobutyl)-[(3S)-3-(3-fluorophenyl)isoxazolidin-2-yl]methanone; hydrochloride is performed as in STEP 1 of Example 11.1 but starting from tert-butyl N-[cis-3-[(3S)-3-(3-flourophenyl)isoxazolidine-2-carbonyl]cyclobutyl]carbamate

STEP 2: Synthesis of cis-2-((3-((S)-3-(3-fluorophenyl)isoxazolidine-2-carbonyl)cyclobutyl)amino)pyrimidine-4-carbonitrile (Compound (16)) is performed in a similar way as for STEP 2 of Example 11.2 but starting respectively from 2-chloropyrimidine-4-carbonitrile.

STEP 2a: Synthesis of cis-6-((3-((S)-3-(3-fluorophenyl)isoxazolidine-2-carbonyl)cyclobutyl)amino)pyrimidine-4-carbonitrile (Compound (17)) is performed in a similar way as for STEP 2 of Example 11.2 but starting respectively from 6-chloropyrimidine-4-carbonitrile.

### Example 11.5: Synthesis of Compound (18)

STEP 1: Synthesis of cis-(3-aminocyclobutyl)-[(3S)-3-(3,5-difluorophenyl)isoxazolidin-2-yl]methanone hydrochloride is performed as in STEP 1 of Example 11.1, but starting from tert-butyl N-[cis-3-[(3S)-3-(3,5-difluorophenyl)isoxazolidine-2-carbonyl]cyclobutyl]carbamate

STEP 2: Synthesis of cis-6-((3-((S)-3-(3,5-difluorophenyl)isoxazolidine-2-carbonyl)cyclobutyl)amino)pyrimidine-4-carbonitrile (Compound (18)) is performed in a similar way as for STEP 2 of Example 11.2 but starting respectively from 6-chloropyrimidine-4-carbonitrile.

### Example 11.6: Synthesis of Compounds (19) and (20)

STEP 1: Synthesis of cis-3-[(3S)-2-(3-aminocyclobutanecarbonyl)isoxazolidin-3-yl]-5-fluoro-benzonitrile; hydrochloride is performed as in STEP 1 of Example 11.1, but starting from N-[cis-3-[(3S)-3-(3-cyano-5-fluorophenyl)isoxazolidine-2-carbonyl]cyclobutyl]carbamate

STEP 2: Synthesis of cis-2-((3-((S)-3-(3-cyano-5-fluorophenyl)isoxazolidine-2-carbonyl)cyclobutyl)amino)pyrimidine-4-carbonitrile (Compound (19)) is performed in a similar way as for STEP 2 of Example 11.2 but starting respectively from 2-chloropyrimidine-4-carbonitrile.

STEP 2a: Synthesis of and cis-6-((3-((S)-3-(3-cyano-5-fluorophenyl)isoxazolidine-2-carbonyl)cyclobutyl)amino)pyrimidine-4-carbonitrile (20)) is performed in a similar way as for STEP 2 of Example 11.2 but starting respectively from 6-chloropyrimidine-4-carbonitrile.

### Example 11.7: Synthesis of Compounds (21) and (22)

STEP 1: Synthesis of cis-(3-aminocyclobutyl)-[(3S)-3-(3,4-difluorophenyl)isoxazolidin-2-yl]methanone; hydrochloride is performed as in STEP 1 of Example 11.1, but starting from tert-butyl N-[cis-3-[(3S)-3-(3,4-difluorophenyl)isoxazolidine-2-carbonyl]cyclobutyl]carbamate

STEP 2: Synthesis of cis-2-((3-((S)-3-(3,4-difluorophenyl)isoxazolidine-2-carbonyl)cyclobutyl)amino)pyrimidine-4-carbonitrile (Compound (21)) is performed in a similar way as for STEP 2 of Example 11.2 but starting respectively from 2-chloropyrimidine-4-carbonitrile.

STEP 2a: Synthesis of cis-6-((3-((S)-3-(3,4-difluorophenyl)isoxazolidine-2-carbonyl)cyclobutyl)amino)pyrimidine-4-carbonitrile (Compound (22)) is performed in a similar way as for STEP 2 of Example 11.2 but starting respectively from 6-chloropyrimidine-4-carbonitrile.

### Example 11.8: Synthesis of Compounds (23) and (24)

STEP 1: Synthesis of cis-(3-aminocyclobutyl)-[(3S)-3-(4-chlorophenyl)isoxazolidin-2-yl]methanone; hydrochloride is performed as in STEP 1 of Example 11.1, but starting from tert-butyl N-[cis-3-[(3S)-3-(4-fluorophenyl)isoxazolidine-2-carbonyl]cyclobutyl]carbamate

STEP 2: Synthesis of cis-6-((3-((S)-3-(4-chlorophenyl)isoxazolidine-2-carbonyl)cyclobutyl)amino)pyrimidine-4-carbonitrile (Compound (23) is performed in a similar way as for STEP 2 of Example 11.2 but starting respectively from 6-chloropyrimidine-4-carbonitrile.

STEP 2a: Synthesis of cis-2-((3-((S)-3-(4-chlorophenyl)isoxazolidine-2-carbonyl)cyclobutyl)amino)pyrimidine-4-carbonitrile (Compound (24)) is performed in a similar way as for STEP 2 of Example 11.2 but starting respectively from 2-chloropyrimidine-4-carbonitrile.

### Example 11.9: Synthesis of Compounds (29), (30) and (31)

STEP 1: Synthesis of trans-3-[(3S)-2-[4-(aminomethyl)cyclohexanecarbonyl]isoxazolidin-3-yl]-5-fluoro-benzonitrile; hydrochloride is performed as in STEP 1 of Example 11.1—compound responding to formula (lie) of scheme 5, but starting from tert-butyl N-[trans-4-[(3S)-3-(3-cyano-5-fluorophenyl)isoxazolidine-2-carbonyl]methylcyclhexyl]carbamate

STEP 2a: Synthesis of trans-6-(((4-((S)-3-(3-cyano-5-fluorophenyl)isoxazolidine-2-carbonyl)cyclohexyl)methyl)amino)pyrimidine-4-carboxamide (Compound (29) is performed in a similar way as for STEP 2 of Example 11.2 but starting respectively from 6-chloropyrimidine-4-carboxamide.

STEP 2b: Synthesis of trans-6-(((4-((S)-3-(3-cyano-5-fluorophenyl)isoxazolidine-2-carbonyl)cyclohexyl)methyl)amino)pyrimidine-4-carbonitrile (Compound (30)) is performed in a similar way as for STEP 2 of Example 11.2 but starting respectively from 6-chloropyrimidine-4-carbonitrile.

STEP 2c: Synthesis of trans-3-fluoro-5-((S)-2-(4-(((7-methyl-7H-pyrrolo[2,3-d]pyrimidin-4-yl)amino)methyl)cyclohexane-1-carbonyl)isoxazolidin-3-yl)benzonitrile compound (31)—route of scheme 5

A suspension of 4-chloro-7-methyl-pyrrolo[2,3-d]pyrimidine (26 mg, 150.7 μmol), Net/Pr₂ (131.5 μl, 753.7 μmol) and 3-((S)-2-(trans-4-(aminomethyl)cyclohexane-1-carbonyl)isoxazolidin-3-yl)-5-fluorobenzonitrile hydrochloride (55.5 mg, 150.7 μmol) in CH₃CN (3 ml) was heated in a micro wave vial to 120° C. and stirred for 1.5 hrs. K₂CO₃ (52.1 mg, 376.9 μmol) was added and the reaction mixture was stirred again at 120° C. for 10 hrs. Volatile components were removed under reduced pressure, the obtained residue dissolved in DMF, filtered and subjected to preparative reversed phase HPLC (12 mg, 17% yield).

### Example 11.10: Synthesis of Compounds (25), (26), (27), (36) and (37)

### Synthesis of cis-3-((3-((S)-3-(4-chlorophenyl)isoxazolidine-2-carbonyl)cyclobutyl)amino)-5-fluorobenzonitrile (Compound (25))

To a stirred suspension of ((cis-3-aminocyclobutyl)((S)-3-(4-chlorophenyl) isoxazolidin-2-yl)methanone hydrochloride (50 mg, 158 μmol) and 3,5-difluorobenzonitrile (44 mg, 315 μmol) in DMSO (1 ml) K₂CO₃ (44 mg, 315 μmol) was added and the suspension was heated at 80° C. for 16 hrs. Water was added at rt and the aqueous layer was extracted with EA, the combined organic layers were washed with water, dried over Na₂SO₄, filtered and concentrated to afford the crude title compound, which was subjected to preparative reversed phase HPLC (4 mg, 9 μmol, 6% yield).

Synthesis of cis-3-fluoro-5-((3-((S)-3-(3-fluorophenyl)isoxazolidine-2-carbonyl)cyclobutyl)amino)benzonitrile (Compound (26)) and cis-3-((3-((S)-3-(3,4-difluorophenyl)isoxazolidine-2-carbonyl)cyclobutyl)amino)-5-fluorobenzonitrile (Compound (27)) are performed in a similar way as in Example 11.10 but starting respectively from ((cis-3-aminocyclobutyl)((S)-3-(2-fluorophenyl) isoxazolidin-2-yl)methanone and ((cis-3-aminocyclobutyl)((S)-3-(2,4-difluorophenyl) isoxazolidin-2-yl)methanone.

Synthesis of cis-3-fluoro-5-((3-((S)-3-(5-fluoropyridin-3-yl)isoxazolidine-2-carbonyl)cyclobutyl)amino)benzonitrile (Compound (36)) is performed in a similar way as in in Example 11.10 but starting from ((cis-3-aminocyclobutyl)((S)-3-(5-fluoropyridin-3-yl) isoxazolidin-2-yl)methanone.

Synthesis of cis-3-((S)-2-(3-((3-cyano-5-fluorophenyl)amino)cyclobutane-1-carbonyl)isoxazolidin-3-yl)-5-fluorobenzonitrile (Compound (37)) is performed in a similar way as in Example 11.10 but starting from cis-3-((S)-2-((3-amino)cyclobutane-1-carbonyl)isoxazolidin-3-yl)-5-fluorobenzonitrile.

### Example 12: Synthesis of Compounds (45), (46), (47), (54), (55), (56)

### Example 12.1: Synthesis of Compounds (43), (45), (46), (47), (48), (54), (55), (56), (57), (58), (59), (60), (62), (63), (64), (66), (67) and (68)

STEP 1: Synthesis of [(3S)-3-(3,5-difluorophenyl)isoxazolidin-2-yl]-[trans-4-(hydroxymethyl)cyclohexyl]methanone

To a stirred solution of trans-4-(hydroxymethyl)cyclohexane-1-carboxylic acid (1.1 g, 6.95 mmol), Net/Pr₂ (3.64 ml, 20.9 mmol) and (S)-3-(3,5-difluorophenyl)isoxazolidine hydrochloride (2.00 g, 9.04 mmol) in DMF (26 ml) HATU (3.17 g, 8.34 mmol) was added at rt. Stirring at rt was continued for 2 hrs. The reaction mixture was diluted with water and the aqueous layer extracted with EA. The combined organic layers were washed with 0.1 N aqueous NaOH and 0.1 N aqueous HCl, dried over Na₂SO₄, filtered and concentrated to afford the crude title compound, which was purified by column chromatography (SiO₂; EA/heptane gradient) (1.75 g, 5.38 mmol, 77% yield).

STEP 2a: Synthesis of trans-6-((4-((S)-3-(3,5-difluorophenyl)isoxazolidine-2-carbonyl)cyclohexyl)methoxy)pyrimidine-4-carbonitrile (Compounds (45) and trans-(4-(((6-chloropyrimidin-4-yl)oxy)methyl)cyclohexyl)((S)-3-(3,5-difluorophenyl)isoxazolidin-2-yl)methanone (46)—route SM4 of scheme 4

To a stirred solution of 6-chloropyrimidine-4-carbonitrile (44 mg, 307 μmol) and [(3S)-3-(3,5-difluorophenyl)isoxazolidin-2-yl]-[trans-4-(hydroxymethyl)cyclohexyl]methanone (100 mg, 307 μmol) in THF (1.2 ml) KotBu was added (41 mg, 368 μmol) at 0° C. The solution was stirred at 0° C. for 1.5 hrs. Water was added at 0° C. and the aqueous layer was extracted with EA. The combined organic layers were dried over Na₂SO₄, filtered and concentrated to afford the crude title compounds, which were subjected to preparative reversed phase HPLC.

STEP 2b: trans-6-((4-((S)-3-(3,5-difluorophenyl)isoxazolidine-2-carbonyl)cyclohexyl)methoxy)pyrimidine-4-carboxamide (Compound (47)), cis-6-((4-((S)-3-(3,5-difluorophenyl)isoxazolidine-2-carbonyl)cyclohexyl)methoxy)pyrimidine-4-carboxamide (Compound (54)), cis-6-((4-((S)-3-(3,5-difluorophenyl)isoxazolidine-2-carbonyl)cyclohexyl)methoxy)pyrimidine-4-carbonitrile (Compound (55)) and cis-(4-(((6-chloropyrimidin-4-yl)oxy)methyl)cyclohexyl)((S)-3-(3,5-difluorophenyl)isoxazolidin-2-yl)methanone (Compound (56)) are synthesized similarly as detailed in STEP 2a of Example 12.1 for compounds (45) and (46), but starting from 6-chloro-pyrimidine-4-carboxamide (47), (54), 6-chloro-pyrimidine-4-carbonitrile (55) and 4,6-dichloropyrimidine (56)

STEP 2c: Synthesis of trans-5-((4-((S)-3-(3,5-difluorophenyl)isoxazolidine-2-carbonyl)cyclohexyl)methoxy)-2-fluorobenzonitrile (Compound (43)), trans-3-((4-((S)-3-(3,5-difluorophenyl)isoxazolidine-2-carbonyl)cyclohexyl)methoxy)-5-fluorobenzonitrile (Compound (48)), trans-3-(4-chloro-3-((4-((S)-3-(3,5-difluorophenyl)isoxazolidine-2-carbonyl)cyclohexyl)methoxy)phenyl)-5,5-dimethyloxazolidine-2,4-dione (Compound (57)), trans-1-(4-chloro-3-((4-((S)-3-(3,5-difluorophenyl)isoxazolidine-2-carbonyl)cyclohexyl)methoxy)phenyl)pyrrolidine-2,5-dione (Compound (58)), trans-3-(3-((4-((S)-3-(3,5-difluorophenyl)isoxazolidine-2-carbonyl)cyclohexyl)methoxy)-4-fluorophenyl)-5,5-dimethyloxazolidine-2,4-dione (Compound (59)), trans-1-(4-chloro-3-((4-((S)-3-(3,5-difluorophenyl)isoxazolidine-2-carbonyl)cyclohexyl)methoxy)phenyl)pyrrolidin-2-one (Compound (60)), trans-((S)-3-(3,5-difluorophenyl)isoxazolidin-2-yl)(4-((4-(3,5-dimethyl-1H-pyrazol-1-yl)-2-fluorophenoxy)methyl)cyclohexyl)methanone (Compound (62)), trans-3-(4-chloro-3-((4-((S)-3-(3,5-difluorophenyl)isoxazolidine-2-carbonyl)cyclohexyl)methoxy)phenyl)-1-methylimidazolidine-2,4-dione (Compound (63)), trans-((S)-3-(3,5-difluorophenyl)isoxazolidin-2-yl)(4-((5-(3,5-dimethyl-4H-1,2,4-triazol-4-yl)-2-fluorophenoxy)methyl)cyclohexyl)methanone (Compound (64)), trans-3-((4-((S)-3-(3,5-difluorophenyl)isoxazolidine-2-carbonyl)cyclohexyl)methoxy)benzenesulfonamide (Compound (66)), trans-((S)-3-(3,5-difluorophenyl)isoxazolidin-2-yl)(4-((4-(3,5-dimethyl-4H-1,2,4-triazol-4-yl)-2-fluorophenoxy)methyl)cyclohexyl)methanone (Compound (67)) and trans-1-(4-((4-((S)-3-(3,5-difluorophenyl)isoxazolidine-2-carbonyl)cyclohexyl)methoxy)-3-fluorophenyl)-3-methyl-1,3-dihydro-2H-imidazol-2-one (Compound (68)) is performed in a similar way as detailed in STEP 2a of Example 12.1. but starting from 5-chloro-2-fluorobenzonitrile (43), 3-chloro-5-fluorobenzonitrile (48), (3,4-dichlorophenyl)-5,5-dimethyloxazolidine-2,4-dione (57), 1-(3,4-dichlorophenyl)pyrrolidine-2,5-dione (58), 3-(3-chloro-4-fluorophenyl)-5,5-dimethyloxazolidine-2,4-dione (59), 1-(3,4-dichlorophenyl)pyrrolidin-2-one (60), 4-(3,5-dimethyl-1H-pyrazol-1-yl)-2-fluoro-chlorophenyl (62), (4-((5-(3,5-dimethyl-4H-1,2,4-triazol-4-yl)-2-fluoro-chlorophenyl (64), 3-chlorobenzenesulfonamide (66), 4-(3,5-dimethyl-4H-1,2,4-triazol-4-yl)-2-fluoro-chlorophenyl (67) and 1-(4-chloro-3-fluorophenyl)-3-methyl-1,3-dihydro-2H-imidazol-2-one (68) respectively

### Example 12.2: Synthesis of Compounds (61), (65)

STEP 1: Synthesis of [(3S)-3-(3,5-difluorophenyl)isoxazolidin-2-yl]-(cis-3-hydroxycyclobutyl)methanone is performed in a similar way as detailed in STEP 1 of Example 12.1, but starting from (3S)-3-(3,5-difluorophenyl)isoxazolidine

STEP 2a: Synthesis of cis-3-(3-((S)-3-(3,5-difluorophenyl)isoxazolidine-2-carbonyl)cyclobutoxy)-5-fluorobenzonitrile (Compound (61))—route SM3 of scheme 4

To a stirred solution of [(3S)-trans-3-(3,5-difluorophenyl)isoxazolidin-2-yl]-(3-hydroxycyclobutyl)methanone (50 mg, 177 μmol), PPh₃ (56 mg, 212 μmol) and 3-fluoro-5-hydroxybenzonitrile (24.2 mg, 177 μmol) in THF (1 ml) at rt a 1 M solution of DIAD (212 μl, 212 μmol) in THF was added and stirring at rt was continued for 2 hrs. Additional 0.6 eq. Of a 1 M DIAD solution in THF were added followed by 1.5 hrs of stirring at rt. Volatile components were removed under reduced pressure, the obtained residue dissolved in DMF, filtered and subjected to preparative reversed phase HPLC to give 41 mg (102 μmol, 58% yield) of the title compound.

STEP 2b: Synthesis of cis-5-(3-((S)-3-(3,5-difluorophenyl)isoxazolidine-2-carbonyl)cyclobutoxy)-2-fluorobenzonitrile (Compound (65)) is performed in a similar way as detailed in STEP 2a of Example 12.2 but starting from 2-fluoro-5-hydroxybenzonitrile.

### Example 12.3: Synthesis of Compounds (44), (49), (76), (77), (78), (79), (80), (91)

STEP 1: Synthesis of 3-fluoro-5-[(3S)-2-[trans-4-(hydroxymethyl)cyclohexanecarbonyl]isoxazolidin-3-yl]benzonitrile is performed in a similar way as detailed in STEP 1 of Example 12.1, but starting from (3S)-3-(3-cyano-5-fluorophenyl)isoxazolidine

STEP 2: Synthesis of trans-5-((4-((S)-3-(3-cyano-5-fluorophenyl)isoxazolidine-2-carbonyl)cyclohexyl)methoxy)-2-fluorobenzonitrile (Compound (44)), trans-3-((S)-2-(4-((3-cyano-5-fluorophenoxy)methyl)cyclohexane-1-carbonyl)isoxazolidin-3-yl)-5-fluorobenzonitrile (Compound (49)), trans-3-((S)-2-(4-((4-(3,5-dimethyl-1H-pyrazol-1-yl)-2-fluorophenoxy)methyl)cyclohexane-1-carbonyl)isoxazolidin-3-yl)-5-fluorobenzonitrile (Compound (76)), trans-3-((4-((S)-3-(3-cyano-5-fluorophenyl)isoxazolidine-2-carbonyl)cyclohexyl)methoxy)benzenesulfonamide (Compound (77)), trans-3-fluoro-5-((S)-2-(4-((2-fluoro-4-(3-methyl-2-oxo-2,3-dihydro-1H-imidazol-1-yl)phenoxy)methyl)cyclohexane-1-carbonyl)isoxazolidin-3-yl)benzonitrile (Compound (78)), trans-3-((S)-2-(4-((5-(5,5-dimethyl-2,4-dioxooxazolidin-3-yl)-2-fluorophenoxy)methyl)cyclohexane-1-carbonyl)isoxazolidin-3-yl)-5-fluorobenzonitrile (Compound (79)), trans-3-((S)-2-(4-((2-chloro-5-(3-methyl-2,5-dioxoimidazolidin-1-yl)phenoxy)methyl)cyclohexane-1-carbonyl)isoxazolidin-3-yl)-5-fluorobenzonitrile (Compound (80)) and trans-2-[[4-[(3S)-3-(3-cyano-5-fluoro-phenyl)isoxazolidine-2-carbonyl]cyclohexyl]methoxy]pyridine-4-carbonitrile (Compound (91)) is performed in a similar way as detailed in STEP 2a of Example 12.2. starting from the 5-chloro-2-fluorobenzonitrile (44), 3-chloro-5-fluorobenzonitrile ((49), (50), (76)), 3-chloro-benzenesulfonamide (77), 2-fluoro-4-(3-methyl-2-oxo-2,3-dihydro-1H-imidazol-1-yl)chlorophenyl (78), 4-(5,5-dimethyl-2,4-dioxooxazolidin-3-yl)-2-fluorochlorophenyl (79), -1,2-dichloro-5-(3-methyl-2,5-dioxoimidazolidin-1-yl)phenyl (80), 2-chloropyridine-4-carbonitrile (91)

### Example 12.4: Synthesis of Compounds (50), (51)

STEP 1: Synthesis of [(3S)-3-(3,5-difluorophenyl)isoxazolidin-2-yl]-(trans-3-hydroxy cyclobutyl)methanone is performed in a similar way as detailed in STEP 1 of Example 12.1, but starting from (3S)-3-(3,5-difluorophenyl)isoxazolidine

STEP 2: Synthesis of trans-3-(3-((S)-3-(3,5-difluorophenyl)isoxazolidine-2-carbonyl)cyclobutoxy)-5-fluorobenzonitrile, (Compound (50) and trans-5-(3-((S)-3-(3,5-difluorophenyl)isoxazolidine-2-carbonyl)cyclobutoxy)-2-fluorobenzonitrile (Compound (51)) is performed in a similar way as detailed in STEP 2a of Example 12.2, starting from 3-chloro-5-fluorobenzonitrile and 5-chloro-2-fluorobenzonitrile respectively

### Example 12.5: Synthesis of Compounds (52), (53)

STEP 1: Synthesis of [(3S)-3-(3,5-difluorophenyl)isoxazolidin-2-yl]-[cis-4-(hydroxymethyl)cyclohexyl]methanone is performed in a similar way as detailed in STEP 1 of Example 12.1, but starting from (3S)-3-(3,5-difluorophenyl)isoxazolidine

STEP 2: Synthesis of cis-3-((4-((S)-3-(3,5-difluorophenyl)isoxazolidine-2-carbonyl)cyclohexyl)methoxy)-5-fluorobenzonitrile (Compound (52) and cis-3-((4-((S)-3-(3,5-difluorophenyl)isoxazolidine-2-carbonyl)cyclohexyl)methoxy)-5-fluorobenzonitrile (Compound (53)) is performed in a similar way as detailed in STEP 2a of Example 12.2. but starting from 5-chloro-2-fluorobenzonitrile and 3-chloro-5-fluorobenzonitrile respectively

### Example 12.6: Synthesis of Compounds (81) and (82)

STEP 1: Synthesis of 3-fluoro-5-[(3S)-2-[cis-4-(hydroxy)cyclohexanecarbonyl]isoxazolidin-3-yl]benzonitrile is performed in a similar way as detailed in STEP 1 of Example 12.1, but starting from (S)-3-(3-cyano-5-fluorophenyl)isoxazolidine

STEP 2: Synthesis of cis-3-((S)-2-(4-(5-(5,5-dimethyl-2,4-dioxooxazolidin-3-yl)-2-fluorophenoxy)cyclohexane-1-carbonyl)isoxazolidin-3-yl)-5-fluorobenzonitrile (Compound (81)) and cis-5-((4-((S)-3-(3-cyano-5-fluorophenyl)isoxazolidine-2-carbonyl)cyclohexyl)oxy)-2-fluorobenzonitrile (Compound (82)) is performed in a similar way as detailed in STEP 2a of Example 12.2. but starting from 5-(5,5-dimethyl-2,4-dioxooxazolidin-3-yl)-2-fluoro-chlorophenyl (81) and 5-chloro-2-fluorobenzonitrile

### Example 13: Biological Activity

### Evaluation of Receptor-Interacting Protein Kinase 1 Inhibition.

The catalytic activity of RIPK1 was measured by monitoring the conversion of Adenosine triphosphate (ATP) to Adenosine diphosphate (ADP) due to autophosphorylation using an ADP-Glo kinase kit (Promega, catalog no. V9104).

In detail, 2 μl recombinantly produced hRIPK1 (aa 1-375) fusion protein (end concentration 3.6 μg/ml) and 2 μl compound (end concentration 33300-1.69 nM; DMSO end concentration 1%) were incubated for 30 minutes at room temperature and then 2 μl ATP (ADP Glo kit, end concentration 50 μM) were added. After another 240 minutes incubation at room temperature, 5 μl Promega ADP-Glo reagent I was added to quench the reaction and deplete unconsumed ATP. After an incubation period of 30 minutes, 10 μl Promega ADP-Glo detection reagent II was added resulting in conversion of ADP to ATP, which generates a light-reaction between luciferase and luciferin. Luminescence was quantified after 30 minutes with a Pherastar FS (BMG LABTECH, Ortenberg).

For the dose response experiments an IC₅₀ value with 95% confidence interval was calculated using the 4-parameter logistic model according to Ratkowsky and Reedy with constraints for lower and upper asymptotes at 0% and 100%. The adjustment was obtained by nonlinear regression using the Levenberg Marquardt algorithm.

### Cellular Assay in U937 Cells to Measure the Activity of RIPK1-Inhibitors on Cell Death (Necroptosis).

Upon TNF-Receptor I ligation, Ser/Thr kinase RIPK1 is recruited to a transient receptor complex I. Upon modification of RIPK1 which promotes activation of RIPK1, complex lib can form, that involves recruitment of RIPK3 and MLKL (mixed lineage-kinase domain-like protein) which then translocates from the cytosol to the plasma membrane to execute cell death (Cai, Z. et al, Nat. Cell Biol. (2014)16:55-65).

Cell death was quantified in 96 well plates by determination of the amount of live cell using a CellTiter 96 Aqueous reagent (Promega), a calorimetric method to measure the number of live cells by reducing tetrazolium compound [3-(4,5-dimethylthiazol-2-yl)-5-(3-carboxymethoxyphenyl)-2-(4-sulfophenyl)-2H-tetrazolium, inner salt; MTS] into formazan. Absorbance of formazan was read at 490 nm. The inhibitory activity of the test compound was quantified in a concentration response curve (CRC) experiment.

Compounds were obtained as 10 mM stock solutions and were diluted 1 to 10 volumes with DMSO to yield a 1 mM solution. From this solution 2 μl were diluted with 998 μl growth medium. 100 μl of 2 μM compound solution was further diluted sequentially with a dilution factor of 2.5 by adding 150 μl growth medium. A total of 10 concentrations were tested ranging from 10 μM to 0.26 nM or from 1 μM to 0.07 nM.

U937 cells were cultured in RPM11640 Glutamax and 10% heat inactivated FBS. 50 μl cell suspension containing 1×106 cells/ml supplemented with 50 μM zVAD.fmk (Benzyloxycarbonyl-Val-Ala-Asp (Ome) fluoromethylketone) and 100 ng/ml recombinant human TNFα were dispensed in each well of a 96-well plate. 50 μl of compound dilutions (described in Cellular assay in U937 cells) were added and the cell suspension incubated overnight (18 to 24 hrs) at 37° C., 5% CO₂ in a humidified atmosphere (95% rH). High (no compound) and low control (no TNFα, zVAD.fmk) were tested with 7 replicates; all compound concentrations were tested in duplicates on each experimental plate.

CellTiter 96 Aqueous reagent was mixed (100 μl PMS (phenazine methosulfate) solution/2 ml MTS (3-(4,5-dimethyldiazol-2-yl)-5-(3-carboxymethoxyphenyl)-2-(4-sulfophenyl)-2H-tetrazolium, inner salt) solution) and 20 μl were added per well. After 4 hrs incubation at 37° C. (5% CO₂ 95% rH) optical density was measured at 490 nm on a microplate reader (Tecan Infinite M1000).

The % inhibition is expressed as percentage of the maximal inhibition value obtained in the absence of TNFα/zVAD.fmc. For each dose response experiment an IC₅₀ value with 95% confidence interval was calculated using the 4-parameter logistic model according to Ratkowsky and Reedy without constraints using an internal application (Biost@t-Speed LTS V2.3).

Results of biological activity are shown in Table 2 (ADP-Glo IC₅₀ (μM) and U937 IC₅₀ (μM)).

TABLE 2
activity data of all examples

| Cpd | ADP Glo assay | U937 cell assay |
|---|---|---|
| No° | IC₅₀ [nM] | IC₅₀ [nM] |
| 1 | 19 | 34 |
| 2 | 22 | 101 |
| 3 | 47 | 536 |
| 4 | 90 | 360 |
| 5 | 38 | 75 |
| 6 | 21 | 6 |
| 7 | 23 | 336 |
| 8 | 25 | 196 |
| 9 | 366 | 757 |
| 10 | 62 | 364 |
| 11 | 62 | 440 |
| 12 | 35 | 560 |
| 13 | 114 | 462 |
| 14 | 33 | 503 |
| 15 | 9 | 192 |
| 16 | 24 | 123 |
| 17 | 56 | 194 |
| 18 | 41 | 162 |
| 19 | 66 | 757 |
| 20 | 69 | 809 |
| 21 | 29 | 212 |
| 22 | 40 | 313 |
| 23 | 44 | 234 |
| 24 | 13 | 264 |
| 25 | 64 | 420 |
| 26 | 52 | 152 |
| 27 | 49 | 170 |
| 28 | 162 | nt |
| 29 | 86 | 561 |
| 30 | 59 | 264 |
| 31 | 110 | 373 |
| 32 | 28 | 50 |
| 33 | 22 | 61 |
| 34 | 20 | 11 |
| 35 | 18 | 4 |
| 36 | 42 | 98 |
| 37 | 34 | 45 |
| 38 | 27 | 22 |
| 39 | 111 | 401 |
| 40 | 18 | 262 |
| 41 | 16 | 61 |
| 42 | 156 | 921 |
| 43 | 11 | 2 |
| 44 | 27 | 5 |
| 45 | 44 | 38 |
| 46 | 41 | 14 |
| 47 | 50 | 21 |
| 48 | 44 | 13 |
| 49 | 44 | 18 |
| 50 | 25 | 130 |
| 51 | 21 | 6 |
| 52 | 18 | 6 |
| 53 | 18 | 17 |
| 54 | 80 | 364 |
| 55 | 56 | 1530 |
| 56 | 47 | 125 |
| 57 | 38 | 37 |
| 58 | 44 | 104 |
| 59 | 29 | 30 |
| 60 | 76 | 112 |
| 61 | 54 | 126 |
| 62 | 28 | 34 |
| 63 | 44 | 45 |
| 64 | 56 | 529 |
| 65 | 18 | 49 |
| 66 | 35 | 8 |
| 67 | 46 | 128 |
| 68 | 19 | 8 |
| 69 | 26 | 35 |
| 70 | 39 | 349 |
| 71 | 14 | 6 |
| 72 | 15 | 13 |
| 73 | 29 | 26 |
| 74 | 24 | 82 |
| 75 | 43 | 224 |
| 76 | 47 | 47 |
| 77 | 53 | 48 |
| 78 | 43 | 9 |
| 79 | 20 | 9 |
| 80 | 164 | 123 |
| 81 | 92 | 855 |
| 82 | 44 | 385 |
| 83 | 34 | 23 |
| 84 | 257 | 428 |
| 85 | 28 | 27 |
| 86 | 22 | 10 |
| 87 | 89 | 551 |
| 88 | 47 | 458 |
| 89 | 18 | 13 |
| 90 | 21 | 23 |
| 91 | 14 | 39 |
| 92 | 42 | 229 |
| 93 | 18 | 13 |
| 94 | 58 | 137 |
| 95 | 15 | 35 |
| 96 | 47 | 906 |
| 97 | 38 | 151 |
| 98 | 41 | 359 |
| 99 | 70 | 209 |
| 100 | 42 | 139 |
| 101 | 40 | 140 |
| 102 | 47 | 257 |
| 103 | 57 | 98 |
| 104 | 24 | 30 |
| 105 | 36 | 202 |
| 106 | 77 | 353 |
| 107 | 19 | 214 |
| 108 | 26 | 94 |
| 109 | 55 | 745 |
| 110 | 62 | 97 |
| 111 | 48 | 153 |
| 112 | 41 | 47 |
| 113 | 38 | 97 |
| 114 | 132 | 816 |
| 115 | 53 | 842 |
| 116 | 23 | 377 |
| 117 | 50 | 675 |
| 118 | 20 | 137 |
| 119 | 29 | 388 |
| 120 | 32 | 336 |
| 121 | 30 | 755 |
| 122 | 17 | 30 |
| 123 | 14 | 24 |
| 124 | 21 | 81 |
| 125 | 39 | 347 |
| 126 | 25 | 54 |
| 127 | 17 | 31 |
| 128 | 20 | 691 |
| 129 | 11 | 28 |
| 130 | 37 | 98 |
| 131 | 47 | 236 |

All the compounds according to the disclosure are potent RIPK1 inhibitors as they exhibit a reducing catalytic activity of RIPK1, highlighted by ADP Glo assay, with IC₅₀ lower than 400 nM. Most of the compounds even exhibit IC₅₀ values below 200 nM. Advantageously, most of the compounds exhibit IC₅₀ values below 150 nM, and even lower than 100 nM.

All the compounds according to the disclosure are potent RIPK1 inhibitors as they exhibit a cell death (necroptosis) in U937 cells, with IC₅₀ lower than 1000 nM. Most of the compounds even exhibit IC₅₀ values below 500 nM, and even lower than 200 nM. Advantageously, most of the compounds exhibit IC₅₀ values below 100 nM, and even lower than or equal to 50 nM.

1. A compound of formula (I) 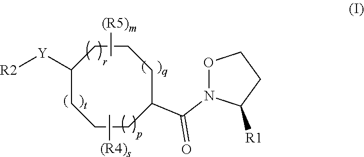
wherein
R1 represents a phenyl or a monocyclic heteroaryl, which is optionally substituted by one, two or three R3;
R2 represents an aryl or a heteroaryl, which is optionally substituted by one, two or three R6;
each R3 is independently chosen from a halogen, a cyano, a (C₁-C₄)alkyl group or a (C₁-C₄)alkoxy group;
R4 and R5 are independently chosen from a halogen, a (C₁-C₄)alkyl group, a (C₁-C₄)alkoxy group, or R4 and R5 form together a (C₁-C₄)alkylene bridge;
m and s are independently 0 or 1;
p, q, r and t are independently 0 or 1;
Y is a bond or a divalent group chosen from —C(O)—NH—, —C(O)—NH—CH₂—, —NH—, —CH₂—NH—, —NH—CH₂—, —O—, CH₂—O— and —O—CH₂—;
each R6 is independently selected from halogen, cyano, —OH, a (C₁-C₄)-alkyl group, —CF₃, —C(O)NH₂, a —C(O)NH—(C₁-C₄)-alkyl group, —C(O)OH, a —C(O)O—(C₁-C₄)-alkyl group, —SO₂NH₂, a (C₁-C₄)-alkoxy group, a —O—(C₁-C₄)alkylene-(C₃-C₆)cycloalkyl group, a —O—(C₃-C₆)cycloalkyl group, a —O—(C₃-C₆)heterocycloalkyl group, a 5- or 6-memembered heterocycloalkyl group, a monocyclic heteroaryl group and oxo, wherein said (C₁-C₄)alkyl group, (C₁-C₄)alkoxy group, —O—(C₃-C₆)cycloalkyl group, (C₁-C₄)alkyl group, 5- or 6-memembered heterocycloalkyl group or monocyclic heteroaryl group is optionally substituted by one, two, three or four R7;
each R7 is independently a halogen, oxo, —OH, a (C₁-C₄)alkyl group or a (C₁-C₄)alkoxy group,
or a pharmaceutically acceptable salt, solvate, or stereoisomer thereof.

2. The compound of formula (I) as defined in claim 1, wherein R1 represents phenyl, thiazolyl, pyridinyl or pyrazinyl, optionally substituted by one or two groups independently selected from a halogen, a cyano, a (C₁-C₂)-alkyl group or a (C₁-C₂)-alkoxy group.

3. The compound of formula (I) as defined in claim 1, wherein R2 represents phenyl, pyrimidinyl, pyridinyl, pyrrolopyrimidinyl, benzimidazolyl, indazolyl or indolyl, optionally substituted by one or two groups independently selected from halogen, cyano, a (C₁-C₂)-alkyl group, —C(O)NH₂, a —C(O)O—(C₁-C₂)-alkyl group, —SO₂NH₂, a 5- or 6-membered heterocycloalkyl group, and a 5-membered heteroaryl, said 5- or 6-membered heterocycloalkyl group and 5-membered heteroaryl being optionally substituted by one, two, three or four groups independently selected from a (C₁-C₂)alkyl group and oxo.

4. The compound of formula (I) as defined in claim 1, wherein Y is a bond or a divalent group chosen from —NH—, —NH—CH₂—, —O— and —O—CH₂—.

5. The compound of formula (I) as defined in claim 1, wherein R4 and R5 are independently chosen from a (C₁-C₂)alkyl group.

6. The compound of formula (I) as defined in claim 1, wherein alternatively:
p, q, r and t are all equal to 0,
r, q and t are all equal to 0 and p is equal to 1,
r and t are both equal to 0 and q and p are both equal to 1, or
q, t and p are all equal to 1 and r is equal to 0.

7. The compound of formula (I) as defined in claim 6, wherein if p, q, r and t are 0, then R4 and R5 do not form together a (C₁-C₄)alkylene bridge.

8. The compound of formula (I) as defined in claim 1, having following formula (Ia): 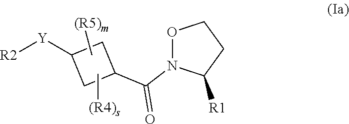
or a pharmaceutically acceptable salt, solvate, or stereoisomer thereof.

9. The compound of formula (I) as defined in claim 1, having following formula (Ib): 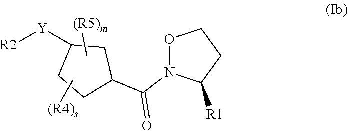
or a pharmaceutically acceptable salt, solvate, or stereoisomer thereof.

10. The compound of formula (I) as defined in claim 1, having following formula (Ic): 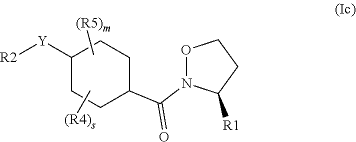
or a pharmaceutically acceptable salt, solvate, or stereoisomer thereof.

11. The compound of formula (I) as defined in claim 1, having following formula (Id): 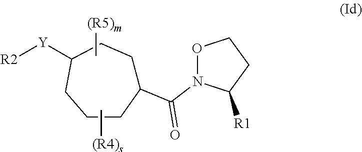
or a pharmaceutically acceptable salt, solvate, or stereoisomer thereof.

12. The compound or formula (I) as defined in claim 1 selected from:
(1) cis-2-((3-((S)-3-(3,5-difluorophenyl)isoxazolidine-2-carbonyl)cyclobutyl)amino)pyrimidine-4-carboxamide,
(2) cis-2-((3-((S)-3-(3,5-difluorophenyl)isoxazolidine-2-carbonyl)cyclobutyl)amino)pyrimidine-4-carbonitrile,
(3) methyl cis-2-((3-((S)-3-(3,5-difluorophenyl)isoxazolidine-2-carbonyl)cyclobutyl)amino)pyrimidine-4-carboxylate,
(4) trans-2-((3-((S)-3-(3,5-difluorophenyl)isoxazolidine-2-carbonyl)cyclobutyl)amino)pyrimidine-4-carboxamide,
(5) cis-2-((3-((S)-3-(3,5-difluorophenyl)isoxazolidine-2-carbonyl)-3-methylcyclobutyl)amino)pyrimidine-4-carbonitrile,
(6) cis-2-((3-((S)-3-(3,5-difluorophenyl)isoxazolidine-2-carbonyl)-3-methylcyclobutyl)amino)pyrimidine-4-carboxamide,
(7) cis-2-((3-((S)-3-(5-cyanopyridin-3-yl)isoxazolidine-2-carbonyl)-3-methylcyclobutyl)amino)pyrimidine-4-carboxamide,
(8) ethyl cis-6-((3-((S)-3-(3,5-difluorophenyl)isoxazolidine-2-carbonyl)-3-methylcyclobutyl)amino)-5-fluoropyrimidine-4-carboxylate,
(9) ethyl cis-6-chloro-5-((3-((S)-3-(3,5-difluorophenyl)isoxazolidine-2-carbonyl)-3-methylcyclobutyl)amino)pyrimidine-4-carboxylate,
(10) cis-6-chloro-5-((3-((S)-3-(3,5-difluorophenyl)isoxazolidine-2-carbonyl)-3-methylcyclobutyl)amino)pyrimidine-4-carboxamide,
(11) cis-6-((3-((S)-3-(3,5-difluorophenyl)isoxazolidine-2-carbonyl)-3-methylcyclobutyl)amino)-5-fluoropyrimidine-4-carboxamide,
(12) cis-2-((4-((S)-3-(4-chlorophenyl)isoxazolidine-2-carbonyl)cyclohexyl)amino)pyrimidine-4-carboxamide,
(13) cis-6-((4-((S)-3-(4-chlorophenyl)isoxazolidine-2-carbonyl)cyclohexyl)amino)pyrimidine-4-carboxamide,
(14) trans-2-(((4-((S)-3-(4-chlorophenyl)isoxazolidine-2-carbonyl)cyclohexyl)methyl)amino)pyrimidine-4-carbonitrile,
(15) trans-6-(((4-((S)-3-(4-chlorophenyl)isoxazolidine-2-carbonyl)cyclohexyl)methyl)amino)pyrimidine-4-carbonitrile,
(16) cis-2-((3-((S)-3-(3-fluorophenyl)isoxazolidine-2-carbonyl)cyclobutyl)amino)pyrimidine-4-carbonitrile,
(17) cis-6-((3-((S)-3-(3-fluorophenyl)isoxazolidine-2-carbonyl)cyclobutyl)amino)pyrimidine-4-carbonitrile,
(18) cis-6-((3-((S)-3-(3,5-difluorophenyl)isoxazolidine-2-carbonyl)cyclobutyl)amino)pyrimidine-4-carbonitrile,
(19) cis-2-((3-((S)-3-(3-cyano-5-fluorophenyl)isoxazolidine-2-carbonyl)cyclobutyl)amino)pyrimidine-4-carbonitrile,
(20) cis-6-((3-((S)-3-(3-cyano-5-fluorophenyl)isoxazolidine-2-carbonyl)cyclobutyl)amino)pyrimidine-4-carbonitrile,
(21) cis-2-((3-((S)-3-(3,4-difluorophenyl)isoxazolidine-2-carbonyl)cyclobutyl)amino)pyrimidine-4-carbonitrile,
(22) cis-6-((3-((S)-3-(3,4-difluorophenyl)isoxazolidine-2-carbonyl)cyclobutyl)amino)pyrimidine-4-carbonitrile,
(23) cis-6-((3-((S)-3-(4-chlorophenyl)isoxazolidine-2-carbonyl)cyclobutyl)amino)pyrimidine-4-carbonitrile,
(24) cis-2-((3-((S)-3-(4-chlorophenyl)isoxazolidine-2-carbonyl)cyclobutyl)amino)pyrimidine-4-carbonitrile,
(25) cis-3-((3-((S)-3-(4-chlorophenyl)isoxazolidine-2-carbonyl)cyclobutyl)amino)-5-fluorobenzonitrile,
(26) cis-3-fluoro-5-((3-((S)-3-(3-fluorophenyl)isoxazolidine-2-carbonyl)cyclobutyl)amino)benzonitrile,
(27) cis-3-((3-((S)-3-(3,4-difluorophenyl)isoxazolidine-2-carbonyl)cyclobutyl)amino)-5-fluorobenzonitrile,
(28) cis-6-((4-((S)-3-(3-cyano-5-fluorophenyl)isoxazolidine-2-carbonyl)cyclohexyl)amino)pyrimidine-4-carbonitrile,
(29) trans-6-(((4-((S)-3-(3-cyano-5-fluorophenyl)isoxazolidine-2-carbonyl)cyclohexyl)methyl)amino)pyrimidine-4-carboxamide,
(30) trans-6-(((4-((S)-3-(3-cyano-5-fluorophenyl)isoxazolidine-2-carbonyl)cyclohexyl)methyl)amino)pyrimidine-4-carbonitrile,
(31) trans-3-fluoro-5-((S)-2-(4-(((7-methyl-7H-pyrrolo[2,3-d]pyrimidin-4-yl)amino)methyl)cyclohexane-1-carbonyl)isoxazolidin-3-yl)benzonitrile,
(32) cis-5-((3-((S)-3-(3-cyano-5-fluorophenyl)isoxazolidine-2-carbonyl)cyclobutyl)amino)-2-fluorobenzonitrile,
(33) cis-2-fluoro-5-((3-((S)-3-(5-fluoropyridin-3-yl)isoxazolidine-2-carbonyl)cyclobutyl)amino)benzonitrile,
(34) trans-2-fluoro-5-(((4-((S)-3-(5-fluoropyridin-3-yl)isoxazolidine-2-carbonyl)cyclohexyl)methyl)amino)benzonitrile,
(35) trans-5-(((4-((S)-3-(3-cyano-5-fluorophenyl)isoxazolidine-2-carbonyl)cyclohexyl)methyl)amino)-2-fluorobenzonitrile,
(36) cis-3-fluoro-5-((3-((S)-3-(5-fluoropyridin-3-yl)isoxazolidine-2-carbonyl)cyclobutyl)amino)benzonitrile,
(37) cis-3-((S)-2-(3-((3-cyano-5-fluorophenyl)amino)cyclobutane-1-carbonyl)isoxazolidin-3-yl)-5-fluorobenzonitrile,
(38) trans-3-((S)-2-(4-(((3-cyano-5-fluorophenyl)amino)methyl)cyclohexane-1-carbonyl)isoxazolidin-3-yl)-5-fluorobenzonitrile,
(39) cis-3-((S)-2-(4-((3-cyano-5-fluorophenyl)amino)cyclohexane-1-carbonyl)isoxazolidin-3-yl)-5-fluorobenzonitrile,
(40) cis-3-((S)-2-(3-(1H-benzo[d]imidazol-1-yl)cyclobutane-1-carbonyl)isoxazolidin-3-yl)-5-fluorobenzonitrile,
(41) cis-(3-(1H-benzo[d]imidazol-1-yl)cyclobutyl)((S)-3-(3,5-difluorophenyl)isoxazolidin-2-yl)methanone,
(42) cis-(3-(1H-benzo[d]imidazol-1-yl)cyclobutyl)((S)-3-(5-fluoropyridin-3-yl)isoxazolidin-2-yl)methanone,
(43) trans-5-((4-((S)-3-(3,5-difluorophenyl)isoxazolidine-2-carbonyl)cyclohexyl)methoxy)-2-fluorobenzonitrile,
(44) trans-5-((4-((S)-3-(3-cyano-5-fluorophenyl)isoxazolidine-2-carbonyl)cyclohexyl)methoxy)-2-fluorobenzonitrile,
(45) trans-6-((4-((S)-3-(3,5-difluorophenyl)isoxazolidine-2-carbonyl)cyclohexyl)methoxy)pyrimidine-4-carbonitrile,
(46) trans-(4-(((6-chloropyrimidin-4-yl)oxy)methyl)cyclohexyl)((S)-3-(3,5-difluorophenyl)isoxazolidin-2-yl)methanone,
(47) trans-6-((4-((S)-3-(3,5-difluorophenyl)isoxazolidine-2-carbonyl)cyclohexyl)methoxy)pyrimidine-4-carboxamide,
(48) trans-3-((4-((S)-3-(3,5-difluorophenyl)isoxazolidine-2-carbonyl)cyclohexyl)methoxy)-5-fluorobenzonitrile,
(49) trans-3-((S)-2-(4-((3-cyano-5-fluorophenoxy)methyl)cyclohexane-1-carbonyl)isoxazolidin-3-yl)-5-fluorobenzonitrile,
(50) trans-3-(3-((S)-3-(3,5-difluorophenyl)isoxazolidine-2-carbonyl)cyclobutoxy)-5-fluorobenzonitrile,
(51) trans-5-(3-((S)-3-(3,5-difluorophenyl)isoxazolidine-2-carbonyl)cyclobutoxy)-2-fluorobenzonitrile,
(52) cis-5-((4-((S)-3-(3,5-difluorophenyl)isoxazolidine-2-carbonyl)cyclohexyl)methoxy)-2-fluorobenzonitrile,
(53) cis-3-((4-((S)-3-(3,5-difluorophenyl)isoxazolidine-2-carbonyl)cyclohexyl)methoxy)-5-fluorobenzonitrile,
(54) cis-6-((4-((S)-3-(3,5-difluorophenyl)isoxazolidine-2-carbonyl)cyclohexyl)methoxy)pyrimidine-4-carboxamide,
(55) cis-6-((4-((S)-3-(3,5-difluorophenyl)isoxazolidine-2-carbonyl)cyclohexyl)methoxy)pyrimidine-4-carbonitrile,
(56) cis-(4-(((6-chloropyrimidin-4-yl)oxy)methyl)cyclohexyl)((S)-3-(3,5-difluorophenyl)isoxazolidin-2-yl)methanone,
(57) trans-3-(4-chloro-3-((4-((S)-3-(3,5-difluorophenyl)isoxazolidine-2-carbonyl)cyclohexyl)methoxy)phenyl)-5,5-dimethyloxazolidine-2,4-dione,
(58) trans-1-(4-chloro-3-((4-((S)-3-(3,5-difluorophenyl)isoxazolidine-2-carbonyl)cyclohexyl)methoxy)phenyl)pyrrolidine-2,5-dione,
(59) trans-3-(3-((4-((S)-3-(3,5-difluorophenyl)isoxazolidine-2-carbonyl)cyclohexyl)methoxy)-4-fluorophenyl)-5,5-dimethyloxazolidine-2,4-dione,
(60) trans-1-(4-chloro-3-((4-((S)-3-(3,5-difluorophenyl)isoxazolidine-2-carbonyl)cyclohexyl)methoxy)phenyl)pyrrolidin-2-one,
(61) cis-3-(3-((S)-3-(3,5-difluorophenyl)isoxazolidine-2-carbonyl)cyclobutoxy)-5-fluorobenzonitrile,
(62) trans-((S)-3-(3,5-difluorophenyl)isoxazolidin-2-yl)(4-((4-(3,5-dimethyl-1H-pyrazol-1-yl)-2-fluorophenoxy)methyl)cyclohexyl)methanone,
(63) trans-3-(4-chloro-3-((4-((S)-3-(3,5-difluorophenyl)isoxazolidine-2-carbonyl)cyclohexyl)methoxy)phenyl)-1-methylimidazolidine-2,4-dione,
(64) trans-((S)-3-(3,5-difluorophenyl)isoxazolidin-2-yl)(4-((5-(3,5-dimethyl-4H-1,2,4-triazol-4-yl)-2-fluorophenoxy)methyl)cyclohexyl)methanone,
(65) cis-5-(3-((S)-3-(3,5-difluorophenyl)isoxazolidine-2-carbonyl)cyclobutoxy)-2-fluorobenzonitrile,
(66) trans-3-((4-((S)-3-(3,5-difluorophenyl)isoxazolidine-2-carbonyl)cyclohexyl)methoxy)benzenesulfonamide,
(67) trans-((S)-3-(3,5-difluorophenyl)isoxazolidin-2-yl)(4-((4-(3,5-dimethyl-4H-1,2,4-triazol-4-yl)-2-fluorophenoxy)methyl)cyclohexyl)methanone,
(68) trans-1-(4-((4-((S)-3-(3,5-difluorophenyl)isoxazolidine-2-carbonyl)cyclohexyl)methoxy)-3-fluorophenyl)-3-methyl-1,3-dihydro-2H-imidazol-2-one,
(69) cis-((S)-3-(3,5-difluorophenyl)isoxazolidin-2-yl)(3-(5-fluoro-1H-benzo[d]imidazol-1-yl)cyclobutyl)methanone,
(70) cis-3-fluoro-5-((S)-2-(3-(5-fluoro-1H-benzo[d]imidazol-1-yl)cyclobutane-1-carbonyl)isoxazolidin-3-yl)benzonitrile,
(71) trans-5-((S)-2-(4-((3-cyanophenoxy)methyl)cyclohexane-1-carbonyl)isoxazolidin-3-yl)nicotinonitrile,
(72) trans-5-((S)-2-(4-((3-cyano-4-fluorophenoxy)methyl)cyclohexane-1-carbonyl)isoxazolidin-3-yl)nicotinonitrile,
(73) trans-3-((4-((S)-3-(5-cyanopyridin-3-yl)isoxazolidine-2-carbonyl)cyclohexyl)methoxy)benzamide,
(74) trans-5-((4-((S)-3-(5-cyanopyridin-3-yl)isoxazolidine-2-carbonyl)cyclohexyl)methoxy)-2-fluorobenzamide,
(75) trans-2-chloro-5-((4-((S)-3-(5-cyanopyridin-3-yl)isoxazolidine-2-carbonyl)cyclohexyl)methoxy)benzamide,
(76) trans-3-((S)-2-(4-((4-(3,5-dimethyl-1H-pyrazol-1-yl)-2-fluorophenoxy)methyl)cyclohexane-1-carbonyl)isoxazolidin-3-yl)-5-fluorobenzonitrile,
(77) trans-3-((4-((S)-3-(3-cyano-5-fluorophenyl)isoxazolidine-2-carbonyl)cyclohexyl)methoxy)benzenesulfonamide,
(78) trans-3-fluoro-5-((S)-2-(4-((2-fluoro-4-(3-methyl-2-oxo-2,3-dihydro-1H-imidazol-1-yl)phenoxy)methyl)cyclohexane-1-carbonyl)isoxazolidin-3-yl)benzonitrile,
(79) trans-3-((S)-2-(4-((5-(5,5-dimethyl-2,4-dioxooxazolidin-3-yl)-2-fluorophenoxy)methyl)cyclohexane-1-carbonyl)isoxazolidin-3-yl)-5-fluorobenzonitrile,
(80) trans-3-((S)-2-(4-((2-chloro-5-(3-methyl-2,5-dioxoimidazolidin-1-yl)phenoxy)methyl)cyclohexane-1-carbonyl)isoxazolidin-3-yl)-5-fluorobenzonitrile,
(81) cis-3-((S)-2-(4-(5-(5,5-dimethyl-2,4-dioxooxazolidin-3-yl)-2-fluorophenoxy)cyclohexane-1-carbonyl)isoxazolidin-3-yl)-5-fluorobenzonitrile,
(82) cis-5-((4-((S)-3-(3-cyano-5-fluorophenyl)isoxazolidine-2-carbonyl)cyclohexyl)oxy)-2-fluorobenzonitrile,
(83) trans-5-((S)-2-(4-((3-cyano-5-fluorophenoxy)methyl)cyclohexane-1-carbonyl)isoxazolidin-3-yl)nicotinonitrile,
(84) trans-(3-(5-fluoro-1H-benzo[d]imidazol-1-yl)cyclobutyl)((S)-3-(3-fluorophenyl)isoxazolidin-2-yl)methanone,
(85) trans-5-((4-((S)-3-(3-cyano-5-fluorophenyl)isoxazolidine-2-carbonyl)cyclohexyl)methoxy)-2-methylbenzamide,
(86) trans-5-((4-((S)-3-(3,5-difluorophenyl)isoxazolidine-2-carbonyl)cyclohexyl)methoxy)-2-methylbenzamide,
(87) cis-((S)-3-(3,5-difluorophenyl)isoxazolidin-2-yl)(3-(6-fluoro-1H-benzo[d]imidazol-1-yl)cyclobutyl)methanone,
(88) trans-5-((S)-2-(4-((2-chloro-5-(3-methyl-2,5-dioxoimidazolidin-1-yl)phenoxy)methyl)cyclohexane-1-carbonyl)isoxazolidin-3-yl)nicotinonitrile,
(89) trans-5-((S)-2-(4-((2-fluoro-4-(3-methyl-2-oxo-2,3-dihydro-1H-imidazol-1-yl)phenoxy)methyl)cyclohexane-1-carbonyl)isoxazolidin-3-yl)nicotinonitrile,
(90) trans-5-((S)-2-(4-((4-(3,5-dimethyl-1H-pyrazol-1-yl)-2-fluorophenoxy)methyl)cyclohexane-1-carbonyl)isoxazolidin-3-yl)nicotinonitrile,
(91) trans-2-[[4-[(3S)-3-(3-cyano-5-fluoro-phenyl)isoxazolidine-2-carbonyl]cyclohexyl]methoxy]pyridine-4-carbonitrile,
(92) trans-5-((S)-2-(4-((5-(5,5-dimethyl-2,4-dioxooxazolidin-3-yl)-2-fluorophenoxy)methyl)cyclohexane-1-carbonyl)isoxazolidin-3-yl)nicotinonitrile,
(93) trans-4-[[4-[(3S)-3-(3-cyano-5-fluoro-phenyl)isoxazolidine-2-carbonyl]cyclohexyl]methoxy]pyridine-2-carbonitrile,
(94) trans-5-((S)-2-(4-(((4-cyanopyridin-2-yl)oxy)methyl)cyclohexane-1-carbonyl)isoxazolidin-3-yl)nicotinonitrile,
(95) trans-5-((S)-2-(4-(((5-cyanopyridin-2-yl)oxy)methyl)cyclohexane-1-carbonyl)isoxazolidin-3-yl)nicotinonitrile,
(96) cis-[(3S)-3-(3,5-difluorophenyl)isoxazolidin-2-yl]-[3-(5-fluoroindazol-1-yl)cyclobutyl]methanone,
(97) trans-[(3S)-3-(3,5-difluorophenyl)isoxazolidin-2-yl]-[3-(5-fluoroindazol-1-yl)cyclobutyl]methanone,
(98) trans-3-fluoro-5-[(3S)-2-[3-(5-fluoroindazol-1-yl)cyclobutanecarbonyl]isoxazolidin-3-yl]benzonitrile,
(99) trans-6-[[4-[(3S)-3-(5-cyano-3-pyridyl)isoxazolidine-2-carbonyl]cyclohexyl]methoxy]pyridine-3-carboxamide,
(100) trans-3-[[4-[(3S)-3-pyrazin-2-ylisoxazolidine-2-carbonyl]cyclohexyl]methoxy]benzamide,
(101) trans-2-fluoro-5-[[4-[(3S)-3-pyrazin-2-ylisoxazolidine-2-carbonyl]cyclohexyl]methoxy]benzamide,
(102) trans-6-[[4-[(3S)-3-pyrazin-2-ylisoxazolidine-2-carbonyl]cyclohexyl]methoxy]pyridine-3-carboxamide,
(103) trans-6-[[4-[(3S)-3-pyrazin-2-ylisoxazolidine-2-carbonyl]cyclohexyl]methoxy]pyridine-3-carbonitrile,
(104) trans-3-fluoro-5-[[4-[(3S)-3-pyrazin-2-ylisoxazolidine-2-carbonyl]cyclohexyl]methoxy]benzonitrile,
(105) trans-3-fluoro-5-[(3S)-2-[3-(5-fluoroindol-1-yl)cyclobutanecarbonyl]isoxazolidin-3-yl]benzonitrile,
(106) trans-[(3S)-3-(3,5-difluorophenyl)isoxazolidin-2-yl]-[3-(5-fluoroindol-1-yl)cyclobutyl]methanone,
(107) cis-3-fluoro-5-[(3S)-2-[3-(5-fluoroindol-1-yl)cyclobutanecarbonyl]isoxazolidin-3-yl]benzonitrile,
(108) cis-[(3S)-3-(3,5-difluorophenyl)isoxazolidin-2-yl]-[3-(5-fluoroindol-1-yl)cyclobutyl]methanone,
(109) cis-3-fluoro-5-[(3S)-2-[3-(5-fluoroindazol-2-yl)cyclobutanecarbonyl]isoxazolidin-3-yl]benzonitrile,
(110) cis-[(3S)-3-(3,5-difluorophenyl)isoxazolidin-2-yl]-[3-(5-fluoroindazol-2-yl)cyclobutyl]methanone,
(111) trans-4-fluoro-3-[[4-[(3S)-3-pyrazin-2-ylisoxazolidine-2-carbonyl]cyclohexyl]methoxy]benzamide,
(112) trans-3-fluoro-5-[[4-[(3S)-3-pyrazin-2-ylisoxazolidine-2-carbonyl]cyclohexyl]methoxy]benzamide,
(113) trans-3-fluoro-4-[[4-[(3S)-3-pyrazin-2-ylisoxazolidine-2-carbonyl]cyclohexyl]methoxy]benzamide,
(114) cis-2-[3-[(3S)-3-(3,5-difluorophenyl)isoxazolidine-2-carbonyl]cyclopentyl]pyridine-4-carboxamide,
(115) cis-6-[[3-[(3S)-3-(3-cyano-5-fluoro-phenyl)isoxazolidine-2-carbonyl]cyclopentyl]methoxy]pyrimidine-4-carboxamide,
(116) trans-6-[[3-[(3S)-3-(3-cyano-5-fluoro-phenyl)isoxazolidine-2-carbonyl]cyclopentyl]methoxy]pyrimidine-4-carboxamide stereoisomer 2,
(117) trans-6-[[3-[(3S)-3-(3-cyano-5-fluoro-phenyl)isoxazolidine-2-carbonyl]cyclopentyl]methoxy]pyrimidine-4-carboxamide stereoisomer 3,
(118) trans-6-[[3-[(3S)-3-(3,5-difluorophenyl)-1,2-oxazolidine-2-carbonyl]cyclopentyl]methoxy]pyrimidine-4-carboxamide stereoisomer 1,
(119) cis-6-[[3-[(3S)-3-(3,5-difluorophenyl)-1,2-oxazolidine-2-carbonyl]cyclopentyl]methoxy]pyrimidine-4-carboxamide stereoisomer 2,
(120) trans-6-[[3-[(3S)-3-(3,5-difluorophenyl)-1,2-oxazolidine-2-carbonyl]cyclopentyl]methoxy]pyrimidine-4-carboxamide stereoisomer 3,
(121) cis-6-[[3-[(3S)-3-(3,5-difluorophenyl)-1,2-oxazolidine-2-carbonyl]cyclopentyl]methoxy]pyrimidine-4-carboxamide stereoisomer 4,
(122) trans-6-[[4-[(3S)-3-(3,5-difluorophenyl)-1,2-oxazolidine-2-carbonyl]cycloheptyl]methoxy]pyrimidine-4-carboxamide stereoisomer 1,
(123) trans-6-[[4-[(3S)-3-(3,5-difluorophenyl)-1,2-oxazolidine-2-carbonyl]cycloheptyl]methoxy]pyrimidine-4-carboxamide stereoisomer 2,
(124) cis-6-[[4-[(3S)-3-(3,5-difluorophenyl)-1,2-oxazolidine-2-carbonyl]cycloheptyl]methoxy]pyrimidine-4-carboxamide stereoisomer 3,
(125) cis-6-[[4-[(3S)-3-(3,5-difluorophenyl)-1,2-oxazolidine-2-carbonyl]cycloheptyl]methoxy]pyrimidine-4-carboxamide stereoisomer 4,
(126) trans-6-[[4-[(3S)-3-(3-cyano-5-fluoro-phenyl)isoxazolidine-2-carbonyl]cycloheptyl]methoxy]pyrimidine-4-carboxamide stereoisomer 1,
(127) trans-6-[[4-[(3S)-3-(3-cyano-5-fluoro-phenyl)isoxazolidine-2-carbonyl]cycloheptyl]methoxy]pyrimidine-4-carboxamide stereoisomer 2,
(128) cis-6-[[4-[(3S)-3-(3-cyano-5-fluoro-phenyl)isoxazolidine-2-carbonyl]cycloheptyl]methoxy]pyrimidine-4-carboxamide,
(129) trans-3-[[4-[(3S)-3-(6-methoxypyrazin-2-yl)isoxazolidine-2-carbonyl]cyclohexyl]methoxy]benzamide,
(130) trans-3-[[4-[(3S)-3-(2-methylthiazol-4-yl)isoxazolidine-2-carbonyl]cyclohexyl]methoxy]benzamide, and
(131) trans-3-[[4-[(3S)-3-(2-pyridyl)isoxazolidine-2-carbonyl]cyclohexyl]methoxy]benzamide,
or a pharmaceutically acceptable salt, solvate, or stereoisomer thereof.

13. A pharmaceutical composition comprising the compound of formula (I) as defined in claim 1 or a pharmaceutically acceptable salt, solvate, or stereoisomer thereof, and at least one pharmaceutically acceptable excipient.

14. (canceled)

15. (canceled)

16. A method of treating or preventing a disease, disorder or condition that is at least partly mediated by receptor-interacting protein kinase 1, the method comprising administering to a subject in need thereof an effective amount of the compound of formula (I) as defined claim 1, or a pharmaceutically acceptable salt, solvate, or stereoisomer thereof.

17. A method of treating or preventing of a disease selected from Parkinson's disease, Alzheimer's disease, amyotrophic lateral sclerosis (ALS), or multiple sclerosis (MS), the method comprising administering to a subject in need thereof an effective amount of the compound of formula (I) as defined in claim 1, or a pharmaceutically acceptable salt, solvate, or stereoisomer thereof.

18. The compound of formula (I) as defined in claim 2, wherein R1 represents (i) a phenyl substituted by one or two groups independently selected from a halogen, a cyano, a (C₁-C₂)-alkyl group or a (C₁-C₂)-alkoxy group; or (ii) a monocyclic heteroaryl selected from a thiazolyl, a pyridinyl and a pyrazinyl, said heteroaryl being optionally substituted by one or two groups independently selected from a halogen, a cyano, a (C₁-C₂)-alkyl group or a (C₁-C₂)-alkoxy group.

19. The compound of formula (Ia) as defined in claim 8, wherein:
Y is —NH—, —O— or a bond;
R1 is phenyl or pyridinyl, which is optionally substituted by one or two R3;
R2 is phenyl, pyrimidinyl, indazolyl, indolyl or benzimidazolyl, which is optionally substituted by one or two R6;
each R3 is independently chosen from a halogen or cyano;
m and s are independently 0 or 1;
each R6 is a halogen, —C(O)NH₂, cyano or a —C(O)O—(C₁-C₂)alkyl, or a pharmaceutically acceptable salt, solvate, or stereoisomer thereof.

20. The compound of formula (Ib) as defined in claim 9, wherein:
Y is a bond or —O—CH₂—;
R1 is phenyl, which is optionally substituted by one or two R3;
R2 is pyridinyl or pyrimidinyl, which is optionally substituted by —C(O)NH₂;
each R3 is independently chosen from a halogen or cyano;
m and s are 0;
or a pharmaceutically acceptable salt, solvate, or stereoisomer thereof.

21. The compound of formula (Ic) as defined in claim 10, wherein:
Y is —NH—, —NH—CH₂—, a bond, —O— or —O—CH₂—;
R1 is phenyl, pyrazinyl, triazolyl or pyridinyl, which is optionally substituted by one or two R3;
R2 is pyridinyl, phenyl, pyrimidinyl, or pyrrolopyrimidinyl, which is optionally substituted by one or two R6;
each R3 is independently chosen from a halogen, —CH₃, —OCH₃ or cyano;
m and s are 0;
each R6 is independently chosen from a halogen, —C(O)NH₂, cyano, a (C₁-C₂)-alkyl group, —SO₂NH₂, a 5-membered heterocycloalkyl selected from imidazolidinyl, dihydroimidazolyl, pyrrolidinyl and oxazolidinyl or a 5-membered heteroaryl selected from pyrazolyl or triazolyl, said heterocycloalkyl and heteroaryl being optionally substituted by one, two, three or four groups independently selected from a (C₁-C₂)alkyl group and oxo,
or a pharmaceutically acceptable salt, solvate, or stereoisomer thereof.

22. The compound of formula (Id) as defined in claim 11, wherein:
Y is a bond or —O—CH₂—;
R1 is phenyl, which is optionally substituted by one or two R3;
R2 is pyridinyl or pyrimidinyl, which is optionally substituted by —C(O)NH₂;
each R3 is independently chosen from a halogen or cyano;
m and s are 0.
